(19) United States
(12) Patent Application Publication
(10) Pub. No.: US 2025/0270737 A1
(43) Pub. Date: Aug. 28, 2025

(54) Antibodies That Bind GLP1R

(71) Applicant: Twist Bioscience Corporation, South San Francisco, CA (US)

(72) Inventors: Ray Tabibiazar, Chattanooga, TN (US); Aaron Sato, Burlingame, CA (US); Pankaj Garg, Burlingame, CA (US); Qiang Liu, Palo Alto, CA (US); Fumiko Axelrod, Palo Alto, CA (US)

(73) Assignee: Twist Bioscience Corporation, South San Francisco, CA (US)

(21) Appl. No.: 19/207,949

(22) Filed: May 14, 2025

Related U.S. Application Data
(63) Continuation of application No. 17/937,377, filed on Sep. 30, 2022, now Pat. No. 12,331,427.
Application No. 17/937,377 is a division of application No. 16/802,423, filed on Feb. 26, 2020, now Pat. No. 11,492,727.
(60) Provisional application No. 62/961,104, filed on Jan. 14, 2020.
(60) Provisional application No. 62/945,049, filed on Dec. 6, 2019.
(60) Provisional application No. 62/904,563, filed on Sep. 23, 2019.
(60) Provisional application No. 62/855,836, filed on May 31, 2019.
(60) Provisional application No. 62/830,316, filed on Apr. 5, 2019.
(60) Provisional application No. 62/810,377, filed on Feb. 26, 2019.

(51) Int. Cl. C40B 40/08; C07K 16/28

(52) U.S. Cl. CPC C40B 40/08; C07K 16/2869; C07K 2317/24; C07K 2317/31; C07K 2317/54; C07K 2317/55; C07K 2317/565; C07K 2317/622; C07K 2317/624; C07K 2317/92

(57) ABSTRACT

Provided herein are methods and compositions relating to glucagon-like peptide-1 receptor (GLP1R) libraries having nucleic acids encoding for a scaffold comprising a GLP1R binding domain. Libraries described herein include variegated libraries comprising nucleic acids each encoding for a predetermined variant of at least one predetermined reference nucleic acid sequence. Further described herein are protein libraries generated when the nucleic acid libraries are translated. Further described herein are cell libraries expressing variegated nucleic acid libraries described herein.

### CROSS-REFERENCE

This application is a Divisional Application of U.S. patent application Ser. No. 16/802,423, filed Feb. 26, 2020, which claims the benefit of U.S. Provisional Patent Application No. 62/810,377 filed on Feb. 26, 2019: U.S. Provisional Patent Application No. 62/830,316 filed on Apr. 5, 2019: U.S. Provisional Patent Application No. 62/855,836 filed on May 31, 2019: U.S. Provisional Patent Application No. 62/904,563 filed on Sep. 23, 2019; U.S. Provisional Patent Application No. 62/945,049 filed on Dec. 6, 2019; and U.S. Provisional Patent Application No. 62/961,104 filed on Jan. 14, 2020, each of which is incorporated by reference in its entirety.

### SEQUENCE LISTING

The instant application contains a Sequence Listing which has been submitted electronically in XML format and is hereby incorporated by reference in its entirety. Said XML copy, created on Sep. 28, 2022, is named 44854-787_401_SL.xml and is 3,612,000 bytes in size.

### BACKGROUND

G protein-coupled receptors (GPCRs) are implicated in a wide variety of diseases. Raising antibodies to GPCRs has been difficult due to problems in obtaining suitable antigen because GPCRs are often expressed at low levels in cells and are very unstable when purified. Thus, there is a need for improved agents for therapeutic intervention which target GPCRs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are antibodies or antibody fragments thereof that binds GLP1R, comprising an immunoglobulin heavy chain and an immunoglobulin light chain: (a) wherein the immunoglobulin heavy chain comprises an amino acid sequence at least about 90%, 95%, 97%, 99%, or 100% identical to that set forth in SEQ ID NO: 2303, 2304, 2305, 2306, 2307, 2308, 2309, 2317, 2318, 2319, 2320, or 2321; and (b) wherein the immunoglobulin light chain comprises an amino acid sequence at least about 90%, 95%, 97%, 99%, or 100% identical to that set forth in SEQ ID NO: 2310, 2311, 2312, 2313, 2314, 2315, or 2316. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2303; and wherein the immunoglobulin light chain comprises an amino acid sequence at least about 90%, 95%, 97%, 99%, or 100% identical to that set forth in SEQ ID NO: 2310. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2304; and wherein the immunoglobulin light chain comprises an amino acid sequence at least about 90%, 95%, 97%, 99%, or 100% identical to that set forth in SEQ ID NO: 2311. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2305; and wherein the immunoglobulin light chain comprises an amino acid sequence at least about 90%, 95%, 97%, 99%, or 100% identical to that set forth in SEQ ID NO: 2312. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2306; and wherein the immunoglobulin light chain comprises an amino acid sequence at least about 90%, 95%, 97%, 99%, or 100% identical to that set forth in SEQ ID NO: 2313. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2307; and wherein the immunoglobulin light chain comprises an amino acid sequence at least about 90%, 95%, 97%, 99%, or 100% identical to that set forth in SEQ ID NO: 2314. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2308; and wherein the immunoglobulin light chain comprises an amino acid sequence at least about 90%, 95%, 97%, 99%, or 100% identical to that set forth in SEQ ID NO: 2315. Further provided herein are antibodies or antibody fragments thereof that binds GLP1R, wherein the immunoglobulin heavy chain comprises an amino acid sequence at least about 90%, 95%, 97%, 99%, or 100% identical to that set forth in SEQ ID NO: 2309, 2317, 2318, 2319; and wherein the immunoglobulin light chain comprises an amino acid sequence at least about 90%, 95%, 97%, 99%, or 100% identical to that set forth in SEQ ID NO: 2316. Further provided herein are antibodies or antibody fragments thereof that binds GLP1R, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments thereof that binds GLP1R, wherein the antibody or antibody fragment thereof is chimeric or humanized. Further provided herein are antibodies or antibody fragments thereof that binds GLP1R, wherein the antibody has an EC50 less than about 25 nanomolar in a CAMP assay. Further provided herein are antibodies or antibody fragments thereof that binds GLP1R, wherein the antibody has an EC50 less than about 20 nanomolar in a cAMP assay. Further provided herein are antibodies or antibody fragments thereof that binds GLP1R, wherein the antibody has an EC50 less than about 10 nanomolar in a cAMP assay. Further provided herein are antibodies or antibody fragments thereof that binds GLP1R, wherein the antibody is an agonist of GLP1R, Further provided herein are antibodies or antibody fragments thereof that binds GLP1R, wherein the antibody is an antagonist of GLP1R, Further provided herein are antibodies or antibody fragments thereof that binds GLP1R, wherein the antibody is an allosteric modulator of GLP1R, Further provided herein are antibodies or antibody fragments thereof that binds GLP1R, wherein the allosteric modulator of GLP1R is a negative allosteric modulator. Further provided herein are antibodies or antibody fragments thereof that binds GLP1R, wherein the antibody or antibody fragment comprises a CDR-H3 comprising a sequence of any one of SEQ ID NOS: 2277, 2278, 2281, 2282, 2283, 2284, 2285, 2286, 2289, 2290, 2291, 2292, 2294, 2295, 2296, 2297, 2298, 2299, 2300, 2301, or 2302.

Provided herein are nucleic acid libraries comprising a plurality of nucleic acids, wherein each nucleic acid encodes for a sequence that when translated encodes for an immunoglobulin scaffold, wherein the immunoglobulin scaffold comprises a CDR-H3 loop that comprises a GLP1R binding domain, and wherein each nucleic acid comprises a sequence encoding for a sequence variant of the GLP1R binding domain. Further provided herein are nucleic acid libraries, wherein a length of the CDR-H3 loop is about 20 to about 80 amino acids. Further provided herein are nucleic acid libraries, wherein a length of the CDR-H3 loop is about 80 to about 230 base pairs. Further provided herein are nucleic acid libraries, wherein the immunoglobulin scaffold further comprises one or more domains selected from variable domain, light chain (VL), variable domain, heavy chain (VH), constant domain, light chain (CL), and constant domain, heavy chain (CH). Further provided herein are nucleic acid libraries, wherein the VH domain is IGHV1-18, IGHV1-69, IGHV1-8 IGHV3-21, IGHV3-23, IGHV3-30/33rn, IGHV3-28, IGHV3-74, IGHV4-39, or IGHV4-59/61. Further provided herein are nucleic acid libraries, wherein the VH domain is IGHV1-69, IGHV3-30, IGHV3-23, IGHV3, IGHV1-46, IGHV3-7. IGHV1, or IGHV1-8. Further provided herein are nucleic acid libraries, wherein the VH domain is IGHV1-69 and IGHV3-30. Further provided herein are nucleic acid libraries, wherein the VL domain is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. Further provided herein are nucleic acid libraries, wherein a length of the VH domain is about 90 to about 100 amino acids. Further provided herein are nucleic acid libraries, wherein a length of the VL domain is about 90 to about 120 amino acids. Further provided herein are nucleic acid libraries, wherein a length of the VH domain is about 280 to about 300 base pairs. Further provided herein are nucleic acid libraries, wherein a length of the VL domain is about 300 to about 350 base pairs. Further provided herein are nucleic acid libraries, wherein the library comprises at least 105 non-identical nucleic acids. Further provided herein are nucleic acid libraries, wherein the immunoglobulin scaffold comprises a single immunoglobulin domain. Further provided herein are nucleic acid libraries, wherein the immunoglobulin scaffold comprises a peptide of at most 100 amino acids.

Provided herein are protein libraries comprising a plurality of proteins, wherein each of the proteins of the plurality of proteins comprise an immunoglobulin scaffold, wherein the immunoglobulin scaffold comprises a CDR-H3 loop that comprises a sequence variant of a GLP1R binding domain. Further provided herein are protein libraries, wherein a length of the CDR-H3 loop is about 20 to about 80 amino acids. Further provided herein are protein libraries, wherein the immunoglobulin scaffold further comprises one or more domains selected from variable domain, light chain (VL), variable domain, heavy chain (VH), constant domain, light chain (CL), and constant domain, heavy chain (CH). Further provided herein are protein libraries, wherein the VH domain is IGHV1-18, IGHV1-69, IGHV1-8 IGHV3-21, IGHV3-23, IGHV3-30/33rn, IGHV3-28. IGHV3-74, IGHV4-39, or IGHV4-59/61. Further provided herein are protein libraries, wherein the VH domain is IGHV1-69, IGHV3-30, IGHV3-23, IGHV3, IGHV1-46, IGHV3-7, IGHV1, or IGHV1-8. Further provided herein are protein libraries, wherein the VH domain is IGHV1-69 and IGHV3-30. Further provided herein are protein libraries, wherein the VL domain is IGKV1-39. IGKV1-9, IGKV2-28, IGKV3-11, IGKV3-15, IGKV3-20, IGKV4-1, IGLV1-51, IGLV2-14. IGLV1-40, or IGLV3-1. Further provided herein are protein libraries, wherein a length of the VH domain is about 90 to about 100 amino acids. Further provided herein are protein libraries, wherein a length of the VL domain is about 90 to about 120 amino acids. Further provided herein are protein libraries, wherein the plurality of proteins are used to generate a peptidomimetic library. Further provided herein are protein libraries, wherein the protein library comprises antibodies.

Provided herein are protein libraries comprising a plurality of proteins, wherein the plurality of proteins comprises sequence encoding for different GPCR binding domains, and wherein the length of each GPCR binding domain is about 20 to about 80 amino acids. Further provided herein are protein libraries, wherein the protein library comprises peptides. Further provided herein are protein libraries, wherein the protein library comprises immunoglobulins. Further provided herein are protein libraries, wherein the protein library comprises antibodies. Further provided herein are protein libraries, wherein the plurality of proteins is used to generate a peptidomimetic library.

Provided herein are vector libraries comprising a nucleic acid library as described herein.

Provided herein are cell libraries comprising a nucleic acid library as described herein.

Provided herein are cell libraries comprising a protein library as described herein.

Provided herein are antibodies, wherein the antibody comprises a CDR-H3 comprising a sequence of any one of SEQ ID NOS: 2277, 2278, 2281, 2282, 2283, 2284, 2285, 2286, 2289, 2290, 2291, 2292, 2294, 2295, 2296, 2297, 2298, 2299, 2300, 2301, or 2302.

Provided herein are antibodies, wherein the antibody comprises a CDR-H3 comprising a sequence of any one of SEQ ID NOS: 2277, 2278, 2281, 2282, 2283, 2284, 2285, 2286, 2289, 2290, 2291, 2292, 2294, 2295, 2296, 2297, 2298, 2299, 2300, 2301, or 2302; and wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

Provided herein are methods of inhibiting GLP1R activity, comprising administering an antibody or antibody fragment as described herein. Further provided herein are methods of inhibiting GLP1R activity, wherein the antibody or antibody fragment is an allosteric modulator. Further provided herein are methods of inhibiting GLP1R activity, wherein the antibody or antibody fragment is a negative allosteric modulator. Further provided herein are methods of treatment of a metabolic disorder, comprising administering to a subject in need thereof an antibody or antibody fragment as described herein. Further provided herein are methods of treatment of a metabolic disorder, wherein the metabolic disorder is Type II diabetes or obesity.

Provided herein are nucleic acid libraries, comprising: a plurality of nucleic acids, wherein each of the nucleic acids encodes for a sequence that when translated encodes for a GLP1R binding immunoglobulin, wherein the GLP1R binding immunoglobulin comprises a variant of a GLP1R binding domain, wherein the GLP1R binding domain is a ligand for the GLP1R, and wherein the nucleic acid library comprises at least 10,000 variant immunoglobulin heavy chains and at least 10,000 variant immunoglobulin light chains. Further provided herein are nucleic acid libraries, wherein the nucleic acid library comprises at least 50,000 variant immunoglobulin heavy chains and at least 50,000 variant immunoglobulin light chains. Further provided herein are nucleic acid libraries, wherein the nucleic acid library comprises at least 100,000 variant immunoglobulin heavy chains and at least 100,000 variant immunoglobulin light chains. Further provided herein are nucleic acid libraries, wherein the nucleic acid library comprises at least 105 non-identical nucleic acids. Further provided herein are nucleic acid libraries, wherein a length of the immunoglobulin heavy chain when translated is about 90 to about 100 amino acids. Further provided herein are nucleic acid libraries, wherein a length of the immunoglobulin heavy chain when translated is about 100 to about 400 amino acids. Further provided herein are nucleic acid libraries, wherein the variant immunoglobulin heavy chain when translated comprises at least 80% sequence identity to SEQ ID NO: 2303, 2304, 2305, 2306, 2307, 2308, 2309, 2317, 2318, 2319, 2320, or 2321. Further provided herein are nucleic acid libraries, wherein the variant immunoglobulin light chain when translated comprises at least 80% sequence identity to SEQ ID NO: 2310, 2311, 2312, 2313, 2314, 2315, or 2316.

Provided herein are nucleic acid libraries comprising: a plurality of nucleic acids, wherein each of the nucleic acids encodes for a sequence that when translated encodes for a GLP1R single domain antibody, wherein each sequence of the plurality of sequences comprises a variant sequence encoding for at least one of a CDR1, CDR2, and CDR3 on a heavy chain: wherein the library comprises at least 30,000 variant sequences; and wherein the antibody or antibody fragments bind to its antigen with a K_{D} of less than 100 nM. Further provided herein are nucleic acid libraries, wherein the nucleic acid library comprises at least 50,000 variant immunoglobulin heavy chains and at least 50,000 variant immunoglobulin light chains. Further provided herein are nucleic acid libraries, wherein the nucleic acid library comprises at least 100,000 variant immunoglobulin heavy chains and at least 100,000 variant immunoglobulin light chains. Further provided herein are nucleic acid libraries, wherein the nucleic acid library comprises at least 105 non-identical nucleic acids. Further provided herein are nucleic acid libraries, wherein a length of the immunoglobulin heavy chain when translated is about 90 to about 100 amino acids. Further provided herein are nucleic acid libraries, wherein a length of the immunoglobulin heavy chain when translated is about 100 to about 400 amino acids. Further provided herein are nucleic acid libraries, wherein the variant immunoglobulin heavy chain when translated comprises at least 80% sequence identity to SEQ ID NO: 2303, 2304, 2305, 2306, 2307, 2308, 2309, 2317, 2318, 2319, 2320, or 2321. Further provided herein are nucleic acid libraries, wherein the variant immunoglobulin light chain when translated comprises at least 80% sequence identity to SEQ ID NO: 2310, 2311, 2312, 2313, 2314, 2315, or 2316.

Provided herein antagonists of GLP1R comprising SEQ ID NO: 2279 or 2320. Further provided herein are antagonists, wherein the antagonist comprises an EC50 of no more than 1.5 nM. Further provided herein are antagonists, wherein the antagonist comprises an EC50 of no more than 1.0 nM. Further provided herein are antagonists, wherein the antagonist comprises an EC50 of no more than 0.5 nM. Further provided herein are antagonists, wherein the antagonist is an antibody or antibody fragment thereof.

Provided herein are nucleic acid libraries, comprising: a plurality of nucleic acids, wherein each of the nucleic acids encodes for a sequence that when translated encodes for a GLP1R binding immunoglobulin, wherein the GLP1R binding immunoglobulin comprises a variant of a GLP1R binding domain, wherein the GLP1R binding domain is a ligand for the GLP1R, and wherein the nucleic acid library comprises at least 10,000 variant immunoglobulin heavy chains and at least 10,000 variant immunoglobulin light chains. Further provided herein are nucleic acid libraries, wherein the nucleic acid library comprises at least 50,000 variant immunoglobulin heavy chains and at least 50,000 variant immunoglobulin light chains. Further provided herein are nucleic acid libraries, wherein the nucleic acid library comprises at least 100,000 variant immunoglobulin heavy chains and at least 100,000 variant immunoglobulin light chains. Further provided herein are nucleic acid libraries, wherein the nucleic acid library comprises at least 105 non-identical nucleic acids. Further provided herein are nucleic acid libraries, wherein a length of the immunoglobulin heavy chain when translated is about 90 to about 100 amino acids. Further provided herein are nucleic acid libraries, wherein a length of the immunoglobulin heavy chain when translated is about 100 to about 400 amino acids. Further provided herein are nucleic acid libraries, wherein the variant immunoglobulin heavy chain when translated comprises at least 90% sequence identity to SEQ ID NO: 2303, 2304, 2305, 2306, 2307, 2308, 2309, 2317, 2318, 2319, 2320, or 2321, Further provided herein are nucleic acid libraries, wherein the variant immunoglobulin light chain when translated comprises at least 90% sequence identity to SEQ ID NO: 2310, 2311, 2312, 2313, 2314, 2315, or 2316.

Provided herein are nucleic acid libraries comprising: a plurality of nucleic acids, wherein each of the nucleic acids encodes for a sequence that when translated encodes for a GLP1R single domain antibody, wherein each sequence of the plurality of sequences comprises a variant sequence encoding for at least one of a CDR1, CDR2, and CDR3 on a heavy chain: wherein the library comprises at least 30,000 variant sequences; and wherein the antibody or antibody fragments bind to its antigen with a K_{D} of less than 100 nM. Further provided herein are nucleic acid libraries, wherein the nucleic acid library comprises at least 50,000 variant immunoglobulin heavy chains and at least 50,000 variant immunoglobulin light chains. Further provided herein are nucleic acid libraries, wherein the nucleic acid library comprises at least 100,000 variant immunoglobulin heavy chains and at least 100,000 variant immunoglobulin light chains. Further provided herein are nucleic acid libraries, wherein the nucleic acid library comprises at least 105 non-identical nucleic acids. Further provided herein are nucleic acid libraries, wherein a length of the immunoglobulin heavy chain when translated is about 90 to about 100 amino acids. Further provided herein are nucleic acid libraries, wherein a length of the immunoglobulin heavy chain when translated is about 100 to about 400 amino acids. Further provided herein are nucleic acid libraries, wherein the variant immunoglobulin heavy chain when translated comprises at least 90% sequence identity to SEQ ID NO: 2303, 2304, 2305, 2306, 2307, 2308, 2309, 2317, 2318, 2319, 2320, or 2321. Further provided herein are nucleic acid libraries, wherein the variant immunoglobulin light chain when translated comprises at least 90% sequence identity to SEQ ID NO: 2310, 2311, 2312, 2313, 2314, 2315, or 2316.

Provided herein are antibodies or antibody fragments that binds GLP1R, comprising an immunoglobulin heavy chain and an immunoglobulin light chain: (a) wherein the immunoglobulin heavy chain comprises an amino acid sequence at least about 90% identical to that set forth in SEQ ID NO: 2303, 2304, 2305, 2306, 2307, 2308, 2309, 2317, 2318, 2319, 2320, or 2321; and (b) wherein the immunoglobulin light chain comprises an amino acid sequence at least about 90% identical to that set forth in SEQ ID NO: 2310, 2311, 2312, 2313, 2314, 2315, or 2316, Further provided herein are antibodies or antibody fragments, wherein the immunoglobulin heavy chain comprises an amino acid sequence at least about 90% identical to that set forth in SEQ ID NO: 2303; and wherein the immunoglobulin light chain comprises an amino acid sequence at least about 90% identical to that set forth in SEQ ID NO: 2310. Further provided herein are antibodies or antibody fragments, wherein the immunoglobulin heavy chain comprises an amino acid sequence at least about 90% identical to that set forth in SEQ ID NO: 2304; and wherein the immunoglobulin light chain comprises an amino acid sequence at least about 90% identical to that set forth in SEQ ID NO: 2311. Further provided herein are antibodies or antibody fragments, wherein the immunoglobulin heavy chain comprises an amino acid sequence at least about 90% identical to that set forth in SEQ ID NO: 2305; and wherein the immunoglobulin light chain comprises an amino acid sequence at least about 90% identical to that set forth in SEQ ID NO: 2312. Further provided herein are antibodies or antibody fragments, wherein the immunoglobulin heavy chain comprises an amino acid sequence at least about 90% identical to that set forth in SEQ ID NO: 2306; and wherein the immunoglobulin light chain comprises an amino acid sequence at least about 90% identical to that set forth in SEQ ID NO: 2313. Further provided herein are antibodies or antibody fragments, wherein the immunoglobulin heavy chain comprises an amino acid sequence at least about 90% identical to that set forth in SEQ ID NO: 2307; and wherein the immunoglobulin light chain comprises an amino acid sequence at least about 90% identical to that set forth in SEQ ID NO: 2314. Further provided herein are antibodies or antibody fragments, wherein the immunoglobulin heavy chain comprises an amino acid sequence at least about 90% identical to that set forth in SEQ ID NO: 2308; and wherein the immunoglobulin light chain comprises an amino acid sequence at least about 90% identical to that set forth in SEQ ID NO: 2315. Further provided herein are antibodies or antibody fragments, wherein the immunoglobulin heavy chain comprises an amino acid sequence at least about 90% identical to that set forth in SEQ ID NO: 2309; and wherein the immunoglobulin light chain comprises an amino acid sequence at least about 90% identical to that set forth in SEQ ID NO: 2316. Further provided herein are antibodies or antibody fragments, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments, wherein the antibody or antibody fragment thereof is chimeric or humanized. Further provided herein are antibodies or antibody fragments, wherein the antibody has an EC50 less than about 25 nanomolar in a cAMP assay. Further provided herein are antibodies or antibody fragments, wherein the antibody has an EC50 less than about 20 nanomolar in a cAMP assay. Further provided herein are antibodies or antibody fragments, wherein the antibody has an EC50 less than about 10 nanomolar in a cAMP assay. Further provided herein are antibodies or antibody fragments, wherein the antibody is an agonist of GLP1R, Further provided herein are antibodies or antibody fragments, wherein the antibody is an antagonist of GLP1R, Further provided herein are antibodies or antibody fragments, wherein the antibody is an allosteric modulator of GLP1R, Further provided herein are antibodies or antibody fragments, wherein the allosteric modulator of GLP1R is a negative allosteric modulator.

Provided herein are antibodies or antibody fragments, wherein the antibody or antibody fragment comprises a sequence of any one of SEQ ID NOS: 2277, 2278, 2281, 2282, 2283, 2284, 2285, 2286, 2289, 2290, 2291, 2292, 2294, 2295, 2296, 2297, 2298, 2299, 2300, 2301, or 2302 or a sequence set forth in Table 27.

Provided herein are antibodies or antibody fragments, wherein the antibody or antibody fragment comprises a sequence of any one of SEQ ID NOS: 2277, 2278, 2281, 2282, 2283, 2284, 2285, 2286, 2289, 2290, 2291, 2292, 2294, 2295, 2296, 2297, 2298, 2299, 2300, 2301, or 2302 or a sequence set forth in Table 27; and wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

Provided herein are antagonists of GLP1R comprising SEQ ID NO: 2279 or 2320. Further provided herein are antagonists of GLP1R, wherein the antagonist comprises an EC50 of no more than 1.5 nM. Further provided herein are antagonists of GLP1R, wherein the antagonist comprises an EC50 of no more than 1.0 nM. Further provided herein are antagonists of GLP1R, wherein the antagonist comprises an EC50 of no more than 0.5 nM. Further provided herein are antagonists of GLP1R, wherein the antagonist is an antibody or antibody fragment.

Provided herein are agonists of GLP1R comprising SEQ ID NO: 2317. Further provided herein are agonists of GLP1R, wherein the agonist comprises an EC50 of no more than 1.5 nM. Further provided herein are agonists of GLP1R, wherein the agonist comprises an EC50 of no more than 1.0 nM. Further provided herein are agonists of GLP1R, wherein the agonist comprises an EC50 of no more than 0.5 nM. Further provided herein are agonists of GLP1R, wherein the agonist is an antibody or antibody fragment.

Provided herein are methods of inhibiting GLP1R activity, comprising administering the antibody or antibody fragment as described herein. Further provided herein are methods of inhibiting GLP1R activity, wherein the antibody or antibody fragment is an allosteric modulator. Further provided herein are methods of inhibiting GLP1R activity, wherein the antibody or antibody fragment is a negative allosteric modulator.

Provided herein are methods for treatment of a metabolic disorder, comprising administering to a subject in need thereof the antibody as described herein. Provided herein are methods for treatment of a metabolic disorder, wherein the metabolic disorder is Type II diabetes or obesity.

Provided herein are protein libraries encoded by the nucleic acid library as described herein, wherein the protein library comprises peptides. Further provided herein are protein libraries, wherein the protein library comprises immunoglobulins. Further provided herein are protein libraries, wherein the protein library comprises antibodies. Further provided herein are protein libraries, wherein the protein library is a peptidomimetic library.

Provided herein are vector libraries comprising the nucleic acid library as described herein. Provided herein are cell libraries comprising the nucleic acid library as described herein. Provided herein are cell libraries comprising the protein library as described herein.

### BRIEF DESCRIPTION OF THE DRAWINGS

FIG. 1A depicts a first schematic of an immunoglobulin scaffold.

FIG. 1B depicts a second schematic of an immunoglobulin scaffold.

FIG. 2 depicts a schematic of a motif for placement in a scaffold.

FIG. 3 presents a diagram of steps demonstrating an exemplary process workflow for gene synthesis as disclosed herein.

FIG. 4 illustrates an example of a computer system.

FIG. 5 is a block diagram illustrating an architecture of a computer system.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space.

FIG. 8A depicts a schematic of an immunoglobulin scaffold comprising a VH domain attached to a VL domain using a linker.

FIG. 8B depicts a schematic of a full-domain architecture of an immunoglobulin scaffold comprising a VH domain attached to a VL domain using a linker, a leader sequence, and pIII sequence.

FIG. 8C depicts a schematic of four framework elements (FW1, FW2, FW3, FW4) and the variable 3 CDR (L1, L2, L3) elements for a VL or VH domain.

FIGS. 9A-90 depict the cell binding data for GLP1R-2 (FIG. 9A). GLP1R-3 (FIG. 9B). GLP1R-8 (FIG. 9C). GLP1R-26 (FIG. 9D). GLP1R-30) (FIG. 9E). GLP1R-56 (FIG. 9F). GLP1R-58 (FIG. 9G). GLP1R-10 (FIG. 9H). GLP1R-25 (FIG. 9I). GLP1R-60 (FIG. 9J). GLP1R-70 (FIG. 9K), GLP1R-72 (FIG. 9L). GLP1R-83 (FIG. 9M). GLP1R-93 (FIG. 9N), and GLP1R-98 (FIG. 9O).

FIGS. 10A-100 depict graphs of GLP1R-2 (FIG. 10A), GLP1R-3 (FIG. 10B). GLP1R-8 (FIG. 10C). GLP1R-26 (FIG. 10D). GLP1R-30 (FIG. 10E), GLP1R-56 (FIG. 10F). GLP1R-58 (FIG. 10G). GLP1R-10 (FIG. 10H). GLP1R-25 (FIG. 10I). GLP1R-60 (FIG. 10J). GLP1R-70 (FIG. 10K). GLP1R-72 (FIG. 10L). GLP1R-83 (FIG. 10M), GLP1R-93 (FIG. 10N), and GLP1R-98 (FIG. 10O) variants on inhibition of GLP1-7-36 peptide induced cAMP activity.

FIGS. 11A-11G depict cell functional data for GLP1R-2 (FIG. 11A), GLP1R-3 (FIG. 11B). GLP1R-8 (FIG. 11C). GLP1R-26 (FIG. 11D). GLP1R-30 (FIG. 11E). GLP1R-56 (FIG. 11F), and GLP1R-58 (FIG. 11G).

FIGS. 12A-12G depict graphs of GLP1R-2 (FIG. 12A). GLP1R-3 (FIG. 12B). GLP1R-8 (FIG. 12C), GLP1R-26 (FIG. 12D). GLP1R-30 (FIG. 12E), GLP1R-56 (FIG. 12F), and GLP1R-58 (FIG. 12G) variants on inhibition of Exendin-4 peptide induced cAMP activity.

FIG. 13 depicts a schematic of glucagon (SEQ ID NO: 2740). GLP1-1 (SEQ ID NO: 6), and (GLP-2 SEQ ID NO: 2741).

FIGS. 14A-14C depict cell-binding affinity of purified immunoglobulins.

FIG. 14D depicts CAMP activity of purified immunoglobulins.

FIGS. 15A-15H depict binding curves plotting IgG concentrations in nanomolar (nM) against MFI (mean fluorescence intensity) for GLP1R-238 (FIG. 15A). GLP1R-240 (FIG. 15B). GLP1R-241 (FIG. 15C). GLP1R-242 (FIG. 15D). GLP1R-243 (FIG. 15E), GLP1R-244 (FIG. 15F), pGPCR-GLP1R-43 (FIG. 15G), and pGPCR-GLP1R-44 (FIG. 15H).

FIGS. 16A-161 depict flow cytometry data of binding assays presented as dot plots with 100 nM IgG of GLP1R-238 (FIG. 16A), GLP1R-240 (FIG. 16B), GLP1R-241 (FIG. 16C). GLP1R-242 (FIG. 16D). GLP1R-243 (FIG. 16E). GLP1R-244 (FIG. 16F), pGPCR-GLP1R-43 (FIG. 16G), pGPCR-GLP1R-44 (FIG. 16H), and GLP1R-239 (FIG. 16I).

FIGS. 17A-17B depict data from cAMP assays with relative luminescence units (RLU) on the y-axis and concentration in nanomolar (nM) on the x-axis, cAMP was measured in response to GLP1 (7-36). GLP1R-238. GLP1R-239, GLP1R-240. GLP1R-241. GLP1R-242. GLP1R-243. GLP1R-244, pGPCR-GLP1R-43, pGPCR-GLP1R-44, and buffer.

FIG. 17C depicts a graph of CAMP allosteric effect of GLP1R-241.

FIG. 17D depicts a graph of beta-arrestin recruitment of GLP1R-241.

FIG. 17E depicts a graph of GLP1R-241 internalization.

FIGS. 18A-18B depict data from cAMP assays with relative luminescence units (RLU) on the y-axis and concentration of GLP1 (7-36) in nanomolar (nM) on the x-axis. Allosteric effects of GLP1R-238. GLP1R-239. GLP1R-240, GLP1R-241. GLP1R-242. GLP1R-243. GLP1R-244, pGPCR-GLP1R-43, pGPCR-GLP1R-44, and no antibody were tested.

FIGS. 19A-19F depict flow cytometry data of binding assays presented as dot plots and histograms for GLP1R-59-2 (FIG. 19A), GLP1R-59-241 (FIG. 19B). GLP1R-59-243 (FIG. 19C), GLP1R-3 (FIG. 19D). GLP1R-241 (FIG. 19E), and GLP1R-2 (FIG. 19F). FIGS. 19A-19F also depict titration curves plotting IgG concentrations in nanomolar (nM) against MFI (mean fluorescence intensity) for GLP1R-59-2 (FIG. 19A), GLP1R-59-241 (FIG. 19B). GLP1R-59-243 (FIG. 19C), GLP1R-3 (FIG. 19D), GLP1R-241 (FIG. 19E), and GLP1R-2 (FIG. 19F).

FIGS. 20A-20F depict data from cAMP assays with relative luminescence units (RLU) on the y-axis and concentration of GLP1 (7-36) in nanomolar (nM) on the x-axis as well as beta-arrestin recruitment and receptor internalization for GLP1R-59-2 (FIG. 20A), GLP1R-59-241 (FIG. 20B), GLP1R-59-243 (FIG. 20C), GLP1R-3 (FIG. 20D), GLP1R-241 (FIG. 20E), and GLP1R-2 (FIG. 20F).

FIGS. 21A-21B depicts graphs of TIGIT affinity distribution for the VHH libraries, depicting either the affinity threshold from 20 to 4000 (FIG. 21A) or the affinity threshold from 20 to 1000 (FIG. 21B). Out of 140 VHH binders, 51 variants were <100 nM and 90 variants were <200 nM.

FIGS. 22A-22B depict graphs of FACs analysis (FIG. 22A) and graphs of a dose curve and specificity (FIG. 22B) of GLP1R-43-77.

FIG. 23A depicts a schema of heavy chain IGHV3-23 design. FIG. 23A discloses SEQ ID NOS 2742-2747, respectively, in order of appearance.

FIG. 23B depicts a schema of heavy chain IGHV1-69 design. FIG. 23B discloses SEQ ID NOS 2748-2753, respectively, in order of appearance.

FIG. 23C depicts a schema of light chains IGKV2-28 and IGLV1-51 design. FIG. 23C discloses SEQ ID NOS 2754-2759, respectively, in order of appearance.

FIG. 23D depicts a schema of the theoretical diversity and final diversity of a GLP1R library.

FIGS. 23E-23F depict graphs of FACS binding of GLP1R IgGs.

FIGS. 23G-23H depict graphs of cAMP assays using purified GLP1R IgGs.

FIG. 24A depicts a graph of GLP1R-3 inhibition as compared to no antibody, Relative luminescence units (RLU) is depicted on the y-axis, and concentration of GLP1 (7-36) is depicted in nanomolar (nM) on the x-axis.

FIG. 24B depicts a graph of GLP1R-3 inhibition at high concentrations following stimulation with 0.05 nM GLP1 (7-36). Relative luminescence units (RLU) is depicted on the y-axis, and concentration of GLP1R-3 is depicted in nanomolar (nM) on the x-axis.

FIG. 24C depicts glucose levels after glucose administration when treated with vehicle (triangles), liraglutide (squares), and GLP1R-3 (circles) in a mouse model of diet induced obesity.

FIG. 24D depicts glucose levels after glucose administration when treated with vehicle (open triangles), liraglutide (squares), and GLP1R-59-2 (closed triangles) in a mouse model of diet induced obesity.

FIG. 25A depicts a graph of the blood glucose levels in mice (mg/dL: y-axis) treated with GLP1R-59-2 (agonist). GLP1R-3 (antagonist), and control over time (in minutes, x-axis).

FIG. 25B depicts a graph of blood glucose levels in mice (mg/dL: y-axis) treated with GLP1R-59-2 (agonist). GLP1R-3 (antagonist), and control.

FIG. 25C depicts a graph of the blood glucose levels (mg/dL: y-axis) in GLP1R-59-2 (agonist) treated mice in both the fasted (p=0.0008) and non-fasted (p<0,0001) mice compared to control.

FIG. 25D depicts a graph of the blood glucose levels (mg/dL/min: y-axis) in pre-dosed GLP1R-59-2 (agonist). GLP1R-3 (antagonist), and control mice.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification: DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers, cDNA encoding for a gene or gene fragment referred herein may comprise at least one region encoding for exon sequences without an intervening intron sequence in the genomic equivalent sequence.

### GPCR Libraries for GLP1 Receptor

Provided herein are methods and compositions relating to G protein-coupled receptor (GPCR) binding libraries for glucagon-like peptide-1 receptor (GLP1R) comprising nucleic acids encoding for a scaffold comprising a GPCR binding domain. Scaffolds as described herein can stably support a GPCR binding domain. The GPCR binding domain may be designed based on surface interactions of a GLP1R ligand and GLP1R, Libraries as described herein may be further variegated to provide for variant libraries comprising nucleic acids each encoding for a predetermined variant of at least one predetermined reference nucleic acid sequence. Further described herein are protein libraries that may be generated when the nucleic acid libraries are translated. In some instances, nucleic acid libraries as described herein are transferred into cells to generate a cell library. Also provided herein are downstream applications for the libraries synthesized using methods described herein. Downstream applications include identification of variant nucleic acids or protein sequences with enhanced biologically relevant functions, e.g., improved stability, affinity, binding, functional activity, and for the treatment or prevention of a disease state associated with GPCR signaling.

### Scaffold Libraries

Provided herein are libraries comprising nucleic acids encoding for a scaffold, wherein sequences for GPCR binding domains are placed in the scaffold. Scaffold described herein allow for improved stability for a range of GPCR binding domain encoding sequences when inserted into the scaffold, as compared to an unmodified scaffold. Exemplary scaffolds include, but are not limited to, a protein, a peptide, an immunoglobulin, derivatives thereof, or combinations thereof. In some instances, the scaffold is an immunoglobulin. Scaffolds as described herein comprise improved functional activity, structural stability, expression, specificity, or a combination thereof. In some instances, scaffolds comprise long regions for supporting a GPCR binding domain.

Provided herein are libraries comprising nucleic acids encoding for a scaffold, wherein the scaffold is an immunoglobulin. In some instances, the immunoglobulin is an antibody. As used herein, the term antibody will be understood to include proteins having the characteristic two-armed. Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CHI domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

In some embodiments, libraries comprise immunoglobulins that are adapted to the species of an intended therapeutic target. Generally, these methods include “mammalization” and comprises methods for transferring donor antigen-binding information to a less immunogenic mammal antibody acceptor to generate useful therapeutic treatments. In some instances, the mammal is mouse, rat, equine, sheep, cow, primate (e.g., chimpanzee, baboon, gorilla, orangutan, monkey), dog, cat, pig, donkey, rabbit, and human. In some instances, provided herein are libraries and methods for felinization and caninization of antibodies.

“Humanized” forms of non-human antibodies can be chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance.

“Caninization” can comprise a method for transferring non-canine antigen-binding information from a donor antibody to a less immunogenic canine antibody acceptor to generate treatments useful as therapeutics in dogs. In some instances, caninized forms of non-canine antibodies provided herein are chimeric antibodies that contain minimal sequence derived from non-canine antibodies. In some instances, caninized antibodies are canine antibody sequences (“acceptor” or “recipient” antibody) in which hypervariable region residues of the recipient are replaced by hypervariable region residues from a non-canine species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the canine antibody are replaced by corresponding non-canine FR residues. In some instances, caninized antibodies include residues that are not found in the recipient antibody or in the donor antibody. In some instances, these modifications are made to further refine antibody performance. The caninized antibody may also comprise at least a portion of an immunoglobulin constant region (Fc) of a canine antibody.

“Felinization” can comprise a method for transferring non-feline antigen-binding information from a donor antibody to a less immunogenic feline antibody acceptor to generate treatments useful as therapeutics in cats. In some instances, felinized forms of non-feline antibodies provided herein are chimeric antibodies that contain minimal sequence derived from non-feline antibodies. In some instances, felinized antibodies are feline antibody sequences (“acceptor” or “recipient” antibody) in which hypervariable region residues of the recipient are replaced by hypervariable region residues from a non-feline species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the feline antibody are replaced by corresponding non-feline FR residues. In some instances, felinized antibodies include residues that are not found in the recipient antibody or in the donor antibody. In some instances, these modifications are made to further refine antibody performance. The felinized antibody may also comprise at least a portion of an immunoglobulin constant region (Fc) of a felinize antibody.

Provided herein are libraries comprising nucleic acids encoding for a scaffold, wherein the scaffold is a non-immunoglobulin. In some instances, the scaffold is a non-immunoglobulin binding domain. For example, the scaffold is an antibody mimetic. Exemplary antibody mimetics include, but are not limited to, anticalins, affilins, affibody molecules, affimers, affitins, alphabodies, avimers, atrimers, DARPins, fynomers, Kunitz domain-based proteins, monobodies, anticalins, knottins, armadillo repeat protein-based proteins, and bicyclic peptides.

Libraries described herein comprising nucleic acids encoding for a scaffold, wherein the scaffold is an immunoglobulin, comprise variations in at least one region of the immunoglobulin. Exemplary regions of the antibody for variation include, but are not limited to, a complementarity-determining region (CDR), a variable domain, or a constant domain. In some instances, the CDR is CDR1, CDR2, or CDR3. In some instances, the CDR is a heavy domain including, but not limited to, CDR-H1, CDR-H2, and CDR-H3. In some instances, the CDR is a light domain including, but not limited to, CDR-L1, CDR-L2, and CDR-L3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH).

Methods described herein provide for synthesis of libraries comprising nucleic acids encoding for a scaffold, wherein each nucleic acid encodes for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the scaffold library comprises varied nucleic acids collectively encoding variations at multiple positions. In some instances, the variant library comprises sequences encoding for variation of at least a single codon of a CDR-H1, CDR-H2, CDR-H3, CDR-L1, CDR-L2, CDR-L3, VL, or VH domain. In some instances, the variant library comprises sequences encoding for variation of multiple codons of a CDR-H1, CDR-H2, CDR-H3, CDR-L1, CDR-L2, CDR-L3, VL, or VH domain. In some instances, the variant library comprises sequences encoding for variation of multiple codons of framework element 1 (FW1), framework element 2 (FW2), framework element 3 (FW3), or framework element 4 (FW4). An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

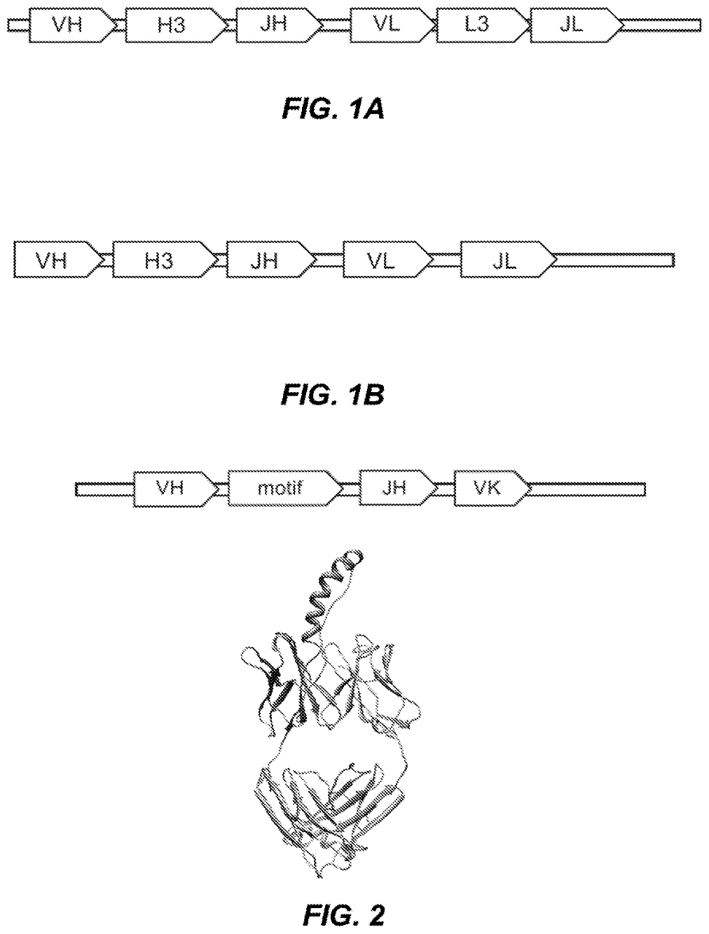

In some instances, the at least one region of the immunoglobulin for variation is from heavy chain V-gene family, heavy chain D-gene family, heavy chain J-gene family, light chain V-gene family, or light chain J-gene family. See FIGS. 1A-1B. In some instances, the light chain V-gene family comprises immunoglobulin kappa (IGK) gene or immunoglobulin lambda (IGL). Exemplary genes include, but are not limited to, IGHV1-18, IGHV1-69, IGHV1-8, IGHV3-21, IGHV3-23, IGHV3-30/33m, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGHV1-69, IGHV3-30, IGHV3-23, IGHV3, IGHV1-46, IGHV3-7, IGHV1, or IGHV1-8. In some instances, the gene is IGHV1-69 and IGHV3-30. In some instances, the gene is IGHJ3, IGHJ6, IGHJ, IGHJ4, IGHJ5, IGHJ2, or IGH1. In some instances, the gene is IGHJ3, IGHJ6, IGHJ, or IGHJ4.

Provided herein are libraries comprising nucleic acids encoding for immunoglobulin scaffolds, wherein the libraries are synthesized with various numbers of fragments. In some instances, the fragments comprise the CDR-H1, CDR-H2, CDR-H3, CDR-L1, CDR-L2, CDR-L3, VL, or VH domain. In some instances, the fragments comprise framework element 1 (FW1), framework element 2 (FW2), framework element 3 (FW3), or framework element 4 (FW4). In some instances, the scaffold libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Libraries comprising nucleic acids encoding for immunoglobulin scaffolds as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the immunoglobulin scaffolds comprise at least or about 100, 200, 300, 400, 500, 600, 700, 800, 900, 1000, 2000, 3000, 4000, 5000, or more than 5000 amino acids.

A number of variant sequences for the at least one region of the immunoglobulin for variation are de novo synthesized using methods as described herein. In some instances, a number of variant sequences is de novo synthesized for CDR-H1, CDR-H2, CDR-H3, CDR-L1, CDR-L2, CDR-L3, VL, VH, or combinations thereof. In some instances, a number of variant sequences is de novo synthesized for framework element 1 (FW1), framework element 2 (FW2), framework element 3 (FW3), or framework element 4 (FW4). The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is at least or about 500, 600, 700, 800, 900, 1000, 2000, 3000, 4000, 5000, 6000, 7000, 8000, or more than 8000 sequences. In some instances, the number of variant sequences is about 10 to 500, 25 to 475, 50 to 450, 75 to 425, 100 to 400, 125 to 375, 150 to 350, 175 to 325, 200 to 300, 225 to 375, 250 to 350, or 275 to 325 sequences.

Variant sequences for the at least one region of the immunoglobulin, in some instances, vary in length or sequence. In some instances, the at least one region that is de novo synthesized is for CDR-H1, CDR-H2, CDR-H3, CDR-L1, CDR-L2, CDR-L3, VL, VH, or combinations thereof. In some instances, the at least one region that is de novo synthesized is for framework element 1 (FW1), framework element 2 (FW2), framework element 3 (FW3), or framework element 4 (FW4). In some instances, the variant sequence comprises at least or about 1, 2, 3, 4, 5, 6, 7, 8, 9, 10, 15, 20, 25, 30, 35, 40, 45, 50, or more than 50 variant nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 additional nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 less nucleotides or amino acids as compared to wild-type. In some instances, the libraries comprise at least or about 10¹, 10², 10³, 10⁴, 10⁵, 10⁶, 10⁷, 10⁸, 10⁹, 10¹⁰, or more than 10¹⁰ variants.

Following synthesis of scaffold libraries, scaffold libraries may be used for screening and analysis. For example, scaffold libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, scaffold libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

In some instances, the scaffold libraries are assayed for functional activity, structural stability (e.g., thermal stable or pH stable), expression, specificity, or a combination thereof. In some instances, the scaffold libraries are assayed for scaffolds capable of folding. In some instances, a region of the antibody is assayed for functional activity, structural stability, expression, specificity, folding, or a combination thereof. For example, a VH region or VL region is assayed for functional activity, structural stability, expression, specificity, folding, or a combination thereof.

### GLPIR Libraries

Provided herein are GLP1R binding libraries comprising nucleic acids encoding for scaffolds comprising sequences for GLP1R binding domains. In some instances, the scaffolds are immunoglobulins. In some instances, the scaffolds comprising sequences for GLP1R binding domains are determined by interactions between the GLP1R binding domains and the GLP1R.

Provided herein are libraries comprising nucleic acids encoding scaffolds comprising GLP1R binding domains, wherein the GLP1R binding domains are designed based on surface interactions on GLP1R. In some instances, the GLP1R comprises a sequence as defined by SEQ ID NO: 1. In some instances, the GLP1R binding domains interact with the amino- or carboxy-terminus of the GLP1R. In some instances, the GLP1R binding domains interact with at least one transmembrane domain including, but not limited to, transmembrane domain 1 (TM1), transmembrane domain 2 (TM2), transmembrane domain 3 (TM3), transmembrane domain 4 (TM4), transmembrane domain 5 (TM5), transmembrane domain 6 (TM6), and transmembrane domain 7 (TM7). In some instances, the GLP1R binding domains interact with an intracellular surface of the GLP1R. For example, the GLPIR binding domains interact with at least one intracellular loop including, but not limited to, intracellular loop 1 (ICL1), intracellular loop 2 (ICL2), and intracellular loop 3 (ICL3). In some instances, the GLP1R binding domains interact with an extracellular surface of the GLP1R. For example, the GLP1R binding domains interact with at least one extracellular domain (ECD) or extracellular loop (ECL) of the GLP1R. The extracellular loops include, but are not limited to, extracellular loop 1 (ECL1), extracellular loop 2 (ECL2), and extracellular loop 3 (ECL3).

Described herein are GLP1R binding domains, wherein the GLP1R binding domains are designed based on surface interactions between a GLP1R ligand and the GLP1R. In some instances, the ligand is a peptide. In some instances, the ligand is glucagon, glucagon-like peptide 1-(7-36) amide, glucagon-like peptide 1-(7-37), liraglutide, exendin-4, lixisenatide, T-0632, GLP1R0017, or BETP. In some instances, the ligand is a GLP1R agonist. In some instances, the ligand is a GLP1R antagonist. In some instances, the ligand is a GLP1R allosteric modulator. In some instances, the allosteric modulator is a negative allosteric modulator. In some instances, the allosteric modulator is a positive allosteric modulator.

Sequences of GLP1R binding domains based on surface interactions between a GLP1R ligand and the GLP1R are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (www.uniprot.org/), and IUPHAR/BPS Guide to PHARMACOLOGY (guidetopharmacology.org/).

Described herein are GLP1R binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human.

Following identification of GLP1R binding domains, libraries comprising nucleic acids encoding for the GLP1R binding domains may be generated. In some instances, libraries of GLP1R binding domains comprise sequences of GLP1R binding domains designed based on conformational ligand interactions, peptide ligand interactions, small molecule ligand interactions, extracellular domains of GLP1R, or antibodies that target GLP1R. In some instances, libraries of GLP1R binding domains comprise sequences of GLP1R binding domains designed based on peptide ligand interactions. Libraries of GLP1R binding domains may be translated to generate protein libraries. In some instances, libraries of GLP1R binding domains are translated to generate peptide libraries, immunoglobulin libraries, derivatives thereof, or combinations thereof. In some instances, libraries of GLP1R binding domains are translated to generate protein libraries that are further modified to generate peptidomimetic libraries. In some instances, libraries of GLP1R binding domains are translated to generate protein libraries that are used to generate small molecules.

Methods described herein provide for synthesis of libraries of GLP1R binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of GLP1R binding domains comprise varied nucleic acids collectively encoding variations at multiple positions. In some instances, the variant library comprises sequences encoding for variation of at least a single codon in a GLP1R binding domain. In some instances, the variant library comprises sequences encoding for variation of multiple codons in a GLP1R binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the GLP1R binding domains, wherein the libraries comprise sequences encoding for variation of length of the GLP1R binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of GLP1R binding domains, the GLP1R binding domains may be placed in scaffolds as described herein. In some instances, the scaffolds are immunoglobulins. In some instances, the GLP1R binding domains are placed in the CDR-H3 region. GPCR binding domains that may be placed in scaffolds can also be referred to as a motif Scaffolds comprising GLP1R binding domains may be designed based on binding, specificity, stability, expression, folding, or downstream activity. In some instances, the scaffolds comprising GLP1R binding domains enable contact with the GLP1R. In some instances, the scaffolds comprising GLP1R binding domains enables high affinity binding with the GLP1R. An exemplary amino acid sequence of GLP1R binding domain is described in Table 1.

TABLE 1
GLP1R amino acid sequences

| SEQ | | |
|---|---|---|
| ID | | |
| NO | GPCR | Amino Acid Sequence |
| 1 | GLP1R | RPQGATVSLWETVQKWREYRRQCQRSLTEDPPPAT |
| | | DLFCNRTFDEYACWPDGEPGSFVNVSCPWYLPWAS |
| | | SVPQGHVYRFCTAEGLWLQKDNSSLPWRDLSECEE |
| | | SKRGERSSPEEQLLFLYIIYTVGYALSFSALVIAS |
| | | AILLGFRHLHCTRNYIHLNLFASFILRALSVFIKD |
| | | AALKWMYSTAAQQHQWDGLLSYQDSLSCRLVFLLM |
| | | QYCVAANYYWLLVEGVYLYTLLAFSVLSEQWIFRL |
| | | YVSIGWGVPLLFVVPWGIVKYLYEDEGCWTRNSNM |
| | | NYWLIIRLPILFAIGVNFLIFVRVICIVVSKLKAN |
| | | LMCKTDIKCRLAKSTLTLIPLLGTHEVIFAFVMDE |
| | | HARGTLRFIKLFTELSFTSFQGLMVAILYCFVNNE |
| | | VQLEFRKSWERWRLEHLHIQRDSSMKPLKCPTSSL |
| | | SSGATAGSSMYTATCQASCS |

Provided herein are scaffolds comprising GLP1R binding domains, wherein the sequences of the GLP1R binding domains support interaction with GLP1R. The sequence may be homologous or identical to a sequence of a GLP1R ligand. In some instances, the GLP1R binding domain sequence comprises at least or about 70%, 80%, 85%, 90%, 91%, 92%, 93%, 94%, 95%, 96%, 97%, 98%, 99%, or 100% sequence identity to SEQ ID NO: 1. In some instances, the GLP1R binding domain sequence comprises at least or about 95% homology to SEQ ID NO: 1. In some instances, the GLP1R binding domain sequence comprises at least or about 97% homology to SEQ ID NO: 1. In some instances, the GLP1R binding domain sequence comprises at least or about 99% homology to SEQ ID NO: 1. In some instances, the GLP1R binding domain sequence comprises at least or about 100% homology to SEQ ID NO: 1. In some instances, the GLP1R binding domain sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NO: 1.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

Provided herein are GLP1R binding libraries comprising nucleic acids encoding for scaffolds comprising GLP1R binding domains comprise variation in domain type, domain length, or residue variation. In some instances, the domain is a region in the scaffold comprising the GLP1R binding domains. For example, the region is the VH, CDR-H3, or VL domain. In some instances, the domain is the GLP1R binding domain.

Methods described herein provide for synthesis of a GLP1R binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the GLP1R binding library comprises varied nucleic acids collectively encoding variations at multiple positions. In some instances, the variant library comprises sequences encoding for variation of at least a single codon of a VH, CDR-H3, or VL domain. In some instances, the variant library comprises sequences encoding for variation of at least a single codon in a GLP1R binding domain. For example, at least one single codon of a GLP1R binding domain as listed in Table 1 is varied. In some instances, the variant library comprises sequences encoding for variation of multiple codons of a VH, CDR-H3, or VL domain. In some instances, the variant library comprises sequences encoding for variation of multiple codons in a GLP1R binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a GLP1R binding library of nucleic acids each encoding for a predetermined variant of at least one predetermined reference nucleic acid sequence, wherein the GLP1R binding library comprises sequences encoding for variation of length of a domain. In some instances, the domain is VH, CDR-H3, or VL domain. In some instances, the domain is the GLP1R binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are GLP1R binding libraries comprising nucleic acids encoding for scaffolds comprising GLP1R binding domains, wherein the GLP1R binding libraries are synthesized with various numbers of fragments. In some instances, the fragments comprise the VH, CDR-H3, or VL domain. In some instances, the GLP1R binding libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

GLP1R binding libraries comprising nucleic acids encoding for scaffolds comprising GLP1R binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

GLP1R binding libraries comprising de novo synthesized variant sequences encoding for scaffolds comprising GLP1R binding domains comprise a number of variant sequences. In some instances, a number of variant sequences is de novo synthesized for a CDR-H1, CDR-H2, CDR-H3, CDR-L1, CDR-L2, CDR-L3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences is de novo synthesized for a GPCR binding domain. For example, the number of variant sequences is about 1 to about 10 sequences for the VH domain, about 10⁸ sequences for the GLP1R binding domain, and about 1 to about 44 sequences for the VK domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

GLP1R binding libraries comprising de novo synthesized variant sequences encoding for scaffolds comprising GLP1R binding domains comprise improved diversity. For example, variants are generated by placing GLP1R binding domain variants in immunoglobulin scaffold variants comprising N-terminal CDR-H3 variations and C-terminal CDR-H3 variations. In some instances, variants include affinity maturation variants. Alternatively or in combination, variants include variants in other regions of the immunoglobulin including, but not limited to, CDR-H1, CDR-H2, CDR-L1, CDR-L2, and CDR-L3. In some instances, the number of variants of the GLP1R binding libraries is least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ non-identical sequences. For example, a library comprising about 10 variant sequences for a VH region, about 237 variant sequences for a CDR-H3 region, and about 43 variant sequences for a VL and CDR-L3 region comprises 105 non-identical sequences (10×237×43).

Provided herein are libraries comprising nucleic acids encoding for a GLP1R antibody comprising variation in at least one region of the antibody, wherein the region is the CDR region. In some instances, the GLP1R antibody is a single domain antibody comprising one heavy chain variable domain such as a VHH antibody. In some instances, the VHH antibody comprises variation in one or more CDR regions. In some instances, libraries described herein comprise at least or about 1, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3. For example, the libraries comprise at least 2000 sequences of a CDR1, at least 1200 sequences for CDR2, and at least 1600 sequences for CDR3. In some instances, each sequence is non-identical.

In some instances, the CDR1, CDR2, or CDR3 is of a variable domain, light chain (VL). CDR1, CDR2, or CDR3 of a variable domain, light chain (VL) can be referred to as CDR-L1, CDR-L2, or CDR-L3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VL. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VL. For example, the libraries comprise at least 20 sequences of a CDR1 of the VL, at least 4 sequences of a CDR2 of the VL, and at least 140 sequences of a CDR3 of the VL. In some instances, the libraries comprise at least 2 sequences of a CDR1 of the VL, at least 1 sequence of CDR2 of the VL, and at least 3000 sequences of a CDR3 of the VL. In some instances, the VL is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. In some instances, the VL is IGKV2-28. In some instances, the VL is IGLV1-51.

In some instances, the CDR1, CDR2, or CDR3 is of a variable domain, heavy chain (VH). CDR1, CDR2, or CDR3 of a variable domain, heavy chain (VH) can be referred to as CDR-H1, CDR-H2, or CDR-H3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VH. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VH. For example, the libraries comprise at least 30 sequences of a CDR1 of the VH, at least 570 sequences of a CDR2 of the VH, and at least 10⁸ sequences of a CDR3 of the VH. In some instances, the libraries comprise at least 30 sequences of a CDR1 of the VH, at least 860 sequences of a CDR2 of the VH, and at least 107 sequences of a CDR3 of the VH. In some instances, the VH is IGHV1-18, IGHV1-69, IGHV1-8 IGHV3-21, IGHV3-23, IGHV3-30/33m, IGHV3-28, IGHV3-74, IGHV4-39, or IGHV4-59/61. In some instances, the VH is IGHV1-69, IGHV3-30, IGHV3-23, IGHV3, IGHV1-46, IGHV3-7, IGHV1, or IGHV1-8. In some instances, the VH is IGHV1-69 and IGHV3-30. In some instances, the VH is IGHV3-23.

Libraries as described herein, in some embodiments, comprise varying lengths of a CDR-L1, CDR-L2, CDR-L3, CDR-H1, CDR-H2, or CDR-H3. In some instances, the length of the CDR-L1, CDR-L2, CDR-L3, CDR-H1, CDR-H2, or CDR-H3 comprises at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length. For example, the CDR-H3 comprises at least or about 12, 15, 16, 17, 20, 21, or 23 amino acids in length. In some instances, the CDR-L1, CDR-L2, CDR-L3, CDR-H1, CDR-H2, or CDR-H3 comprises a range of about 1 to about 10, about 5 to about 15, about 10 to about 20, or about 15 to about 30 amino acids in length.

Libraries comprising nucleic acids encoding for antibodies having variant CDR sequences as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

Ratios of the lengths of a CDR-L1, CDR-L2, CDR-L3, CDR-H1, CDR-H2, or CDR-H3 may vary in libraries described herein. In some instances, a CDR-L1, CDR-L2, CDR-L3, CDR-H1, CDR-H2, or CDR-H3 comprising at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length comprises about 10%, 20%, 30%, 40%, 50%, 60%, 70%, 80%, 90%, or more than 90% of the library. For example, a CDR-H3 comprising about 23 amino acids in length is present in the library at 40%, a CDR-H3 comprising about 21 amino acids in length is present in the library at 30%, a CDR-H3 comprising about 17 amino acids in length is present in the library at 20%, and a CDR-H3 comprising about 12 amino acids in length is present in the library at 10%. In some instances, a CDR-H3 comprising about 20 amino acids in length is present in the library at 40%, a CDR-H3 comprising about 16 amino acids in length is present in the library at 30%, a CDR-H3 comprising about 15 amino acids in length is present in the library at 20%, and a CDR-H3 comprising about 12 amino acids in length is present in the library at 10%.

Libraries as described herein encoding for a VHH antibody comprise variant CDR sequences that are shuffled to generate a library with a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences. In some instances, the library has a final library diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences.

Provided herein are GLP1R binding libraries encoding for an immunoglobulin. In some instances, the GLP1R immunoglobulin is an antibody. In some instances, the GLP1R immunoglobulin is a VHH antibody. In some instances, the GLP1R immunoglobulin comprises a binding affinity (e.g., kD) to GLP1R of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the GLP1R immunoglobulin comprises a kD of less than 1 nM. In some instances, the GLP1R immunoglobulin comprises a kD of less than 1.2 nM. In some instances, the GLP1R immunoglobulin comprises a kD of less than 2 nM. In some instances, the GLP1R immunoglobulin comprises a kD of less than 5 nM. In some instances, the GLP1R immunoglobulin comprises a kD of less than 10 nM. In some instances, the GLP1R immunoglobulin comprises a kD of less than 13.5 nM. In some instances, the GLP1R immunoglobulin comprises a kD of less than 15 nM. In some instances, the GLP1R immunoglobulin comprises a kD of less than 20 nM. In some instances, the GLP1R immunoglobulin comprises a kD of less than 25 nM. In some instances, the GLP1R immunoglobulin comprises a kD of less than 30 nM.

In some instances, the GLP1R immunoglobulin is a GLP1R agonist. In some instances, the GLP1R immunoglobulin is a GLP1R antagonist. In some instances, the GLP1R immunoglobulin is a GLP1R allosteric modulator. In some instances, the allosteric modulator is a negative allosteric modulator. In some instances, the allosteric modulator is a positive allosteric modulator. In some instances, the GLP1R immunoglobulin results in agonistic, antagonistic, or allosteric effects at a concentration of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some instances, the GLP1R immunoglobulin is a negative allosteric modulator. In some instances, the GLP1R immunoglobulin is a negative allosteric modulator at a concentration of at least or about 0.001, 0.005, 0.01, 0.05, 0.1, 0.5, 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM. In some instances, the GLP1R immunoglobulin is a negative allosteric modulator at a concentration in a range of about 0.001 to about 100, 0.01 to about 90, about 0.1 to about 80, 1 to about 50, about 10 to about 40 nM, or about 1 to about 10 nM. In some instances, the GLP1R immunoglobulin comprises an EC50 or IC50 of at least or about 0.001, 0.0025, 0.005, 0.01, 0.025, 0.05, 0.06, 0.07, 0.08, 0.9, 0.1, 0.5, 1, 2, 3, 4, 5, 6, or more than 6 nM. In some instances, the GLP1R immunoglobulin comprises an EC50 or IC50 of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM.

Provided herein are GLP1R binding libraries encoding for an immunoglobulin, wherein the immunoglobulin comprises a long half-life. In some instances, the half-life of the GLP1R immunoglobulin is at least or about 12 hours, 24 hours 36 hours, 48 hours, 60 hours, 72 hours, 84 hours, 96 hours, 108 hours, 120 hours, 140 hours, 160 hours, 180 hours, 200 hours, or more than 200 hours. In some instances, the half-life of the GLP1R immunoglobulin is in a range of about 12 hours to about 300 hours, about 20 hours to about 280 hours, about 40 hours to about 240 hours, or about 60 hours to about 200 hours.

GLP1R immunoglobulins as described herein may comprise improved properties. In some instances, the GLP1R immunoglobulins are monomeric. In some instances, the GLP1R immunoglobulins are not prone to aggregation. In some instances, at least or about 70%, 75%, 80%, 85%, 90%, 95%, or 99% of the GLP1R immunoglobulins are monomeric. In some instances, the GLP1R immunoglobulins are thermostable. In some instances, the GLP1R immunoglobulins result in reduced non-specific binding.

Following synthesis of GLP1R binding libraries comprising nucleic acids encoding scaffolds comprising GLP1R binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, the GLP1R binding libraries comprises nucleic acids encoding scaffolds comprising GPCR binding domains with multiple tags such as GFP, FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

### Expression Systems

Provided herein are libraries comprising nucleic acids encoding for scaffolds comprising GLP1R binding domains, wherein the libraries have improved specificity, stability, expression, folding, or downstream activity. In some instances, libraries described herein are used for screening and analysis.

Provided herein are libraries comprising nucleic acids encoding for scaffolds comprising GLP1R binding domains, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, rabbit, primate, and insect.

Nucleic acid libraries or protein libraries encoded thereof described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Pharmacological or pharmacokinetic properties that may be screened include, but are not limited to, cell binding affinity and cell activity. For example, cell binding affinity assays or cell activity assays are performed to determine agonistic, antagonistic, or allosteric effects of libraries described herein. In some instances, the cell activity assay is a cAMP assay. In some instances, libraries as described herein are compared to cell binding or cell activity of ligands of GLP1R.

Libraries as described herein may be screened in cell based assays or in non-cell based assays. Examples of non-cell based assays include, but are not limited to, using viral particles, using in vitro translation proteins, and using protealiposomes with GLP1R.

Nucleic acid libraries as described herein may be screened by sequencing. In some instances, next generation sequence is used to determine sequence enrichment of GLP1R binding variants. In some instances, V gene distribution, J gene distribution, V gene family, CDR3 counts per length, or a combination thereof is determined. In some instances, clonal frequency, clonal accumulation, lineage accumulation, or a combination thereof is determined. In some instances, number of sequences, sequences with VH clones, clones, clones greater than 1, clonotypes, clonotypes greater than 1, lineages, simpsons, or a combination thereof is determined. In some instances, a percentage of non-identical CDR3s is determined. For example, the percentage of non-identical CDR3s is calculated as the number of non-identical CDR3s in a sample divided by the total number of sequences that had a CDR3 in the sample.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3XFLAG, pSF-CMV-NEO-COOH-3XFLAG, pSF-CMV-PURO-NH2-GST-TEV, pSF-OXB20-COOH-TEV-FLAG(R)-6His, (6His” disclosed as SEQ ID NO: 2410), pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising a scaffold comprising sequences of GLP1R binding domains. In some instances, a size of the construct varies. In some instances, the construct comprises at least or about 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for scaffolds comprising GPCR binding domains, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

### Diseases and Disorders

Provided herein are GLP1R binding libraries comprising nucleic acids encoding for scaffolds comprising GLP1R binding domains that may have therapeutic effects. In some instances, the GLP1R binding libraries result in protein when translated that is used to treat a disease or disorder. In some instances, the protein is an immunoglobulin. In some instances, the protein is a peptidomimetic.

GLP1R libraries as described herein may comprise modulators of GLP1R. In some instances, the modulator of GLP1R is an inhibitor. In some instances, the modulator of GLP1R is an activator. In some instances, the GLP1R inhibitor is a GLP1R antagonist. In some instances, the GLP1R antagonist is GLP1R-3. In some instances, GLP1R-3 comprises SEQ ID NO: 2279. In some instances, GLP1R-3 comprises SEQ ID NO: 2320. Modulators of GLP1R, in some instances, are used for treating various diseases or disorders.

Exemplary diseases include, but are not limited to, cancer, inflammatory diseases or disorders, a metabolic disease or disorder, a cardiovascular disease or disorder, a respiratory disease or disorder, pain, a digestive disease or disorder, a reproductive disease or disorder, an endocrine disease or disorder, or a neurological disease or disorder. In some instances, the cancer is a solid cancer or a hematologic cancer. In some instances, a modulator of GLP1R as described herein is used for treatment of weight gain (or for inducing weight loss), treatment of obesity, or treatment of Type II diabetes. In some instances, the GLP1R modulator is used for treating hypoglycemia. In some instances, the GLP1R modulator is used for treating post-bariatric hypoglycemia. In some instances, the GLP1R modulator is used for treating severe hypoglycemia. In some instances, the GLP1R modulator is used for treating hyperinsulinism. In some instances, the GLP1R modulator is used for treating congenital hyperinsulinism.

In some instances, the subject is a mammal. In some instances, the subject is a mouse, rabbit, dog, or human. Subjects treated by methods described herein may be infants, adults, or children. Pharmaceutical compositions comprising antibodies or antibody fragments as described herein may be administered intravenously or subcutaneously.

Described herein are pharmaceutical compositions comprising antibodies or antibody fragment thereof that binds GLP1R. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2303, 2304, 2305, 2306, 2307, 2308, 2309, 2317, 2318, 2319, 2320, or 2321; and wherein the immunoglobulin light chain comprises an amino acid sequence at least about 90%, 95%, 97%, 99%, or 100% identical to that set forth in SEQ ID NO: 2310, 2311, 2312, 2313, 2314, 2315, or 2316. In some embodiments, the antibody or antibody fragment thereof comprises an immunoglobulin heavy chain and an immunoglobulin light chain: wherein the immunoglobulin heavy chain comprises an amino acid sequence set forth in SEQ ID NO: 2303, 2304, 2305, 2306, 2307, 2308, 2309, 2317, 2318, 2319, 2320, or 2321; and wherein the immunoglobulin light chain comprises an amino acid sequence set forth in SEQ ID NO: 2310, 2311, 2312, 2313, 2314, 2315, or 2316.

In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2303; and wherein the immunoglobulin light chain comprises an amino acid sequence at least about 90%, 95%, 97%, 99%, or 100% identical to that set forth in SEQ ID NO: 2310. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2304; and wherein the immunoglobulin light chain comprises an amino acid sequence at least about 90%, 95%, 97%, 99%, or 100% identical to that set forth in SEQ ID NO: 2311. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2305; and wherein the immunoglobulin light chain comprises an amino acid sequence at least about 90%, 95%, 97%, 99%, or 100% identical to that set forth in SEQ ID NO: 2312. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2306; and wherein the immunoglobulin light chain comprises an amino acid sequence at least about 90%, 95%, 97%, 99%, or 100% identical to that set forth in SEQ ID NO: 2313. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2307; and wherein the immunoglobulin light chain comprises an amino acid sequence at least about 90%, 95%, 97%, 99%, or 100% identical to that set forth in SEQ ID NO: 2314. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2308; and wherein the immunoglobulin light chain comprises an amino acid sequence at least about 90%, 95%, 97%, 99%, or 100% identical to that set forth in SEQ ID NO: 2315. In some embodiments, the antibody or antibody fragment thereof comprises an immunoglobulin heavy chain and an immunoglobulin light chain: wherein the immunoglobulin heavy chain comprises an amino acid sequence at least about 90%, 95%, 97%, 99%, or 100% identical to that set forth in SEQ ID NO: 2309; and wherein the immunoglobulin light chain comprises an amino acid sequence at least about 90%, 95%, 97%, 99%, or 100% identical to that set forth in SEQ ID NO: 2316.

In some instances, a pharmaceutical composition comprises an antibody or antibody fragment described herein comprising a CDR-H3 comprising a sequence of any one of SEQ ID NOS: 2260-2276. In some instances, a pharmaceutical composition comprises an antibody or antibody fragment described herein comprise a sequence of any one of SEQ ID NOS: 2277-2295. In some instances, a pharmaceutical composition comprises an antibody or antibody fragment described herein comprise a sequence of any one of SEQ ID NOS: 2277, 2278, 2281, 2282, 2283, 2284, 2285, 2286, 2289, 2290, 2291, 2292, 2294, or 2295. In further instances, the pharmaceutical composition is used for treatment of a metabolic disorder.

### Variant Libraries

### Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 3 provides a listing of each codon possible (and the representative amino acid) for a variant site.

TABLE 2
List of codons and amino acids

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |

| | | | | | | |
|---|---|---|---|---|---|---|
| Threonine | T | Thr | ACA | ACC | ACG | ACT |
| Valine | V | Val | GTA | GTC | GTG | GTT |

| Tryptophan | W | Trp | TGG | |
|---|---|---|---|---|
| Tyrosine | Y | Tyr | TAC | TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

### Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing-in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (VH or VL), and specific complementarity-determining regions (CDRs) of VH or VL.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

### Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 μm. In some instances, the distance between the centers of two adjacent loci is less than about 200, 150, 100, 80, 70, 60, 50, 40, 30, 20 or 10 μm. In some instances, each locus has a width of about 0.5, 1, 2, 3, 4, 5, 6, 7, 8, 9, 10, 20, 30, 40, 50, 60, 70, 80, 90 or 100 μm. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 μm. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

### Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetraflouroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

### Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 μm. In some instances, the diameter of a channel, locus, or both channel and locus is less than about 100, 90, 80, 70, 60, 50, 40, 30, 20 or 10 μm. In some instances, the distance between the center of two adjacent channels, loci, or channels and loci is from about 1-500, 1-200, 1-100, 5-200, 5-100, 5-50, or 5-30, for example, about 20 um.

### Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with 12/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

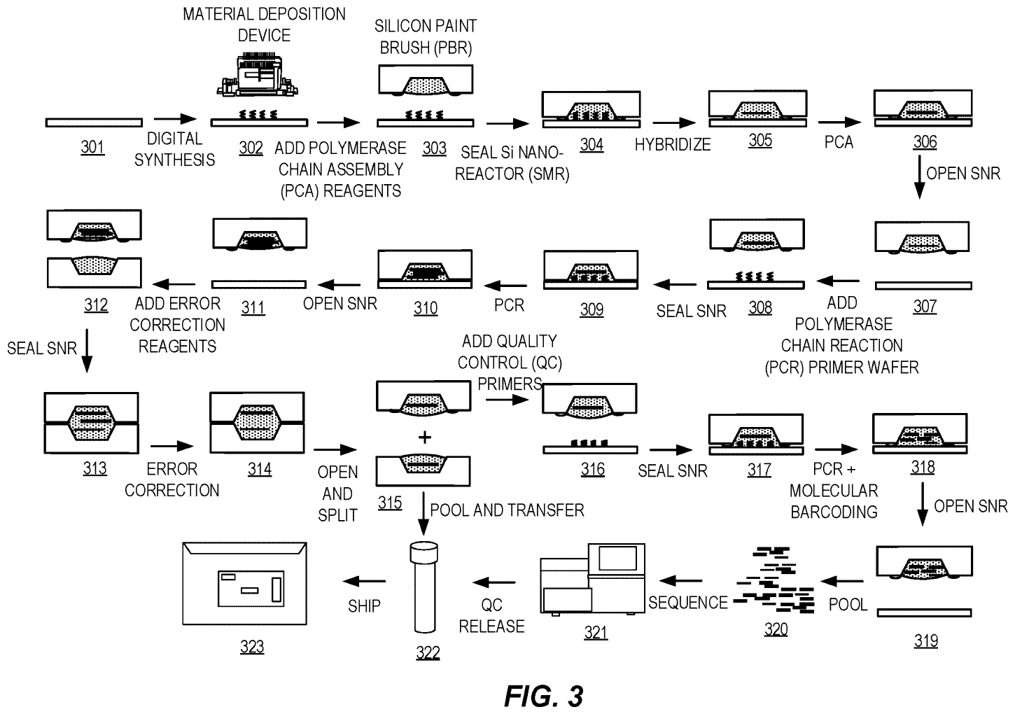

Referring to the Figures, FIG. 3 illustrates an exemplary process workflow for synthesis of nucleic acids (e.g., genes) from shorter nucleic acids. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the workflow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 303. Prior to or after the sealing 304 of the polynucleotides, a reagent is added to release the polynucleotides from the substrate. In the exemplary workflow, the polynucleotides are released subsequent to sealing of the nanoreactor 305. Once released, fragments of single stranded polynucleotides hybridize in order to span an entire long range sequence of DNA. Partial hybridization 305 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 306.

After PCA is complete, the nanoreactor is separated from the device 307 and positioned for interaction with a device having primers for PCR 308. After sealing, the nanoreactor is subject to PCR 309 and the larger nucleic acids are amplified. After PCR 310, the nanochamber is opened 311, error correction reagents are added 312, the chamber is sealed 313 and an error correction reaction occurs to remove mismatched base pairs and/or strands with poor complementarity from the double stranded PCR amplification products 314. The nanoreactor is opened and separated 315. Error corrected product is next subject to additional processing steps, such as PCR and molecular bar coding, and then packaged 322 for shipment 323.

In some instances, quality control measures are taken. After error correction, quality control steps include for example interaction with a wafer having sequencing primers for amplification of the error corrected product 316, sealing the wafer to a chamber containing error corrected amplification product 317, and performing an additional round of amplification 318. The nanoreactor is opened 319 and the products are pooled 320 and sequenced 321. After an acceptable quality control determination is made, the packaged product 322 is approved for shipment 323.

In some instances, a nucleic acid generated by a workflow such as that in FIG. 3 is subject to mutagenesis using overlapping primers disclosed herein. In some instances, a library of primers are generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302.

### Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

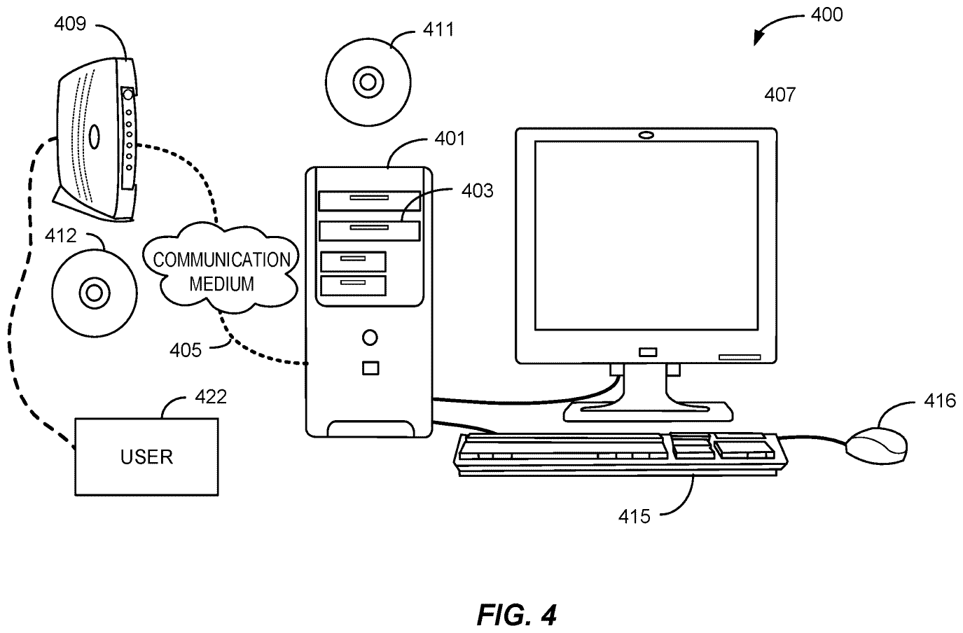

The computer system 400 illustrated in FIG. 4 may be understood as a logical apparatus that can read instructions from media 411 and/or a network port 405, which can optionally be connected to server 409 having fixed media 412. The system, such as shown in FIG. 4 can include a CPU 401, disk drives 403, optional input devices such as keyboard 415 and/or mouse 416 and optional monitor 407. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 422 as illustrated in FIG. 4.

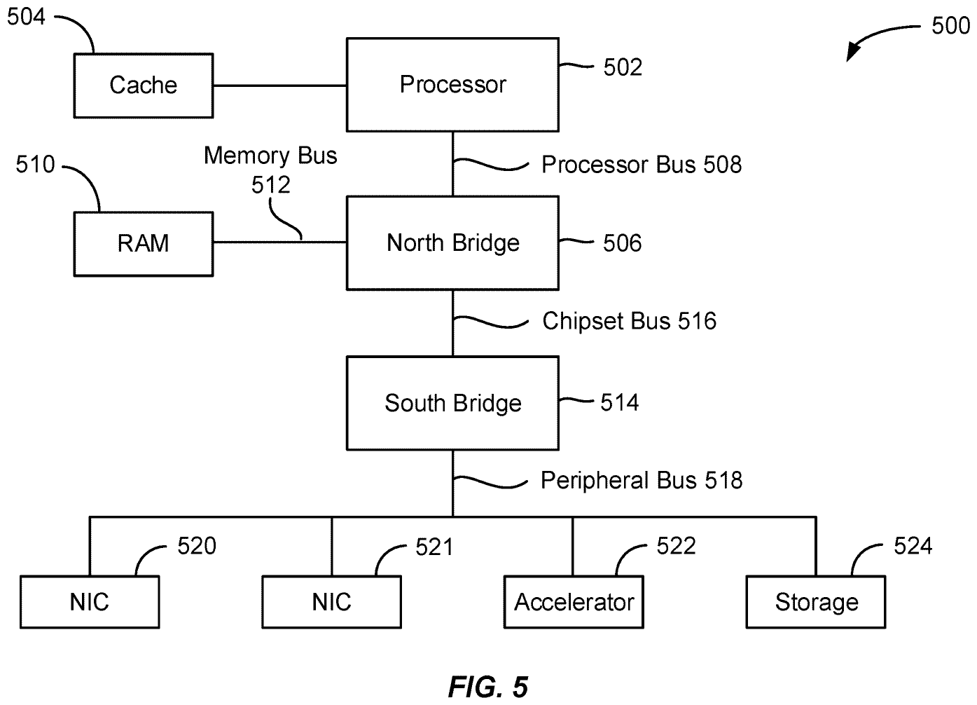

FIG. 5 is a block diagram illustrating a first example architecture of a computer system 500 that can be used in connection with example instances of the present disclosure. As depicted in FIG. 5, the example computer system can include a processor 502 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 5, a high speed cache 504 can be connected to, or incorporated in, the processor 502 to provide a high speed memory for instructions or data that have been recently, or are frequently, used by the processor 502. The processor 502 is connected to a north bridge 506 by a processor bus 508. The north bridge 506 is connected to random access memory (RAM) 510 by a memory bus 512 and manages access to the RAM 510 by the processor 502. The north bridge 506 is also connected to a south bridge 514 by a chipset bus 516. The south bridge 514 is, in turn, connected to a peripheral bus 518. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 518. In some alternative architectures, the functionality of the north bridge can be incorporated into the processor instead of using a separate north bridge chip. In some instances, system 500 can include an accelerator card 522 attached to the peripheral bus 518. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 524 and can be loaded into RAM 510 and/or cache 504 for use by the processor. The system 500 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 500 also includes network interface cards (NICs) 520 and 521 connected to the peripheral bus for providing network interfaces to external storage, such as Network Attached Storage (NAS) and other computer systems that can be used for distributed parallel processing.

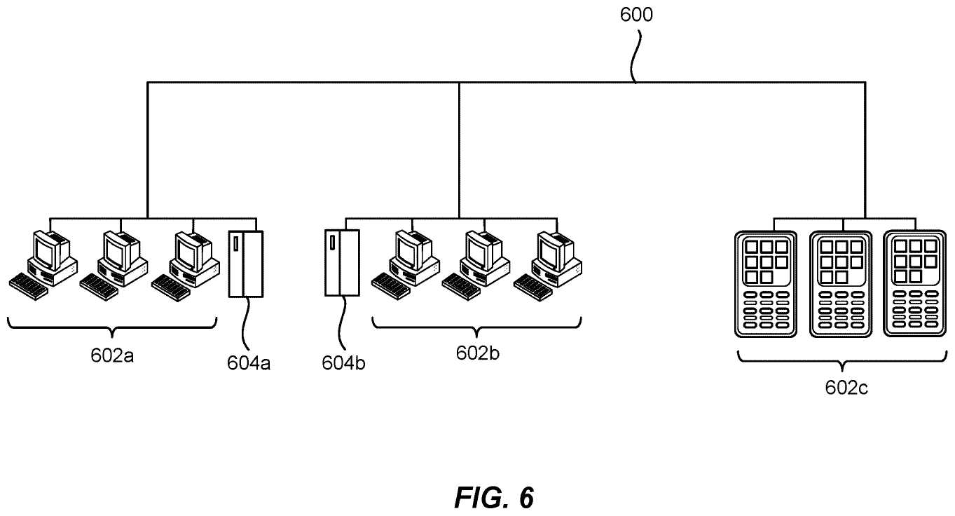

FIG. 6 is a diagram showing a network 600 with a plurality of computer systems 602a, and 602b, a plurality of cell phones and personal data assistants 602c, and Network Attached Storage (NAS) 604a, and 604b. In example instances, systems 602a, 602b, and 602c can manage data storage and optimize data access for data stored in Network Attached Storage (NAS) 604a and 604b. A mathematical model can be used for the data and be evaluated using distributed parallel processing across computer systems 602a, and 602b, and cell phone and personal data assistant systems 602c. Computer systems 602a, and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

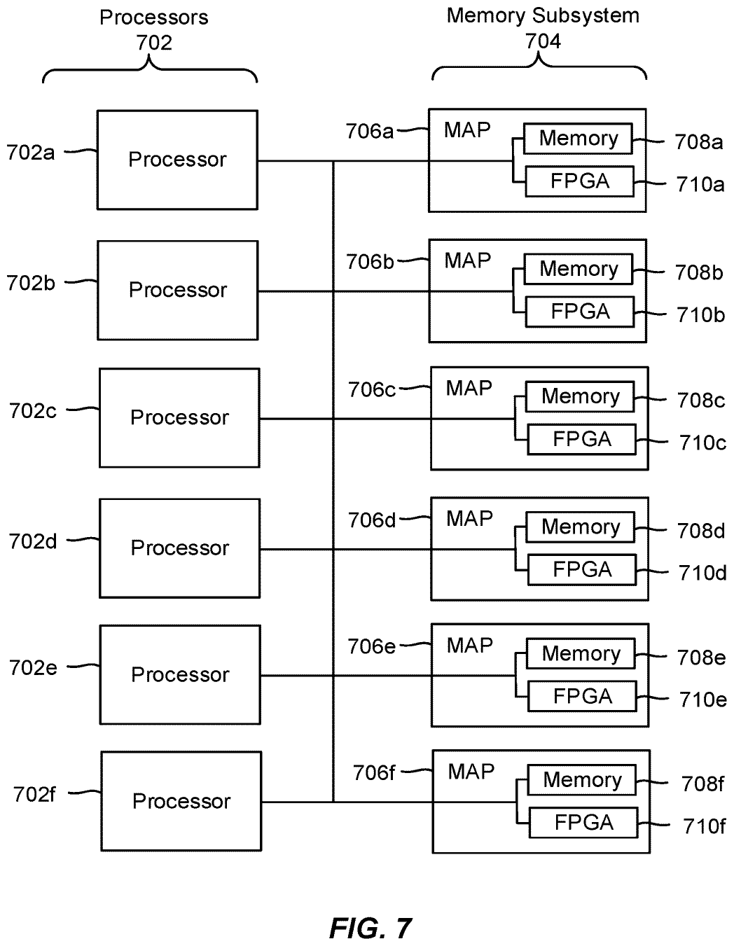

FIG. 7 is a block diagram of a multiprocessor computer system 700 using a shared virtual address memory space in accordance with an example instance. The system includes a plurality of processors 702a-f that can access a shared memory subsystem 704. The system incorporates a plurality of programmable hardware memory algorithm processors (MAPs) 706a-f in the memory subsystem 704. Each MAP 706a-f can comprise a memory 708a-f and one or more field programmable gate arrays (FPGAs) 710a-f. The MAP provides a configurable functional unit and particular algorithms or portions of algorithms can be provided to the FPGAs 710a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 708a-f, allowing it to execute tasks independently of, and asynchronously from the respective microprocessor 702a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 5, system on chips (SOCs), application specific integrated circuits (ASICs), or other processing and logic elements. For example, the Set Processor and Optimizer can be implemented with hardware acceleration through the use of a hardware accelerator card, such as accelerator card 522 illustrated in FIG. 5.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch 02 at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-mer Sequence on an Oligonucleotide Synthesis Device

A two dimensional oligonucleotide synthesis device was assembled into a flowcell, which was connected to a flowcell (Applied Biosystems (AB1394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 2. 5′AGACAATCAACCATTTGGGGTGGACAGCCTTGACCTCTAGACTTCGGCAT ##TTTTTTT TTT3′ (SEQ ID NO.: 2), where #denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 3 and an ABI synthesizer.

TABLE 3

| General DNA Synthesis | | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% o pyridine, 1000 water, and 70% o THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip.

### Example 3: Synthesis of a 100-mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT ##TTTTTTTTTT3′, where #denotes Thymidine-succinyl hexamide CED phosphoramidite (CLP-2244 from ChemGenes); SEQ ID NO.: 3) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument.

All ten samples from the two chips were further PCR amplified using a forward (5′ATGCGGGGTTCTCATCATC3′; SEQ ID NO.: 4) and a reverse (5′CGGGATCCTTATCGTCATCG3′; SEQ ID NO.: 5) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program: 98° C., 30 sec 98° C., 10 sec; 63° C., 10 sec; 72° C., 10 sec; repeat 12 cycles 72° C., 2 min

The PCR products were also run on a BioAnalyzer, demonstrating sharp peaks at the 100-mer position. Next, the PCR amplified samples were cloned, and Sanger sequenced. Table 4 summarizes the results from the Sanger sequencing for samples taken from spots 1-5 from chip 1 and for samples taken from spots 6-10 from chip 2.

TABLE 4
Sequencing results

| Spot | Error rate | | Cycle efficiency |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89% o of the 100-mers that were sequenced were perfect sequences with no errors, corresponding to 233 out of 262.

Table 5 summarizes error characteristics for the sequences obtained from the polynucleotide samples from spots 1-10.

TABLE 5
Error characteristics

| Sample | | | | | |
|---|---|---|---|---|---|
| ID/Spot | | | | | |
| no. | OSA_0046/1 | OSA_0047/2 | OSA_0048/3 | OSA_0049/4 | OSA_0050/5 |
| Total | 32 | 32 | 32 | 32 | 32 |
| Sequences | | | | | |
| Sequencing | 25 of 28 | 27 of 27 | 26 of 30 | 21 of 23 | 25 of 26 |
| Quality | | | | | |
| Oligo | 23 of 25 | 25 of 27 | 22 of 26 | 18 of 21 | 24 of 25 |
| Quality | | | | | |
| ROI | 2500 | 2698 | 2561 | 2122 | 2499 |
| Match | | | | | |
| Count | | | | | |
| ROI | 2 | 2 | 1 | 3 | 1 |
| Mutation | | | | | |
| ROI Multi | 0 | 0 | 0 | 0 | 0 |
| Base | | | | | |
| Deletion | | | | | |
| ROI Small | 1 | 0 | 0 | 0 | 0 |
| Insertion | | | | | |
| ROI | 0 | 0 | 0 | 0 | 0 |
| Single | | | | | |
| Base | | | | | |
| Deletion | | | | | |
| Large | 0 | 0 | 1 | 0 | 0 |
| Deletion | | | | | |
| Count | | | | | |
| Mutation: | 2 | 2 | 1 | 2 | 1 |
| G > A | | | | | |
| Mutation: | 0 | 0 | 0 | 1 | 0 |
| T > C | | | | | |
| ROI Error | 3 | 2 | 2 | 3 | 1 |
| Count | | | | | |
| ROI Error | Err: ~1 | Err: ~1 | Err: ~1 | Err: ~1 | Err: ~1 |
| Rate | in 834 | in 1350 | in 1282 | in 708 | in 2500 |
| ROI | MP Err: | MP Err: | MP Err: | MP Err: | MP Err: |
| Minus | ~1 in | ~1 in | ~1 in | ~1 in | ~1 in |
| Primer | 763 | 824 | 780 | 429 | 1525 |
| Error Rate | | | | | |
| Sample | | | | | |
| ID/Spot | | | | | |
| no. | OSA_0051/6 | OSA_0052/7 | OSA_0053/8 | OSA_0054/9 | OSA_0055/10 |
| Total | 32 | 32 | 32 | 32 | 32 |
| Sequences | | | | | |
| Sequencing | 29 of 30 | 27 of 31 | 29 of 31 | 28 of 29 | 25 of 28 |
| Quality | | | | | |
| Oligo | 25 of 29 | 22 of 27 | 28 of 29 | 26 of 28 | 20 of 25 |
| Quality | | | | | |
| ROI | 2666 | 2625 | 2899 | 2798 | 2348 |
| Match | | | | | |
| Count | | | | | |
| ROI | 0 | 2 | 1 | 2 | 1 |
| Mutation | | | | | |
| ROI Multi | 0 | 0 | 0 | 0 | 0 |
| Base | | | | | |
| Deletion | | | | | |
| ROI Small | 0 | 0 | 0 | 0 | 0 |
| Insertion | | | | | |
| ROI | 0 | 0 | 0 | 0 | 0 |
| Single | | | | | |
| Base | | | | | |
| Deletion | | | | | |
| Large | 1 | 1 | 0 | 0 | 0 |
| Deletion | | | | | |
| Count | | | | | |
| Mutation: | 0 | 2 | 1 | 2 | 1 |
| G > A | | | | | |
| Mutation: | 0 | 0 | 0 | 0 | 0 |
| T > C | | | | | |
| ROI Error | 1 | 3 | 1 | 2 | 1 |
| Count | | | | | |
| ROI Error | Err: ~1 | Err: ~1 | Err: ~1 | Err: ~1 | Err: ~1 |
| Rate | in 2667 | in 876 | in 2900 | in 1400 | in 2349 |
| ROI | MP Err: | MP Err: | MP Err: | MP Err: | MP Err: |
| Minus | ~1 in | ~1 in | ~1 in | ~1 in | ~1 in |
| Primer | 1615 | 531 | 1769 | 854 | 1451 |
| Error Rate | | | | | |

### Example 4: Design of GLP1R Binding Domains Based on Peptide Ligand Interactions

GLP1R binding domains were designed based on interaction surfaces between peptide ligands that interact with GLP1R. Motif variants were generated based on the interaction surface of the peptides with the ECD as well as with the N-terminal GLP1R ligand interaction surface. This was done using structural modeling. Exemplary motif variants were created based on glucagon like peptide's interaction with GLP1R as seen in Table 6. The motif variant sequences were generated using the following sequence from glucagon like peptide: HAEGTFTSDVSSYLEGQAAKEFIAWLVKGRG (SEQ ID NO: 6).

TABLE 6
Variant amino acid sequences for
glucagon like peptide

| SEQ | | |
|---|---|---|
| ID | | |
| NO. | Variant | Amino Acid Sequence |
| 7 | 1 | sggggsggggsggggHAEGTFTSDVSSY |
| | | LEGQAAKEFIAWLV |
| 8 | 2 | sggggsggggsggggAEGTFTSDVSSYL |
| | | EGQAAKEFIAWLV |
| 9 | 3 | sggggsggggsggggEGTFTSDVSSYLE |
| | | GQAAKEFIAWLV |
| 10 | 4 | sggggsggggsggggGTFTSDVSSYLEG |
| | | QAAKEFIAWLV |
| 11 | 5 | sggggsggggsggggTFTSDVSSYLEGQ |
| | | AAKEFIAWLV |
| 12 | 6 | sggggsggggsggggFTSDVSSYLEGQA |
| | | AKEFIAWLV |
| 13 | 7 | sggggsggggsggggTSDVSSYLEGQAA |
| | | KEFIAWLV |
| 14 | 8 | sggggsggggsggggSDVSSYLEGQAAK |
| | | EFIAWLV |
| 15 | 9 | sggggsggggsggggDVSSYLEGQAAKE |
| | | FIAWLV |

### Example 5: Design of Antibody Scaffolds

To generate scaffolds, structural analysis, repertoire sequencing analysis of the heavy chain, and specific analysis of heterodimer high-throughput sequencing datasets were performed. Each heavy chain was associated with each light chain scaffold. Each heavy chain scaffold was assigned 5 different long CDR-H3 loop options. Each light chain scaffold was assigned 5 different L3 scaffolds. The heavy chain CDR-H3 stems were chosen from the frequently observed long H3 loop stems (10 amino acids on the N-terminus and the C-terminus) found both across individuals and across V-gene segments. The light chain scaffold L3s were chosen from heterodimers comprising long H3s. Direct heterodimers based on information from the Protein Data Bank (PDB) and deep sequencing datasets were used in which CDR H1, H2, L1, L2, L3, and CDR-H3 stems were fixed. The various scaffolds were then formatted for display on phage to assess for expression.

### Structural Analysis

About 2,017 antibody structures were analyzed from which 22 structures with long CDR-H3s of at least 25 amino acids in length were observed. The heavy chains included the following: IGHV1-69, IGHV3-30, IGHV4-49, and IGHV3-21. The light chains identified included the following: IGLV3-21, IGKV3-11, IGKV2-28, IGKV1-5, IGLV1-51, IGLV1-44, and IGKV1-13. In the analysis, four heterodimer combinations were observed multiple times including: IGHV4-59/61-IGLV3-21, IGHV3-21-IGKV2-28, IGHV1-69-IGKV3-11, and IGHV1-69-IGKV1-5. An analysis of sequences and structures identified intra-CDR-H3 disulfide bonds in a few structures with packing of bulky side chains such as tyrosine in the stem providing support for long H3 stability. Secondary structures including beta-turn-beta sheets and a “hammerhead” subdomain were also observed.

### Repertoire Analysis

A repertoire analysis was performed on 1,083,875 IgM+/CD27−naïve B cell receptor (BCR) sequences and 1,433,011 CD27+ sequences obtained by unbiased 5′RACE from 12 healthy controls. The 12 healthy controls comprised equal numbers of male and female and were made up of 4 Caucasian, 4 Asian, and 4 Hispanic individuals. The repertoire analysis demonstrated that less than 1% of the human repertoire comprises BCRs with CDR-H3s longer than 21 amino acids. A V-gene bias was observed in the long CDR3 subrepertoire, with IGHV1-69, IGHV4-34, IGHV1-18, and IGHV1-8 showing preferential enrichment in BCRs with long H3 loops. A bias against long loops was observed for IGHV3-23, IGHV4-59/61, IGHV5-51, IGHV3-48, IGHV3-53/66, IGHV3-15, IGHV3-74, IGHV3-73, IGHV3-72, and IGHV2-70. The IGHV4-34 scaffold was demonstrated to be autoreactive and had a short half-life.

Viable N-terminal and C-terminal CDR-H3 scaffold variation for long loops were also designed based on the 5′RACE reference repertoire. About 81,065 CDR-H3s of amino acid length 22 amino acids or greater were observed. By comparing across V-gene scaffolds, scaffold-specific H3 stem variation was avoided as to allow the scaffold diversity to be cloned into multiple scaffold references.

### Heterodimer Analysis

Heterodimer analysis was performed on scaffolds and variant sequences and lengths of the scaffolds were assayed.

### Structural Analysis

Structural analysis was performed using GPCR scaffolds of variant sequences and lengths were assayed.

### Example 6: Generation of GPCR Antibody Libraries

Based on GPCR-ligand interaction surfaces and scaffold arrangements, libraries were designed and de novo synthesized. See Example 4. 10 variant sequences were designed for the variable domain, heavy chain, 237 variant sequences were designed for the heavy chain complementarity determining region 3, and 44 variant sequences were designed for the variable domain, light chain. The fragments were synthesized as three fragments following similar methods as described in Examples 1-3.

Following de novo synthesis, 10 variant sequences were generated for the variable domain, heavy chain, 236 variant sequences were generated for the heavy chain complementarity determining region 3, and 43 variant sequences were designed for a region comprising the variable domain, light chain and CDR-L3 and of which 9 variants for variable domain, light chain were designed. This resulted in a library with about 105 diversity (10×236×43). This was confirmed using next generation sequencing (NGS) with 16 million reads. The normalized sequencing reads for each of the 10 variants for the variable domain, heavy chain was about 1 (data not shown). The normalized sequencing reads for each of the 43 variants for the variable domain, light chain was about 1 (data not shown). The normalized sequencing reads for 236 variant sequences for the heavy chain complementarity determining region 3 were about 1 (data not shown).

The various light and heavy chains were then tested for expression and protein folding. The 10 variant sequences for variable domain, heavy chain included the following: IGHV1-18, IGHV1-69, IGHV1-8 IGHV3-21, IGHV3-23, IGHV3-30/33m, IGHV3-28, IGHV3-74, IGHV4-39, and IGHV4-59/61. Of the 10 variant sequences, IGHV1-18, IGHV1-69, and IGHV3-30/33m exhibited improved characteristics such as improved thermostability. 9 variant sequences for variable domain, light chain included the following: IGKV1-39, IGKV1-9, IGKV2-28, IGKV3-11, IGKV3-15, IGKV3-20, IGKV4-1, IGLV1-51, and IGLV2-14. Of the 9 variant sequences, IGKV1-39, IGKV3-15, IGLV1-51, and IGLV2-14 exhibited improved characteristics such as improved thermostability.

### Example 7: Expression of GPCR Antibody Libraries in HEK293 Cells

Following generation of GPCR antibody libraries, about 47 GPCRs were selected for screening. GPCR constructs about 1.8 kb to about 4.5 kb in size were designed in a pCDNA3.1 vector. The GPCR constructs were then synthesized following similar methods as described in Examples 2-4 including hierarchal assembly. Of the 47 GPCR constructs, 46 GPCR constructs were synthesized.

The synthesized GPCR constructs were transfected in HEK293 and assayed for expression using immunofluorescence. HEK293 cells were transfected with the GPCR constructs comprising an N-terminally hemagglutinin (HA)-tagged human Yi receptor. Following 24-48 hours of transfection, cells were washed with phosphate buffered saline (PBS) and fixed with 4% paraformaldehyde. Cells were stained using fluorescent primary antibody directed towards the HA tag or secondary antibodies comprising a fluorophore and DAPI to visualize the nuclei in blue. Human Yi receptor was visualized on the cell surface in non-permeabilized cells and on the cell surface and intracellularly in permeabilized cells.

GPCR constructs were also visualized by designing GPCR constructs comprising auto-fluorescent proteins. Human Yi receptor comprised EYFP fused to its C-terminus, and human Y5 receptor comprised ECFP fused to its C-terminus. HEK293 cells were transfected with human Yi receptor or co-transfected with human Yi receptor and human Y5 receptor. Following transfection cells were washed and fixed with 4% paraformaldehyde. Cells were stained with DAPI. Localization of human Yi receptor and human Y5 receptor were visualized by fluorescence microscopy.

### Example 8: Design of Immunoglobulin Library

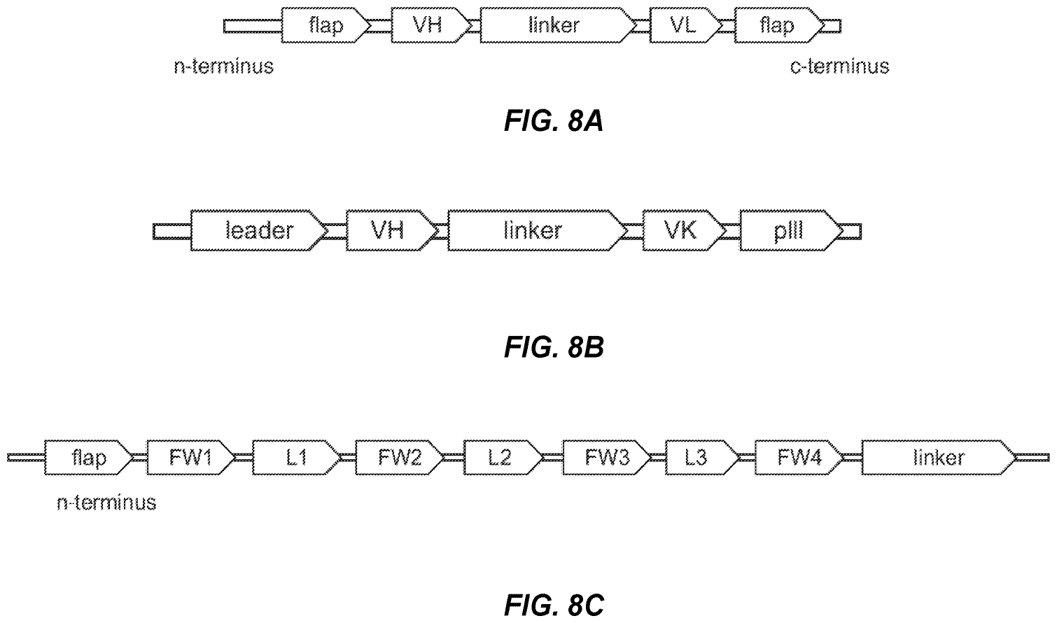

An immunoglobulin scaffold library was designed for placement of GPCR binding domains and for improving stability for a range of GPCR binding domain encoding sequences. The immunoglobulin scaffold included a VH domain attached with a VL domain with a linker. Variant nucleic acid sequences were generated for the framework elements and CDR elements of the VH domain and VL domain. The structure of the design is shown in FIG. 8A. A full domain architecture is shown in FIG. 8B. Sequences for the leader, linker, and pIII are listed in Table 7.

TABLE 7
Nucleotide sequences

| SEQ | | |
|---|---|---|
| ID NO | Domain | Sequence |
| 16 | Leader | GCAGCCGCTGGCTTGCTGCTGCTGGCAGCTCA |
| | | GCCGGCCATGGCC |
| 17 | Linker | GCTAGCGGTGGAGGCGGTTCAGGCGGAGGTGG |
| | | CTCTGGCGGTGGCGGATCGCATGCATCC |
| 18 | pIII | CGCGCGGCCGCTGGAAGCGGCTCCCACCATCA |
| | | CCATCACCAT |

The VL domains that were designed include IGKV1-39, IGKV3-15, IGLV1-51, and IGLV2-14. Each of four VL domains were assembled with their respective invariant four framework elements (FW, FW2, FW3, FW4) and variable 3 CDR (Li, L2, L3) elements. For IGKV1-39, there was 490 variants designed for Li, 420 variants designed for L2, and 824 variants designed for L3 resulting in a diversity of 1.7×10⁸ (490*420*824). For IGKV3-15, there was 490 variants designed for Li, 265 variants designed for L2, and 907 variants designed for L3 resulting in a diversity of 1.2×10⁸ (490*265*907). For IGLV1-5, there was 184 variants designed for Li, 151 variants designed for L2, and 824 variants designed for L3 resulting in a diversity of 2.3×10⁷ (184*151*824). IGLV2-14, 967 variants designed for Li, 535 variants designed for L2, and 922 variants designed for L3 resulting in a diversity of 4.8 10⁸ (967*535*922). Table 8 lists the amino acid sequences and nucleotide sequences for the four framework elements (FW1, FW2, FW3, FW4) for IGLV1-51. Table 9 lists the variable 3 CDR (L1, L2, L3) elements for IGLV1-51. Variant amino acid sequences and nucleotide sequences for the four framework elements (FW1, FW2, FW3, FW4) and the variable 3 CDR (Li, L2, L3) elements were also designed for IGKV1-39, IGKV3-15, and IGLV2-14.

TABLE 8
Sequences for IGLV1-51 framework elements

| | | | | |
|---|---|---|---|---|
| | SEQ | | SEQ | |
| | ID | Amino Acid | ID | Nucleotide |
| Element | NO | Sequence | NO | Sequence |

IGLV1-51

| | | | | |
|---|---|---|---|---|
| FW1 | 19 | QSVLTQPPSVSA | 20 | CAGTCTGTGTTGACGCAGC |
| | | APGQKVTISC | | CGCCCTCAGTGTCTGCGGC |
| | | | | CCCAGGACAGAAGGTCACC |
| | | | | ATCTCCTGC |
| FW2 | 21 | WYQQLPGTAPKL | 22 | TGGTATCAGCAGCTCCCAG |
| | | LIY | | GAACAGCCCCCAAACTCCT |
| | | | | CATTTAT |
| FW3 | 23 | GIPDRFSGSKSG | 24 | GGGATTCCTGACCGATTCT |
| | | TSATLGITGLQT | | CTGGCTCCAAGTCTGGCAC |
| | | GDEADYY | | GTCAGCCACCCTGGGCATC |
| | | | | ACCGGACTCCAGACTGGGG |
| | | | | ACGAGGCCGATTATTAC |
| FW4 | 25 | GGGTKLTVL | 26 | GGCGGAGGGACCAAGCTGA |
| | | | | CCGTCCTA |

TABLE 9
Sequences for IGLV1-51 CDR elements

| | | | |
|---|---|---|---|
| SEQ ID | | SEQ | |
| NO | Amino Acid Sequence | ID NO | Nucleotide Sequence |

IGLV1-51-L1

| | | | |
|---|---|---|---|
| 27 | SGSSSNIGSNHVS | 210 | TCTGGAAGCAGCTCCAACATTGGGAGTAATCATGTATCC |
| 28 | SGSSSNIGNNYLS | 211 | TCTGGAAGCAGCTCCAACATTGGGAATAATTATCTATCC |
| 29 | SGSSSNIANNYVS | 212 | TCTGGAAGCAGCTCCAACATTGCGAATAATTATGTATCC |
| 30 | SGSSPNIGNNYVS | 213 | TCTGGAAGCAGCCCCAACATTGGGAATAATTATGTATCG |
| 31 | SGSRSNIGSNYVS | 214 | TCTGGAAGCAGATCCAATATTGGGAGTAATTATGTTTCG |
| 32 | SGSSSNVGDNYVS | 215 | TCTGGAAGCAGCTCCAACGTTGGCGATAATTATGTTTCC |
| 33 | SGSSSNIGIQYVS | 216 | TCTGGAAGCAGCTCCAACATTGGGATTCAATATGTATCC |
| 34 | SGSSSNVGNNFVS | 217 | TCTGGAAGCAGCTCCAATGTTGGTAACAATTTTGTCTCC |
| 35 | SGSASNIGNNYVS | 218 | TCTGGAAGCGCCTCCAACATTGGGAATAATTATGTATCC |
| 36 | SGSGSNIGNNDVS | 219 | TCTGGAAGCGGCTCCAATATTGGGAATAATGATGTGTCC |
| 37 | SGSISNIGNNYVS | 220 | TCTGGAAGCATCTCCAACATTGGTAATAATTATGTATCC |
| 38 | SGSISNIGKNYVS | 221 | TCTGGAAGCATCTCCAACATTGGGAAAAATTATGTGTCG |
| 39 | SGSSSNIGHNYVS | 222 | TCTGGAAGCAGCTCCAACATTGGGCATAATTATGTATCG |
| 40 | PGSSSNIGNNYVS | 223 | CCTGGAAGCAGCTCCAACATTGGGAATAATTATGTATCC |
| 41 | SGSTSNIGIHYVS | 224 | TCTGGAAGCACCTCCAACATTGGAATTCATTATGTATCC |
| 42 | SGSSSNIGSHYVS | 225 | TCTGGAAGCAGCTCCAACATTGGCAGTCATTATGTTTCC |
| 43 | SGSSSNIGNEYVS | 226 | TCCGGAAGCAGCTCCAACATTGGAAATGAATATGTATCC |
| 44 | SGSTSNIGNNYIS | 227 | TCTGGAAGCACCTCCAACATTGGAAATAATTATATATCG |
| 45 | SGSSSNIGNHFVS | 228 | TCTGGAAGCAGCTCCAATATTGGGAATCATTTTGTATCG |
| 46 | SGSSSNIGNNYVA | 229 | TCTGGAAGCAGCTCCAACATTGGGAATAATTATGTGGCC |
| 47 | SGSSSNIGSYYVS | 230 | TCTGGAAGCAGCTCCAACATTGGAAGTTATTATGTATCC |
| 48 | SGSGFNIGNNYVS | 231 | TCTGGAAGTGGTTTCAACATTGGGAATAATTATGTCTCT |
| 49 | SGSTSNIGNNYVS | 232 | TCTGGAAGCACCTCCAACATTGGGAATAATTATGTGTCC |
| 50 | SGSSSDIGNNYVS | 233 | TCTGGAAGCAGCTCCGACATTGGCAATAATTATGTATCC |
| 51 | SGSSSNIGNNVVS | 234 | TCTGGAAGCAGCTCCAACATTGGGAATAATGTTGTATCC |
| 52 | SGSKSNIGKNYVS | 235 | TCTGGAAGCAAGTCTAACATTGGGAAAAATTATGTATCC |
| 53 | SGSSTNIGNNYVS | 236 | TCTGGAAGCAGCACCAACATTGGGAATAATTATGTATCC |
| 54 | SGSISNIGDNYVS | 237 | TCTGGAAGCATCTCCAACATTGGGGATAATTATGTATCC |
| 55 | SGSSSNIGSKDVS | 238 | TCTGGAAGCAGCTCCAACATTGGGAGTAAGGATGTATCA |
| 56 | SGSSSNIENNDVS | 239 | TCTGGAAGCAGCTCCAACATTGAGAATAATGATGTATCG |
| 57 | SGSSSNIGNHYVS | 240 | TCTGGAAGCAGCTCCAACATTGGGAATCATTATGTATCC |
| 58 | SGSSSNIGKDFVS | 241 | TCTGGAAGCAGCTCCAACATTGGGAAGGATTTTGTCTCC |
| 59 | SGSTSNIGSNFVS | 242 | TCTGGCAGTACTTCCAACATCGGAAGTAATTTTGTTTCC |
| 60 | SGSTSNIGHNYVS | 243 | TCTGGAAGCACCTCCAACATTGGGCATAATTATGTATCC |
| 61 | SASSSNIGNNYVS | 244 | TCTGCAAGCAGCTCCAACATTGGGAATAATTATGTATCC |
| 62 | SGSSSSIGNNYVS | 245 | TCTGGAAGCAGCTCCAGCATTGGCAATAATTATGTATCC |
| 63 | SGSSSTIGNNYVS | 246 | TCTGGAAGCAGCTCCACCATTGGGAATAATTATGTATCC |
| 64 | SGSSSNIENNYVS | 247 | TCTGGAAGCAGCTCCAACATTGAAAATAATTATGTATCC |
| 65 | SGSSSNIGNQYVS | 248 | TCTGGAAGCAGCTCCAACATTGGGAATCAGTATGTATCC |
| 66 | SGSSSNIGNNYVF | 249 | TCTGGAAGCAGCTCCAACATTGGGAATAATTATGTATTC |
| 67 | SGSSSNIGRNYVS | 250 | TCTGGAAGCAGCTCCAACATTGGGAGGAATTATGTCTCC |
| 68 | SGGSSNIGNYYVS | 251 | TCTGGAGGCAGCTCCAACATTGGAAATTATTATGTATCG |
| 69 | SGSSSNIGDNYVS | 252 | TCTGGAAGCAGCTCCAACATTGGAGATAATTATGTCTCC |
| 70 | SGGSSNIGINYVS | 253 | TCTGGAGGCAGCTCCAACATTGGAATTAATTATGTATCC |
| 71 | SGGSSNIGKNYVS | 254 | TCTGGAGGCAGCTCCAACATTGGGAAGAATTATGTATCC |
| 72 | SGSSSNIGKRSVS | 255 | TCTGGAAGCAGCTCCAACATTGGGAAGAGATCTGTATCG |
| 73 | SGSRSNIGNNYVS | 256 | TCTGGAAGCAGATCCAACATTGGGAATAACTATGTATCC |
| 74 | SGSSSNIGNNLVS | 257 | TCGGGAAGCAGCTCCAACATTGGGAATAATCTTGTTTCC |
| 75 | SGSSSNIGINYVS | 258 | TCTGGAAGCAGCTCCAACATTGGGATCAATTATGTATCC |
| 76 | SGSSSNIGNNFVS | 259 | TCTGGAAGCAGCTCCAACATCGGGAATAATTTTGTATCC |
| 77 | SGTSSNIGRNFVS | 260 | TCTGGAACCAGCTCCAACATTGGCAGAAATTTTGTATCC |
| 78 | SGRRSNIGNNYVS | 261 | TCTGGAAGGAGGTCCAACATTGGAAATAATTATGTGTCC |
| 79 | SGGSFNIGNNYVS | 262 | TCTGGAGGCAGCTTCAATATTGGGAATAATTATGTATCC |
| 80 | SGSTSNIGENYVS | 263 | TCTGGAAGCACTTCCAACATTGGGGAGAATTATGTGTCC |
| 81 | SGSSSNIGSDYVS | 264 | TCTGGAAGCAGCTCCAATATTGGGAGTGATTATGTATCC |
| 82 | SGTSSNIGSNYVS | 265 | TCTGGAACCAGCTCCAACATTGGGAGTAATTATGTATCC |
| 83 | SGSSSNIGTNFVS | 266 | TCTGGAAGCAGCTCCAACATTGGGACTAATTTTGTATCC |
| 84 | SGSSSNFGNNYVS | 267 | TCTGGAAGCAGCTCCAACTTTGGGAATAATTATGTATCC |
| 85 | SGSTSNIGNNHVS | 268 | TCTGGAAGCACCTCCAACATTGGGAATAATCATGTATCC |
| 86 | SGSSSNIGNDFVS | 269 | TCTGGAAGCAGCTCCAACATTGGGAATGATTTTGTATCC |
| 87 | SGSSSDIGDNYVS | 270 | TCTGGAAGCAGCTCCGACATTGGCGATAATTATGTGTCC |
| 88 | SGSSSNIGKYYVS | 271 | TCTGGAAGCAGCTCCAACATTGGGAAATATTATGTATCC |
| 89 | SGSSSNIGGNYVS | 272 | TCTGGAAGCAGCTCCAACATTGGCGGTAATTATGTATCC |
| 90 | SGSSSNTGNNYVS | 273 | TCTGGAAGCAGCTCCAACACTGGGAATAATTATGTATCC |
| 91 | SGSSSNVGNNYVS | 274 | TCTGGAAGCAGCTCCAACGTTGGGAATAATTATGTGTCT |
| 92 | SGSSSNIANNFVS | 275 | TCTGGAAGCAGCTCCAACATTGCGAATAATTTTGTATCC |
| 93 | SGSSSNIGNDYVS | 276 | TCTGGAAGCAGCTCCAACATTGGGAATGATTATGTATCC |
| 94 | SGSTSNIENNYVS | 277 | TCTGGAAGCACCTCCAATATTGAGAATAATTATGTTTCC |
| 95 | SGGSSNIGNNDVS | 278 | TCTGGAGGCAGCTCCAATATTGGCAATAATGATGTGTCC |
| 96 | SGSTSNIGNHYVS | 279 | TCTGGAAGCACCTCCAACATTGGGAATCATTATGTATCC |
| 97 | SGSSSNIGDNDVS | 280 | TCAGGAAGCAGCTCCAATATTGGGGATAATGATGTATCC |
| 98 | SGYSSNIGNNYVS | 281 | TCTGGATACAGCTCCAACATTGGGAATAATTATGTATCC |
| 99 | SGSGSNIGNNFVS | 282 | TCTGGAAGCGGCTCCAACATTGGAAATAATTTTGTATCC |
| 100 | SGSSSNIWNNYVS | 283 | TCTGGAAGCAGCTCCAACATTTGGAATAATTATGTATCC |
| 101 | FGSSSNIGNNYVS | 284 | TTTGGAAGCAGCTCCAACATTGGGAATAATTATGTATCC |
| 102 | SGSSSNIEKNYVS | 285 | TCTGGAAGCAGCTCCAACATTGAGAAGAATTATGTATCC |
| 103 | SGSRSNIGNYYVS | 286 | TCTGGAAGTAGATCCAATATTGGAAATTATTATGTATCC |
| 104 | SGTKSNIGNNYVS | 287 | TCTGGAACCAAGTCAAACATTGGGAATAATTATGTATCT |
| 105 | SGSTSNIGNYYVS | 288 | TCTGGAAGCACCTCCAACATTGGGAATTATTATGTATCC |
| 106 | SGTSSNIGNNYVA | 289 | TCTGGAACCAGCTCCAACATTGGGAATAATTATGTGGCC |
| 107 | PGTSSNIGNNYVS | 290 | CCTGGAACCAGCTCCAACATTGGGAATAATTATGTATCC |
| 108 | SGSTSNIGINYVS | 291 | TCCGGAAGCACCTCCAACATTGGGATTAATTATGTATCC |
| 109 | SGSSSNIGSNLVS | 292 | TCTGGAAGCAGCTCCAACATTGGGAGTAATCTGGTATCC |
| 110 | SGSSSNIENNHVS | 293 | TCTGGAAGCAGCTCCAACATTGAGAATAATCATGTATCC |
| 111 | SGTRSNIGNNYVS | 294 | TCTGGAACCAGGTCCAACATCGGCAATAATTATGTTTCG |
| 112 | SGSTSNIGDNYVS | 295 | TCTGGAAGCACCTCCAACATTGGGGACAATTATGTTTCC |
| 113 | SGGSSNIGKNFVS | 296 | TCTGGAGGCAGTTCCAACATTGGGAAGAATTTTGTATCC |
| 114 | SGSRSDIGNNYVS | 297 | TCTGGAAGCAGGTCCGACATTGGGAATAATTATGTATCC |
| 115 | SGTSSNIGNNDVS | 298 | TCTGGAACTAGCTCCAACATTGGGAATAATGATGTATCC |
| 116 | SGSSSNIGSKYVS | 299 | TCTGGAAGCAGCTCCAACATTGGGAGTAAATATGTATCA |
| 117 | SGSSFNIGNNYVS | 300 | TCTGGAAGCAGCTTCAACATTGGGAATAATTATGTATCC |
| 118 | SGSSSNIGNTYVS | 301 | TCTGGAAGCAGCTCCAACATTGGGAATACTTATGTATCC |
| 119 | SGSSSNIGDNHVS | 302 | TCTGGAAGCAGCTCCAATATTGGGGATAATCATGTATCC |
| 120 | SGSSSNIGNNHVS | 303 | TCTGGAAGCAGCTCCAACATTGGCAATAATCATGTTTCC |
| 121 | SGSTSNIGNNDVS | 304 | TCTGGAAGCACCTCCAACATTGGGAATAATGATGTATCC |
| 122 | SGSRSNVGNNYVS | 305 | TCTGGAAGCAGATCCAACGTTGGCAATAATTATGTTTCA |
| 123 | SGGTSNIGKNYVS | 306 | TCCGGAGGCACCTCCAACATTGGGAAGAATTATGTGTCT |
| 124 | SGSSSNIADNYVS | 307 | TCTGGAAGCAGCTCCAACATTGCCGATAATTATGTTTCC |
| 125 | SGSSSNIGANYVS | 308 | TCTGGAAGCAGCTCCAACATTGGCGCCAATTATGTATCC |
| 126 | SGSSSNIGSNYVA | 309 | TCTGGAAGCAGCTCCAACATTGGGAGTAATTATGTGGCC |
| 127 | SGSSSNIGNNFLS | 310 | TCTGGAAGCAGCTCCAACATTGGGAACAATTTTCTCTCC |
| 128 | SGRSSNIGKNYVS | 311 | TCTGGAAGAAGCTCCAACATTGGGAAGAATTATGTATCC |
| 129 | SGSSPNIGANYVS | 312 | TCTGGAAGCAGCCCCAACATTGGGGCTAATTATGTATCC |
| 130 | SGSSSNIGPNYVS | 313 | TCCGGAAGCAGCTCCAACATTGGGCCTAATTATGTGTCC |
| 131 | SGSSSTIGNNYIS | 314 | TCTGGAAGCAGCTCCACCATTGGGAATAATTATATATCC |
| 132 | SGSSSNIGNYFVS | 315 | TCTGGAAGCAGCTCCAACATTGGGAATTATTTTGTATCC |
| 133 | SGSRSNIGNNFVS | 316 | TCTGGAAGCCGCTCCAACATTGGTAATAATTTTGTATCC |
| 134 | SGGSSNIGSNFVS | 317 | TCTGGAGGCAGCTCCAACATTGGGAGTAATTTTGTATCC |
| 135 | SGSSSNIGYNYVS | 318 | TCTGGAAGCAGCTCCAACATTGGGTATAATTATGTATCC |
| 136 | SGTSSNIENNYVS | 319 | TCTGGAACCAGCTCGAACATTGAGAACAATTATGTATCC |
| 137 | SGSSSNIGNYYVS | 320 | TCTGGAAGTAGCTCCAACATTGGGAATTATTATGTATCC |
| 138 | SGSTSNIGKNYVS | 321 | TCTGGAAGCACCTCCAACATTGGGAAGAATTATGTATCC |
| 139 | SGSSSNIGTYYVS | 322 | TCTGGAAGCAGTTCCAACATTGGGACTTATTATGTCTCT |
| 140 | SGSSSNVGKNYVS | 323 | TCTGGAAGCAGCTCCAACGTTGGGAAAAATTATGTATCT |
| 141 | SGSTSNIGDNFVS | 324 | TCTGGAAGCACCTCCAACATTGGGGATAATTTTGTATCC |
| 142 | SGSTSNIGTNYVS | 325 | TCTGGAAGCACCTCCAACATTGGAACTAATTATGTTTCC |
| 143 | SGGTSNIGNNYVS | 326 | TCTGGAGGTACTTCCAACATTGGGAATAATTATGTCTCC |
| 144 | SGSYSNIGNNYVS | 327 | TCTGGAAGCTACTCCAATATTGGGAATAATTATGTATCC |
| 145 | SGSSSNIEDNYVS | 328 | TCTGGAAGCAGCTCCAACATTGAAGATAATTATGTATCC |
| 146 | SGSSSNIGKHYVS | 329 | TCTGGAAGCAGCTCCAACATTGGGAAACATTATGTATCC |
| 147 | SGSGSNIGSNYVS | 330 | TCCGGTTCCGGCTCAAACATTGGAAGTAATTATGTCTCC |
| 148 | SGSSSNIGNNYIS | 331 | TCTGGAAGCAGCTCCAACATTGGAAATAATTATATATCA |
| 149 | SGASSNIGNNYVS | 332 | TCTGGAGCCAGTTCCAACATTGGGAATAATTATGTTTCC |
| 150 | SGRTSNIGNNYVS | 333 | TCTGGACGCACCTCCAACATCGGGAACAATTATGTATCC |
| 151 | SGGSSNIGSNYVS | 334 | TCTGGAGGCAGCTCCAATATTGGGAGTAATTACGTATCC |
| 152 | SGSGSNIGNNYVS | 335 | TCTGGAAGCGGCTCCAACATTGGGAATAATTATGTATCC |
| 153 | SGSTSNIGSNYVS | 336 | TCTGGAAGCACCTCCAACATTGGGAGTAATTATGTATCC |
| 154 | SGSSSSIGNNYVA | 337 | TCTGGAAGCAGCTCCAGCATTGGGAATAATTATGTGGCG |
| 155 | SGSSSNLGNNYVS | 338 | TCTGGAAGCAGTTCCAACCTTGGAAATAATTATGTATCC |
| 156 | SGTSSNIGKNYVS | 339 | TCTGGAACCAGCTCCAACATTGGGAAAAATTATGTATCC |
| 157 | SGSSSDIGNKYIS | 340 | TCTGGAAGCAGCTCCGATATTGGGAACAAGTATATATCC |
| 158 | SGSSSNIGSNYIS | 341 | TCTGGAAGCAGCTCCAACATTGGAAGTAATTACATATCC |
| 159 | SGSTSNIGANYVS | 342 | TCTGGAAGCACCTCCAACATTGGGGCTAACTATGTGTCC |
| 160 | SGSSSNIGNKYVS | 343 | TCTGGAAGCAGCTCCAACATTGGGAATAAGTATGTATCC |
| 161 | SGSSSNIGNNYGS | 344 | TCTGGAAGCAGCTCCAACATTGGGAATAATTATGGATCC |
| 162 | SGSTSNIANNYVS | 345 | TCTGGAAGCACCTCCAACATTGCGAATAATTATGTATCC |
| 163 | SGSYSNIGSNYVS | 346 | TCTGGAAGCTACTCCAATATTGGGAGTAATTATGTATCC |
| 164 | SGSSSNIGSNFVS | 347 | TCTGGAAGCAGCTCCAACATTGGGAGTAATTTTGTATCC |
| 165 | SGSSSNLENNYVS | 348 | TCTGGAAGCAGCTCCAATCTTGAGAATAATTATGTATCC |
| 166 | SGSISNIGSNYVS | 349 | TCTGGAAGCATCTCCAATATTGGCAGTAATTATGTATCC |
| 167 | SGSSSDIGSNYVS | 350 | TCTGGAAGCAGCTCCGACATTGGGAGTAATTATGTATCC |
| 168 | SGSSSNIGTNYVS | 351 | TCTGGAAGCAGCTCCAACATTGGGACTAATTATGTATCC |
| 169 | SGSSSNIGKNFVS | 352 | TCTGGAAGCAGCTCCAACATTGGGAAGAATTTTGTATCC |
| 170 | SGSSSNIGNNFIS | 353 | TCTGGAAGCAGCTCCAACATTGGGAATAATTTTATATCC |
| 171 | SGGSSNIGNNYVS | 354 | TCTGGAGGCAGCTCCAACATTGGCAATAATTATGTTTCC |
| 172 | SGSSSNIGENYVS | 355 | TCTGGAAGCAGCTCCAACATTGGGGAGAATTATGTATCC |
| 173 | SGSSSNIGNNFVA | 356 | TCTGGAAGCAGCTCCAATATTGGGAATAATTTTGTGGCC |
| 174 | SGGSSNIGNNYVA | 357 | TCTGGAGGCAGCTCCAACATTGGGAATAATTATGTAGCC |
| 175 | SGSSSHIGNNYVS | 358 | TCTGGAAGCAGCTCCCACATTGGAAATAATTATGTATCC |
| 176 | SGSSSNIGSNDVS | 359 | TCTGGAAGCAGCTCCAATATTGGAAGTAATGATGTATCG |
| 177 | SGSSSNIGNNYVT | 360 | TCTGGAAGCAGCTCCAACATTGGGAATAATTATGTAACC |
| 178 | SGSSSNIGNNPVS | 361 | TCTGGAAGCAGCTCCAACATTGGGAATAATCCTGTATCC |
| 179 | SGGSSNIGNHYVS | 362 | TCTGGAGGCAGCTCCAATATTGGGAATCATTATGTATCC |
| 180 | SGTSSNIGNNYVS | 363 | TCTGGAACCAGCTCCAACATTGGGAATAATTATGTATCC |
| 181 | SGSSSNIGSNYVS | 364 | TCTGGAAGCAGCTCCAACATTGGAAGTAATTATGTCTCG |
| 182 | SGGTSNIGSNYVS | 365 | TCTGGAGGCACCTCCAACATTGGAAGTAATTATGTATCC |
| 183 | SGSKSNIGNNYVS | 366 | TCTGGAAGCAAGTCCAACATTGGGAATAATTATGTATCC |
| 184 | SGRSSNIGNNYVS | 367 | TCTGGAAGAAGCTCCAACATTGGGAATAATTATGTATCG |
| 185 | SGSSSNVGSNYVS | 368 | TCTGGAAGCAGCTCCAACGTTGGGAGTAATTATGTTTCC |
| 186 | SGSTSNIGNNFVS | 369 | TCTGGAAGCACCTCCAATATTGGGAATAATTTTGTATCC |
| 187 | SGSNFNIGNNYVS | 370 | TCTGGAAGCAACTTCAACATTGGGAATAATTATGTCTCC |
| 188 | SGSTSNIGYNYVS | 371 | TCTGGAAGCACCTCCAATATTGGATATAATTATGTATCC |
| 189 | SGSSSNIVSNYVS | 372 | TCTGGAAGCAGCTCCAATATTGTAAGTAATTATGTATCC |
| 190 | SGTSSNIGNNFVS | 373 | TCTGGAACCAGCTCCAACATTGGGAATAATTTTGTATCC |
| 191 | SGSSSNIGRNFVS | 374 | TCTGGAAGCAGCTCCAACATTGGGAGGAATTTTGTGTCC |
| 192 | SGTTSNIGNNYVS | 375 | TCTGGAACGACCTCCAACATTGGGAATAATTATGTCTCC |
| 193 | SGSSSNIGNNDVS | 376 | TCTGGAAGCAGCTCCAACATTGGGAATAATGATGTATCC |
| 194 | SGSSSNIGNHDVS | 377 | TCTGGAAGCAGCTCCAACATTGGGAATCATGATGTATCC |
| 195 | SGSSSNIGSSHVS | 378 | TCTGGAAGCAGCTCCAACATTGGAAGTAGTCATGTATCC |
| 196 | SGSSSNIGIHYVS | 379 | TCTGGAAGCAGCTCCAACATTGGGATTCATTATGTATCC |
| 197 | SGGGSNIGYNYVS | 380 | TCTGGAGGCGGCTCCAACATTGGCTATAATTATGTCTCC |
| 198 | SGSSSNIGDHYVS | 381 | TCTGGAAGCAGCTCCAACATTGGGGATCATTATGTGTCG |
| 199 | SGSSSNLGKNYVS | 382 | TCTGGAAGCAGCTCCAACCTTGGGAAGAATTATGTATCT |
| 200 | SGSSSNIGDNFVS | 383 | TCTGGAAGCAGCTCCAACATTGGCGATAATTTTGTATCC |
| 201 | SGSTSNIEKNYVS | 384 | TCTGGAAGCACCTCCAACATTGAGAAAAACTATGTATCG |
| 202 | SGSSSNIGKDYVS | 385 | TCTGGAAGCAGCTCCAACATTGGGAAGGATTATGTATCC |
| 203 | SGSSSNIGKNYVS | 386 | TCTGGAAGCAGCTCCAACATTGGGAAGAATTATGTATCC |
| 204 | SGSSSNIGNNYVS | 387 | TCTGGAAGCAGCTCCAACATTGGGAATAATTATGTATCC |
| 205 | SGSSSNIGNNYAS | 388 | TCTGGAAGCAGCTCCAACATTGGGAATAATTATGCCTCC |
| 206 | SGISSNIGNNYVS | 389 | TCTGGAATCAGCTCCAACATTGGGAATAATTATGTATCC |
| 207 | TGSSSNIGNNYVS | 390 | ACTGGAAGCAGCTCCAACATTGGGAATAATTATGTATCC |
| 208 | SGTSSNIGNNHVS | 391 | TCTGGAACCAGCTCCAACATTGGGAATAATCATGTTTCC |
| 209 | SGSRSNIGKNYVS | 392 | TCTGGAAGTCGTTCCAACATTGGGAAAAATTATGTATCC |

IGLV1-51-L2

| | | | |
|---|---|---|---|
| 393 | DNNKRPP | 544 | GACAATAATAAGCGACCCCCA |
| 394 | ENNRRPS | 545 | GAGAATAATAGGCGACCCTCA |
| 395 | DNNKQPS | 546 | GACAATAATAAGCAACCCTCA |
| 396 | DNNKRPL | 547 | GACAATAACAAGCGACCCTTG |
| 397 | DNDKRPA | 548 | GACAATGATAAGCGACCCGCA |
| 398 | DNHERPS | 549 | GACAATCATGAGCGACCCTCA |
| 399 | ENRKRPS | 550 | GAAAACCGTAAGCGACCCTCA |
| 400 | DNDQRPS | 551 | GACAATGATCAGCGACCCTCA |
| 401 | ENYKRPS | 552 | GAGAATTATAAGCGACCCTCA |
| 402 | ENTKRPS | 553 | GAAAATACTAAGCGACCCTCA |
| 403 | DTEKRPS | 554 | GACACTGAGAAGAGGCCCTCA |
| 404 | DNDKRPP | 555 | GACAATGATAAGCGACCCGCA |
| 405 | DHNKRPS | 556 | GACCATAATAAGCGACCCTCA |
| 406 | GNNERPS | 557 | GGCAATAATGAGCGACCCTCA |
| 407 | DTSKRPS | 558 | GACACTAGTAAGCGACCCTCA |
| 408 | EYNKRPS | 559 | GAATATAATAAGCGCCCCTCA |
| 409 | ENIKRPS | 560 | GAAAATATTAAGCGACCCTCA |
| 410 | DNVKRPS | 561 | GACAATGTTAAGCGACCCTCA |
| 411 | ENDKRSS | 562 | GAAAACGATAAACGATCCTCA |
| 412 | ENNKRHS | 563 | GAAAATAATAAGCGACACTCA |
| 413 | GNDQRPS | 564 | GGAAATGATCAGCGACCCTCA |
| 414 | DNDRRPS | 565 | GACAATGATAGGCGACCCTCA |
| 415 | DNHKRPS | 566 | GACAATCATAAGCGGCCCTCA |
| 416 | DNNDRPS | 567 | GACAATAATGACCGACCCTCA |
| 417 | ENNQRPS | 568 | GAGAATAATCAGCGACCCTCA |
| 418 | DNNQRPS | 569 | GACAATAATCAGCGACCCTCA |
| 419 | ENVKRPS | 570 | GAGAATGTTAAGCGACCCTCA |
| 420 | DTYKRPS | 571 | GACACTTATAAGAGACCCTCA |
| 421 | NNNNRPS | 572 | AACAATAATAACCGACCCTCA |
| 422 | GNNNRPS | 573 | GGCAATAATAATCGACCCTCA |
| 423 | ENDQRPS | 574 | GAAAATGATCAGCGACCCTCA |
| 424 | DNNKRAS | 575 | GACAATAATAAGCGAGCCTCA |
| 425 | DNDKRPL | 576 | GACAATGATAAGCGACCCTTA |
| 426 | DTDERPS | 577 | GACACTGATGAGCGACCTTCA |
| 427 | DNRKRPS | 578 | GACAATAGGAAGCGACCCTCA |
| 428 | DNDARPS | 579 | GACAATGATGCTCGACCCTCA |
| 429 | DNNKRLS | 580 | GACAATAATAAGCGACTCTCA |
| 430 | DNDKRAS | 581 | GACAATGATAAGCGAGCCTCA |
| 431 | DNTERPS | 582 | GACAATACTGAGCGACCCTCA |
| 432 | DNNIRPS | 583 | GACAATAATATTCGACCCTCA |
| 433 | DNKRRPS | 584 | GACAATAAGAGGCGACCCTCA |
| 434 | DDNNRPS | 585 | GACGATAATAACCGACCCTCA |
| 435 | ANNRRPS | 586 | GCGAATAATCGACGACCCTCA |
| 436 | DNDKRLS | 587 | GACAATGATAAGCGACTGTCA |
| 437 | DNNKRPA | 588 | GACAATAATAAGCGACCCGCA |
| 438 | DNYRRPS | 589 | GACAATTATAGACGTCCCTCA |
| 439 | ANDQRPS | 590 | GCCAATGATCAGCGACCCTCA |
| 440 | DNDKRRS | 591 | GACAATGATAAGCGACGCTCA |
| 441 | DKNERPS | 592 | GACAAGAATGAGCGACCCTCA |
| 442 | DNKERPS | 593 | GACAATAAGGAGCGACCCTCA |
| 443 | DNNKGPS | 594 | GACAATAATAAGGGACCCTCA |
| 444 | ENDRRPS | 595 | GAAAATGATAGACGACCCTCA |
| 445 | ENDERPS | 596 | GAAAATGATCAGCGACCCTCA |
| 446 | QNNKRPS | 597 | CAAAATAATAAGCGACCCTCA |
| 447 | DNRERPS | 598 | GACAATCGTGAGCGACCCTCA |
| 448 | DNNRRPS | 599 | GACAATAATAGACGACCCTCA |
| 449 | GNNRRPS | 600 | GGAAATAATAGGCGACCCTCA |
| 450 | DNDNRPS | 601 | GACAATGATAACCGACCCTCA |
| 451 | EDNKRPS | 602 | GAAGATAATAAGCGACCCTCA |
| 452 | DDDERPS | 603 | GACGATGATGAGCGGCCCTCA |
| 453 | ASNKRPS | 604 | GCAAGTAATAAGCGACCCTCA |
| 454 | DNNKRSS | 605 | GACAATAATAAGCGATCCTCA |
| 455 | QNNERPS | 606 | CAAAATAATGAGCGACCCTCA |
| 456 | DDDRRPS | 607 | GACGATGATAGGCGACCCTCA |
| 457 | NNDKRPS | 608 | AACAATGATAAGCGACCCTCA |
| 458 | DNNNRPS | 609 | GACAATAATAACCGACCCTCA |
| 459 | DNNVRPS | 610 | GACAATAATGTGCGACCCTCA |
| 460 | ENNERPS | 611 | GAAAATAATGAGCGACCCTCA |
| 461 | DNNHRPS | 612 | GACAATAATCACCGACCCTCA |
| 462 | DNDERPS | 613 | GACAATGATGAGCGCCCCTCG |
| 463 | DNIRRPS | 614 | GACAATATCCGGCGACCCTCA |
| 464 | DFNKRPS | 615 | GACTTTAATAAGCGACCCTCA |
| 465 | ETNKRPS | 616 | GAAACTAATAAGCGACCCTCA |
| 466 | NDNKRPS | 617 | AACGATAATAAGCGACCCTCA |
| 467 | DDNKRPS | 618 | GACGATAATAAGCGACCCTCA |
| 468 | DNYKRPS | 619 | GACAATTATAAGCGACCCTCA |
| 469 | HNNKRPS | 620 | CACAATAATAAGCGACCCTCA |
| 470 | DNHQRPS | 621 | GACAATCATCAGCGACCCTCA |
| 471 | DNYKRAS | 622 | GACAATTATAAGCGACCCTCA |
| 472 | DNIKRPS | 623 | GACAATATTAAGCGACCCTCA |
| 473 | DTHKRPS | 624 | GACACTCATAAGCGACCCTCA |
| 474 | DTNRRPS | 625 | GACACTAATAGGCGACCCTCT |
| 475 | DTNQRPS | 626 | GACACTAATCAGCGACCCTCA |
| 476 | ESDKRPS | 627 | GAAAGTGATAAGCGACCCTCA |
| 477 | DNDKRSS | 628 | GACAATGATAAGCGATCTTCG |
| 478 | GSNKRPS | 629 | GGCAGTAATAAGCGACCCTCA |
| 479 | DNNKRVS | 630 | GACAATAACAAGCGAGTTTCA |
| 480 | NNNRRPS | 631 | AACAATAATAGGCGACCCTCA |
| 481 | DNFKRPS | 632 | GACAATTTTAAGCGACCCTCA |
| 482 | ENDKRPS | 633 | GAAAATGATAAACGACCCTCA |
| 483 | ENNKRLS | 634 | GAAAATAATAAGCGACTCTCA |
| 484 | ADNKRPS | 635 | GCAGATAATAAGCGACCCTCA |
| 485 | EDNERPS | 636 | GAAGATAATGAGCGCCCCTCA |
| 486 | DTDQRPS | 637 | GACACTGATCAGCGACCCTCA |
| 487 | DNYQRPS | 638 | GACAATTATCAGCGACCCTCA |
| 488 | DENKRPS | 639 | GACGAGAATAAGCGACCCTCA |
| 489 | DTNKRPS | 640 | GACACTAATAAGCGACCCTCA |
| 490 | DDYRRPS | 641 | GACGATTATCGGCGACCCTCA |
| 491 | DNDKRHS | 642 | GACAACGATAAGCGGCACTCA |
| 492 | ENDNRPS | 643 | GAAAATGATAATCGACCCTCA |
| 493 | DDNERPS | 644 | GACGATAATGAGCGCCCCTCA |
| 494 | DNKKRPS | 645 | GACAATAAGAAGCGACCCTCA |
| 495 | DVDKRPS | 646 | GACGTTGATAAGCGACCCTCA |
| 496 | ENKKRPS | 647 | GAAAATAAAAAACGACCCTCT |
| 497 | VNDKRPS | 648 | GTCAATGATAAGCGACCCTCA |
| 498 | DNDHRPS | 649 | GACAATGATCACCGACCCTCA |
| 499 | DINKRPS | 650 | GACATTAATAAGCGACCCTCA |
| 500 | ANNERPS | 651 | GCCAATAATGAGCGACCCTCA |
| 501 | DNENRPS | 652 | GACAATGAAAACCGACCGTCA |
| 502 | GDDKRPS | 653 | GGCGATGATAAGCGACCCTCA |
| 503 | ANNQRPS | 654 | GCCAATAATCAGCGACCTTCA |
| 504 | DDDKRPS | 655 | GACGATGATAAGCGACCCTCA |
| 505 | YNNKRPS | 656 | TACAATAATAAGCGGCCCTCA |
| 506 | EDDKRPS | 657 | GAAGATGATAAGCGACCCTCA |
| 507 | ENNNRPS | 658 | GAAAACAATAACCGACCCTCG |
| 508 | DNNLRPS | 659 | GACAATAATCTGCGACCCTCA |
| 509 | ESNKRPS | 660 | GAGAGTAACAAGCGACCCTCA |
| 510 | DTDKRPS | 661 | GACACTGATAAGCGGCCCTCA |
| 511 | DDDQRPS | 662 | GACGATGATCAGCGACCCTCA |
| 512 | VNNKRPS | 663 | GTGAATAATAAGAGACCCTCC |
| 513 | DDYKRPS | 664 | GACGATTATAAGCGACCCTCA |
| 514 | DNTKRPS | 665 | GACAATACTAAGCGACCCTCA |
| 515 | DDTERPS | 666 | GACGATACTGAGCGACCCTCA |
| 516 | GNDKRPS | 667 | GGCAATGATAAGCGACCCTCA |
| 517 | DNEKRPS | 668 | GACAATGAAAAGCGACCCTCA |
| 518 | DNDDRPS | 669 | GACAATGATGACCGACCCTCA |
| 519 | DDNRRPS | 670 | GACGATAATAGGCGTCCCTCA |
| 520 | GNNKRPS | 671 | GGCAATAATAAGCGACCCTCA |
| 521 | ANDKRPS | 672 | GGCAATGATAAGCGACCCTCA |
| 522 | DNNKRHS | 673 | GACAATAATAAGCGACACTCA |
| 523 | DDNQRPS | 674 | GACGACAATCAGCGACCCTCA |
| 524 | GNDRRPS | 675 | GGCAATGATAGGCGACCCTCA |
| 525 | DNHNRPS | 676 | GACAATCATAACCGACCCTCA |
| 526 | DNYERPS | 677 | GACAATTATGAGCGACCCTCA |
| 527 | ENNKRSS | 678 | GAAAATAATAAGCGATCCTCA |
| 528 | DDHKRPS | 679 | GACGATCATAAGCGGCCCTCA |
| 529 | DNNKRRS | 680 | GACAATAATAAACGACGTTCA |
| 530 | DNDKRPS | 681 | GACAATGATAAGCGACCGTCA |
| 531 | DKNKRPS | 682 | GACAAGAATAAGCGACCCTCA |
| 532 | DNNKRPS | 683 | GACAATAATAAGCGACCCTCA |
| 533 | DIDKRPS | 684 | GACATTGATAAGCGACCCTCA |
| 534 | DDKKRPS | 685 | GACGATAAGAAGCGACCCTCA |
| 535 | ANNKRPS | 686 | GGCAATAATAAGCGACCCTCA |
| 536 | DNDKGPS | 687 | GACAATGATAAGGGACCCTCA |
| 537 | EDNRRPS | 688 | GAAGATAATAGGCGACCCTCA |
| 538 | ENNKRPS | 689 | GAGAATAATAAGCGACCCTCA |
| 539 | NNNKRPS | 690 | AACAATAATAAGCGACCCTCA |
| 540 | DNNERPS | 691 | GACAATAATGAGCGACCCTCA |
| 541 | DNIQRPS | 692 | GACAATATTCAGCGACCCTCA |
| 542 | DNNYRPS | 693 | GACAATAATTACCGACCCTCA |
| 543 | DNYNRPS | 694 | GACAATTATAACCGACCCTCA |

IGLV1-51-L3

| | | | |
|---|---|---|---|
| 695 | CGTWDTSLSAVVF | 1431 | TGCGGAACATGGGATACCAGCCTGAGTGCTGTGGTGTTC |
| 696 | CGTWDTSLSAGVF | 1432 | TGCGGAACATGGGATACCAGCCTGAGTGCTGGGGTGTTC |
| 697 | CGTWDTSLSAWVF | 1433 | TGCGGAACATGGGATACCAGCCTGAGTGCTTGGGTGTTC |
| 698 | CGTWDRSLSAGVF | 1434 | TGCGGAACATGGGATAGGAGCCTGAGTGCGGGGGTGTTC |
| 699 | CGTWDRSLSAWVF | 1435 | TGCGGAACATGGGATAGGAGCCTGAGTGCTTGGGTATTT |
| 700 | CGTWDTSLSGGVF | 1436 | TGCGGAACATGGGATACCAGCCTGAGTGGTGGGGTGTTC |
| 701 | CGTWDTSLRAGVF | 1437 | TGCGGAACATGGGATACTAGCCTGCGTGCTGGCGTCTTC |
| 702 | CGTWDRSLSVWVF | 1438 | TGCGGAACATGGGATAGGAGCCTGAGTGTTTGGGTGTTC |
| 703 | CGTWDTSLSVVVF | 1439 | TGCGGAACATGGGATACCAGTCTGAGTGTTGTGGTCTTC |
| 704 | CGTWDTSLSAAVF | 1440 | TGCGGAACGTGGGATACCAGCCTGAGTGCTGCGGTGTTC |
| 705 | CGAWDTSLSAGVF | 1441 | TGCGGAGCATGGGATACCAGCCTGAGTGCTGGAGTGTTC |
| 706 | CATWDTSLSAVVF | 1442 | TGCGCAACATGGGATACCAGCCTGAGTGCTGTGGTATTC |
| 707 | CATWDTSLSAGVF | 1443 | TGCGCAACATGGGATACCAGCCTGAGTGCTGGTGTGTTC |
| 708 | CGTWESSLSAWVF | 1444 | TGTGGAACATGGGAGAGCAGCCTGAGTGCTTGGGTGTTC |
| 709 | CGTWDTTLSAGVF | 1445 | TGCGGAACATGGGATACCACCCTGAGTGCGGGTGTCTTC |
| 710 | CGTWDTSLSVWVF | 1446 | TGCGGAACATGGGATACTAGCCTGAGTGTGTGGGTGTTC |
| 711 | CGTWDTSLSVGVF | 1447 | TGCGGAACATGGGATACTAGCCTGAGTGTTGGGGTGTTC |
| 712 | CGTWDTSLSTGVF | 1448 | TGCGGAACATGGGACACCAGTCTGAGCACTGGCGTCTTC |
| 713 | CGTWDTSLSGVVF | 1449 | TGCGGAACATGGGATACCAGCCTGAGTGGTGTGGTCTTC |
| 714 | CGTWDTSLSAYVF | 1450 | TGCGGAACATGGGATACCAGCCTGAGTGCTTATGTCTTC |
| 715 | CGTWDTSLSAEVF | 1451 | TGCGGAACATGGGATACCAGCCTGAGTGCTGAGGTGTTC |
| 716 | CGTWDTGLSAGVF | 1452 | TGCGGAACATGGGATACCGGCCTGAGTGCTGGGGTATTC |
| 717 | CGTWDRSLSAYVF | 1453 | TGCGGAACGTGGGATAGGAGCCTGAGTGCTTATGTCTTC |
| 718 | CGTWDRSLSAVVF | 1454 | TGCGGAACATGGGATAGGAGCCTCAGTGCCGTGGTATTC |
| 719 | CGTWDNTLSAWVF | 1455 | TGCGGAACATGGGATAACACCCTGAGTGCGTGGGTGTTC |
| 720 | CGTWDNRLSAGVF | 1456 | TGCGGAACATGGGATAACAGGCTGAGTGCTGGGGTGTTC |
| 721 | CGTWDISLSAWVF | 1457 | TGCGGAACATGGGACATCAGCCTGAGTGCTTGGGTGTTC |
| 722 | CGTWHSSLSAGVF | 1458 | TGCGGAACATGGCATAGCAGCCTGAGTGCTGGGGTATTC |
| 723 | CGTWGSSLSAWVF | 1459 | TGCGGAACATGGGGTAGCAGTTTGAGTGCTTGGGTGTTC |
| 724 | CGTWESSLSGWVF | 1460 | TGCGGAACATGGGAGAGCAGCCTGAGTGGTTGGGTGTTC |
| 725 | CGTWESSLSAVVF | 1461 | TGCGGAACATGGGAGAGCAGCCTGAGTGCTGTGGTTTTC |
| 726 | CGTWDYSLSAVVF | 1462 | TGCGGAACATGGGATTACAGCCTGAGTGCTGTGGTATTC |
| 727 | CGTWDYSLSAGVF | 1463 | TGCGGAACATGGGATTACAGCCTGAGTGCTGGGGTATTC |
| 728 | CGTWDVSLSVGVF | 1464 | TGCGGAACATGGGATGTCAGCCTGAGTGTTGGAGTGTTC |
| 729 | CGTWDTTLSAVVF | 1465 | TGCGGAACATGGGATACCACCCTGAGTGCTGTGGTTTTC |
| 730 | CGTWDTTLNIGVF | 1466 | TGCGGAACATGGGATACCACTCTGAATATTGGGGTGTTC |
| 731 | CGTWDTSLTAVVF | 1467 | TGCGGAACATGGGATACCAGCCTGACTGCTGTGGTATTC |
| 732 | CGTWDTSLTAAVF | 1468 | TGCGGAACCTGGGATACCAGCCTGACTGCTGCTGTGTTC |
| 733 | CGTWDTSLSVGLF | 1469 | TGCGGCACATGGGATACCAGCCTGAGTGTGGGGCTATTC |
| 734 | CGTWDTSLSGRVF | 1470 | TGCGGAACCTGGGATACCAGCCTGAGTGGTAGGGTGTTC |
| 735 | CGTWDTSLSGAVF | 1471 | TGCGGAACATGGGATACCAGCCTGAGTGGTGCAGTGTTC |
| 736 | CGTWDTSLSAGLF | 1472 | TGCGGAACATGGGATACCAGCCTGAGTGCTGGCCTGTTC |
| 737 | CGTWDTSLSAGGVF | 1473 | TGCGGAACATGGGATACCAGCCTGAGTGCTGGAGGGGTCTTC |
| 738 | CGTWDTSLRAYVF | 1474 | TGCGGAACATGGGATACCAGCCTGCGTGCTTATGTCTTC |
| 739 | CGTWDTSLRAWVF | 1475 | TGCGGAACATGGGATACTAGTTTGCGTGCTTGGGTATTC |
| 740 | CGTWDTSLNTGVF | 1476 | TGCGGAACATGGGATACCAGCCTGAATACTGGGGTATTC |
| 741 | CGTWDTSLNIWVF | 1477 | TGCGGAACATGGGATACCAGCCTGAATATTTGGGTGTTC |
| 742 | CGTWDTSLNIGVF | 1478 | TGCGGAACATGGGATACAAGCCTGAATATTGGGGTGTTC |
| 743 | CGTWDTSLIAVVF | 1479 | TGCGGAACATGGGATACCAGCCTGATTGCTGTGGTGTTC |
| 744 | CGTWDRSLSGWVF | 1480 | TGCGGAACGTGGGATAGGAGCCTGAGTGGTTGGGTGTTC |
| 745 | CGTWDNRLSGWVF | 1481 | TGCGGAACATGGGATAACAGGCTGAGTGGTTGGGTGTTC |
| 746 | CGTWDKSLSAVVF | 1482 | TGCGGAACGTGGGATAAGAGCCTGAGTGCTGTGGTCTTC |
| 747 | CGTWDKGLSAWVF | 1483 | TGCGGAACATGGGATAAAGGCCTGAGTGCTTGGGTGTTC |
| 748 | CGTWDISLSAGVF | 1484 | TGCGGAACATGGGATATCAGCCTGAGTGCTGGGGTGTTC |
| 749 | CGTWDESLSGGEVVF | 1485 | TGCGGAACATGGGATGAGAGCCTGAGTGGTGGCGAGGTGGTCTTC |
| 750 | CGTWDASLSAWVF | 1486 | TGCGGAACATGGGATGCCAGCCTGAGTGCCTGGGTGTTC |
| 751 | CGTWDAGLSAWVF | 1487 | TGCGGAACTTGGGATGCCGGCCTGAGTGCTTGGGTGTTC |
| 752 | CGAWDTSLSAWVF | 1488 | TGCGGAGCATGGGATACCAGCCTGAGTGCTTGGGTGTTC |
| 753 | CGAWDTSLSAVVF | 1489 | TGCGGAGCATGGGATACCAGCCTGAGTGCTGTGGTGTTC |
| 754 | CGAWDTSLRAGVF | 1490 | TGCGGAGCATGGGATACCAGCCTGCGTGCTGGGGTTTTC |
| 755 | CATWDTSVSAWVF | 1491 | TGCGCAACATGGGATACCAGCGTGAGTGCTTGGGTGTTC |
| 756 | CATWDTSLSAWVF | 1492 | TGCGCAACATGGGATACCAGCCTGAGTGCGTGGGTGTTC |
| 757 | CATWDNTLSAGVF | 1493 | TGCGCAACATGGGACAACACCCTGAGTGCTGGGGTGTTC |
| 758 | CAAWDRSLSVWVF | 1494 | TGCGCAGCATGGGATAGGAGCCTGAGTGTTTGGGTGTTC |
| 759 | CYTWHSSLRGGVF | 1495 | TGCTACACATGGCATTCCAGTCTGCGTGGTGGGGTGTTC |
| 760 | CVTWTSSPSAWVF | 1496 | TGCGTAACGTGGACTAGTAGCCCGAGTGCTTGGGTGTTC |
| 761 | CVTWRGGLVLF | 1497 | TGCGTGACATGGCGTGGTGGCCTTGTGTTGTTC |
| 762 | CVTWDTSLTSVVL | 1498 | TGCGTAACATGGGATACCAGCCTGACTTCTGTGGTACTC |
| 763 | CVTWDTSLSVYWVF | 1499 | TGCGTAACATGGGATACCAGCCTGAGTGTTTATTGGGTGTTC |
| 764 | CVTWDTSLSAWVF | 1500 | TGCGTTACATGGGATACCAGCCTGAGTGCCTGGGTGTTC |
| 765 | CVTWDTDLSVALF | 1501 | TGCGTCACATGGGATACCGACCTCAGCGTTGCGCTCTTC |
| 766 | CVTWDRSLSGWVF | 1502 | TGCGTAACATGGGATAGGAGCCTGAGTGGTTGGGTGTTC |
| 767 | CVTWDRSLREVLF | 1503 | TGCGTAACATGGGATCGCAGCCTGAGAGAGGTGTTATTC |
| 768 | CVTWDRSLRAVVF | 1504 | TGCGTAACATGGGATCGCAGCCTGAGAGCGGTGGTATTC |
| 769 | CVTWDRSLDAGVF | 1505 | TGCGTAACATGGGACAGGAGCCTCGATGCTGGGGTTTTC |
| 770 | CVTWDNTLSAGVF | 1506 | TGCGTGACATGGGATAACACCCTGAGTGCTGGGGTCTTC |
| 771 | CVTWDNNLFGVVF | 1507 | TGCGTAACATGGGATAACAACCTGTTTGGTGTGGTCTTC |
| 772 | CVSWDTSLSGAVF | 1508 | TGCGTATCATGGGATACCAGCCTGAGTGGTGCGGTATTC |
| 773 | CVSWDTSLSAGVF | 1509 | TGCGTCTCATGGGATACCAGCCTGAGTGCTGGGGTATTC |
| 774 | CTTWFRTPSDVVF | 1510 | TGCACAACATGGTTTAGGACTCCGAGTGATGTGGTCTTC |
| 775 | CTTWFRTASDVVF | 1511 | TGCACAACATGGTTTAGGACTGCGAGTGATGTGGTCTTC |
| 776 | CTTWDYGLSVVF | 1512 | TGCACAACGTGGGATTACGGTCTGAGTGTCGTCTTC |
| 777 | CTARDTSLSPGGVF | 1513 | TGCACAGCAAGGGATACCAGCCTGAGTCCTGGCGGGGTCTTC |
| 778 | CSTWNTRPSDVVF | 1514 | TGCTCAACATGGAATACGAGGCCGAGTGATGTGGTGTTC |
| 779 | CSTWESSLTTVVF | 1515 | TGTTCAACATGGGAGAGCAGTTTGACTACTGTGGTCTTC |
| 780 | CSTWDTSLTNVLF | 1516 | TGCTCAACATGGGATACCAGCCTCACTAATGTGCTATTC |
| 781 | CSTWDTSLSGVVF | 1517 | TGCTCAACATGGGATACCAGCCTGAGTGGAGTAGTCTTC |
| 782 | CSTWDHSLKAALF | 1518 | TGCTCAACATGGGATCACAGCCTGAAAGCTGCACTGTTC |
| 783 | CSTWDARLSVRVF | 1519 | TGCTCAACCTGGGATGCGAGGCTGAGTGTCCGGGTGTTC |
| 784 | CSSYTSSSTWVF | 1520 | TGCTCCTCATATACAAGCAGCAGCACTTGGGTGTTC |
| 785 | CSSYATRGLRVLF | 1521 | TGCAGCTCATACGCAACCCGCGGCCTTCGTGTGTTGTTC |
| 786 | CSSWDATLSVRIF | 1522 | TGTTCATCATGGGACGCCACCCTGAGTGTTCGCATATTC |
| 787 | CQVWEGSSDHWVF | 1523 | TGTCAGGTGTGGGAGGGTAGTAGTGATCATTGGGTGTTC |
| 788 | CQTWDNRLSAVVF | 1524 | TGCCAAACCTGGGATAACAGACTGAGTGCTGTGGTGTTC |
| 789 | CQTWDHSLHVGVF | 1525 | TGTCAAACGTGGGATCACAGCCTGCATGTTGGGGTGTTC |
| 790 | CQSYDDILNVWVL | 1526 | TGCCAGTCCTATGACGACATCTTGAATGTTTGGGTCCTT |
| 791 | CNTWDKSLTSELF | 1527 | TGCAATACATGGGATAAGAGTTTGACTTCTGAACTCTTC |
| 792 | CLTWDRSLNVRVF | 1528 | TGCTTAACATGGGATCGCAGCCTGAATGTGAGGGTGTTC |
| 793 | CLTWDHSLTAYVF | 1529 | TGCCTAACATGGGACCACAGCCTGACTGCTTATGTCTTC |
| 794 | CLTRDTSLSAPVF | 1530 | TGCTTAACAAGGGATACCAGTCTGAGTGCCCCTGTGTTC |
| 795 | CKTWESGLNFGHVF | 1531 | TGCAAAACATGGGAAAGTGGCCTTAATTTTGGCCACGTCTTC |
| 796 | CKTWDTSLSAVVF | 1532 | TGCAAAACATGGGATACCAGCCTGAGTGCTGTGGTCTTC |
| 797 | CGVWDVSLGAGVF | 1533 | TGCGGAGTCTGGGATGTCAGTCTGGGTGCTGGGGTGTTC |
| 798 | CGVWDTTPSAVLF | 1534 | TGCGGAGTCTGGGATACCACCCCGAGTGCCGTTCTTTTC |
| 799 | CGVWDTTLSAVLF | 1535 | TGCGGAGTCTGGGATACCACCCTGAGTGCCGTTCTTTTC |
| 800 | CGVWDTSLGVF | 1536 | TGCGGAGTATGGGATACCAGCCTGGGGGTCTTC |
| 801 | CGVWDTNLGKWVF | 1537 | TGCGGGGTATGGGATACCAACCTGGGTAAATGGGTTTTC |
| 802 | CGVWDTGLDAGWVF | 1538 | TGTGGAGTTTGGGATACTGGCCTGGATGCTGGTTGGGTGTTC |
| 803 | CGVWDNVLEAYVF | 1539 | TGCGGAGTGTGGGATAACGTCCTGGAGGCCTATGTCTTC |
| 804 | CGVWDISLSANWVF | 1540 | TGCGGAGTCTGGGATATCAGCCTGAGTGCTAATTGGGTGTTC |
| 805 | CGVWDHSLGIWAF | 1541 | TGCGGAGTATGGGATCACAGCCTGGGGATTTGGGCCTTC |
| 806 | CGVWDDILTAEVF | 1542 | TGCGGAGTTTGGGATGATATTCTGACTGCTGAAGTGTTC |
| 807 | CGVRDTSLGVF | 1543 | TGCGGAGTTCGGGATACCAGCCTGGGGGTCTTC |
| 808 | CGTYDTSLPAWVF | 1544 | TGCGGAACATACGATACGAGCCTGCCTGCTTGGGTGTTT |
| 809 | CGTYDNLVFGYVF | 1545 | TGCGGAACTTACGATAATCTTGTATTTGGTTATGTCTTC |
| 810 | CGTYDDRLREVF | 1546 | TGCGGAACATACGATGATAGACTCAGAGAGGTGTTC |
| 811 | CGTWVTSLSAGVF | 1547 | TGCGGAACGTGGGTTACCAGCCTGAGTGCTGGGGTGTTC |
| 812 | CGTWVSSLTTVVF | 1548 | TGCGGAACATGGGTTAGCAGCCTGACTACTGTAGTATTC |
| 813 | CGTWVSSLNVWVF | 1549 | TGCGGAACATGGGTTAGCAGCCTGAACGTCTGGGTGTTC |
| 814 | CGTWVGRFWVF | 1550 | TGCGGAACATGGGTTGGCAGGTTTTGGGTATTC |
| 815 | CGTWSGGPSGHWLF | 1551 | TGCGGAACATGGTCTGGCGGCCCGAGTGGCCATTGGTTGTTC |
| 816 | CGTWSGGLSGHWLF | 1552 | TGCGGAACATGGTCTGGCGGCCTGAGTGGCCATTGGTTGTTC |
| 817 | CGTWQTGREAVLF | 1553 | TGCGGAACGTGGCAGACCGGCCGGGAGGCTGTCCTATTT |
| 818 | CGTWQSRLRWVF | 1554 | TGCGGAACGTGGCAGAGCAGGCTGAGGTGGGTGTTC |
| 819 | CGTWQSRLGWVF | 1555 | TGCGGAACGTGGCAGAGCAGGCTGGGGTGGGTGTTC |
| 820 | CGTWPRSLSAVWVF | 1556 | TGCGGAACATGGCCTAGGAGCCTGAGTGCTGTTTGGGTGTTC |
| 821 | CGTWNNYLSAGDVVF | 1557 | TGCGGAACATGGAATAACTACCTGAGTGCTGGCGATGTGGTTTTC |
| 822 | CGTWLGSQSPYWVF | 1558 | TGCGGAACATGGCTTGGCAGCCAGAGTCCTTATTGGGTCTTC |
| 823 | CGTWHTGLSAYVF | 1559 | TGCGGAACATGGCATACCGGCCTGAGTGCTTATGTCTTC |
| 824 | CGTWHSTLSAGHWVF | 1560 | TGCGGAACATGGCATAGTACCCTGAGTGCTGGCCATTGGGTGTTC |
| 825 | CGTWHSSLSTWVF | 1561 | TGCGGAACATGGCATAGTAGCCTGAGTACTTGGGTGTTC |
| 826 | CGTWHSSLSAYVF | 1562 | TGCGGAACATGGCATAGCAGCCTGAGTGCCTATGTCTTC |
| 827 | CGTWHSSLSAVVF | 1563 | TGCGGAACATGGCATAGCAGCCTGAGTGCTGTGGTATTC |
| 828 | CGTWHSGLSGWVF | 1564 | TGCGGAACGTGGCATTCCGGCCTGAGTGGGTGGGTTTTC |
| 829 | CGTWHNTLRNVIF | 1565 | TGCGGAACATGGCATAACACCCTGCGTAATGTGATATTC |
| 830 | CGTWHASLTAVF | 1566 | TGCGGAACATGGCATGCCAGCCTGACTGCTGTGTTC |
| 831 | CGTWGWYGSQRGVVF | 1567 | TGCGGGACATGGGGATGGTATGGCAGCCAGAGAGGCGTCGTCTTC |
| 832 | CGTWGWYGGQRGVVF | 1568 | TGCGGGACATGGGGATGGTATGGCGGCCAGAGAGGCGTCGTCTTC |
| 833 | CGTWGTSLSAWVF | 1569 | TGCGGAACCTGGGGAACCAGCCTGAGTGCTTGGGTGTTC |
| 834 | CGTWGSSLTTGLF | 1570 | TGCGGAACCTGGGGTAGCAGCCTGACTACTGGCCTGTTC |
| 835 | CGTWGSSLTAYVF | 1571 | TGCGGAACATGGGGTAGCAGCCTGACTGCCTATGTCTTC |
| 836 | CGTWGSSLSVVF | 1572 | TGCGGAACATGGGGTAGCAGCCTGAGTGTTGTGTTC |
| 837 | CGTWGSSLSGGVF | 1573 | TGCGGAACATGGGGTAGCAGCCTGAGTGGTGGGGTGTTC |
| 838 | CGTWGSSLSAYWVF | 1574 | TGCGGAACATGGGGTAGCAGCCTGAGTGCTTATTGGGTGTTC |
| 839 | CGTWGSSLSAYVVF | 1575 | TGCGGAACATGGGGTAGCAGCCTGAGTGCTTATGTGGTGTTC |
| 840 | CGTWGSSLSAYVF | 1576 | TGCGGAACATGGGGTAGCAGCCTGAGTGCTTATGTCTTC |
| 841 | CGTWGSSLSAVVF | 1577 | TGCGGAACGTGGGGTAGTAGCCTGAGTGCTGTGGTGTTC |
| 842 | CGTWGSSLSAPYVF | 1578 | TGCGGAACATGGGGTAGCAGCCTGAGTGCTCCTTATGTCTTC |
| 843 | CGTWGSSLSAPVF | 1579 | TGCGGAACATGGGGTAGCAGCCTGAGTGCCCCGGTGTTC |
| 844 | CGTWGSSLSAGVF | 1580 | TGCGGAACATGGGGTAGCAGCCTGAGTGCTGGGGTGTTC |
| 845 | CGTWGSSLSAGLF | 1581 | TGCGGAACTTGGGGTAGCAGCCTGAGTGCTGGACTGTTC |
| 846 | CGTWGSSLSAGALF | 1582 | TGCGGAACATGGGGTAGCAGCCTGAGTGCTGGGGCACTCTTC |
| 847 | CGTWGSSLRAWVF | 1583 | TGCGGAACATGGGGCAGTAGCCTGCGTGCTTGGGTGTTC |
| 848 | CGTWFTSLASGVF | 1584 | TGCGGAACCTGGTTTACTAGTCTGGCTAGTGGGGTTTTC |
| 849 | CGTWETSLSVVVI | 1585 | TGCGGAACTTGGGAGACCAGTCTGAGTGTCGTGGTCATC |
| 850 | CGTWETSLSGVF | 1586 | TGCGGAACATGGGAGACCAGCCTGAGTGGTGTCTTC |
| 851 | CGTWETSLSDWVF | 1587 | TGCGGAACATGGGAAACCAGCCTGAGTGATTGGGTATTC |
| 852 | CGTWETSLSAGVF | 1588 | TGCGGAACATGGGAGACCAGCCTGAGTGCTGGGGTATTC |
| 853 | CGTWETSLNYVAF | 1589 | TGCGGAACATGGGAAACCAGCCTTAATTATGTGGCCTTC |
| 854 | CGTWETSLNTWLL | 1590 | TGCGGAACATGGGAGACCAGCCTGAATACTTGGTTGCTC |
| 855 | CGTWETSESGNYIF | 1591 | TGCGGAACATGGGAGACCAGCGAGAGTGGTAATTACATCTTC |
| 856 | CGTWETRLGTWVI | 1592 | TGCGGAACATGGGAAACCAGACTGGGTACTTGGGTGATC |
| 857 | CGTWETQLYWVF | 1593 | TGCGGAACATGGGAGACCCAGTTATATTGGGTGTTC |
| 858 | CGTWETGLSAGEVF | 1594 | TGCGGAACATGGGAGACTGGCCTAAGTGCTGGAGAGGTGTTC |
| 859 | CGTWESTLSVFLF | 1595 | TGCGGAACTTGGGAAAGCACCCTGAGTGTTTTCCTATTC |
| 860 | CGTWESSLTVVVF | 1596 | TGCGGGACATGGGAAAGTAGCCTGACTGTTGTGGTCTTC |
| 861 | CGTWESSLTGVVF | 1597 | TGCGGAACATGGGAAAGTAGCCTGACTGGAGTGGTATTC |
| 862 | CGTWESSLTGFVF | 1598 | TGCGGAACATGGGAAAGCAGCCTGACTGGTTTTGTCTTC |
| 863 | CGTWESSLSVGVF | 1599 | TGTGGAACATGGGAGAGCAGCCTGAGTGTTGGGGTGTTC |
| 864 | CGTWESSLSEWVF | 1600 | TGCGGAACCTGGGAAAGTAGCCTCAGTGAATGGGTGTTC |
| 865 | CGTWESSLSAVF | 1601 | TGCGGAACATGGGAGAGCAGCCTGAGTGCTGTATTC |
| 866 | CGTWESSLSAGYIF | 1602 | TGCGGAACATGGGAGAGCAGCCTGAGTGCTGGTTATATCTTC |
| 867 | CGTWESSLSAGVF | 1603 | TGCGGAACATGGGAGAGCAGCCTGAGTGCTGGAGTGTTC |
| 868 | CGTWESSLSAGPVF | 1604 | TGCGGAACATGGGAAAGCAGCCTGAGCGCTGGCCCGGTGTTC |
| 869 | CGTWESSLSAGGQVF | 1605 | TGCGGAACATGGGAAAGCAGCCTGAGTGCTGGAGGCCAGGTGTTC |
| 870 | CGTWESSLSAFGGYVF | 1606 | TGCGGAACATGGGAGAGCAGCCTGAGTGCCTTCGGCGGTTATGTC |
| | | | TTC |
| 871 | CGTWESSLRVWVF | 1607 | TGCGGAACATGGGAAAGCAGCCTGAGGGTTTGGGTGTTC |
| 872 | CGTWESSLFTGPWVF | 1608 | TGCGGAACATGGGAAAGCAGCCTCTTTACTGGGCCTTGGGTGTTC |
| 873 | CGTWESLSATYVF | 1609 | TGCGGAACATGGGAGAGCCTGAGTGCCACCTATGTCTTC |
| 874 | CGTWESGLSAGVF | 1610 | TGCGGAACATGGGAGAGCGGCCTGAGTGCTGGTGTCTTC |
| 875 | CGTWESDFWVF | 1611 | TGCGGAACATGGGAAAGCGACTTTTGGGTGTTT |
| 876 | CGTWENRLSAVVF | 1612 | TGCGGTACATGGGAAAACAGACTGAGTGCTGTGGTCTTC |
| 877 | CGTWENRLSAGVF | 1613 | TGCGGAACATGGGAAAACAGACTGAGTGCCGGGGTATTC |
| 878 | CGTWEISLTTSVVF | 1614 | TGCGGAACATGGGAAATCAGCCTGACTACTTCTGTGGTATTC |
| 879 | CGTWEISLSTSVVF | 1615 | TGCGGAACATGGGAAATCAGCCTGAGTACTTCTGTGGTATTC |
| 880 | CGTWEGSLSVVF | 1616 | TGCGGAACATGGGAAGGCAGCCTCAGTGTTGTTTTC |
| 881 | CGTWEGSLRVF | 1617 | TGCGGAACATGGGAAGGCAGCCTGAGGGTGTTC |
| 882 | CGTWEGSLRHVF | 1618 | TGCGGAACATGGGAGGGCAGCCTGAGGCACGTGTTC |
| 883 | CGTWDYSPVRAGVF | 1619 | TGCGGAACATGGGATTACAGCCCTGTACGTGCTGGGGTGTTC |
| 884 | CGTWDYSLSVYLF | 1620 | TGCGGAACGTGGGATTACAGCCTGAGTGTTTATCTCTTC |
| 885 | CGTWDYSLSSGVVF | 1621 | TGCGGAACATGGGATTACAGCCTGAGTTCTGGCGTGGTATTC |
| 886 | CGTWDYSLSAWVF | 1622 | TGCGGAACATGGGATTACAGCCTGAGTGCCTGGGTGTTC |
| 887 | CGTWDYSLSAEVF | 1623 | TGCGGAACATGGGATTACAGTCTGAGTGCTGAGGTGTTC |
| 888 | CGTWDYSLRRAIF | 1624 | TGCGGAACATGGGATTACAGCCTGCGTCGTGCGATATTC |
| 889 | CGTWDWSLILQLF | 1625 | TGCGGAACATGGGATTGGAGCCTCATTCTTCAATTGTTC |
| 890 | CGTWDVTLHTGVF | 1626 | TGCGGAACATGGGATGTCACCTTGCATACTGGGGTGTTC |
| 891 | CGTWDVTLHIGVF | 1627 | TGCGGAACATGGGATGTCACCTTGCATATTGGGGTGTTC |
| 892 | CGTWDVTLHAGVF | 1628 | TGCGGAACATGGGATGTCACCTTGCATGCTGGGGTGTTC |
| 893 | CGTWDVSLYSGGVF | 1629 | TGCGGAACATGGGATGTCAGTTTGTATAGTGGCGGGGTCTTC |
| 894 | CGTWDVSLTSFVF | 1630 | TGTGGAACATGGGATGTCAGCCTGACTTCTTTCGTCTTC |
| 895 | CGTWDVSLSVGVL | 1631 | TGCGGAACATGGGATGTCAGCCTGAGTGTTGGGGTGCTC |
| 896 | CGTWDVSLSAGDWF | 1632 | TGCGGAACGTGGGATGTCAGCCTGAGTGCTGGCGATGTAGTTTTC |
| 897 | CGTWDVSLNVVVF | 1633 | TGCGGAACATGGGATGTCAGCCTGAATGTCGTGGTTTTC |
| 898 | CGTWDVSLNTQVF | 1634 | TGCGGAACATGGGATGTCAGCCTGAATACTCAGGTGTTC |
| 899 | CGTWDVSLGALF | 1635 | TGCGGCACATGGGATGTGAGCCTGGGTGCGCTGTTC |
| 900 | CGTWDVNLKTVVF | 1636 | TGCGGAACGTGGGACGTTAATCTGAAAACTGTCGTTTTC |
| 901 | CGTWDVILSAEVF | 1637 | TGCGGAACATGGGATGTCATCCTGAGTGCTGAGGTATTC |
| 902 | CGTWDTTVSAVVF | 1638 | TGCGGAACATGGGATACCACCGTGAGTGCTGTGGTTTTC |
| 903 | CGTWDTTLTAWVF | 1639 | TGCGGAACATGGGATACCACCCTGACTGCCTGGGTGTTC |
| 904 | CGTWDTTLSVFLF | 1640 | TGCGGAACATGGGACACCACCTTGAGTGTTTTCCTATTC |
| 905 | CGTWDTSVSAGVF | 1641 | TGCGGGACTTGGGATACCAGTGTGAGTGCTGGGGTGTTC |
| 906 | CGTWDTSVISWVF | 1642 | TGCGGAACATGGGATACCAGTGTGATTTCTTGGGTTTTC |
| 907 | CGTWDTSRSSLYVVF | 1643 | TGCGGAACATGGGATACCAGTCGGAGTTCTCTCTATGTGGTCTTC |
| 908 | CGTWDTSRSAWVF | 1644 | TGCGGAACATGGGATACCAGCCGGAGTGCTTGGGTATTC |
| 909 | CGTWDTSRNPGGIF | 1645 | TGCGGAACATGGGATACCAGCCGGAATCCTGGAGGAATTTTC |
| 910 | CGTWDTSRGHVF | 1646 | TGCGGAACATGGGACACCAGTCGGGGTCATGTTTTC |
| 911 | CGTWDTSPSTGQVLF | 1647 | TGCGGAACATGGGATACCAGCCCGAGTACTGGCCAGGTGCTTTTC |
| 912 | CGTWDTSPSAWVF | 1648 | TGCGGAACATGGGATACCAGCCCGAGTGCCTGGGTGTTC |
| 913 | CGTWDTSLTWVF | 1649 | TGCGGAACATGGGATACTAGCCTGACCTGGGTGTTC |
| 914 | CGTWDTSLTWFAVF | 1650 | TGCGGAACATGGGATACCAGCCTGACGTGGTTCGCAGTGTTC |
| 915 | CGTWDTSLTWVF | 1651 | TGCGGAACATGGGATACCAGCCTGACTGTTGTGGTATTC |
| 916 | CGTWDTSLTTSWVF | 1652 | TGCGGAACATGGGATACCAGCCTGACTACTTCTTGGGTGTTC |
| 917 | CGTWDTSLTTGPFWVF | 1653 | TGCGGAACATGGGATACCAGCCTGACCACTGGTCCTTTTTGGGTGT |
| | | | TC |
| 918 | CGTWDTSLTPFYVF | 1654 | TGCGGAACATGGGATACCAGCCTGACTCCTTTTTATGTCTTC |
| 919 | CGTWDTSLTAYVF | 1655 | TGCGGAACATGGGATACCAGCCTGACTGCTTATGTCTTC |
| 920 | CGTWDTSLTAWVF | 1656 | TGCGGAACATGGGATACCAGCCTGACTGCTTGGGTGTTC |
| 921 | CGTWDTSLTAWGVF | 1657 | TGCGGAACATGGGATACCAGCCTGACTGCGTGGGGGGTGTTC |
| 922 | CGTWDTSLTAVVL | 1658 | TGCGGCACATGGGATACCAGCCTGACTGCGGTGGTTCTC |
| 923 | CGTWDTSLTARVF | 1659 | TGCGGAACCTGGGATACCAGCCTGACTGCTCGGGTTTTC |
| 924 | CGTWDTSLTAIVF | 1660 | TGCGGAACATGGGATACCAGCCTGACTGCGATTGTCTTC |
| 925 | CGTWDTSLTAGVF | 1661 | TGCGGAACATGGGATACCAGCCTGACTGCTGGTGTCTTC |
| 926 | CGTWDTSLSVYVF | 1662 | TGCGGAACATGGGATACCAGCCTGAGTGTTTATGTCTTC |
| 927 | CGTWDTSLSVVF | 1663 | TGCGGAACATGGGATACCAGCCTGAGTGTGGTGTTC |
| 928 | CGTWDTSLSVGEF | 1664 | TGCGGGACATGGGATACCAGCCTGAGTGTTGGGGAATTC |
| 929 | CGTWDTSLSTWVF | 1665 | TGCGGAACATGGGATACCAGCCTGAGTACTTGGGTGTTC |
| 930 | CGTWDTSLSTVVF | 1666 | TGCGGAACATGGGATACCAGCCTGAGTACTGTGGTATTC |
| 931 | CGTWDTSLSTGQVLF | 1667 | TGCGGAACATGGGATACCAGCCTGAGTACTGGCCAGGTGCTTTTC |
| 932 | CGTWDTSLSTGPLWVF | 1668 | TGCGGCACATGGGATACCAGCCTGAGCACTGGTCCTCTTTGGGTGT |
| | | | TC |
| 933 | CGTWDTSLSSYVF | 1669 | TGCGGAACTTGGGATACCAGCCTGAGTTCTTATGTCTTC |
| 934 | CGTWDTSLSSVVF | 1670 | TGCGGAACATGGGATACCAGCCTGAGTTCTGTGGTCTTC |
| 935 | CGTWDTSLSSRYIF | 1671 | TGCGGAACATGGGATACCAGCCTGAGTTCTAGATACATATTC |
| 936 | CGTWDTSLSSRFIF | 1672 | TGCGGAACATGGGATACCAGCCTGAGTTCTAGATTCATATTC |
| 937 | CGTWDTSLSSGWVF | 1673 | TGCGGAACATGGGATACCAGCCTGAGTTCTGGGTGGGTGTTC |
| 938 | CGTWDTSLSRYVF | 1674 | TGCGGAACATGGGATACCAGCCTGAGTCGGTATGTGTTC |
| 939 | CGTWDTSLSQWLF | 1675 | TGCGGAACTTGGGATACCAGTCTGAGTCAATGGCTGTTC |
| 940 | CGTWDTSLSPGLWVF | 1676 | TGCGGAACATGGGATACCAGCCTGAGTCCTGGCCTTTGGGTGTTC |
| 941 | CGTWDTSLSNYVF | 1677 | TGCGGAACATGGGATACCAGCCTGAGTAATTATGTCTTC |
| 942 | CGTWDTSLSIWVF | 1678 | TGCGGAACATGGGATACCAGCCTAAGTATTTGGGTGTTC |
| 943 | CGTWDTSLSIGPFWVF | 1679 | TGCGGCACATGGGATACCAGCCTGAGCATTGGTCCTTTTTGGGTGT |
| | | | TC |
| 944 | CGTWDTSLSGWVF | 1680 | TGCGGAACATGGGATACCAGCCTGAGTGGTTGGGTGTTC |
| 945 | CGTWDTSLSGTVF | 1681 | TGCGGAACATGGGATACCAGCCTGAGTGGTACAGTGTTC |
| 946 | CGTWDTSLSGGQVF | 1682 | TGCGGAACATGGGATACTAGTCTGAGTGGTGGCCAGGTGTTC |
| 947 | CGTWDTSLSGGIF | 1683 | TGCGGAACATGGGATACCAGCCTGAGTGGTGGGATATTC |
| 948 | CGTWDTSLSGEDVVI | 1684 | TGCGGAACATGGGATACCAGCCTGAGTGGTGAGGATGTGGTAATC |
| 949 | CGTWDTSLSFLYAF | 1685 | TGCGGAACATGGGATACCAGCCTGAGTTTCCTTTATGCTTTC |
| 950 | CGTWDTSLSEVVF | 1686 | TGCGGAACATGGGATACCAGCCTGAGTGAGGTCGTATTC |
| 951 | CGTWDTSLSEVF | 1687 | TGCGGAACATGGGATACCAGCCTGAGTGAAGTGTTC |
| 952 | CGTWDTSLSENWVF | 1688 | TGCGGAACATGGGATACTAGCCTGAGTGAAAATTGGGTGTTC |
| 953 | CGTWDTSLSAYIF | 1689 | TGCGGAACATGGGATACCAGCCTGAGTGCCTACATATTC |
| 954 | CGTWDTSLSAVVL | 1690 | TGCGGAACATGGGATACCAGCCTGAGTGCTGTGGTACTC |
| 955 | CGTWDTSLSAVF | 1691 | TGCGGAACATGGGATACCAGCCTGAGTGCTGTTTTC |
| 956 | CGTWDTSLSARVF | 1692 | TGCGGAACATGGGATACCAGCCTGAGTGCCCGGGTGTTC |
| 957 | CGTWDTSLSARQVF | 1693 | TGCGGCACATGGGATACCAGCCTGAGTGCCCGCCAGGTATTC |
| 958 | CGTWDTSLSALVF | 1694 | TGCGGAACATGGGATACCAGCCTGAGTGCTTTGGTTTTC |
| 959 | CGTWDTSLSAKVF | 1695 | TGCGGAACATGGGATACCAGCCTGAGTGCTAAGGTGTTC |
| 960 | CGTWDTSLSAKIF | 1696 | TGCGGAACATGGGATACCAGCCTGAGTGCGAAAATCTTC |
| 961 | CGTWDTSLSAKAVF | 1697 | TGCGGAACATGGGATACCAGCCTGAGTGCCAAGGCGGTATTC |
| 962 | CGTWDTSLSAHAVF | 1698 | TGCGGAACATGGGATACCAGCCTGAGTGCCCATGCTGTGTTC |
| 963 | CGTWDTSLSAGYVF | 1699 | TGCGGAACATGGGATACCAGCCTGAGTGCTGGCTATGTCTTC |
| 964 | CGTWDTSLSAGRWVF | 1700 | TGCGGAACATGGGACACCAGTCTGAGTGCTGGCCGCTGGGTGTTC |
| 965 | CGTWDTSLSAGIF | 1701 | TGCGGAACATGGGATACCAGCCTGAGTGCTGGGATATTC |
| 966 | CGTWDTSLSAGGFRVF | 1702 | TGCGGAACATGGGATACCAGCCTGAGTGCTGGTGGGTTCCGGGTC |
| | | | TTC |
| 967 | CGTWDTSLSAGAF | 1703 | TGCGGAACATGGGATACCAGCCTGAGTGCTGGGGCATTC |
| 968 | CGTWDTSLSADWFF | 1704 | TGCGGAACATGGGATACCAGTCTGAGTGCTGATTGGTTTTTC |
| 969 | CGTWDTSLSADEYVF | 1705 | TGCGGAACATGGGATACCAGCCTGAGTGCTGATGAATATGTCTTC |
| 970 | CGTWDTSLSAAWVF | 1706 | TGCGGCACATGGGATACCAGCCTGAGTGCGGCTTGGGTGTTC |
| 971 | CGTWDTSLSAALF | 1707 | TGCGGAACATGGGATACCAGCCTGAGTGCTGCGCTATTC |
| 972 | CGTWDTSLSAAGVF | 1708 | TGCGGAACATGGGATACCAGCCTGAGTGCTGCGGGGGTTTTC |
| 973 | CGTWDTSLRVVVF | 1709 | TGCGGAACATGGGATACCAGCCTGAGAGTTGTGGTTTTC |
| 974 | CGTWDTSLRTWVF | 1710 | TGCGGAACATGGGATACCAGCCTGAGAACCTGGGTATTC |
| 975 | CGTWDTSLRGAVF | 1711 | TGCGGAACGTGGGATACCAGCCTGAGGGGTGCAGTGTTC |
| 976 | CGTWDTSLRAVVF | 1712 | TGCGGAACATGGGATACCAGCCTGCGTGCTGTGGTATTC |
| 977 | CGTWDTSLNVVYVF | 1713 | TGCGGAACATGGGATACAAGCCTGAATGTAGTTTATGTCTTC |
| 978 | CGTWDTSLNTYLF | 1714 | TGCGGAACATGGGATACCAGCCTCAACACCTACCTGTTC |
| 979 | CGTWDTSLNFAWLF | 1715 | TGCGGAACATGGGATACTAGCCTGAACTTCGCTTGGCTGTTC |
| 980 | CGTWDTSLLVWLF | 1716 | TGCGGCACATGGGATACCAGCCTTCTTGTGTGGCTTTTC |
| 981 | CGTWDTSLKTWVF | 1717 | TGCGGAACATGGGATACCAGTCTGAAGACGTGGGTGTTC |
| 982 | CGTWDTSLIVWVF | 1718 | TGCGGAACATGGGATACCAGTCTGATTGTCTGGGTGTTC |
| 983 | CGTWDTSLITGVF | 1719 | TGCGGAACATGGGATACCAGCCTAATTACTGGGGTGTTC |
| 984 | CGTWDTSLISVVF | 1720 | TGCGGAACATGGGATACCAGCCTGATTAGCGTGGTATTC |
| 985 | CGTWDTSLIAYVF | 1721 | TGCGGAACATGGGATACCAGCCTGATTGCTTATGTCTTC |
| 986 | CGTWDTSLHTELF | 1722 | TGCGGAACATGGGATACCAGCCTGCACACTGAGTTGTTC |
| 987 | CGTWDTSLGSYVF | 1723 | TGCGGAACTTGGGATACCAGCCTGGGTTCTTATGTCTTC |
| 988 | CGTWDTSLGSLWVF | 1724 | TGCGGAACATGGGATACCAGCCTGGGTTCTCTTTGGGTGTTC |
| 989 | CGTWDTSLGSGVF | 1725 | TGCGGTACATGGGATACCAGCCTGGGTTCTGGGGTATTC |
| 990 | CGTWDTSLGGRGVF | 1726 | TGCGGAACTTGGGATACCAGTCTGGGTGGTAGAGGGGTCTTC |
| 991 | CGTWDTSLGAWVF | 1727 | TGCGGAACATGGGATACCAGCCTGGGTGCTTGGGTGTTC |
| 992 | CGTWDTSLGAVVF | 1728 | TGCGGAACATGGGATACCAGCCTGGGTGCCGTGGTATTC |
| 993 | CGTWDTSLGAGVF | 1729 | TGCGGAACATGGGATACCAGCCTGGGTGCTGGGGTATTC |
| 994 | CGTWDTSLGAGLF | 1730 | TGCGGAACATGGGATACCAGCCTGGGTGCTGGCCTATTC |
| 995 | CGTWDTSLDAVVF | 1731 | TGCGGAACATGGGATACCAGTCTGGATGCTGTGGTTTTC |
| 996 | CGTWDTSLDAVLF | 1732 | TGCGGGACTTGGGATACCAGCCTGGATGCTGTGCTGTTC |
| 997 | CGTWDTSLAWVF | 1733 | TGCGGAACATGGGATACCAGCCTGGCTTGGGTGTTC |
| 998 | CGTWDTSLATGLF | 1734 | TGCGGAACATGGGATACCAGCCTGGCGACTGGACTGTTC |
| 999 | CGTWDTSLAPVVF | 1735 | TGCGGGACATGGGATACCAGCCTGGCCCCTGTAGTCTTC |
| 1000 | CGTWDTRLTIVIF | 1736 | TGCGGAACATGGGACACCCGCCTGACTATTGTGATCTTC |
| 1001 | CGTWDTRLSVWLF | 1737 | TGTGGAACATGGGACACCAGGCTGAGTGTTTGGCTGTTC |
| 1002 | CGTWDTRLSVGVF | 1738 | TGCGGAACGTGGGACACCAGACTGAGTGTTGGGGTTTTC |
| 1003 | CGTWDTRLSTVIF | 1739 | TGCGGCACATGGGATACCAGACTGAGTACTGTAATTTTC |
| 1004 | CGTWDTRLSSVVF | 1740 | TGCGGAACATGGGATACCCGCCTGAGTTCTGTGGTCTTC |
| 1005 | CGTWDTRLSIVVF | 1741 | TGCGGAACATGGGATACCCGCCTGAGTATTGTGGTTTTC |
| 1006 | CGTWDTRLSAYVVF | 1742 | TGCGGAACATGGGATACCAGACTGAGTGCCTATGTGGTATTC |
| 1007 | CGTWDTRLSAWVF | 1743 | TGCGGAACCTGGGACACCCGCCTGAGTGCGTGGGTGTTC |
| 1008 | CGTWDTRLSAVVF | 1744 | TGCGGAACATGGGATACCAGACTGAGTGCTGTGGTGTTC |
| 1009 | CGTWDTRLSAGLF | 1745 | TGCGGAACATGGGATACCCGCCTGAGTGCTGGGTTGTTC |
| 1010 | CGTWDTRLSAGGVF | 1746 | TGCGGAACATGGGATACCAGACTGAGTGCTGGTGGGGTGTTC |
| 1011 | CGTWDTRLNVWLF | 1747 | TGCGGAACATGGGATACCAGATTGAATGTGTGGCTATTC |
| 1012 | CGTWDTNREVVLL | 1748 | TGCGGAACATGGGATACCAACCGGGAAGTTGTGCTCCTC |
| 1013 | CGTWDTNLRAHVF | 1749 | TGCGGAACATGGGATACCAACCTGCGTGCCCATGTCTTC |
| 1014 | CGTWDTNLPAVVF | 1750 | TGCGGAACATGGGATACTAATCTGCCCGCTGTAGTGTTC |
| 1015 | CGTWDTNLGGVF | 1751 | TGCGGAACATGGGACACCAATTTGGGTGGGGTGTTC |
| 1016 | CGTWDTIVSIGVF | 1752 | TGCGGAACATGGGATACCATCGTGAGTATTGGGGTGTTC |
| 1017 | CGTWDTILSAVVF | 1753 | TGCGGAACATGGGATACCATCCTGAGTGCGGTGGTGTTC |
| 1018 | CGTWDTILSAEVF | 1754 | TGCGGCACATGGGATACCATCCTGAGTGCTGAGGTGTTC |
| 1019 | CGTWDTHLGVVF | 1755 | TGCGGAACATGGGATACCCACCTGGGTGTGGTTTTC |
| 1020 | CGTWDTGPSPHWLF | 1756 | TGCGGAACATGGGATACCGGCCCGAGCCCTCATTGGCTGTTC |
| 1021 | CGTWDTGLTFGGVF | 1757 | TGCGGAACATGGGATACCGGCCTGACTTTTGGAGGCGTGTTC |
| 1022 | CGTWDTGLTAFVF | 1758 | TGCGGAACATGGGATACCGGCCTGACTGCTTTTGTCTTC |
| 1023 | CGTWDTGLSVWVF | 1759 | TGCGGAACATGGGATACCGGCCTGAGTGTTTGGGTGTTC |
| 1024 | CGTWDTGLSTGIF | 1760 | TGCGGAACATGGGATACCGGCCTGAGTACTGGGATTTTC |
| 1025 | CGTWDTGLSSLLF | 1761 | TGCGGAACATGGGATACCGGCCTGAGTTCCCTGCTCTTC |
| 1026 | CGTWDTGLSIVVF | 1762 | TGCGGAACGTGGGACACCGGCCTGAGTATTGTGGTGTTC |
| 1027 | CGTWDTGLSFVVF | 1763 | TGCGGAACGTGGGACACCGGCCTGAGTTTTGTGGTGTTC |
| 1028 | CGTWDTGLSAWVF | 1764 | TGCGGAACATGGGATACCGGCCTGAGTGCTTGGGTGTTC |
| 1029 | CGTWDTGLSAGVVF | 1765 | TGCGGAACATGGGATACCGGCCTGAGTGCTGGTGTGGTATTC |
| 1030 | CGTWDTGLRGWIF | 1766 | TGCGGAACATGGGATACCGGTCTGAGGGGTTGGATTTTC |
| 1031 | CGTWDTELSAGVF | 1767 | TGCGGAACATGGGATACCGAGCTAAGTGCGGGGGTCTTC |
| 1032 | CGTWDTALTAGVF | 1768 | TGCGGAACGTGGGATACCGCCCTGACTGCTGGGGTGTTC |
| 1033 | CGTWDTALSLVVF | 1769 | TGCGGAACATGGGATACTGCCCTGAGTCTTGTGGTCTTC |
| 1034 | CGTWDTALSAWLF | 1770 | TGCGGAACATGGGATACCGCCCTGAGTGCCTGGCTGTTC |
| 1035 | CGTWDTALSAGVF | 1771 | TGCGGCACATGGGATACCGCCCTGAGTGCTGGGGTGTTC |
| 1036 | CGTWDTALRGVLF | 1772 | TGCGGAACATGGGATACCGCCCTGCGTGGCGTGCTGTTC |
| 1037 | CGTWDTALKEWLF | 1773 | TGCGGAACATGGGATACCGCCCTGAAAGAATGGCTGTTC |
| 1038 | CGTWDRTLTAGDVLF | 1774 | TGCGGAACATGGGATAGGACCCTGACTGCTGGCGATGTGCTCTTC |
| 1039 | CGTWDRSVTYVF | 1775 | TGCGGAACATGGGATAGAAGCGTGACTTATGTCTTC |
| 1040 | CGTWDRSRNEWVF | 1776 | TGCGGAACATGGGATCGCAGCCGAAATGAATGGGTGTTC |
| 1041 | CGTWDRSLTVWVF | 1777 | TGCGGAACATGGGATCGCAGTCTGACTGTTTGGGTCTTC |
| 1042 | CGTWDRSLTPGWLF | 1778 | TGCGGAACATGGGATCGCAGCCTGACTCCTGGGTGGTTGTTC |
| 1043 | CGTWDRSLTAWVF | 1779 | TGCGGAACATGGGATAGAAGCCTGACTGCTTGGGTGTTC |
| 1044 | CGTWDRSLSVVVF | 1780 | TGCGGAACATGGGACCGCAGCCTGAGTGTTGTGGTATTC |
| 1045 | CGTWDRSLSVVF | 1781 | TGCGGCACATGGGATCGCAGCCTGAGTGTAGTCTTC |
| 1046 | CGTWDRSLSVQLF | 1782 | TGCGGAACATGGGATAGGAGCCTGAGTGTTCAATTGTTC |
| 1047 | CGTWDRSLSVLWVF | 1783 | TGCGGAACATGGGATCGCAGCCTCAGTGTTCTTTGGGTGTTC |
| 1048 | CGTWDRSLSVGLF | 1784 | TGCGGAACATGGGATCGCAGCCTGAGTGTTGGATTATTC |
| 1049 | CGTWDRSLSTWVF | 1785 | TGCGGAACATGGGATCGCAGCCTGAGTACTTGGGTGTTC |
| 1050 | CGTWDRSLSTHWVL | 1786 | TGCGGAACATGGGATAGAAGCCTGAGTACTCATTGGGTGCTC |
| 1051 | CGTWDRSLSTHWVF | 1787 | TGCGGAACATGGGATAGAAGCCTGAGTACTCATTGGGTGTTC |
| 1052 | CGTWDRSLSSAVF | 1788 | TGCGGAACCTGGGATCGAAGCCTGAGTTCTGCGGTGTTC |
| 1053 | CGTWDRSLSPSYVF | 1789 | TGCGGAACATGGGACAGAAGCCTGAGTCCCTCTTATGTCTTC |
| 1054 | CGTWDRSLSGEVF | 1790 | TGCGGAACATGGGATAGGAGCCTGAGTGGTGAGGTGTTC |
| 1055 | CGTWDRSLSGAVF | 1791 | TGCGGAACATGGGATAGGAGCCTGAGTGGTGCGGTGTTC |
| 1056 | CGTWDRSLSAVAF | 1792 | TGCGGAACATGGGATCGCAGCCTGAGTGCTGTGGCATTC |
| 1057 | CGTWDRSLSAGGEF | 1793 | TGCGGAACATGGGATAGGAGCCTGAGTGCCGGGGGGGAATTC |
| 1058 | CGTWDRSLSAFWVF | 1794 | TGCGGAACATGGGATCGCAGCCTGAGTGCTTTTTGGGTGTTC |
| 1059 | CGTWDRSLSAAVF | 1795 | TGCGGAACATGGGATAGGAGCCTGAGTGCTGCGGTGTTC |
| 1060 | CGTWDRSLSAALF | 1796 | TGCGGAACATGGGATAGGAGCCTGAGTGCTGCACTCTTC |
| 1061 | CGTWDRSLRVF | 1797 | TGCGGAACATGGGATCGCAGCCTGAGAGTGTTC |
| 1062 | CGTWDRSLNWVF | 1798 | TGCGGTACATGGGACAGAAGCCTTAATTGGGTGTTC |
| 1063 | CGTWDRSLNVYVF | 1799 | TGCGGAACATGGGATCGCAGCCTGAATGTTTATGTCTTC |
| 1064 | CGTWDRSLNVGVF | 1800 | TGCGGAACATGGGATAGGAGCCTGAATGTTGGGGTGTTC |
| 1065 | CGTWDRSLHVVF | 1801 | TGCGGAACATGGGATCGGAGCCTGCATGTGGTCTTC |
| 1066 | CGTWDRSLGGWVF | 1802 | TGTGGAACATGGGATCGCAGCCTGGGTGGTTGGGTGTTC |
| 1067 | CGTWDRSLGAFWVF | 1803 | TGCGGAACATGGGATCGCAGCCTGGGTGCTTTTTGGGTGTTC |
| 1068 | CGTWDRSLFWVF | 1804 | TGCGGAACATGGGATAGAAGCCTGTTTTGGGTGTTC |
| 1069 | CGTWDRSLAAGVF | 1805 | TGCGGAACGTGGGATCGCAGCCTGGCTGCTGGGGTGTTC |
| 1070 | CGTWDRRLSGVVF | 1806 | TGCGGAACATGGGATAGGAGGTTGAGTGGTGTCGTATTC |
| 1071 | CGTWDRRLSDVVF | 1807 | TGCGGAACGTGGGATCGCCGCCTAAGTGATGTGGTATTC |
| 1072 | CGTWDRRLSAVVF | 1808 | TGCGGAACATGGGATAGGAGGCTGAGTGCTGTGGTATTC |
| 1073 | CGTWDRRLNVAFF | 1809 | TGCGGAACATGGGATAGACGCCTGAATGTTGCGTTCTTC |
| 1074 | CGTWDRRLLAVF | 1810 | TGTGGAACATGGGATAGGAGGCTGCTTGCTGTTTTC |
| 1075 | CGTWDRNLRAVVF | 1811 | TGCGGAACTTGGGATAGGAACCTGCGCGCCGTGGTCTTC |
| 1076 | CGTWDRLSAGVF | 1812 | TGCGGAACATGGGATAGGCTGAGTGCTGGGGTGTTC |
| 1077 | CGTWDRGPNTGVF | 1813 | TGCGGAACATGGGATAGAGGCCCGAATACTGGGGTATTC |
| 1078 | CGTWDRGLNTVYVF | 1814 | TGCGGAACATGGGATAGAGGCCTGAATACTGTTTACGTCTTC |
| 1079 | CGTWDNYVSAPWVF | 1815 | TGCGGAACATGGGATAACTATGTGAGTGCCCCTTGGGTGTTC |
| 1080 | CGTWDNYLSAGDVVF | 1816 | TGCGGAACATGGGATAACTACCTGAGTGCTGGCGATGTGGTTTTC |
| 1081 | CGTWDNYLRAGVF | 1817 | TGCGGAACATGGGATAACTACCTGAGAGCTGGGGTCTTC |
| 1082 | CGTWDNYLGAVVF | 1818 | TGCGGAACATGGGACAATTATCTGGGTGCCGTGGTTTTC |
| 1083 | CGTWDNYLGAGVF | 1819 | TGCGGAACATGGGATAACTACCTGGGTGCGGGGGTGTTC |
| 1084 | CGTWDNTVSAPWVF | 1820 | TGCGGAACATGGGATAACACCGTGAGTGCCCCTTGGGTTTTC |
| 1085 | CGTWDNTLSLWVF | 1821 | TGCGGAACATGGGATAACACCCTGAGTCTTTGGGTGTTC |
| 1086 | CGTWDNTLSAGVF | 1822 | TGCGGAACATGGGATAACACCCTGAGTGCTGGGGTCTTC |
| 1087 | CGTWDNTLLTVLF | 1823 | TGCGGAACATGGGACAACACTCTGCTTACTGTGTTATTC |
| 1088 | CGTWDNRLSSVIF | 1824 | TGCGGAACATGGGATAACAGACTGAGTAGTGTGATTTTC |
| 1089 | CGTWDNRLSAVVF | 1825 | TGCGGAACATGGGATAACAGGTTGAGTGCTGTGGTCTTC |
| 1090 | CGTWDNRLSAGGIF | 1826 | TGCGGAACATGGGATAACAGGCTGAGTGCTGGTGGGATATTC |
| 1091 | CGTWDNRLSAEVF | 1827 | TGCGGAACATGGGATAACAGACTGAGTGCTGAGGTGTTC |
| 1092 | CGTWDNRLRVGVL | 1828 | TGTGGAACATGGGATAACAGACTGCGTGTTGGGGTTCTC |
| 1093 | CGTWDNRLLENVF | 1829 | TGCGGAACATGGGATAATCGCCTGCTTGAGAATGTCTTC |
| 1094 | CGTWDNNLRAVF | 1830 | TGCGGAACATGGGATAACAACCTGCGTGCTGTCTTC |
| 1095 | CGTWDNNLRAGVF | 1831 | TGCGGAACTTGGGATAATAACCTGCGTGCTGGAGTGTTC |
| 1096 | CGTWDNNLGGGRVF | 1832 | TGCGGAACATGGGACAACAATTTGGGCGGTGGCCGGGTGTTC |
| 1097 | CGTWDNNLGAGVL | 1833 | TGCGGAACATGGGATAACAACCTGGGTGCTGGCGTCCTC |
| 1098 | CGTWDNNLGAGVF | 1834 | TGCGGAACATGGGATAACAACCTGGGTGCTGGCGTCTTC |
| 1099 | CGTWDNILSAAVF | 1835 | TGCGGAACTTGGGATAACATCCTGAGCGCTGCGGTGTTC |
| 1100 | CGTWDNILDAGVF | 1836 | TGCGGAACCTGGGATAACATCTTGGATGCAGGGGTTTTC |
| 1101 | CGTWDNDLSGWLF | 1837 | TGCGGAACATGGGATAACGACCTGAGTGGTTGGCTGTTC |
| 1102 | CGTWDNDLSAWVF | 1838 | TGCGGAACATGGGATAACGACCTGAGTGCCTGGGTGTTC |
| 1103 | CGTWDLTLGGVVF | 1839 | TGCGGAACATGGGATCTCACCCTGGGTGGTGTGGTGTTC |
| 1104 | CGTWDLSLSAGVF | 1840 | TGCGGAACATGGGATCTCAGCCTGAGTGCTGGGGTATTC |
| 1105 | CGTWDLSLKEWVF | 1841 | TGCGGAACATGGGATCTCAGCCTGAAAGAATGGGTGTTC |
| 1106 | CGTWDLSLDAVVF | 1842 | TGCGGAACGTGGGATCTCAGCCTGGATGCTGTTGTTTTC |
| 1107 | CGTWDLKVF | 1843 | TGCGGAACCTGGGACCTGAAGGTTTTC |
| 1108 | CGTWDKTLSVWVF | 1844 | TGCGGAACATGGGATAAGACTCTGAGTGTTTGGGTGTTC |
| 1109 | CGTWDKSLSVWVF | 1845 | TGCGGAACATGGGATAAGAGCCTGAGTGTTTGGGTGTTC |
| 1110 | CGTWDKSLSGVVF | 1846 | TGCGGAACATGGGATAAGAGCCTGAGTGGTGTGGTATTT |
| 1111 | CGTWDKSLSDWVF | 1847 | TGCGGAACATGGGATAAGAGCCTGAGTGATTGGGTGTTC |
| 1112 | CGTWDKSLSALVF | 1848 | TGCGGAACATGGGATAAGAGCCTGAGTGCTTTGGTTTTC |
| 1113 | CGTWDKSLSAGVF | 1849 | TGCGGAACATGGGATAAGAGCCTGAGTGCTGGCGTCTTC |
| 1114 | CGTWDKSLSADVF | 1850 | TGCGGAACATGGGATAAGAGCCTGAGTGCCGACGTCTTC |
| 1115 | CGTWDKRLTIVVF | 1851 | TGCGGAACATGGGATAAACGCCTGACTATTGTGGTCTTC |
| 1116 | CGTWDKRLSAWVL | 1852 | TGCGGAACATGGGATAAACGCCTGAGTGCCTGGGTGCTC |
| 1117 | CGTWDKNLRAVVF | 1853 | TGCGGAACATGGGATAAGAACCTGCGTGCTGTGGTCTTC |
| 1118 | CGTWDITLSGFVF | 1854 | TGCGGAACATGGGATATCACCCTGAGTGGGTTTGTCTTC |
| 1119 | CGTWDITLHTGVF | 1855 | TGCGGAACATGGGATATCACCTTGCATACTGGAGTATTC |
| 1120 | CGTWDISVTVVF | 1856 | TGCGGAACATGGGATATCAGTGTGACTGTGGTGTTC |
| 1121 | CGTWDISVRGYAF | 1857 | TGCGGAACATGGGATATCAGTGTGAGGGGTTATGCCTTC |
| 1122 | CGTWDISRWVF | 1858 | TGCGGAACATGGGATATCAGCCGTTGGGTTTTC |
| 1123 | CGTWDISPSAWVF | 1859 | TGCGGAACATGGGATATCAGCCCGAGTGCTTGGGTGTTC |
| 1124 | CGTWDISLSVWVF | 1860 | TGCGGAACATGGGATATTAGCCTGAGTGTCTGGGTGTTC |
| 1125 | CGTWDISLSVVF | 1861 | TGCGGAACATGGGATATCAGCCTGAGTGTTGTATTC |
| 1126 | CGTWDISLSSVVF | 1862 | TGCGGAACTTGGGATATCAGCCTGAGTTCTGTGGTGTTC |
| 1127 | CGTWDISLSHWLF | 1863 | TGCGGAACATGGGATATCAGCCTGAGTCACTGGTTGTTC |
| 1128 | CGTWDISLSGWVF | 1864 | TGCGGAACATGGGATATCAGTCTGAGTGGTTGGGTGTTC |
| 1129 | CGTWDISLSGRVF | 1865 | TGCGGAACATGGGATATCAGCCTGAGTGGTCGAGTGTTC |
| 1130 | CGTWDISLSAWAF | 1866 | TGCGGAACATGGGACATCAGCCTGAGTGCTTGGGCGTTC |
| 1131 | CGTWDISLSAVVF | 1867 | TGCGGAACATGGGATATCAGCCTGAGTGCTGTGGTTTTC |
| 1132 | CGTWDISLSAVIF | 1868 | TGCGGGACATGGGACATCAGCCTGAGTGCTGTGATATTC |
| 1133 | CGTWDISLSAVF | 1869 | TGCGGAACATGGGATATCAGCCTGAGTGCTGTGTTC |
| 1134 | CGTWDISLSARVF | 1870 | TGCGGAACATGGGATATCAGCCTGAGTGCCCGGGTGTTC |
| 1135 | CGTWDISLSALVF | 1871 | TGCGGAACATGGGATATCAGCCTGAGTGCCCTGGTGTTC |
| 1136 | CGTWDISLSAHVF | 1872 | TGCGGAACATGGGATATTAGCCTGAGTGCCCATGTCTTC |
| 1137 | CGTWDISLSAGVVF | 1873 | TGCGGAACATGGGATATCAGCCTGAGTGCTGGGGTGGTATTC |
| 1138 | CGTWDISLSAGPYVF | 1874 | TGCGGAACATGGGATATCAGCCTGAGTGCCGGCCCTTATGTCTTC |
| 1139 | CGTWDISLSAGGVF | 1875 | TGCGGCACATGGGATATCAGCCTGAGTGCTGGAGGGGTGTTC |
| 1140 | CGTWDISLSAEVF | 1876 | TGCGGAACATGGGATATCAGCCTGAGTGCTGAGGTTTTC |
| 1141 | CGTWDISLSAAVF | 1877 | TGCGGAACATGGGATATCAGCCTGAGTGCTGCTGTGTTC |
| 1142 | CGTWDISLRAVF | 1878 | TGCGGAACATGGGATATCAGCCTGCGTGCTGTGTTC |
| 1143 | CGTWDISLNTGVF | 1879 | TGCGGAACATGGGATATTAGCCTGAATACTGGGGTGTTC |
| 1144 | CGTWDISLNNYVF | 1880 | TGCGGAACATGGGATATCAGCCTAAATAATTATGTCTTC |
| 1145 | CGTWDISLIAGVF | 1881 | TGCGGAACATGGGATATCAGCCTAATTGCTGGGGTATTC |
| 1146 | CGTWDISLHTWLF | 1882 | TGCGGAACATGGGATATCAGCCTGCATACTTGGCTGTTC |
| 1147 | CGTWDIRLTDELLF | 1883 | TGCGGAACATGGGATATCCGCCTGACCGATGAGCTGTTATTC |
| 1148 | CGTWDIRLSGFVF | 1884 | TGCGGAACATGGGATATCAGACTGAGCGGTTTTGTTTTC |
| 1149 | CGTWDINLGAGGLYVF | 1885 | TGCGGAACATGGGATATCAACCTGGGTGCTGGGGGCCTTTATGTC |
| | | | TTC |
| 1150 | CGTWDIILSAEVF | 1886 | TGCGGAACATGGGATATCATCCTGAGTGCTGAGGTATTC |
| 1151 | CGTWDHTLSAVF | 1887 | TGCGGAACATGGGATCACACCCTGAGTGCTGTCTTC |
| 1152 | CGTWDHTLLTVLF | 1888 | TGCGGAACATGGGACCACACTCTGCTTACTGTGTTATTC |
| 1153 | CGTWDHSLTAVVF | 1889 | TGCGGAACATGGGATCACAGCCTGACTGCTGTGGTATTC |
| 1154 | CGTWDHSLTAGIF | 1890 | TGCGGAACCTGGGATCACAGCCTGACTGCTGGGATATTC |
| 1155 | CGTWDHSLSVVLF | 1891 | TGCGGAACATGGGATCACAGCCTGAGTGTTGTATTATTC |
| 1156 | CGTWDHSLSLVF | 1892 | TGCGGAACATGGGATCACAGCCTGAGTTTGGTATTC |
| 1157 | CGTWDHSLSIGVF | 1893 | TGCGGAACATGGGATCACAGCCTGTCTATTGGGGTTTTC |
| 1158 | CGTWDHSLSAGVF | 1894 | TGCGGAACATGGGATCACAGCCTGAGTGCTGGGGTGTTC |
| 1159 | CGTWDHSLSAFVF | 1895 | TGTGGAACTTGGGATCACAGCCTGAGTGCTTTCGTGTTC |
| 1160 | CGTWDHSLSAAVF | 1896 | TGCGGAACATGGGATCACAGTCTGAGTGCTGCTGTTTTC |
| 1161 | CGTWDHNLRAVF | 1897 | TGCGGAACATGGGACCACAATCTGCGTGCTGTCTTC |
| 1162 | CGTWDFTLSVGRF | 1898 | TGCGGGACATGGGATTTCACCCTGAGTGTTGGGCGCTTC |
| 1163 | CGTWDFTLSAPVF | 1899 | TGCGGAACATGGGATTTCACCCTGAGTGCTCCTGTCTTC |
| 1164 | CGTWDFSVSAGWVF | 1900 | TGCGGAACGTGGGATTTCAGCGTGAGTGCTGGGTGGGTGTTC |
| 1165 | CGTWDFSLTTWLF | 1901 | TGCGGAACGTGGGATTTCAGTCTTACTACCTGGTTATTC |
| 1166 | CGTWDFSLSVWVF | 1902 | TGCGGAACATGGGATTTCAGCCTGAGTGTTTGGGTGTTC |
| 1167 | CGTWDFSLSTGVF | 1903 | TGCGGAACATGGGATTTCAGCCTGAGTACTGGGGTTTTC |
| 1168 | CGTWDFSLSGVVF | 1904 | TGCGGCACATGGGATTTCAGCCTGAGTGGTGTGGTATTC |
| 1169 | CGTWDFSLSGFVF | 1905 | TGCGGAACATGGGATTTCAGCCTGAGTGGTTTCGTGTTC |
| 1170 | CGTWDFSLSAGVF | 1906 | TGCGGAACATGGGATTTCAGCCTGAGTGCTGGGGTGTTC |
| 1171 | CGTWDETVRGWVF | 1907 | TGCGGAACATGGGATGAAACCGTGAGAGGTTGGGTGTTC |
| 1172 | CGTWDESLRSWVF | 1908 | TGCGGAACATGGGATGAAAGTCTGAGAAGCTGGGTGTTC |
| 1173 | CGTWDERQTDESYVF | 1909 | TGCGGAACTTGGGATGAGAGGCAGACTGATGAGTCCTATGTCTTC |
| 1174 | CGTWDERLVAGQVF | 1910 | TGCGGAACATGGGATGAGAGACTCGTTGCTGGCCAGGTCTTC |
| 1175 | CGTWDERLSPGAFF | 1911 | TGCGGAACATGGGATGAGAGACTGAGTCCTGGAGCTTTTTTC |
| 1176 | CGTWDEKVF | 1912 | TGCGGAACATGGGATGAGAAGGTGTTC |
| 1177 | CGTWDEGQTTDFFVF | 1913 | TGCGGAACCTGGGATGAAGGCCAGACTACTGATTTCTTTGTCTTC |
| 1178 | CGTWDDTLAGVVF | 1914 | TGCGGAACATGGGATGACACCCTGGCTGGTGTGGTCTTC |
| 1179 | CGTWDDRLTSAVF | 1915 | TGCGGAACATGGGATGACAGGCTGACTTCTGCGGTCTTC |
| 1180 | CGTWDDRLFVVVF | 1916 | TGCGGAACATGGGATGACAGACTGTTTGTTGTGGTATTC |
| 1181 | CGTWDDNLRGWVF | 1917 | TGCGGAACATGGGATGATAACCTGAGAGGTTGGGTGTTC |
| 1182 | CGTWDDNLRGVVF | 1918 | TGCGGAACATGGGATGACAACCTGCGTGGTGTCGTGTTC |
| 1183 | CGTWDDNLNIGRVF | 1919 | TGCGGAACCTGGGATGACAATTTGAATATTGGAAGGGTGTTC |
| 1184 | CGTWDDILSAVIF | 1920 | TGCGGAACATGGGATGACATCCTGAGTGCTGTGATATTC |
| 1185 | CGTWDDILRGWVF | 1921 | TGCGGAACATGGGATGATATCCTGAGAGGTTGGGTGTTC |
| 1186 | CGTWDATLSPGWLF | 1922 | TGCGGAACATGGGATGCCACCCTGAGTCCTGGGTGGTTATTC |
| 1187 | CGTWDASVTSWVF | 1923 | TGCGGAACATGGGATGCCAGCGTGACTTCTTGGGTGTTC |
| 1188 | CGTWDASLTSVVF | 1924 | TGCGGAACATGGGATGCCAGCCTGACTTCTGTGGTCTTC |
| 1189 | CGTWDASLSVWVF | 1925 | TGCGGAACATGGGATGCCAGCCTGAGTGTTTGGGTGTTC |
| 1190 | CGTWDASLSVPWVF | 1926 | TGCGGAACATGGGATGCCAGCCTGAGTGTTCCTTGGGTGTTC |
| 1191 | CGTWDASLSVAVF | 1927 | TGCGGAACATGGGATGCCAGCCTGAGTGTGGCGGTATTC |
| 1192 | CGTWDASLSTWVF | 1928 | TGCGGAACATGGGATGCCAGCCTGAGTACCTGGGTATTC |
| 1193 | CGTWDASLSGVVF | 1929 | TGCGGAACATGGGATGCCAGCCTGAGTGGTGTGGTATTC |
| 1194 | CGTWDASLSGGGEF | 1930 | TGCGGAACATGGGATGCCAGCCTGAGTGGTGGGGGAGAATTC |
| 1195 | CGTWDASLSAGVF | 1931 | TGCGGAACATGGGATGCCAGCCTGAGTGCTGGGGTGTTC |
| 1196 | CGTWDASLSAGLF | 1932 | TGCGGAACATGGGATGCCAGCCTGAGTGCTGGGCTTTTC |
| 1197 | CGTWDASLSAEVF | 1933 | TGTGGCACATGGGATGCCAGCCTGAGTGCTGAAGTCTTC |
| 1198 | CGTWDASLSADFWVF | 1934 | TGCGGAACATGGGATGCCAGCCTGAGTGCTGACTTTTGGGTGTTC |
| 1199 | CGTWDASLRVFF | 1935 | TGCGGAACATGGGATGCCAGCCTGAGAGTCTTCTTC |
| 1200 | CGTWDASLRAVVL | 1936 | TGCGGAACATGGGATGCCAGTCTGAGGGCTGTGGTACTC |
| 1201 | CGTWDASLNIWVF | 1937 | TGCGGAACATGGGATGCCAGCCTGAATATTTGGGTTTTC |
| 1202 | CGTWDASLKNLVF | 1938 | TGCGGGACATGGGATGCCAGCCTGAAGAATCTGGTCTTC |
| 2322 | CGTWDASLGAWVF | 1939 | TGCGGAACATGGGATGCCAGCCTGGGTGCCTGGGTATTC |
| 2323 | CGTWDASLGAVVF | 1940 | TGCGGAACATGGGATGCCAGCCTGGGTGCTGTGGTCTTC |
| 2324 | CGTWDASLGAGVF | 1941 | TGCGGAACATGGGATGCCAGCCTGGGTGCGGGGGTCTTC |
| 2325 | CGTWDARLSGLYVF | 1942 | TGCGGAACATGGGATGCTAGGCTGAGTGGCCTTTATGTCTTC |
| 2326 | CGTWDARLGGAVF | 1943 | TGTGGAACCTGGGATGCGAGACTGGGTGGTGCAGTCTTC |
| 2327 | CGTWDANLRAGVF | 1944 | TGCGGAACATGGGATGCCAATCTGCGTGCTGGGGTCTTC |
| 2328 | CGTWDAIISGWVF | 1945 | TGCGGAACATGGGATGCTATCATAAGTGGTTGGGTGTTC |
| 2329 | CGTWDAGQSVWVF | 1946 | TGCGGAACATGGGATGCCGGCCAGAGTGTTTGGGTGTTC |
| 2330 | CGTWDAGLTGLYVF | 1947 | TGCGGCACATGGGATGCCGGGCTGACTGGCCTTTATGTCTTC |
| 2331 | CGTWDAGLSVYVF | 1948 | TGCGGAACTTGGGATGCCGGTCTGAGTGTTTATGTCTTC |
| 2332 | CGTWDAGLSTGVF | 1949 | TGCGGGACATGGGATGCCGGCCTGAGTACTGGGGTCTTC |
| 2333 | CGTWDAGLSGDVF | 1950 | TGCGGAACATGGGATGCCGGCCTGAGTGGGGACGTTTTC |
| 2334 | CGTWDAGLSAGYVF | 1951 | TGCGGAACATGGGATGCCGGCCTGAGTGCTGGTTATGTCTTC |
| 2335 | CGTWDAGLRVWVF | 1952 | TGCGGAACATGGGATGCCGGCCTGCGTGTTTGGGTGTTC |
| 2336 | CGTWDAGLREIF | 1953 | TGCGGAACATGGGATGCCGGCCTGAGGGAAATTTTC |
| 2337 | CGTWASSLSSWVF | 1954 | TGCGGAACATGGGCCAGCAGCCTGAGTTCTTGGGTGTTC |
| 2338 | CGTWAGSLSGHVF | 1955 | TGCGGAACATGGGCTGGCAGCCTGAGTGGTCATGTCTTC |
| 2339 | CGTWAGSLSAAWVF | 1956 | TGCGGAACATGGGCTGGCAGCCTGAGTGCCGCTTGGGTGTTC |
| 2340 | CGTWAGSLNVYWVF | 1957 | TGCGGAACATGGGCTGGCAGCCTGAATGTTTATTGGGTGTTC |
| 2341 | CGTWAGNLRPNWVF | 1958 | TGCGGAACATGGGCTGGCAACCTGAGACCTAATTGGGTGTTC |
| 2342 | CGTRGSLGGAVF | 1959 | TGCGGAACAAGGGGTAGCCTGGGTGGTGCGGTGTTC |
| 2343 | CGTRDTTLSVPVF | 1960 | TGCGGAACAAGGGATACCACCCTGAGTGTCCCGGTGTTC |
| 2344 | CGTRDTSLNIEIF | 1961 | TGCGGAACACGGGATACCAGCCTCAATATTGAAATCTTC |
| 2345 | CGTRDTSLNDVF | 1962 | TGTGGAACACGGGATACCAGCCTGAATGATGTCTTC |
| 2346 | CGTRDTRLSIVVF | 1963 | TGCGGAACACGGGATACCCGCCTGAGTATTGTGGTTTTC |
| 2347 | CGTRDTILSAEVF | 1964 | TGCGGCACACGGGATACCATCCTGAGTGCTGAGGTGTTC |
| 2348 | CGTRDRSLSGWVF | 1965 | TGCGGAACACGGGATAGAAGCCTGAGTGGTTGGGTGTTC |
| 2349 | CGSWYYNVFLF | 1966 | TGCGGATCATGGTATTACAATGTCTTCCTTTTC |
| 2350 | CGSWHSSLNLVVF | 1967 | TGCGGATCTTGGCATAGCAGCCTCAACCTTGTCGTCTTC |
| 2351 | CGSWGSGLSAPYVF | 1968 | TGCGGATCATGGGGTAGTGGCCTGAGTGCCCCTTATGTCTTC |
| 2352 | CGSWESGLGAWLF | 1969 | TGCGGTTCGTGGGAAAGCGGCCTGGGTGCTTGGCTGTTC |
| 2353 | CGSWDYGLLLF | 1970 | TGCGGATCCTGGGATTACGGCCTCCTACTCTTC |
| 2354 | CGSWDVSLTAVF | 1971 | TGCGGTTCATGGGATGTCAGCCTGACTGCTGTTTTC |
| 2355 | CGSWDVSLNVGIF | 1972 | TGCGGATCCTGGGATGTCAGTCTCAATGTTGGCATTTTC |
| 2356 | CGSWDTTLRAWVF | 1973 | TGCGGATCATGGGATACCACCCTGCGTGCTTGGGTGTTC |
| 2357 | CGSWDTSPVRAWVF | 1974 | TGCGGCTCGTGGGATACCAGCCCTGTCCGTGCTTGGGTGTTC |
| 2358 | CGSWDTSLSVWVF | 1975 | TGCGGATCATGGGATACCAGCCTGAGTGTTTGGGTGTTC |
| 2359 | CGSWDTSLSAEVF | 1976 | TGCGGATCATGGGATACCAGCCTGAGTGCTGAGGTGTTC |
| 2360 | CGSWDTSLRAWVF | 1977 | TGCGGCTCGTGGGATACCAGCCTGCGTGCTTGGGTGTTC |
| 2361 | CGSWDTSLRAWAF | 1978 | TGCGGCTCGTGGGATACCAGCCTGCGTGCTTGGGCGTTC |
| 2362 | CGSWDTSLDARLF | 1979 | TGCGGATCATGGGATACCAGCCTGGATGCTAGGCTGTTC |
| 2363 | CGSWDTILLVYVF | 1980 | TGCGGATCATGGGATACCATCCTGCTTGTCTATGTCTTC |
| 2364 | CGSWDRWQAAVF | 1981 | TGCGGATCATGGGATCGCTGGCAGGCTGCTGTCTTC |
| 2365 | CGSWDRSLSGYVF | 1982 | TGCGGATCATGGGATAGGAGCCTGAGTGGGTATGTCTTC |
| 2366 | CGSWDRSLSAYVF | 1983 | TGCGGATCATGGGATAGAAGCCTGAGTGCTTATGTCTTC |
| 2367 | CGSWDRSLSAVVF | 1984 | TGCGGATCATGGGATAGGAGCCTGAGTGCCGTGGTTTTC |
| 2368 | CGSWDNTLGVVLF | 1985 | TGCGGATCATGGGATAACACCTTGGGTGTTGTTCTCTTC |
| 2369 | CGSWDNRLSTVIF | 1986 | TGCGGATCGTGGGATAACAGACTAAGTACTGTCATCTTC |
| 2370 | CGSWDNRLNTVIF | 1987 | TGCGGAAGCTGGGATAATCGATTGAACACTGTGATTTTC |
| 2371 | CGSWDLSPVRVLVF | 1988 | TGCGGTTCATGGGATCTCAGCCCTGTACGTGTCCTTGTGTTC |
| 2372 | CGSWDLSLSAVVF | 1989 | TGCGGATCATGGGATCTCAGCCTGAGTGCTGTCGTTTTC |
| 2373 | CGSWDKNLRAVLF | 1990 | TGCGGATCATGGGATAAAAACCTGCGTGCTGTGCTGTTC |
| 2374 | CGSWDISLSAGVF | 1991 | TGCGGCTCATGGGATATCAGCCTGAGTGCTGGGGTGTTC |
| 2375 | CGSWDIRLSAEVF | 1992 | TGCGGATCATGGGATATCAGACTGAGTGCAGAGGTCTTC |
| 2376 | CGSWDIKLNIGVF | 1993 | TGCGGATCATGGGACATCAAACTGAATATTGGGGTATTC |
| 2377 | CGSWDFSLNYFVF | 1994 | TGCGGATCATGGGATTTCAGTCTCAATTATTTTGTCTTC |
| 2378 | CGSWDASLSTEVF | 1995 | TGCGGATCATGGGATGCCAGCCTGAGTACTGAGGTGTTC |
| 2379 | CGSWDAGLRGWVF | 1996 | TGCGGATCCTGGGATGCCGGCCTGCGTGGCTGGGTTTTC |
| 2380 | CGRWESSLGAVVF | 1997 | TGCGGAAGATGGGAGAGCAGCCTGGGTGCTGTGGTTTTC |
| 2381 | CGRWDFSLSAYVF | 1998 | TGCGGAAGATGGGATTTTAGTCTGAGTGCTTATGTCTTC |
| 2382 | CGQWDNDLSVWVF | 1999 | TGCGGACAATGGGATAACGACCTGAGTGTTTGGGTGTTC |
| 2383 | CGPWHSSVTSGHVL | 2000 | TGCGGACCCTGGCATAGCAGCGTGACTAGTGGCCACGTGCTC |
| 2384 | CGLWDASLSAPTWVF | 2001 | TGCGGATTATGGGATGCCAGCCTGAGTGCTCCTACTTGGGTGTTC |
| 2385 | CGIWHTSLSAWVF | 2002 | TGTGGAATATGGCACACTAGCCTGAGTGCTTGGGTGTTC |
| 2386 | CGIWDYSLDTWVF | 2003 | TGCGGAATATGGGATTACAGCCTGGATACTTGGGTGTTC |
| 2387 | CGIWDTSLSAWVF | 2004 | TGCGGCATATGGGATACCAGCCTGAGTGCTTGGGTGTTC |
| 2388 | CGIWDTRLSVYVF | 2005 | TGCGGAATTTGGGATACCAGGCTGAGTGTTTATGTCTTC |
| 2389 | CGIWDTRLSVYIF | 2006 | TGCGGAATTTGGGATACCAGGCTGAGTGTTTATATCTTC |
| 2390 | CGIWDTNLGYLF | 2007 | TGTGGAATATGGGATACGAATCTGGGTTATCTCTTC |
| 2391 | CGIWDTGLSAVVF | 2008 | TGCGGTATATGGGATACCGGCCTGAGTGCTGTGGTATTC |
| 2392 | CGIWDRSLSAWVF | 2009 | TGCGGAATATGGGATCGCAGCCTGAGTGCTTGGGTGTTT |
| 2393 | CGIRDTRLSVYVF | 2010 | TGCGGAATTCGGGATACCAGGCTGAGTGTTTATGTCTTC |
| 2394 | CGGWSSRLGVGPVF | 2011 | TGCGGAGGATGGAGTAGCAGACTGGGTGTTGGCCCAGTGTTT |
| 2395 | CGGWGSGLSAWVF | 2012 | TGCGGAGGATGGGGTAGCGGCCTGAGTGCTTGGGTGTTC |
| 2396 | CGGWDTSLSAWVF | 2013 | TGCGGAGGATGGGATACCAGCCTGAGTGCTTGGGTGTTC |
| 2397 | CGGWDRGLDAWVF | 2014 | TGCGGAGGATGGGATAGGGGCCTGGATGCTTGGGTTTTC |
| 2398 | CGAWRNNVWVF | 2015 | TGCGGAGCATGGCGTAATAACGTGTGGGTGTTC |
| 2399 | CGAWNRRLNPHSHWVF | 2016 | TGCGGAGCATGGAACAGGCGCCTGAATCCTCATTCTCATTGGGTG |
| | | | TTC |
| 2400 | CGAWHNKLSAVF | 2017 | TGCGGAGCCTGGCACAACAAACTGAGCGCGGTCTTC |
| 2401 | CGAWGSSLRASVF | 2018 | TGCGGAGCATGGGGTAGCAGCCTGAGAGCTAGTGTCTTC |
| 2402 | CGAWGSGLSAWVF | 2019 | TGCGGAGCATGGGGTAGCGGCCTGAGTGCTTGGGTGTTC |
| 2403 | CGAWESSLSAPYVF | 2020 | TGCGGAGCATGGGAAAGTAGCCTGAGTGCCCCTTATGTCTTC |
| 2404 | CGAWESSLNVGLI | 2021 | TGCGGAGCATGGGAGAGCAGCCTCAATGTTGGACTGATC |
| 2405 | CGAWESGRSAGVVF | 2022 | TGCGGAGCATGGGAGAGCGGCCGGAGTGCTGGGGTGGTGTTC |
| 2406 | CGAWDYSVSGWVF | 2023 | TGCGGAGCTTGGGATTACAGTGTGAGTGGTTGGGTGTTC |
| 2407 | CGAWDYSLTAGVF | 2024 | TGCGGAGCATGGGATTACAGCCTGACTGCCGGAGTATTC |
| 2408 | CGAWDYRLSAVLF | 2025 | TGCGGAGCCTGGGATTACAGACTGAGTGCCGTGCTATTC |
| 2409 | CGAWDVRLDVGVF | 2026 | TGCGGAGCGTGGGATGTTCGTCTGGATGTTGGGGTGTTC |
| 1203 | CGAWDTYSYVF | 2027 | TGCGGAGCATGGGATACCTACAGTTATGTCTTC |
| 1204 | CGAWDTTLSGVVF | 2028 | TGCGGAGCATGGGATACGACCCTGAGTGGTGTGGTATTC |
| 1205 | CGAWDTTLSAVIF | 2029 | TGCGGAGCGTGGGATACTACCCTGAGTGCTGTGATATTC |
| 1206 | CGAWDTSQGASYVF | 2030 | TGCGGCGCATGGGATACCAGCCAGGGTGCGTCTTATGTCTTT |
| 1207 | CGAWDTSPVRAGVF | 2031 | TGCGGAGCATGGGATACCAGCCCTGTACGTGCTGGGGTGTTC |
| 1208 | CGAWDTSLWLF | 2032 | TGCGGAGCATGGGATACCAGCCTGTGGCTTTTC |
| 1209 | CGAWDTSLTVYVF | 2033 | TGCGGAGCATGGGATACCAGCCTGACTGTTTATGTCTTC |
| 1210 | CGAWDTSLTAGVF | 2034 | TGCGGAGCATGGGACACCAGTCTGACTGCTGGGGTGTTC |
| 1211 | CGAWDTSLSTVVF | 2035 | TGCGGAGCTTGGGATACCAGCCTGAGTACTGTGGTTTTC |
| 1212 | CGAWDTSLSSRYIF | 2036 | TGCGGAGCATGGGATACCAGCCTGAGTTCTAGATACATATTC |
| 1213 | CGAWDTSLSGYVF | 2037 | TGCGGAGCATGGGATACCAGCCTGAGTGGTTATGTCTTC |
| 1214 | CGAWDTSLSGWVF | 2038 | TGCGGAGCCTGGGATACCAGCCTGAGTGGCTGGGTGTTC |
| 1215 | CGAWDTSLSGVLF | 2039 | TGCGGAGCATGGGATACCAGTCTGAGTGGTGTGCTATTC |
| 1216 | CGAWDTSLSGLVF | 2040 | TGCGGAGCTTGGGATACCAGCTTGAGTGGTCTTGTTTTC |
| 1217 | CGAWDTSLSGFVF | 2041 | TGCGGAGCTTGGGATACCAGCTTGAGTGGTTTTGTTTTC |
| 1218 | CGAWDTSLSGEVF | 2042 | TGCGGAGCATGGGATACCAGCCTGAGTGGTGAGGTCTTT |
| 1219 | CGAWDTSLSDFVF | 2043 | TGCGGAGCTTGGGATACCAGCTTGAGTGATTTTGTTTTC |
| 1220 | CGAWDTSLRTAIF | 2044 | TGCGGAGCATGGGATACCAGCCTGCGAACTGCGATATTC |
| 1221 | CGAWDTSLRLF | 2045 | TGCGGAGCATGGGATACCAGCCTGCGGCTTTTC |
| 1222 | CGAWDTSLNVHVF | 2046 | TGCGGAGCATGGGATACCAGCCTGAATGTTCATGTCTTC |
| 1223 | CGAWDTSLNKWVF | 2047 | TGCGGAGCATGGGATACCAGCCTCAATAAATGGGTGTTC |
| 1224 | CGAWDTRLSARLF | 2048 | TGCGGAGCATGGGATACCCGCCTCAGTGCGCGGCTGTTC |
| 1225 | CGAWDTRLRGFIF | 2049 | TGCGGAGCATGGGATACCAGACTGAGGGGTTTTATTTTC |
| 1226 | CGAWDTNLGNVLL | 2050 | TGCGGAGCATGGGATACTAATTTGGGGAATGTTCTCCTC |
| 1227 | CGAWDTNLGKWVF | 2051 | TGCGGGGCATGGGATACCAACCTGGGTAAATGGGTTTTC |
| 1228 | CGAWDTGLEWYVF | 2052 | TGCGGAGCATGGGATACCGGCCTTGAGTGGTATGTTTTT |
| 1229 | CGAWDRTSGLWLF | 2053 | TGCGGAGCATGGGATAGGACTTCTGGATTGTGGCTTTTC |
| 1230 | CGAWDRSLVAGLF | 2054 | TGCGGAGCGTGGGATCGTAGCCTGGTTGCTGGACTCTTC |
| 1231 | CGAWDRSLTVYVF | 2055 | TGCGGAGCGTGGGATAGAAGCCTGACTGTTTATGTCTTC |
| 1232 | CGAWDRSLSGYVF | 2056 | TGCGGAGCATGGGATAGAAGCCTGAGTGGTTATGTCTTC |
| 1233 | CGAWDRSLSAYVF | 2057 | TGCGGAGCATGGGATAGAAGCCTGAGTGCTTATGTCTTC |
| 1234 | CGAWDRSLSAVVF | 2058 | TGCGGAGCATGGGATAGAAGCCTGAGTGCGGTGGTATTC |
| 1235 | CGAWDRSLSAGVF | 2059 | TGCGGAGCATGGGATCGCAGCCTGAGTGCTGGGGTTTTC |
| 1236 | CGAWDRSLRIVVF | 2060 | TGCGGAGCGTGGGATCGCAGCCTGCGTATTGTGGTATTC |
| 1237 | CGAWDRSLRAYVF | 2061 | TGCGGAGCATGGGATAGAAGTCTGAGGGCTTACGTCTTC |
| 1238 | CGAWDRSLNVWLF | 2062 | TGCGGAGCATGGGATAGAAGTCTGAATGTTTGGCTGTTC |
| 1239 | CGAWDRGLNVGWLF | 2063 | TGCGGCGCCTGGGATAGGGGCCTGAATGTCGGTTGGCTTTTC |
| 1240 | CGAWDNRLSILAF | 2064 | TGCGGCGCATGGGATAATAGACTGAGTATTTTGGCCTTC |
| 1241 | CGAWDNDLTAYVF | 2065 | TGCGGAGCTTGGGATAATGACCTGACAGCTTATGTCTTC |
| 1242 | CGAWDFSLTPLF | 2066 | TGCGGGGCATGGGATTTCAGCCTGACTCCTCTCTTC |
| 1243 | CGAWDDYRGVSIYVF | 2067 | TGCGGAGCCTGGGATGACTATCGGGGTGTGAGTATTTATGTCTTC |
| 1244 | CGAWDDRPSSAVVF | 2068 | TGTGGAGCATGGGATGACCGGCCTTCGAGTGCCGTGGTTTTC |
| 1245 | CGAWDDRLTVVVF | 2069 | TGCGGAGCATGGGATGACAGACTGACTGTCGTTGTTTTC |
| 1246 | CGAWDDRLGAVF | 2070 | TGCGGAGCGTGGGATGACAGGCTGGGTGCTGTGTTC |
| 1247 | CGAWDASLNPGRAF | 2071 | TGCGGAGCGTGGGATGCCAGCCTGAATCCTGGCCGGGCATTC |
| 1248 | CGAWDAGLREIF | 2072 | TGCGGAGCATGGGATGCCGGCCTGAGGGAAATTTTC |
| 1249 | CGAWAGSPSPWVF | 2073 | TGCGGAGCTTGGGCTGGCAGTCCGAGTCCTTGGGTTTTC |
| 1250 | CGAFDTTLSAGVF | 2074 | TGCGGAGCATTCGACACCACCCTGAGTGCTGGCGTTTTC |
| 1251 | CETWESSLSVGVF | 2075 | TGCGAAACATGGGAGAGCAGCCTGAGTGTTGGGGTCTTC |
| 1252 | CETWESSLRVWVF | 2076 | TGCGAAACATGGGAAAGCAGCCTGAGGGTTTGGGTGTTC |
| 1253 | CETWDTSLSGGVF | 2077 | TGCGAAACGTGGGATACCAGCCTGAGTGGTGGGGTGTTC |
| 1254 | CETWDTSLSDFYVF | 2078 | TGCGAAACATGGGATACCAGCCTGAGTGACTTTTATGTCTTC |
| 1255 | CETWDTSLSALF | 2079 | TGCGAAACATGGGATACCAGCCTGAGTGCCCTCTTC |
| 1256 | CETWDTSLRAEVF | 2080 | TGCGAAACATGGGATACCAGCCTGCGTGCTGAAGTCTTC |
| 1257 | CETWDTSLNVVVF | 2081 | TGCGAAACATGGGATACCAGCCTGAATGTTGTGGTATTC |
| 1258 | CETWDTSLGAVVF | 2082 | TGCGAAACATGGGATACCAGCCTGGGTGCCGTGGTGTTC |
| 1259 | CETWDRSLSGVVF | 2083 | TGCGAAACATGGGATAGAAGCCTGAGTGGTGTGGTATTC |
| 1260 | CETWDRSLSAWVF | 2084 | TGCGAAACATGGGATAGGAGCCTGAGTGCTTGGGTGTTT |
| 1261 | CETWDRSLSAVVF | 2085 | TGCGAAACATGGGATCGCAGCCTGAGTGCTGTGGTCTTC |
| 1262 | CETWDRGLSVVVF | 2086 | TGCGAGACGTGGGATAGAGGCCTGAGTGTTGTGGTTTTC |
| 1263 | CETWDRGLSAVVF | 2087 | TGCGAAACATGGGATAGGGGCCTGAGTGCAGTGGTATTC |
| 1264 | CETWDHTLSVVIF | 2088 | TGCGAAACATGGGATCACACCCTGAGTGTTGTGATATTC |
| 1265 | CETWDASLTVVLF | 2089 | TGCGAAACATGGGATGCCAGCCTGACTGTTGTGTTATTC |
| 1266 | CETWDASLSAGVF | 2090 | TGCGAAACATGGGATGCCAGCCTGAGTGCTGGGGTGTTC |
| 1267 | CETWDAGLSEVVF | 2091 | TGCGAAACGTGGGATGCCGGCCTGAGTGAGGTGGTGTTC |
| 1268 | CETFDTSLSVVVF | 2092 | TGCGAAACATTTGATACCAGCCTGAGTGTTGTAGTCTTC |
| 1269 | CETFDTSLNIVVF | 2093 | TGCGAAACATTTGATACCAGCCTAAATATTGTAGTCTTT |
| 1270 | CESWDRSRIGVVF | 2094 | TGCGAATCATGGGATAGAAGCCGGATTGGTGTGGTCTTC |
| 1271 | CESWDRSLSARVY | 2095 | TGCGAAAGTTGGGACAGGAGTCTGAGTGCCCGGGTGTAC |
| 1272 | CESWDRSLRAVVF | 2096 | TGCGAATCCTGGGATAGGAGCCTGCGTGCCGTGGTCTTC |
| 1273 | CESWDRSLIVVF | 2097 | TGCGAATCTTGGGATCGTAGTTTGATTGTGGTGTTC |
| 1274 | CESWDNNLNEVVF | 2098 | TGCGAAAGTTGGGATAACAATTTAAATGAGGTGGTTTTC |
| 1275 | CEIWESSPSADDLVF | 2099 | TGCGAAATATGGGAGAGCAGCCCGAGTGCTGACGATTTGGTGTTC |
| 1276 | CEAWDTSLSGAVF | 2100 | TGCGAAGCATGGGATACCAGCCTGAGTGGTGCGGTGTTC |
| 1277 | CEAWDTSLSAGVF | 2101 | TGCGAAGCATGGGATACCAGCCTGAGTGCCGGGGTGTTC |
| 1278 | CEAWDTSLGGGVF | 2102 | TGCGAAGCATGGGATACCAGCCTGGGTGGTGGGGTGTTC |
| 1279 | CEAWDRSLTGSLF | 2103 | TGCGAAGCATGGGATCGCAGCCTGACTGGTAGCCTGTTC |
| 1280 | CEAWDRGLSAVVF | 2104 | TGCGAAGCGTGGGATAGGGGCCTGAGTGCAGTGGTATTC |
| 1281 | CEAWDNILSTVVF | 2105 | TGCGAAGCCTGGGATAACATCCTGAGTACTGTGGTGTTC |
| 1282 | CEAWDISLSAGVF | 2106 | TGCGAAGCATGGGACATCAGCCTGAGTGCTGGGGTGTTC |
| 1283 | CEAWDADLSGAVF | 2107 | TGCGAAGCATGGGATGCCGACCTGAGTGGTGCGGTGTTC |
| 1284 | CATWTGSFRTGHYVF | 2108 | TGCGCAACATGGACTGGTAGTTTCAGAACTGGCCATTATGTCTTC |
| 1285 | CATWSSSPRGWVF | 2109 | TGCGCAACATGGAGTAGCAGTCCCAGGGGGTGGGTGTTC |
| 1286 | CATWHYSLSAGRVF | 2110 | TGCGCAACATGGCATTACAGCCTGAGTGCTGGCCGAGTGTTC |
| 1287 | CATWHTSLSIVQF | 2111 | TGCGCAACATGGCATACCAGCCTGAGTATTGTGCAGTTC |
| 1288 | CATWHSTLSADVLF | 2112 | TGCGCAACATGGCATAGCACCCTGAGTGCTGATGTGCTTTTC |
| 1289 | CATWHSSLSAGRLF | 2113 | TGCGCAACATGGCATAGCAGCCTGAGTGCTGGCCGACTCTTC |
| 1290 | CATWHIARSAWVF | 2114 | TGCGCAACATGGCATATCGCTCGGAGTGCCTGGGTGTTC |
| 1291 | CATWGSSQSAVVF | 2115 | TGCGCAACATGGGGTAGTAGTCAGAGTGCCGTGGTATTC |
| 1292 | CATWGSSLSAGGVF | 2116 | TGCGCAACATGGGGTAGCAGCCTGAGTGCTGGGGGTGTTTTC |
| 1293 | CATWEYSLSVVLF | 2117 | TGTGCAACATGGGAATACAGCCTGAGTGTTGTGCTGTTC |
| 1294 | CATWETTRRASFVF | 2118 | TGCGCAACATGGGAGACCACCCGACGTGCCTCTTTTGTCTTC |
| 1295 | CATWETSLNVYVF | 2119 | TGCGCAACATGGGAGACCAGCCTGAATGTTTATGTCTTC |
| 1296 | CATWETSLNVVVF | 2120 | TGCGCAACATGGGAAACTAGCCTGAATGTTGTGGTCTTC |
| 1297 | CATWETSLNLYVF | 2121 | TGCGCAACATGGGAGACCAGCCTGAATCTTTATGTCTTC |
| 1298 | CATWETGLSAGEVF | 2122 | TGCGCAACATGGGAGACTGGCCTAAGTGCTGGAGAGGTGTTC |
| 1299 | CATWESTLSVVVF | 2123 | TGCGCGACGTGGGAGAGTACCCTAAGTGTTGTGGTTTTC |
| 1300 | CATWESSLSIFVF | 2124 | TGCGCAACGTGGGAGAGCAGCCTGAGTATTTTTGTCTTC |
| 1301 | CATWESSLNTFYVF | 2125 | TGCGCAACATGGGAAAGCAGCCTCAACACTTTTTATGTCTTC |
| 1302 | CATWESRVDTRGLLF | 2126 | TGCGCAACATGGGAGAGTAGGGTGGATACTCGAGGGTTGTTATTC |
| 1303 | CATWESGLSGAGVF | 2127 | TGCGCAACATGGGAGAGCGGCCTGAGTGGTGCGGGGGTGTTC |
| 1304 | CATWEGSLNTFYVF | 2128 | TGCGCAACATGGGAAGGCAGCCTCAACACTTTTTATGTCTTC |
| 1305 | CATWDYSLSAVVF | 2129 | TGCGCAACTTGGGATTATAGCCTGAGTGCTGTGGTGTTC |
| 1306 | CATWDYRLSIVVF | 2130 | TGCGCAACATGGGATTACAGACTGAGTATTGTGGTATTC |
| 1307 | CATWDYNLGAAVF | 2131 | TGCGCAACATGGGATTATAACCTGGGAGCTGCGGTGTTC |
| 1308 | CATWDVTLGVLHF | 2132 | TGCGCCACATGGGATGTCACCCTGGGTGTCTTGCATTTC |
| 1309 | CATWDTTLSVWVF | 2133 | TGCGCAACATGGGATACAACACTGAGTGTCTGGGTCTTC |
| 1310 | CATWDTTLSVVLF | 2134 | TGCGCAACATGGGATACCACCCTGAGTGTAGTACTTTTC |
| 1311 | CATWDTTLSVEVF | 2135 | TGCGCAACATGGGATACCACCCTGAGTGTTGAGGTCTTC |
| 1312 | CATWDTSPSLSGFWVF | 2136 | TGCGCAACATGGGATACCAGCCCCAGCCTGAGTGGTTTTTGGGTG |
| | | | TTC |
| 1313 | CATWDTSLTGVVF | 2137 | TGCGCAACATGGGATACCAGCCTGACTGGTGTGGTATTC |
| 1314 | CATWDTSLTGAVF | 2138 | TGCGCAACATGGGATACCAGCCTGACTGGTGCGGTGTTC |
| 1315 | CATWDTSLTAWVF | 2139 | TGCGCAACATGGGATACCAGCCTGACTGCCTGGGTATTC |
| 1316 | CATWDTSLTAVVF | 2140 | TGCGCAACATGGGATACCAGCCTGACTGCTGTGGTTTTC |
| 1317 | CATWDTSLTAKVF | 2141 | TGCGCAACATGGGATACTAGCCTGACTGCTAAGGTGTTC |
| 1318 | CATWDTSLSVVVF | 2142 | TGCGCAACATGGGACACCAGCCTGAGTGTTGTGGTTTTC |
| 1319 | CATWDTSLSVGVF | 2143 | TGCGCTACTTGGGATACCAGCCTGAGTGTTGGGGTATTT |
| 1320 | CATWDTSLSSWVF | 2144 | TGCGCAACATGGGATACCAGCCTGAGTTCTTGGGTGTTC |
| 1321 | CATWDTSLSGGVL | 2145 | TGCGCAACATGGGATACCAGCCTGAGTGGTGGGGTACTC |
| 1322 | CATWDTSLSGGVF | 2146 | TGCGCAACATGGGATACCAGCCTGAGTGGTGGGGTGTTC |
| 1323 | CATWDTSLSGGRVF | 2147 | TGCGCAACATGGGATACCAGCCTGAGTGGTGGCCGAGTGTTC |
| 1324 | CATWDTSLSGDRVF | 2148 | TGCGCAACATGGGATACCAGCCTGAGTGGTGACCGAGTGTTC |
| 1325 | CATWDTSLSEGVF | 2149 | TGCGCAACGTGGGATACTAGCCTGAGTGAAGGGGTGTTC |
| 1326 | CATWDTSLSAWL | 2150 | TGCGCAACCTGGGATACCAGCCTGAGTGCCGTGGTGCTC |
| 1327 | CATWDTSLSAVF | 2151 | TGCGCAACATGGGATACCAGCCTGAGTGCTGTCTTC |
| 1328 | CATWDTSLSARVF | 2152 | TGCGCGACATGGGATACCAGCCTGAGTGCTCGGGTGTTC |
| 1329 | CATWDTSLSALF | 2153 | TGCGCAACATGGGATACCAGCCTGAGTGCCTTATTC |
| 1330 | CATWDTSLSAHVF | 2154 | TGCGCAACATGGGATACCAGCCTGAGTGCTCATGTCTTC |
| 1331 | CATWDTSLSAGRVF | 2155 | TGCGCAACATGGGATACCAGCCTGAGTGCTGGCCGGGTGTTC |
| 1332 | CATWDTSLSAEVF | 2156 | TGCGCAACATGGGATACCAGCCTGAGTGCGGAGGTCTTC |
| 1333 | CATWDTSLSADAGGGV | 2157 | TGCGCAACATGGGATACCAGCCTGAGTGCTGATGCTGGTGGGGGG |
| | F | | GTCTTC |
| 1334 | CATWDTSLRVVVF | 2158 | TGCGCAACATGGGATACCAGCCTGCGTGTCGTGGTATTC |
| 1335 | CATWDTSLRGVF | 2159 | TGCGCAACATGGGATACCAGCCTGAGAGGGGTGTTC |
| 1336 | CATWDTSLPAWVF | 2160 | TGCGCAACATGGGATACCAGCCTGCCTGCGTGGGTGTTC |
| 1337 | CATWDTSLNVGVF | 2161 | TGTGCAACATGGGATACCAGCCTGAATGTTGGGGTATTC |
| 1338 | CATWDTSLGIVLF | 2162 | TGCGCAACATGGGATACCAGCCTGGGTATTGTGTTATTT |
| 1339 | CATWDTSLGARVVF | 2163 | TGCGCAACATGGGACACCAGCCTGGGTGCGCGTGTGGTCTTC |
| 1340 | CATWDTSLGALF | 2164 | TGTGCAACGTGGGATACCAGTCTAGGTGCCTTGTTC |
| 1341 | CATWDTSLATGLF | 2165 | TGCGCAACATGGGATACCAGCCTGGCGACTGGACTGTTC |
| 1342 | CATWDTSLAAWVF | 2166 | TGCGCAACATGGGATACCAGCCTGGCTGCCTGGGTATTC |
| 1343 | CATWDTRLSAVVF | 2167 | TGCGCAACCTGGGATACCAGGCTGAGTGCTGTGGTCTTC |
| 1344 | CATWDTRLSAGVF | 2168 | TGCGCAACATGGGATACCAGGCTGAGTGCTGGGGTGTTC |
| 1345 | CATWDTRLLITVF | 2169 | TGTGCAACGTGGGACACACGTCTACTTATTACGGTTTTC |
| 1346 | CATWDTLLSVELF | 2170 | TGCGCAACATGGGACACCCTCCTGAGTGTTGAACTCTTC |
| 1347 | CATWDTGRNPHVVF | 2171 | TGCGCAACATGGGATACTGGCCGCAATCCTCATGTGGTCTTC |
| 1348 | CATWDTGLSSVLF | 2172 | TGCGCAACATGGGATACCGGCCTGTCTTCGGTGTTGTTC |
| 1349 | CATWDTGLSAVF | 2173 | TGCGCAACGTGGGATACCGGCCTGAGTGCGGTTTTC |
| 1350 | CATWDRTLSIGVF | 2174 | TGCGCTACGTGGGATAGGACCCTGAGTATTGGAGTCTTC |
| 1351 | CATWDRSVTAVLF | 2175 | TGCGCAACGTGGGATCGCAGTGTGACTGCTGTGCTCTTC |
| 1352 | CATWDRSLSGVVF | 2176 | TGCGCAACCTGGGATAGGAGCCTGAGTGGTGTGGTGTTC |
| 1353 | CATWDRSLSAVVF | 2177 | TGCGCAACATGGGATAGAAGCCTGAGTGCTGTGGTCTTC |
| 1354 | CATWDRSLSAVPWVF | 2178 | TGCGCAACATGGGATAGAAGCCTGAGTGCTGTTCCTTGGGTGTTC |
| 1355 | CATWDRSLSAGVF | 2179 | TGCGCAACATGGGATCGCAGCCTGAGTGCTGGGGTGTTC |
| 1356 | CATWDRSLRAGVF | 2180 | TGCGCAACGTGGGATAGGAGCCTGCGTGCTGGGGTGTTC |
| 1357 | CATWDRSLNVYVL | 2181 | TGCGCAACATGGGATCGCAGTCTGAATGTTTATGTCCTC |
| 1358 | CATWDRILSAEVF | 2182 | TGCGCAACGTGGGATCGCATCCTGAGCGCTGAGGTGTTC |
| 1359 | CATWDRGLSTGVF | 2183 | TGCGCAACGTGGGATAGAGGCCTGAGTACTGGGGTGTTC |
| 1360 | CATWDNYLGAAVF | 2184 | TGCGCAACATGGGATAACTACCTGGGTGCTGCCGTGTTC |
| 1361 | CATWDNTPSNIVVF | 2185 | TGCGCAACATGGGATAACACGCCTTCGAATATTGTGGTATTC |
| 1362 | CATWDNTLSVWVF | 2186 | TGCGCAACATGGGATAATACACTGAGTGTGTGGGTCTTC |
| 1363 | CATWDNTLSVNWVF | 2187 | TGCGCAACATGGGATAACACCCTGAGTGTCAATTGGGTGTTC |
| 1364 | CATWDNTLNVFYVF | 2188 | TGCGCAACCTGGGATAACACACTGAATGTCTTTTATGTTTTC |
| 1365 | CATWDNRLSSVVF | 2189 | TGTGCGACATGGGATAATCGGCTCAGTTCTGTGGTCTTC |
| 1366 | CATWDNRLSAGVL | 2190 | TGCGCAACATGGGATAACCGCCTGAGTGCTGGGGTGCTC |
| 1367 | CATWDNRLSAGVF | 2191 | TGCGCAACGTGGGATAACAGGCTGAGTGCTGGGGTGTTC |
| 1368 | CATWDNRDWVF | 2192 | TGCGCAACATGGGATAACAGGGATTGGGTCTTC |
| 1369 | CATWDNNLGAGVF | 2193 | TGCGCAACATGGGATAACAACCTGGGTGCTGGGGTGTTC |
| 1370 | CATWDNKLTSGVF | 2194 | TGCGCAACATGGGATAACAAGCTGACTTCTGGGGTCTTC |
| 1371 | CATWDNILSAWVF | 2195 | TGCGCAACATGGGATAACATCCTGAGTGCCTGGGTGTTT |
| 1372 | CATWDNDIHSGLF | 2196 | TGCGCAACCTGGGACAACGATATACATTCTGGGCTGTTC |
| 1373 | CATWDLSLSALF | 2197 | TGCGCAACTTGGGATCTCAGCCTGAGTGCCCTGTTC |
| 1374 | CATWDITLSAEVF | 2198 | TGCGCAACATGGGATATCACCCTGAGTGCTGAGGTGTTC |
| 1375 | CATWDISPSAGGVF | 2199 | TGCGCAACGTGGGATATCAGCCCGAGTGCTGGCGGGGTGTTC |
| 1376 | CATWDISLSTGRAVF | 2200 | TGCGCAACATGGGATATCAGTCTAAGTACTGGCCGGGCTGTGTTC |
| 1377 | CATWDISLSQVF | 2201 | TGCGCAACATGGGATATCAGTCTGAGTCAGGTATTC |
| 1378 | CATWDIRLSSGVF | 2202 | TGCGCAACATGGGATATCAGGCTGAGTAGTGGAGTGTTC |
| 1379 | CATWDIGPSAGGVF | 2203 | TGCGCAACGTGGGATATCGGCCCGAGTGCTGGCGGGGTGTTC |
| 1380 | CATWDHSRAGVLF | 2204 | TGCGCAACATGGGATCACAGCCGGGCTGGTGTGCTATTC |
| 1381 | CATWDHSPSVGEVF | 2205 | TGCGCAACATGGGATCACAGTCCGAGTGTTGGAGAAGTCTTC |
| 1382 | CATWDHSLRVGVF | 2206 | TGCGCAACATGGGATCACAGCCTGCGTGTTGGGGTGTTC |
| 1383 | CATWDHSLNIGVF | 2207 | TGCGCAACATGGGATCACAGCCTGAACATTGGGGTGTTC |
| 1384 | CATWDHSLGLWAF | 2208 | TGCGCAACATGGGATCACAGCCTGGGTCTTTGGGCATTC |
| 1385 | CATWDHNLRLVF | 2209 | TGCGCCACATGGGATCACAATCTGCGTCTTGTTTTC |
| 1386 | CATWDHILASGVF | 2210 | TGCGCGACTTGGGATCACATCCTGGCTTCTGGGGTGTTC |
| 1387 | CATWDFSLSVWVF | 2211 | TGCGCAACATGGGATTTCAGCCTGAGTGTTTGGGTGTTC |
| 1388 | CATWDFSLSAWVF | 2212 | TGCGCAACATGGGATTTCAGCCTGAGTGCTTGGGTGTTC |
| 1389 | CATWDDTLTAGVF | 2213 | TGCGCAACATGGGATGACACCCTCACTGCTGGTGTGTTC |
| 1390 | CATWDDRLSAVLF | 2214 | TGCGCAACATGGGACGACAGGCTGAGTGCTGTGCTTTTC |
| 1391 | CATWDDRLDAAVF | 2215 | TGCGCAACATGGGATGACAGGCTGGATGCTGCGGTGTTC |
| 1392 | CATWDATLNTGVF | 2216 | TGCGCAACATGGGATGCGACCCTGAATACTGGGGTGTTC |
| 1393 | CATWDASLSVWLL | 2217 | TGCGCAACATGGGATGCCAGCCTGAGTGTTTGGCTGCTC |
| 1394 | CATWDASLSGGVF | 2218 | TGCGCGACATGGGATGCCAGCCTGAGTGGTGGGGTGTTC |
| 1395 | CATRDTTLSAVLF | 2219 | TGCGCAACACGGGATACCACCCTCAGCGCCGTTCTGTTC |
| 1396 | CATLGSSLSLWVF | 2220 | TGCGCTACATTGGGTAGTAGCCTGAGTCTCTGGGTGTTC |
| 1397 | CATIETSLPAWVF | 2221 | TGCGCAACAATCGAAACTAGCCTGCCTGCCTGGGTATTC |
| 1398 | CATGDRSLTVEVF | 2222 | TGCGCAACAGGGGACAGAAGCCTGACTGTTGAGGTATTC |
| 1399 | CATGDLGLTIVF | 2223 | TGCGCTACAGGGGATCTCGGCCTGACCATAGTCTTC |
| 1400 | CASWDYRGRSGWVF | 2224 | TGCGCATCATGGGATTACAGGGGGAGATCTGGTTGGGTGTTC |
| 1401 | CASWDTTLNVGVF | 2225 | TGCGCATCATGGGATACCACCCTGAATGTTGGGGTGTTC |
| 1402 | CASWDTTLGFVLF | 2226 | TGCGCTTCATGGGATACCACCCTGGGTTTTGTGTTATTC |
| 1403 | CASWDTSLSGGYVF | 2227 | TGCGCATCATGGGATACCAGCCTGAGTGGTGGTTATGTCTTC |
| 1404 | CASWDTSLRAGVF | 2228 | TGCGCATCATGGGATACCAGCCTCCGTGCTGGGGTGTTC |
| 1405 | CASWDTSLGAGVF | 2229 | TGCGCATCATGGGATACCAGCCTGGGTGCTGGGGTGTTC |
| 1406 | CASWDRGLSAVVF | 2230 | TGCGCATCATGGGACAGAGGCCTGAGTGCAGTGGTGTTC |
| 1407 | CASWDNVLRGVVF | 2231 | TGTGCTAGTTGGGATAACGTCCTGCGTGGTGTGGTATTC |
| 1408 | CASWDNRLTAVVF | 2232 | TGCGCGTCATGGGATAACAGGCTGACTGCCGTGGTTTTC |
| 1409 | CASWDASLSVAF | 2233 | TGCGCATCATGGGATGCAAGCCTGTCCGTCGCTTTC |
| 1410 | CASWDAGLSSYVF | 2234 | TGCGCTTCGTGGGATGCCGGCCTGAGTTCTTATGTCTTC |
| 1411 | CASGDTSLSGVIF | 2235 | TGCGCATCCGGGGATACCAGCCTGAGTGGTGTGATATTC |
| 1412 | CARWHTSLSIWVF | 2236 | TGCGCAAGATGGCATACGAGCCTAAGTATTTGGGTCTTC |
| 1413 | CAIWDTGLSPGQVAF | 2237 | TGCGCAATATGGGATACCGGCCTGAGTCCTGGCCAAGTTGCCTTC |
| 1414 | CAAWHSGLGLPVF | 2238 | TGCGCAGCATGGCATAGCGGCCTGGGTCTCCCGGTCTTC |
| 1415 | CAAWDYSLSAGVF | 2239 | TGCGCAGCATGGGATTACAGCCTGAGTGCTGGGGTGTTC |
| 1416 | CAAWDTTLRVRLF | 2240 | TGCGCAGCCTGGGATACTACCCTGCGTGTTAGGCTGTTC |
| 1417 | CAAWDTSLTAWVF | 2241 | TGCGCAGCATGGGATACCAGCCTGACTGCCTGGGTTTTC |
| 1418 | CAAWDTSLSGGVF | 2242 | TGCGCAGCATGGGATACCAGCTTGAGTGGTGGGGTGTTC |
| 1419 | CAAWDTSLSGEAVF | 2243 | TGCGCAGCATGGGATACCAGCCTGAGTGGCGAGGCTGTGTTC |
| 1420 | CAAWDTSLSGAVF | 2244 | TGCGCAGCATGGGATACCAGCTTGAGTGGTGCGGTGTTC |
| 1421 | CAAWDTSLSAWVF | 2245 | TGCGCAGCATGGGATACCAGCCTGAGTGCCTGGGTGTTC |
| 1422 | CAAWDTSLSAGVF | 2246 | TGCGCAGCATGGGATACCAGCCTGAGTGCTGGGGTATTC |
| 1423 | CAAWDTSLDTYVF | 2247 | TGCGCAGCATGGGATACCAGCCTGGATACTTATGTCTTC |
| 1424 | CAAWDTRLSGVLF | 2248 | TGCGCTGCATGGGATACCCGTCTGAGTGGTGTGTTATTC |
| 1425 | CAAWDTRLSAGVF | 2249 | TGCGCAGCATGGGATACCAGGCTGAGTGCTGGGGTGTTC |
| 1426 | CAAWDRSLSTGVF | 2250 | TGCGCAGCATGGGATCGCAGTCTGAGTACTGGAGTTTTC |
| 1427 | CAAWDIRRSVLF | 2251 | TGCGCAGCGTGGGATATCCGCCGGTCTGTCCTTTTC |
| 1428 | CAAWDHTQRLSF | 2252 | TGCGCTGCGTGGGATCACACTCAGCGTCTTTCCTTC |
| 1429 | CAAWDHSLSAGQVF | 2253 | TGCGCAGCATGGGATCACAGCCTGAGTGCTGGCCAGGTGTTC |
| 1430 | CAAVDTGLKEWVF | 2254 | TGCGCAGCAGTCGATACTGGTCTGAAAGAATGGGTGTTC |

The CDRs were prescreened to contain no amino acid liabilities, cryptic splice sites or nucleotide restriction sites. The CDR variation was observed in at least two individuals and comprises the near-germline space of single, double and triple mutations. The order of assembly is seen in FIG. 8C.

The VH domains that were designed include IGHV1-69 and IGHV3-30. Each of two heavy chain VH domains are assembled with their respective invariant 4 framework elements (FW1, FW2, FW3, FW4) and variable 3 CDR (H1, H2, H3) elements. For IGHV1-69, 417 variants were designed for H1 and 258 variants were designed for H2. For IGHV3-30, 535 variants were designed for H1 and 165 variants were designed for H2. For the CDR H3, the same cassette was used in both IGHV1-69 and IGHV-30 since both designed use an identical FW4, and because the edge of FW3 is also identical for both IGHV1-69 and IGHV3-30. The CDR H3 comprises an N-terminus and C-terminus element that are combinatorially joined to a central middle element to generate 1×10¹ diversity. The N-terminal and middle element overlap with a “GGG” glycine codon. The middle and C-terminal element overlap with a “GGT” glycine codon. The CDR H3 comprises 5 subpools that were assembled separately. The various N-terminus and C-terminus elements comprise sequences as seen in Table 10.

TABLE 10
Sequences for N-terminus and C-terminus elements

| Element | SEQ ID NO | Sequence |
|---|---|---|
| Stem A | 2255 | CARDLRELECEEWT XXX |
| | | SRGPCVDPRGVAGSFDVW |
| Stem B | 2256 | CARDMYYDF XXX EVVPADDAFDIW |
| Stem C | 2257 | CARDGRGSLPRPKGGP XXX |
| | | YDSSEDSGGAFDIW |
| Stem D | 2258 | CARANQHF XXX GYHYYGMDVW |
| Stem E | 2259 | CAKHMSMQ XXX RADLVGDAFDVW |

### Example 9. Enrichment for GPCR GLP1R Binding Proteins

Antibodies having CDR-H3 regions with a variant fragments of GPCR binding protein that were generated by methods described herein were panned using cell-based methods to identify variants which are enriched for binding to particular GPCRs, as described in Example 4.

Variants of the GLP C-terminus peptide were identified (listed in Table 11) that when embedded in the CDR-H3 region of an antibody, were repeatedly and selectively enriched for binding to GPCR GLP1R.

TABLE 11
Sequences of GLP1 embedded in CDR-H3

| SEQ ID | |
|---|---|
| NO | Sequence |
| 2260 | CAKHMSMQEGAVTGEGQAAKEFIAWLVKGRVRADLVGDAFDVW |
| 2261 | CARDGRGSLPRPKGGPQTVGEGQAAKEFIAWLVKGGLTYDSSEDSGGAFDIW |
| 2262 | CAKHMSMQDYLVIGEGQAAKEFIAWLVKGGPARADLVGDAFDVW |
| 2263 | CAKHMSMQEGAVTGEGQDAKEFIAWLVKGRVRADLVGDAFDVW |
| 2264 | WAKHMSMQEGAVTGEGQAAKEFIAWLVKGRVRADLVGDAFDVW |
| 2265 | CARDGRGSLPRPKGGPQTVGEGQAAKEFIAWLVKGRVRADLVGDAFDVW |
| 2266 | CARANQHFYEQEGTFTSDVSSYLEGQAAKEFIAWLVKGGIRGYHYYGMDVW |
| 2267 | CARANQHFTELHGEGQAAKEFIAWLVKGRGQIDIGYHYYGMDVW |
| 2268 | CARANQHFLGAGVSSYLEGQAAKEFIAWLVKGDTTGYHYYGMDVW |
| 2269 | CARANQHFLDKGTFTSDVSSYLEGQAAKEFIAWLVKGIYPGYHYYGMDVW |
| 2270 | CARANQHFGTLSAGEGQAAKEFIAWLVKGGSQYDSSEDSGGAFDIW |
| 2271 | CARANQHFGLHAQGEGQAAKEFIAWLVKGSGTYGYHYYGMDVW |
| 2272 | CARANQHFGGKGEGQAAKEFIAWLVKGGGSGAGYHYYGMDVW |
| 2273 | CAKQMSMQEGAVTGEGQAAKEFIAWLVKGRVRADLVGDAFDVW |
| 2274 | CAKHMSMQEGAVTGEGQAAKEFIAWLVKGGPARADLVGDAFDVW |
| 2275 | CAKHMSMQEGAVTGEGQAAKEFIAWLVKGGLTYDSSEDSGGAFDIW |
| 2276 | CAKHMSMQDYLVIGEGQAAKEFIAWLVKGRVRADLVGDAFDVW |

### Example 10. Analysis of GLP1R Binding Protein Variants

Antibodies having CDR-H3 regions with variant fragments of GLP1R binding protein were generated by methods described herein were panned using cell-based methods to identify variants which are enriched for binding to GLPIR, as described in Example 4.

Next generation sequence (NGS) enrichment for variants of the GLPIR peptides was performed (data not shown). Briefly, phage populations were deep-sequenced after each round of selection are deep-sequenced to follow enrichment and identify cross-sample clones. Target specific clones were selected after filtering out CHO background clones from the NGS data. For GLP1R peptides, about 2000 VH and VL pairs were barcoded directly from a bacterial colony and sequenced to identify non-identical clones.

GLPIR-1 variant was analyzed for V gene distribution, J gene distribution, V gene family, and CDR3 counts per length. Frequency of V genes IGHV1-69, IGHV3-30, IGHV3-23, IGHV3, IGHV3-53, IGHV3-NL1, IGHV3-d, IGHV1-46, IGHV3-h, IGHV1, IGHV3-38, IGHV3-48, IGHV1-18, IGHV1-3, and IGHV3-15 was determined (data not shown). High frequency of IGHV1-69 and IGHV3-30 were observed. Frequency of J genes IGHJ3, IGHJ6, IGHJ, IGHJ4, IGHJ5, mIGHJ, IGHJ2, and IGH1 was also determined (data not shown). High frequency of IGHJ3 and IGHJ6 were observed with less frequency of IGHJ and IGHJ4 observed. Frequency of V genes IGHV1-69, IGHV3-30, IGHV3-23, IGHV3, IGHV1-46, IGHV3-7, IGHV1, and IGHV1-8 was determined (data not shown). High frequency of IGHV1-69 and IGHV3-30 was observed. Frequency of J genes IGHJ3, IGHJ6, IGHJ, IGHJ4, IGHJ5, IGHJ2, and IGH1 was determined (data not shown). High frequency of IGHJ3 and IGHJ6 was observed with less frequency of IGHJ and IGHJ4 observed.

H accumulation and frequency were determined for GLP1R-1, GLP1R-2, GLP1R-3, GLP1R-4, and GLP1R-5 (data not shown).

Sequence analytics were performed for GLP1R-1, GLP1R-2, GLP1R-3, GLP1R-4, and GLP1R-5 variants (data not shown).

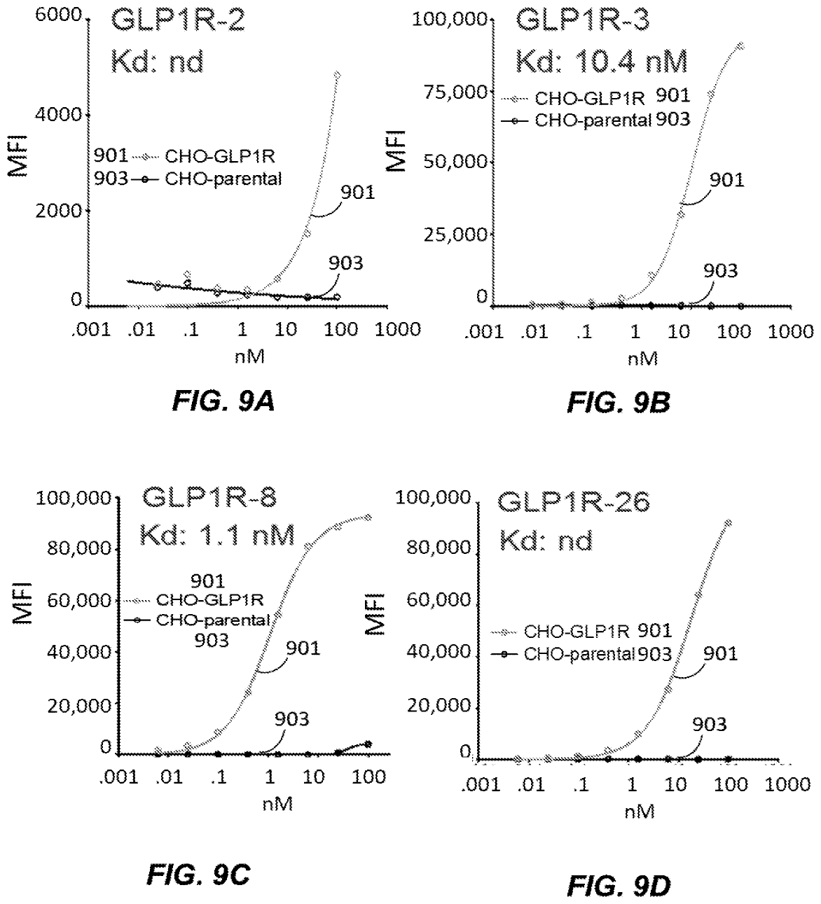

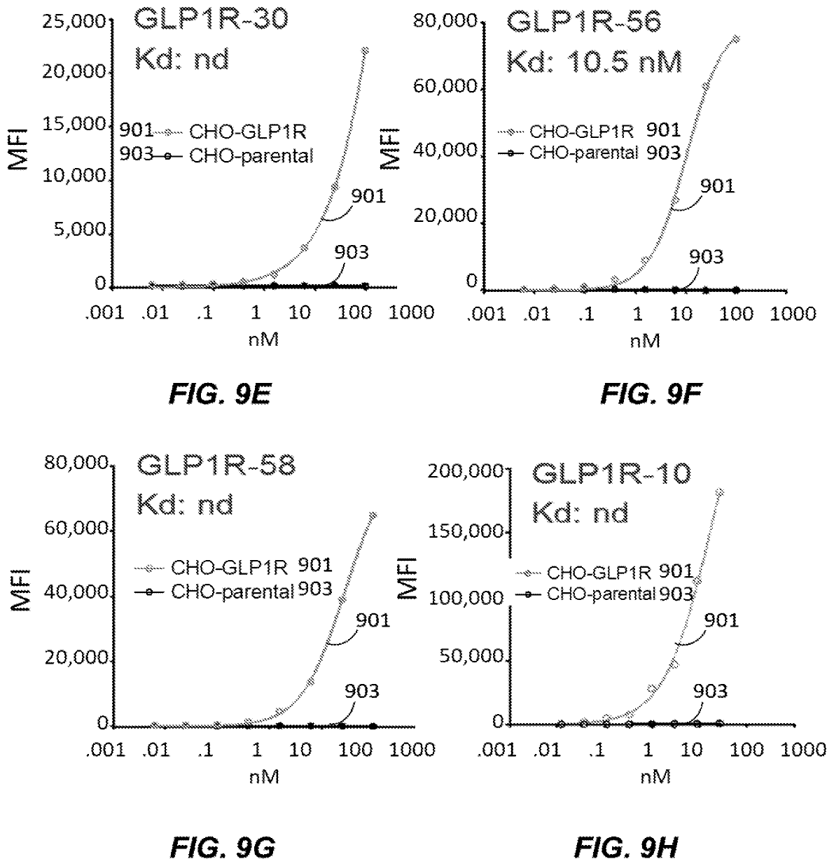

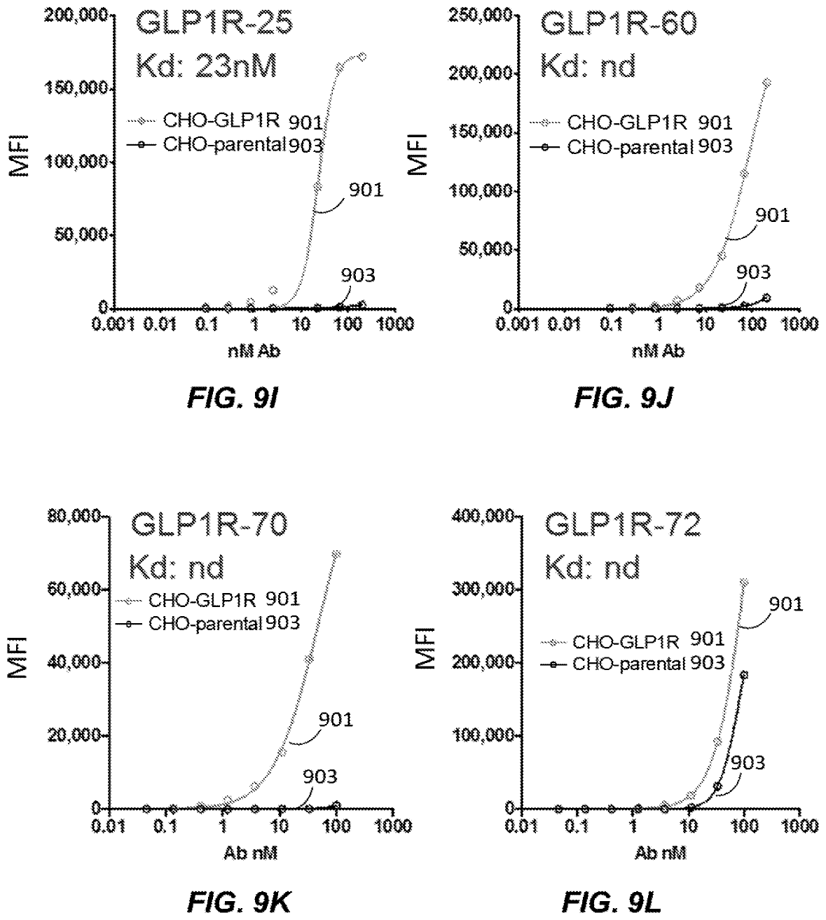

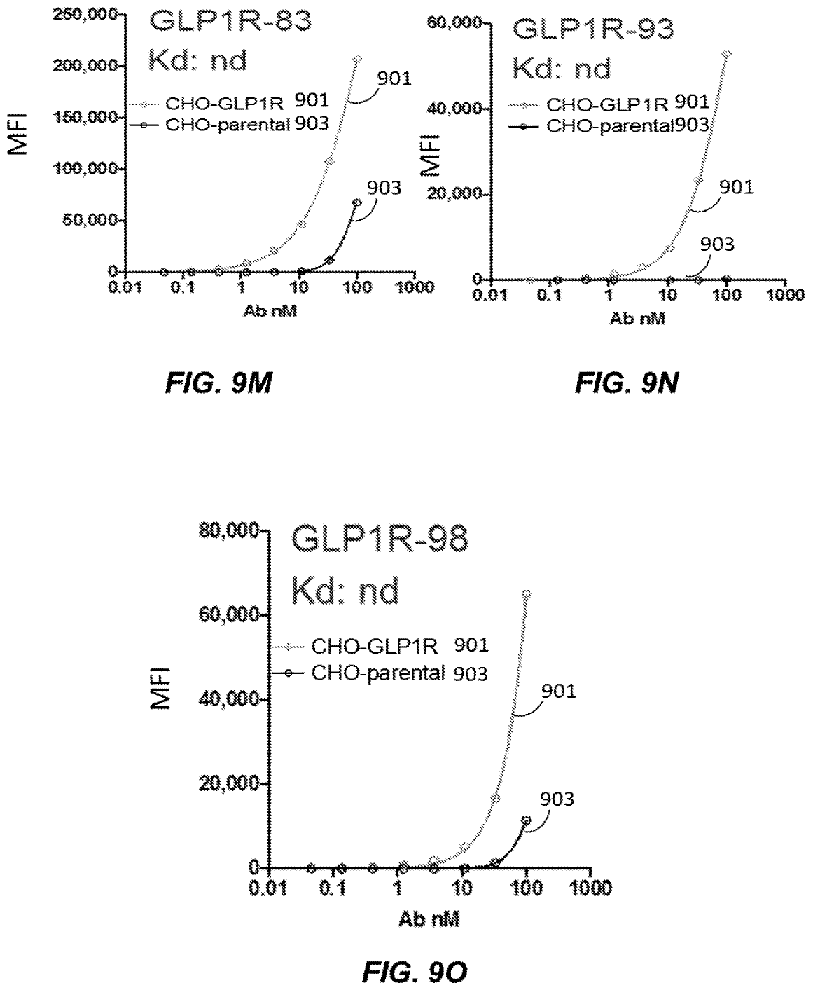

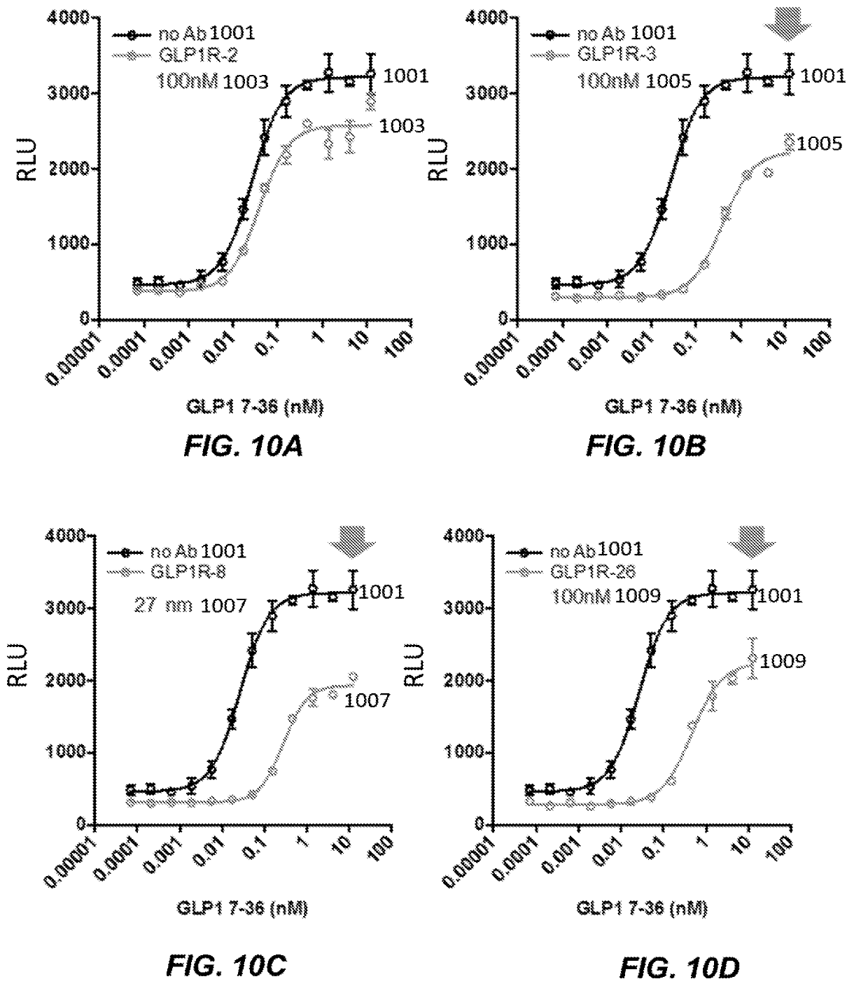

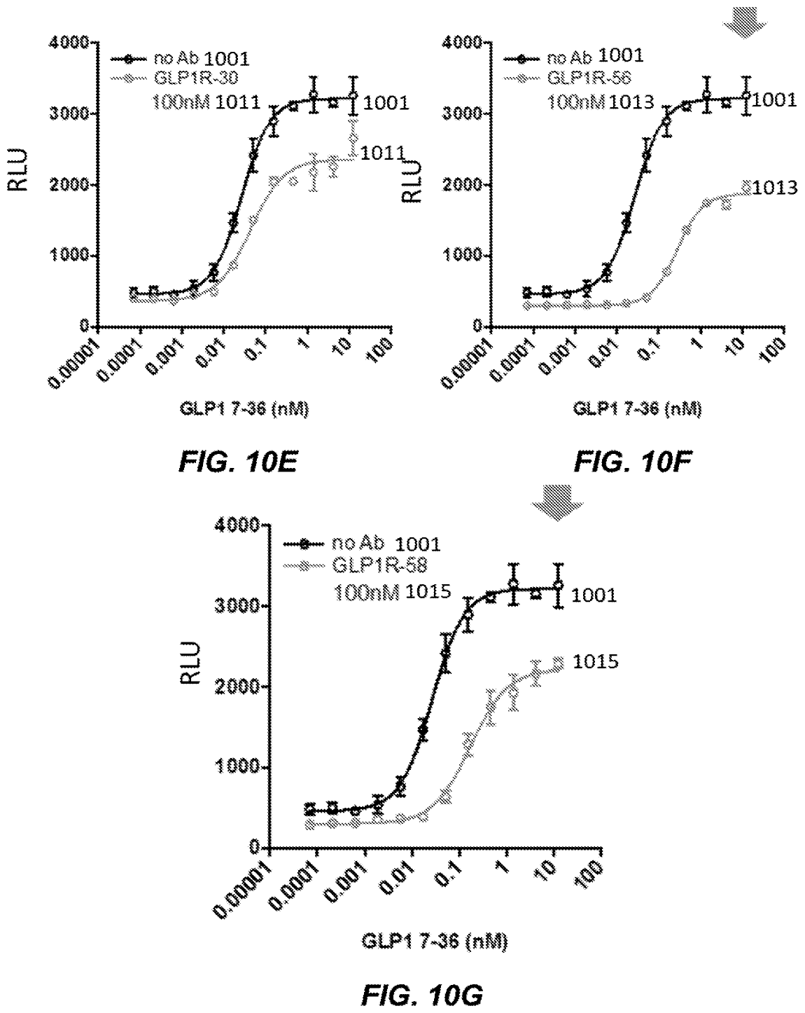

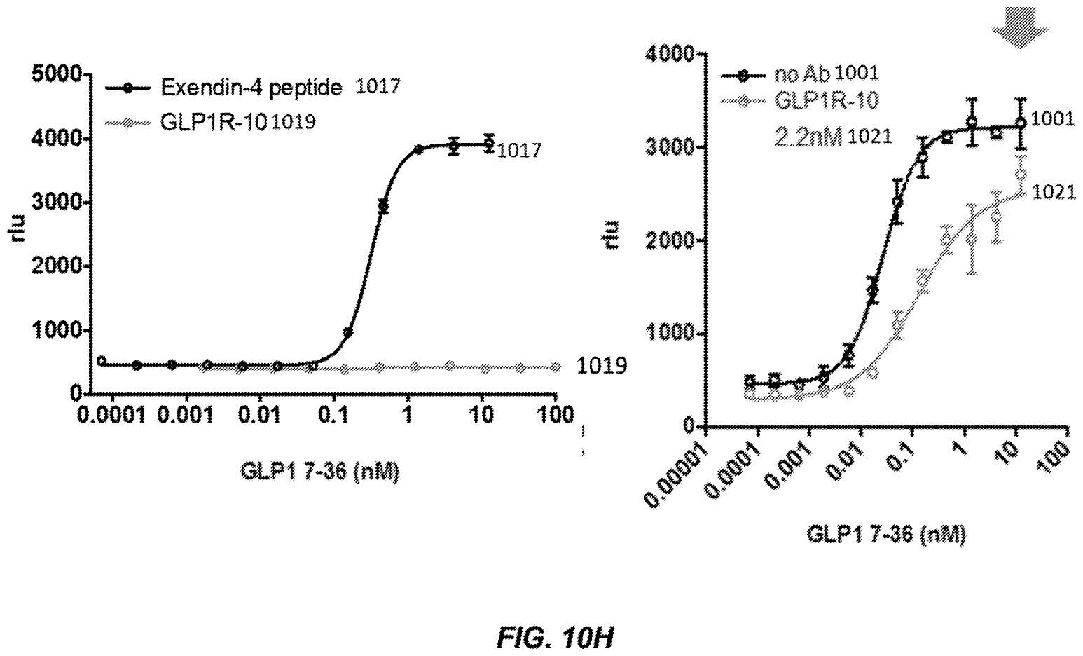

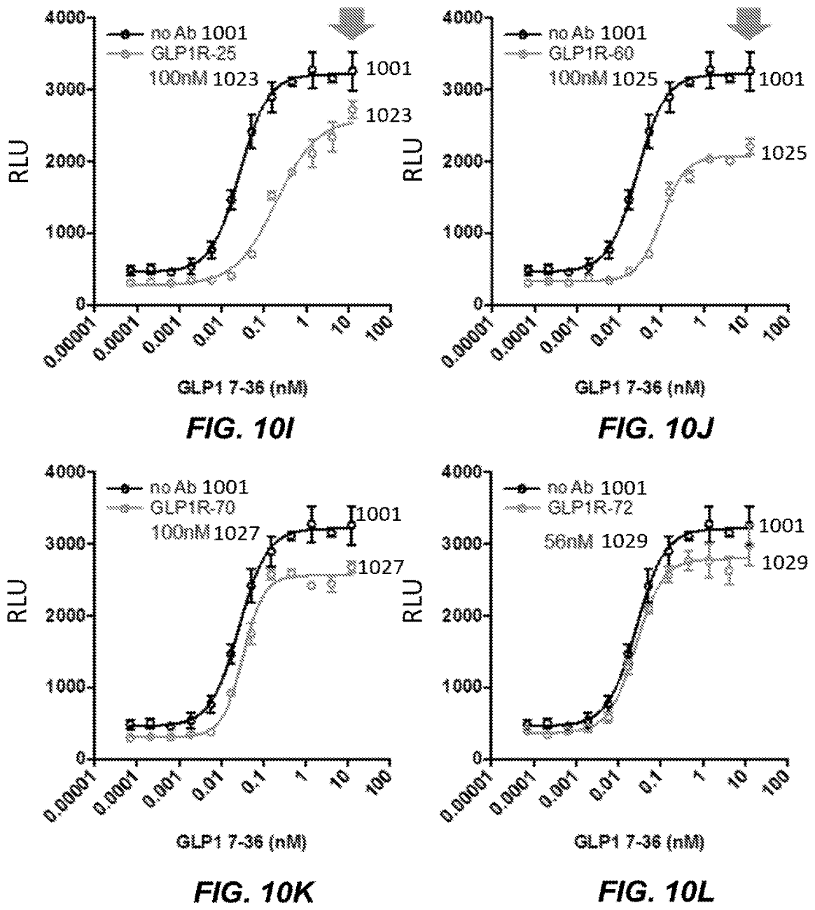

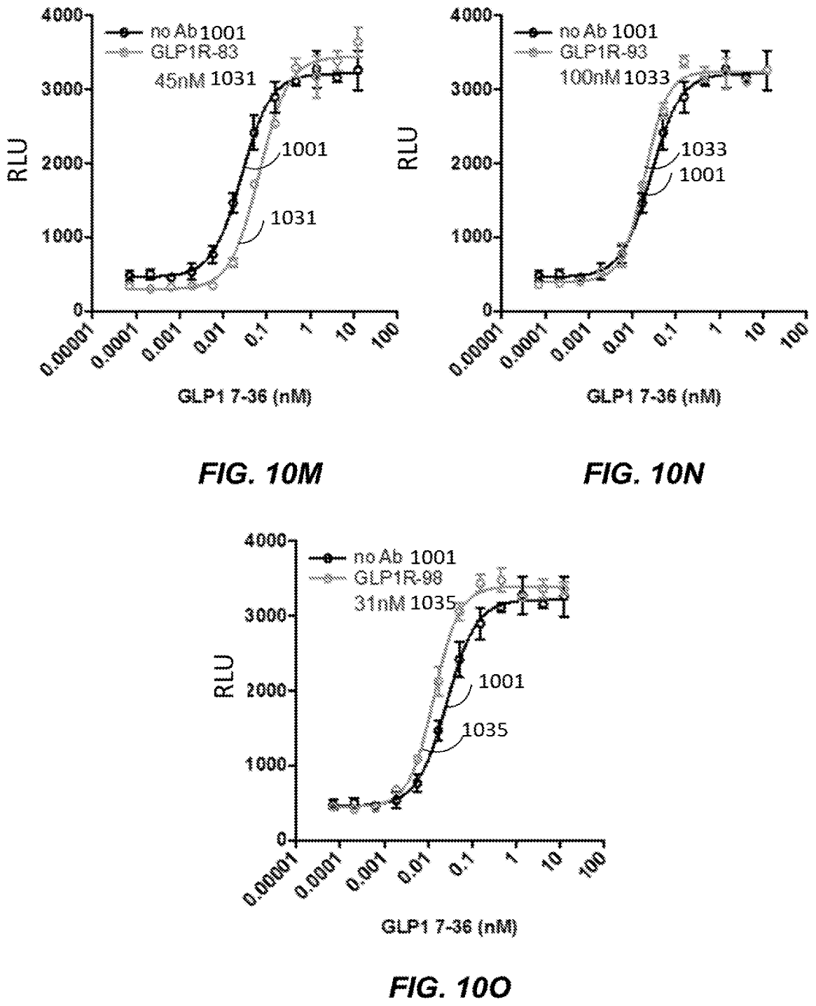

Cell binding was determined for the GLP1R variants. FIGS. 9A-9O show the cell binding data for GLP1R-2 (FIG. 9A), GLP1R-3 (FIG. 9B), GLP1R-8 (FIG. 9C), GLP1R-26 (FIG. 9D), GLP1R-30 (FIG. 9E), GLP1R-56 (FIG. 9F), GLP1R-58 (FIG. 9G), GLP1R-10 (FIG. 9H), GLP1R-25 (FIG. 9I), GLP1R-60 (FIG. 9J), GLP1R-70 (FIG. 9K), GLP1R-72 (FIG. 9L), GLP1R-83 (FIG. 9M), GLP1R-93 (FIG. 9N), and GLP1R-98 (FIG. 9O). GLP1R-3, GLP1R-8, GLP1R-26, GLP1R-56, GLP1R-58 and GLP1R-10 were then analyzed for allosteric effects on GLP1-7-36 peptide in a cAMP assay. FIGS. 10A-100 show graphs of the GLP1R variants on inhibition of GLP1-7-36 peptide induced cAMP activity. GLP1R-3 (FIG. 10B), GLP1R-8 (FIG. 10C), GLP1R-26 (FIG. 10D), GLP1R-30 (FIG. 10E), GLP1R-56 (FIG. 10F), GLP1R-58 (FIG. 10G), GLP1R-10 (FIG. 10H, right graph), GLP1R-25 (FIG. 10I), and GLP1R-60 (FIG. 10J) show allosteric inhibition of GLP1-7-36 peptide induced cAMP activity. FIG. 10H further shows effects of GLPR-10 on cAMP signal as compared to exendin-4 (FIG. 10H, left graph).

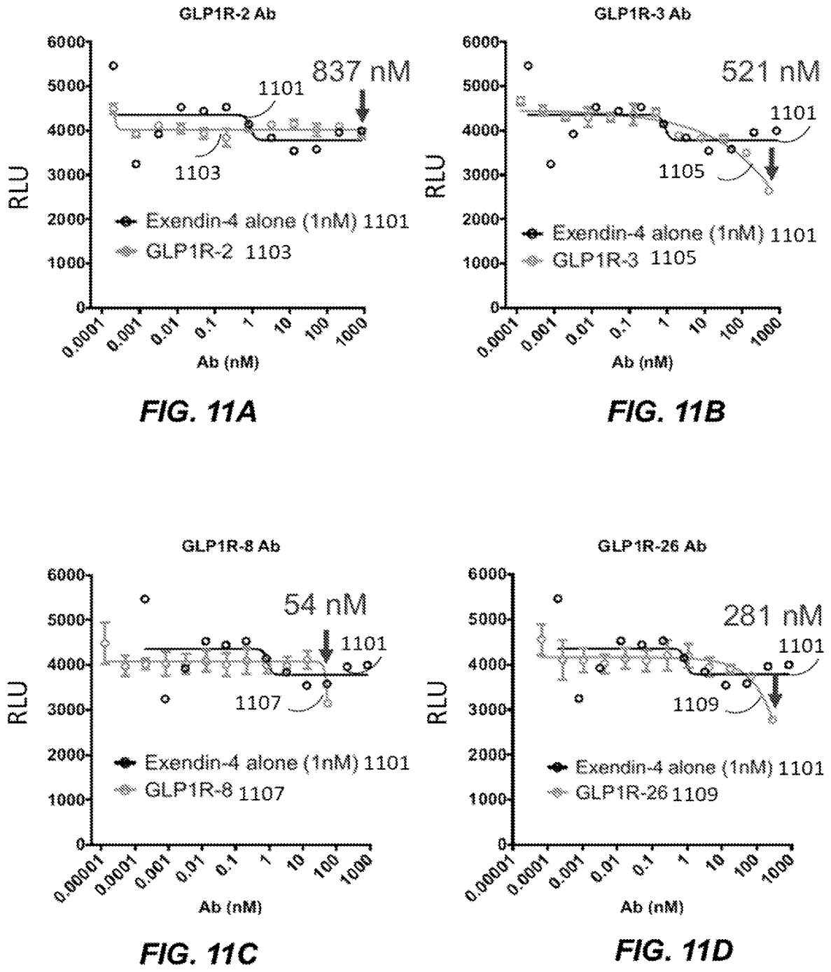

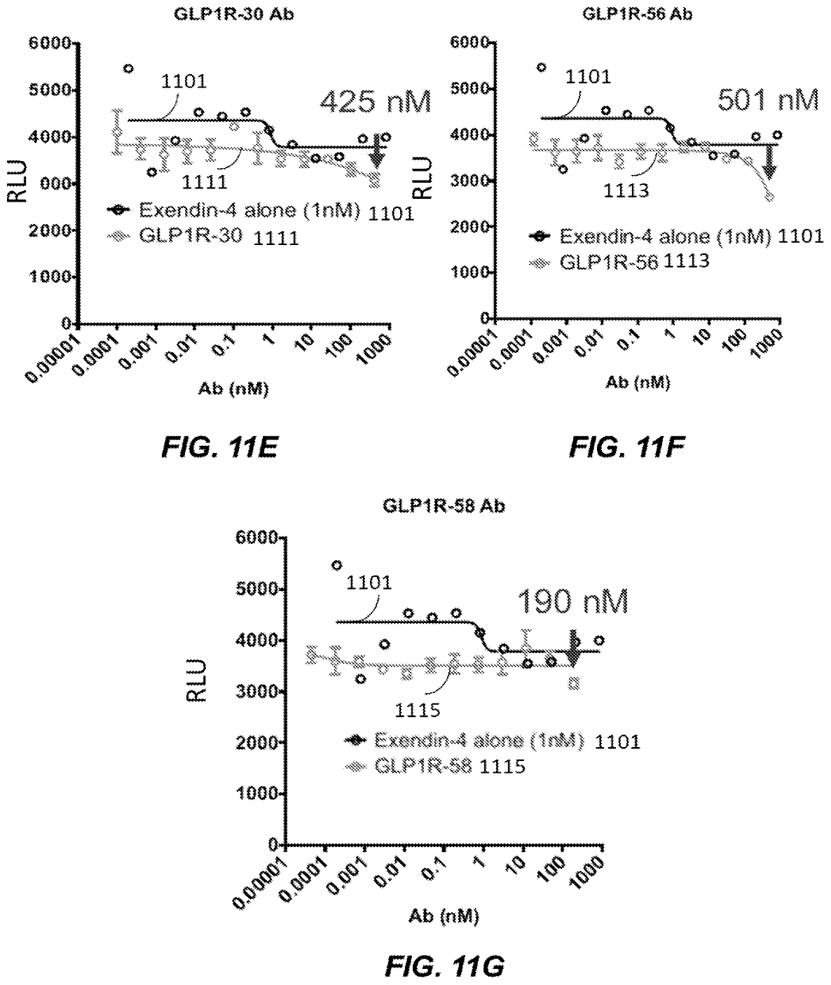

GLP1R variants were tested in a cAMP assay to determine if the variants were antagonists in blocking exendin-4 induced cAMP activity. FIGS. 11A-11G depict cell functional data for GLP1R-2 (FIG. 11A), GLP1R-3 (FIG. 11B), GLP1R-8 (FIG. 11C), GLP1R-26 (FIG. 11D), GLP1R-30 (FIG. 11E), GLP1R-56 (FIG. 11F), and GLP1R-58 (FIG. 11G).

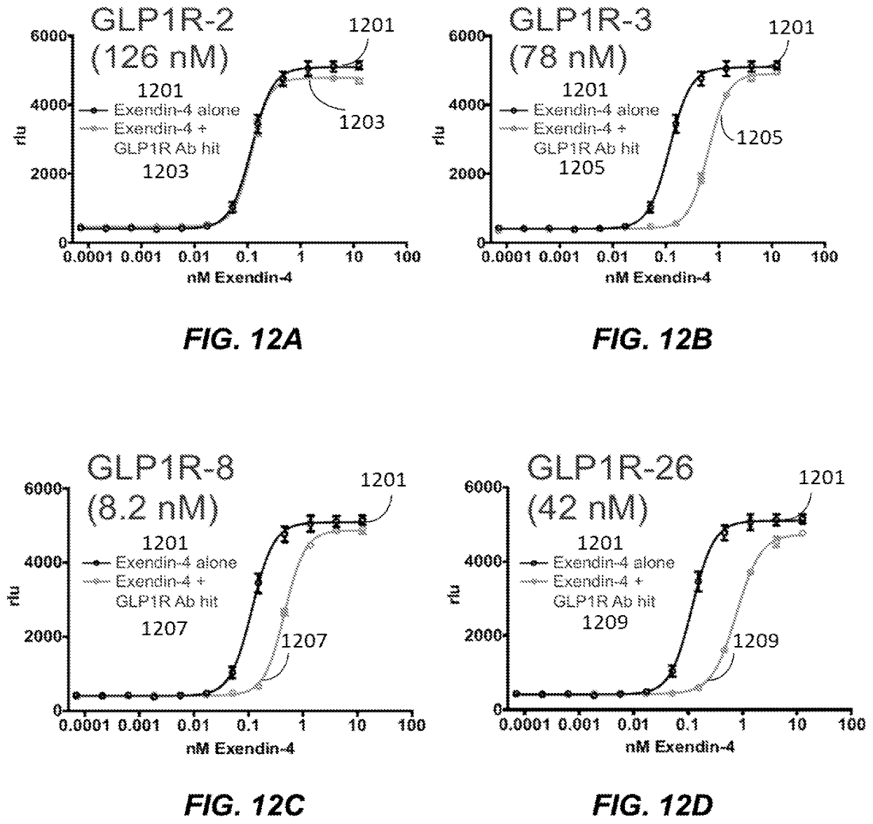

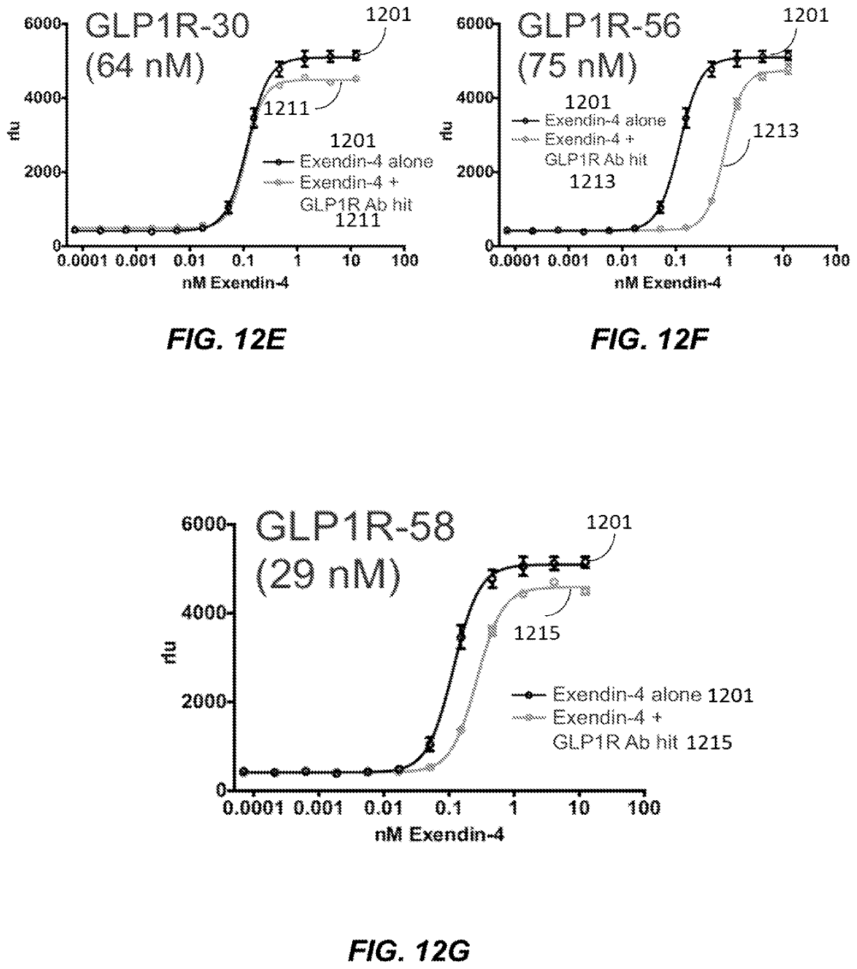

GLP1R-2, GLP1R-3, GLP1R-8, GLP1R-26, GLP1R-30, GLP1R-56, and GLP1R-58 were then analyzed for allosteric effects on exendin-4 in a cAMP assay. FIGS. 12A-12G depict graphs of GLP1R-2 (FIG. 12A), GLP1R-3 (FIG. 12B), GLP1R-8 (FIG. 12C), GLP1R-26 (FIG. 12D), GLP1R-30 (FIG. 12E), GLP1R-56 (FIG. 12F), and GLP1R-58 (FIG. 12G) variants on inhibition of Exendin-4 peptide induced cAMP activity. Table 12 shows the EC50 (nM) data for Exendin-4 alone or with GLP1R-2, GLP1R-3, GLP1R-8, GLP1R-26, GLP1R-30, GLP1R-56, and GLP1R-58.

TABLE 12
EC50 (nM) Data

| | EC50 | fold-diff |
|---|---|---|
| Exendin-4 alone | 0.12 | |
| +GLP1R-2 | 0.12 | 1.0 |
| +GLP1R-3 | 0.63 | 5.4 |
| +GLP1R-8 | 0.47 | 4.0 |
| +GLP1R-26 | 0.77 | 6.5 |
| +GLP1R-30 | 0.11 | 1.0 |
| +GLP1R-56 | 0.82 | 7.0 |
| +GLP1R-58 | 0.27 | 2.3 |

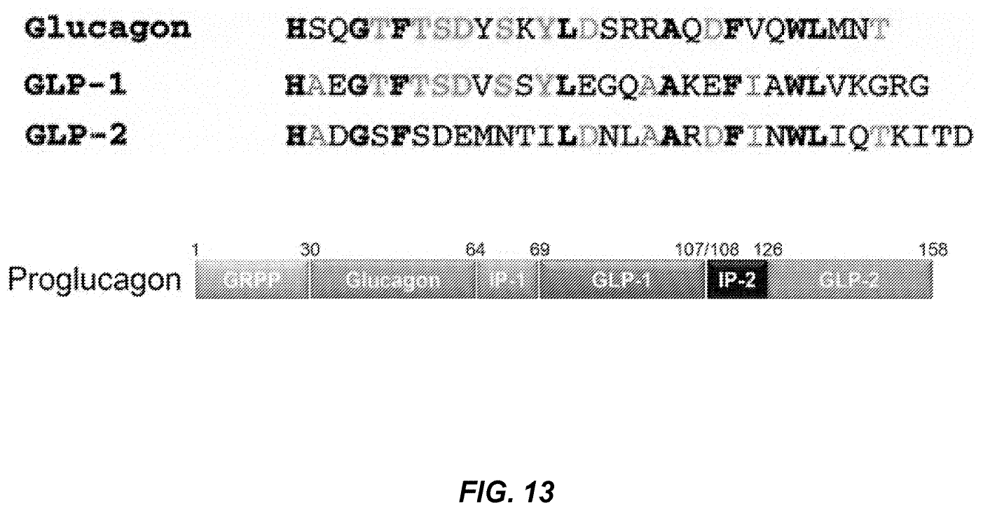

FACS screening was performed on GLP1R variants. GLP1R-2, GLP1R-3, GLP1R-8, GLP1R-10, GLP1R-25, GLP1R-26, GLP1R-30, GLP1R-56, GLP1R-58, GLP1R-60, GLP1R-70, GLP1R-72, GLP1R-83, GLP1R-93, and GLP1R-98 were identified as seen in Table 13. GLP1R-3, GLP1R-8, GLP1R-56, GLP1R-58, GLP1R-60, GLP1R-72, and GLP1R-83 comprise the GLP1 motif See FIG. 13. GLP1R-25, GLP1R-30, GLP1R-70, GLP1R-93, and GLP1R-98 comprise the GLP2 motif See FIG. 13. GLP1R-50 and GLP1R-71 comprise the CC chemokine 28 motif

TABLE 13
GLP1R Variants

| SEQ ID | | |
|---|---|---|
| NO | Variant | Sequence |
| 2277 | GLP1R-1 | CARANQHFVDLYGWHGVPKGYHYYGMDVW |
| 2278 | GLP1R-2 | CARDMYYDFETVVEGIQWYEALKAGKLGEVVPADDAFDIW |
| 2279 | GLP1R-3 | CAKHMSMQEGAVTGEGQAAKEFIAWLVKGRVRADLVGDAFDVW |
| 2280 | GLP1R-8 | CARDGRGSLPRPKGGPQTVGEGQAAKEFIAWLVKGGLTYDSSEDSGGAFDIW |
| 2281 | GLP1R-10 | CARANQHFFVPGSLKVWLKGVAPESSSEYDSSEDSGGAFDIW |
| 2282 | GLP1R-25 | CARANQHFLSHAGAARDFINWLIQTKITGLGSGYHYYGMDVW |
| 2283 | GLP1R-26 | CAKHMSMQEGVLQGQIPSTIDWEGLLHLIRADLVGDAFDVW |
| 2284 | GLP1R-30 | CARDMYYDFLKIGDNLAARDFINWLIQTKITDGTDTEVVPADDAFDIW |
| 2285 | GLP1R-50 | CARDGRGSLPRPKGGPKFVPGKHETYGHKTGYRLRPGYHYYGMDVW |
| 2286 | GLP1R-56 | CARANQHFFSGAEGEGQAAKEFIAWLVKGIIPGYHYYGMDVW |
| 2287 | GLP1R-58 | CARANQHFGLHAQGEGQAAKEFIAWLVKGSGTYGYHYYGMDVW |
| 2288 | GLP1R-60 | CAKHMSMQDYLVIGEGQAAKEFIAWLVKGGPARADLVGDAFDVW |
| 2289 | GLP1R-70 | CARDGRGSLPRPKGGPPSSGRDFINWLIQTKITDGFRYDSSEDSGGAFDIW |
| 2290 | GLP1R-71 | CARDLRELECEEWTRHGGKKHHGKRQSNRAHQGKHETYGHKTGSLVPSRGPCVDPR |
| | | GVAGSFDVW |
| 2291 | GLP1R-72 | CARDMYYDFHPEGTFTSDVSSYLEGQAAKEFIAWLVKGSLIYEVVPADDAFDIW |
| 2292 | GLP1R-80 | CARANQHFGPVAGGATPSEEPGSQLTRAELGWDAPPGQESLADELLQLGTEHGYHYY |
| | | GMDVW |
| 2293 | GLP1R-83 | CAKHMSMQEGAVTGEGQAAKEFIAWLVKGRVRADLVGDAFDVW |
| 2294 | GLP1R-93 | CARANQHFLSHAGAARDFINWLIQTKITGLGSGYHYYGMDVW |
| 2295 | GLP1R-98 | CARDGRGSLPRPKGGPHSGRLGSGYKSYDSSEDSGGAFDIW |

*bold corresponds to GLP1 or GLP2 motif

The GLP1R variants were assed for aggregation. Size exclusion chromatography (SEC) was performed on GLP1R-30 and GLP1R-56 variants. 82.64% of GLP1R-30 was monomeric (˜150 Kd). 97.4% of GLP1R-56 was monomeric (˜150 Kd).

### Example 11. GPCR Binding Protein Functionality

For a GPCR binding protein, the top 100-200 scFvs from phage-selections were converted to full-length immunoglobulins. After immunoglobulin conversion, the clones were transiently transfected in ExpiCHO to produce immunoglobulins. Kingfisher and Hamilton were used for batch IgG purifications followed by lab-chip to collect purity data for all purified immunoglobulins. High yields and purities were obtained from 10 mL cultures as seen in Table 14.

TABLE 14
Immunoglobulin Purity Percentage

| | IgG % |
|---|---|
| Name | Purity |
| mAb1 | 100 |
| mAb2 | 100 |
| mAb3 | 100 |
| mAb4 | 100 |
| mAb5 | 98 |
| mAb6 | 100 |
| mAb7 | 97 |
| mAb8 | 100 |
| mAb9 | 100 |
| mAb10 | 100 |
| mAb11 | 100 |
| mAb12 | 100 |
| mAb13 | 100 |
| mAb14 | 100 |
| mAb15 | 100 |

Stable cell lines expressing GPCR targets were then generated and confirmed by FACS (data not shown). Cells expressing >80% of the target were then directly used for cell-based selections. Five rounds of selections were carried out against cells overexpressing target of interest. 10⁸ cells were used for each round of selection. Before selection on target expressing cells, phage from each round was first depleted on 10⁸ CHO background cells. Stringency of selections was increased by increasing the number of washes in subsequent rounds of selection. Enrichment ratios were monitored for each round of selection.

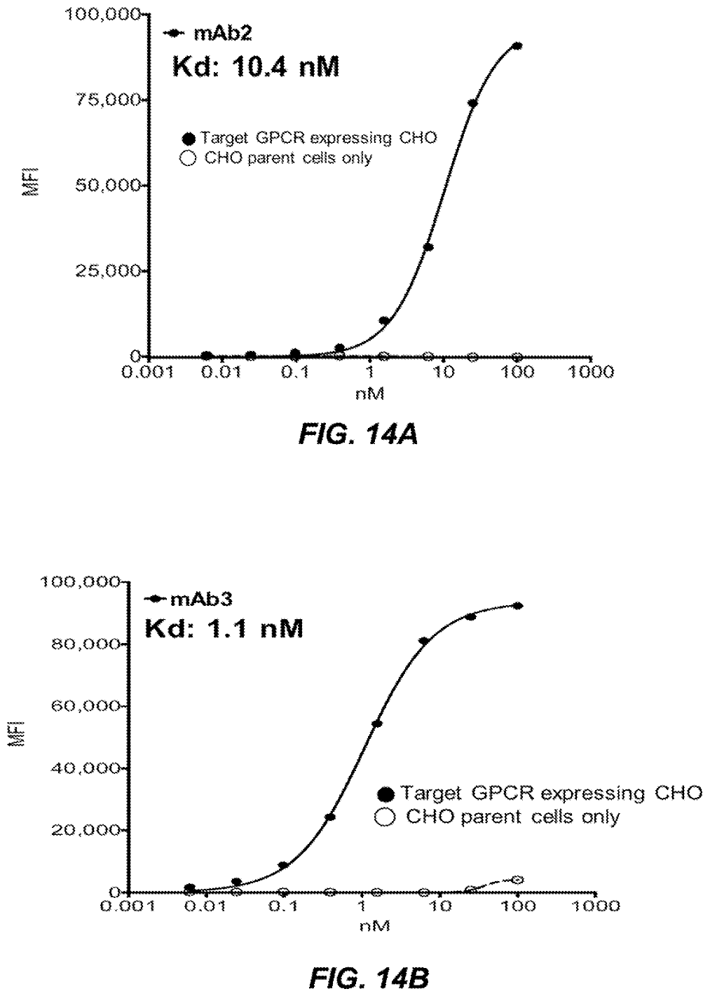

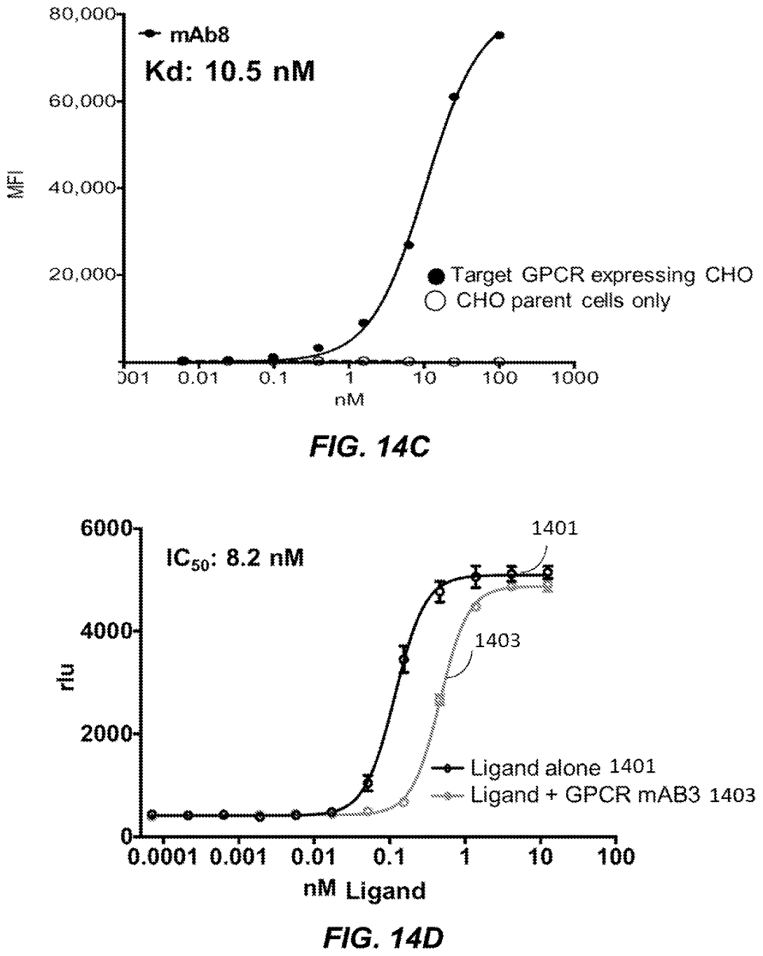

Purified IgGs were tested for cell-binding affinity using FACS (FIGS. 14A-14C) and cAMP activity (FIG. 14D). Allosteric inhibition was observed.

Purified IgGs were tested using BVP ELISA. BVP ELISA showed some clones comprising BVP scores comparable to comparator antibodies (data not shown).

### Example 12. GLP1R scFv Modulators

This example illustrates identification of GLP1R modulators.

### Library Panning

The GPCR1.0/2.0 scFv-phage library was incubated with CHO cells for 1 hour at room temperature (RT) to deplete CHO cell binders. After incubation, the cells were pelleted by centrifuging at 1,200 rpm for 10 minutes to remove the non-specific CHO cell binders. The phage supernatant, which has been depleted of CHO cell binders, was then transferred to GLP1R expressing CHO cells. The phage supernatant and GLP1R expressing CHO cells were incubated for 1 hour at RT to select for GLP1R binders. After incubation, the non-binding clones were washed away by washing with PBS several times. Finally, to selectively elute the agonist clones, the phage bound to the GLP1R cells were competed off with 1 μM of the natural ligand of GLP1R, GLP1 peptide (residues 7 to 36). The clones that eluted off the cells were likely binding to the GLP1 ligand binding epitope on GLP1R. Cells were pelleted by centrifuging at 1,200 rpm for 10 minutes to remove clones that were still binding to GLP1R on the cells, and were not binding to the endogenous GLP1 ligand binding site (orthosteric site). The supernatant was amplified in TG1 E. coli cells for the next round of selection. This selection strategy was repeated for five rounds. Amplified phage from a round was used as the input phage for the subsequent round, and the stringency of washes were increased in each subsequent round of selections. After five rounds of selection, 500 clones from each of round 4 and round 5 were Sanger sequenced to identify clones of GLP1R modulators. Seven unique clones were reformatted to IgG2, purified and tested in binding by FACS and functional assays.

### Binding Assays

Seven GLP1R scFv clones (GLP1R-238, GLP1R-239, GLP1R-240, GLP1R-241, GLP1R-242, GLP1R-243, and GLP1R-244) and two GLP1R IgGs (pGPCR-GLP1R-43 and pGPCR-GLP1R-44, Janssen Biotech, J&J) used as controls were tested in binding assays coupled to flow cytometry analysis. CDR3 sequences (Table 15), heavy chain sequences (Table 16), and light chain sequences (Table 17) for GLP1R-238, GLP1R-239, GLP1R-240, GLP1R-241, GLP1R-242, GLP1R-243, and GLP1R-244 are seen below.

TABLE 15
CDR3 sequences

| SEQ | | |
|---|---|---|
| ID NO. | Variant | CDR-H3 Sequence |
| 2296 | GLP1R-238 | CARANQHFSQAGRAARVPGPSSSLGPRGYH |
| | | YYGMDVW |
| 2297 | GLP1R-239 | CAKHMSMQSQGLDNLAARDFINWLIQTKIT |
| | | DGFELSRADLVGDAFDVW |
| 2298 | GLP1R-240 | CARDMYYDFFGLGTFTSDVSSYLEGQAAKE |
| | | FIAWLVKGVSPEVVPADDAFDIW |
| 2299 | GLP1R-241 | CAKHMSMQGSVAGGTFTSDVSSYLEGQAAK |
| | | EFIAWLVKGGPSFIRADLVGDAFDVW |
| 2300 | GLP1R-242 | CAKHMSMQADTGTFTSDVSSYLEGQAAKEF |
| | | IAWLVKGEFSSRADLVGDAFDVW |
| 2301 | GLP1R-243 | CARANQHFFGKGDNLAARDFINWLIQTKIT |
| | | DGSNPGYHYYGMDVW |
| 2302 | GLP1R-244 | CARANQHFAATGAGEGQAAKEFIAWLVKGR |
| | | VEIGYHYYGMDVW |

*bold correspond to GLP-1 or GLP-2 motif

TABLE 16
Variable Heavy Chain Sequences

| SEQ | | |
|---|---|---|
| ID NO. | Variant | Variable Heavy Chain Sequence |
| 2303 | GLP1R- | MEWSWVFLFFLSVTTGVHSQVQLVQSGAEVKK |
| | 238 | PGSSVKVSCKASGGSFSSHAISWVRQAPGQGL |
| | | EWMGGIIPIFGAPNYAQKFQGRVTITADESTS |
| | | TAYMELSSLRSEDTAVYYCARANQHFSQAGRA |
| | | ARVPGPSSSLGPRGYHYYGMDVWGQGTLVTVS |
| | | SASASTKGPSVFPLAPCSRSTSESTAALGCLV |
| | | KDYFPEPVTVSWNSGALTSGVHTFPAVLQSSG |
| | | LYSLSSVVTVPSSNFGTQTYTCNVDHKPSNTK |
| | | VDKTVERKCCVECPPCPAPPVAGPSVFLFPPK |
| | | PKDTLMISRTPEVTCVVVDVSHEDPEVQFNWY |
| | | VDGVEVHNAKTKPREEQFNSTFRVVSVLTVVH |
| | | QDWLNGKEYKCKVSNKGLPAPIEKTISKTKGQ |
| | | PREPQVYTLPPSREEMTKNQVSLTCLVKGFYP |
| | | SDIAVEWESNGQPENNYKTTPPMLDSDGSFFL |
| | | YSKLTVDKSRWQQGNVFSCSVMHEALHNHYTQ |
| | | KSLSLSPG |
| 2304 | GLP1R- | MEWSWVFLFFLSVTTGVHSQVQLVESGGGVVQ |
| | 239 | PGRSLRLSCAASGFDFSNYGMHWVRQAPGKGL |
| | | EWVADISYEGSNKYYADSVKGRFTISRDNSKN |
| | | TLYLQMNSLRAEDTAVYYCAKHMSMQSQGLDN |
| | | LAARDFINWLIQTKITDGFELSRADLVGDAFD |
| | | VWGQGTLVTVSSASASTKGPSVFPLAPCSRST |
| | | SESTAALGCLVKDYFPEPVTVSWNSGALTSGV |
| | | HTFPAVLQSSGLYSLSSVVTVPSSNFGTQTYT |
| | | CNVDHKPSNTKVDKTVERKCCVECPPCPAPPV |
| | | AGPSVFLFPPKPKDTLMISRTPEVTCVVVDVS |
| | | HEDPEVQFNWYVDGVEVHNAKTKPREEQFNST |
| | | FRVVSVLTVVHQDWLNGKEYKCKVSNKGLPAP |
| | | IEKTISKTKGQPREPQVYTLPPSREEMTKNQV |
| | | SLTCLVKGFYPSDIAVEWESNGQPENNYKTTP |
| | | PMLDSDGSFFLYSKLTVDKSRWQQGNVFSCSV |
| | | MHEALHNHYTQKSLSLSPG |
| 2305 | GLP1R- | MEWSWVFLFFLSVTTGVHSQVQLVQSGAEVKK |
| | 240 | PGSSVKVSCKASGGTFNNYGISWVRQAPGQGL |
| | | EWMGGIIPVFGTANYAQKFQGRVTITADESTS |
| | | TAYMELSSLRSEDTAVYYCARDMYYDFFGLGT |
| | | FTSDVSSYLEGQAAKEFIAWLVKGVSPEVVPA |
| | | DDAFDIWGQGTLVTVSSASASTKGPSVFPLAP |
| | | CSRSTSESTAALGCLVKDYFPEPVTVSWNSGA |
| | | LTSGVHTFPAVLQSSGLYSLSSVVTVPSSNFG |
| | | TQTYTCNVDHKPSNTKVDKTVERKCCVECPPC |
| | | PAPPVAGPSVFLFPPKPKDTLMISRTPEVTCV |
| | | VVDVSHEDPEVQFNWYVDGVEVHNAKTKPREE |
| | | QFNSTFRVVSVLTVVHQDWLNGKEYKCKVSNK |
| | | GLPAPIEKTISKTKGQPREPQVYTLPPSREEM |
| | | TKNQVSLTCLVKGFYPSDIAVEWESNGQPENN |
| | | YKTTPPMLDSDGSFFLYSKLTVDKSRWQQGNV |
| | | FSCSVMHEALHNHYTQKSLSLSPG |
| 2306 | GLP1R- | MEWSWVFLFFLSVTTGVHSQVQLVQSGAEVKK |
| | 241 | PGSSVKVSCKASGGTFSDYAISWVRQAPGQGL |
| | | EWMGGIIPIFGTTNYAQKFQGRVTITADESTS |
| | | TAYMELSSLRSEDTAVYYCAKHMSMQGSVAGG |
| | | TFTSDVSSYLEGQAAKEFIAWLVKGGPSFIRA |
| | | DLVGDAFDVWGQGTLVTVSSASASTKGPSVFP |
| | | LAPCSRSTSESTAALGCLVKDYFPEPVTVSWN |
| | | SGALTSGVHTFPAVLQSSGLYSLSSVVTVPSS |
| | | NFGTQTYTCNVDHKPSNTKVDKTVERKCCVEC |
| | | PPCPAPPVAGPSVFLFPPKPKDTLMISRTPEV |
| | | TCVVVDVSHEDPEVQFNWYVDGVEVHNAKTKP |
| | | REEQFNSTFRVVSVLTVVHQDWLNGKEYKCKV |
| | | SNKGLPAPIEKTISKTKGQPREPQVYTLPPSR |
| | | EEMTKNQVSLTCLVKGFYPSDIAVEWESNGQP |
| | | ENNYKTTPPMLDSDGSFFLYSKLTVDKSRWQQ |
| | | GNVFSCSVMHEALHNHYTQKSLSLSPG |
| 2307 | GLP1R- | MEWSWVFLFFLSVTTGVHSQVQLVQSGAEVKK |
| | 242 | PGSSVKVSCKASGGTFSSYEISWVRQAPGQGL |
| | | EWMGGIIPILGIANYAQKFQGRVTITADESTS |
| | | TAYMELSSLRSEDTAVYYCAKHMSMQADTGTF |
| | | TSDVSSYLEGQAAKEFIAWLVKGEFSSRADLV |
| | | GDAFDVWGQGTLVTVSSASASTKGPSVFPLAP |
| | | CSRSTSESTAALGCLVKDYFPEPVTVSWNSGA |
| | | LTSGVHTFPAVLQSSGLYSLSSVVTVPSSNFG |
| | | TQTYTCNVDHKPSNTKVDKTVERKCCVECPPC |
| | | PAPPVAGPSVFLFPPKPKDTLMISRTPEVTCV |
| | | VVDVSHEDPEVQFNWYVDGVEVHNAKTKPREE |
| | | QFNSTFRVVSVLTVVHQDWLNGKEYKCKVSNK |
| | | GLPAPIEKTISKTKGQPREPQVYTLPPSREEM |
| | | TKNQVSLTCLVKGFYPSDIAVEWESNGQPENN |
| | | YKTTPPMLDSDGSFFLYSKLTVDKSRWQQGNV |
| | | FSCSVMHEALHNHYTQKSLSLSPG |
| 2308 | GLP1R- | MEWSWVFLFFLSVTTGVHSQVQLVQSGAEVKK |
| | 243 | PGSSVKVSCKASGGTFSTYGINWVRQAPGQGL |
| | | EWMGGIIPIFGTANYAQKFQGRVTITADESTS |
| | | TAYMELSSLRSEDTAVYYCARANQHFFGKGDN |
| | | LAARDFINWLIQTKITDGSNPGYHYYGMDVWG |
| | | QGTLVTVSSASASTKGPSVFPLAPCSRSTSES |
| | | TAALGCLVKDYFPEPVTVSWNSGALTSGVHTF |
| | | PAVLQSSGLYSLSSVVTVPSSNFGTQTYTCNV |
| | | DHKPSNTKVDKTVERKCCVECPPCPAPPVAGP |
| | | SVFLFPPKPKDTLMISRTPEVTCVVVDVSHED |
| | | PEVQFNWYVDGVEVHNAKTKPREEQFNSTFRV |
| | | VSVLTVVHQDWLNGKEYKCKVSNKGLPAPIEK |
| | | TISKTKGQPREPQVYTLPPSREEMTKNQVSLT |
| | | CLVKGFYPSDIAVEWESNGQPENNYKTTPPML |
| | | DSDGSFFLYSKLTVDKSRWQQGNVFSCSVMHE |
| | | ALHNHYTQKSLSLSPG |
| 2309 | GLP1R- | MEWSWVFLFFLSVTTGVHSQVQLVQSGAEVKK |
| | 244 | PGSSVKVSCKASGGTFSSYAISWVRQAPGQGL |
| | | EWMGGIIPIFGTANYAQKFQGRVTITADESTS |
| | | TAYMELSSLRSEDTAVYYCARANQHFAATGAG |
| | | EGQAAKEFIAWLVKGRVEIGYHYYGMDVWGQG |
| | | TLVTVSSASASTKGPSVFPLAPCSRSTSESTA |
| | | ALGCLVKDYFPEPVTVSWNSGALTSGVHTFPA |
| | | VLQSSGLYSLSSVVTVPSSNFGTQTYTCNVDH |
| | | KPSNTKVDKTVERKCCVECPPCPAPPVAGPSV |
| | | FLFPPKPKDTLMISRTPEVTCVVVDVSHEDPE |
| | | VQFNWYVDGVEVHNAKTKPREEQFNSTFRVVS |
| | | VLTVVHQDWLNGKEYKCKVSNKGLPAPIEKTI |
| | | SKTKGQPREPQVYTLPPSREEMTKNQVSLTCL |
| | | VKGFYPSDIAVEWESNGQPENNYKTTPPMLDS |
| | | DGSFFLYSKLTVDKSRWQQGNVFSCSVMHEAL |
| | | HNHYTQKSLSLSPG |

TABLE 17
Variable Light Chain Sequences

| SEQ | | |
|---|---|---|
| ID NO. | Variant | Variable Light Chain Sequence |
| 2310 | GLP1R- | MSVPTQVLGLLLLWLTDARCQSVLTQPPSVSA |
| | 238 | APGQKVTISCSGSTSNIANNYVSWYQQLPGTA |
| | | PKLLIYANNRRPSGIPDRFSGSKSGTSATLGI |
| | | TGLQTGDEADYYCGAWDVRLDVGVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLS |
| 2311 | GLP1R- | MSVPTQVLGLLLLWLTDARCQSVLTQPPSVSA |
| | 239 | APGQKVTISCSGSTSNIEKNYVSWYQQLPGTA |
| | | PKLLIYGNDQRPSGIPDRFSGSKSGTSATLGI |
| | | TGLQTGDEADYYCGTWENRLSAVVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLSLTPEQWKSHRSYSCQVTHEGS |
| | | TVEKTVAPTECS |
| 2312 | GLP1R- | MSVPTQVLGLLLLWLTDARCQSVLTQPPSVSA |
| | 240 | APGQKVTISCSGSSSSIGNNYVSWYQQLPGTA |
| | | PKLLIYANNKRPSGIPDRFSGSKSGTSATLGI |
| | | TGLQTGDEADYYCATWSSSPRGWVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLSLTPEQWKSHRSYSCQVTHEGS |
| | | TVEKTVAPTECS |
| 2313 | GLP1R- | MSVPTQVLGLLLLWLTDARCQSVLTQPPSVSA |
| | 241 | APGQKVTISCSGISSNIGNNYVSWYQQLPGTA |
| | | PKLLIYDDDQRPSGIPDRFSGSKSGTSATLGI |
| | | TGLQTGDEADYYCGTWDNILSAAVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLSLTPEQWKSHRSYSCQVTHEGS |
| | | TVEKTVAPTECS |
| 2314 | GLP1R- | MSVPTQVLGLLLLWLTDARCQSVLTQPPSVSA |
| | 242 | APGQKVTISCSGSSSNIENNDVSWYQQLPGTA |
| | | PKLLIYGNDQRPSGIPDRFSGSKSGTSATLGI |
| | | TGLQTGDEADYYCGTWDNTLSAGVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLSLTPEQWKSHRSYSCQVTHEGS |
| | | TVEKTVAPTECS |
| 2315 | GLP1R- | MSVPTQVLGLLLLWLTDARCQSVLTQPPSVSA |
| | 243 | APGQKVTISCSGSRSNIGKNYVSWYQQLPGTA |
| | | PKLLIYENNERPSGIPDRFSGSKSGTSATLGI |
| | | TGLQTGDEADYYCSSYTTSNTQVFGGGTKLTV |
| | | LGQPKAAPSVTLFPPSSEELQANKATLVCLIS |
| | | DFYPGAVTVAWKADSSPVKAGVETTTPSKQSN |
| | | NKYAASSYLSLTPEQWKSHRSYSCQVTHEGST |
| | | VEKTVAPTECS |
| 2316 | GLP1R- | MSVPTQVLGLLLLWLTDARCQSVLTQPPSVSA |
| | 244 | APGQKVTISCSGSSSNIGNNVVSWYQQLPGTA |
| | | PKLLIYDNDKRRSGIPDRFSGSKSGTSATLGI |
| | | TGLQTGDEADYYCGSWDTSLSVWVFGGGTKLT |
| | | VLGQPKAAPSVTLFPPSSEELQANKATLVCLI |
| | | SDFYPGAVTVAWKADSSPVKAGVETTTPSKQS |
| | | NNKYAASSYLSLTPEQWKSHRSYSCQVTHEGS |
| | | TVEKTVAPTECS |

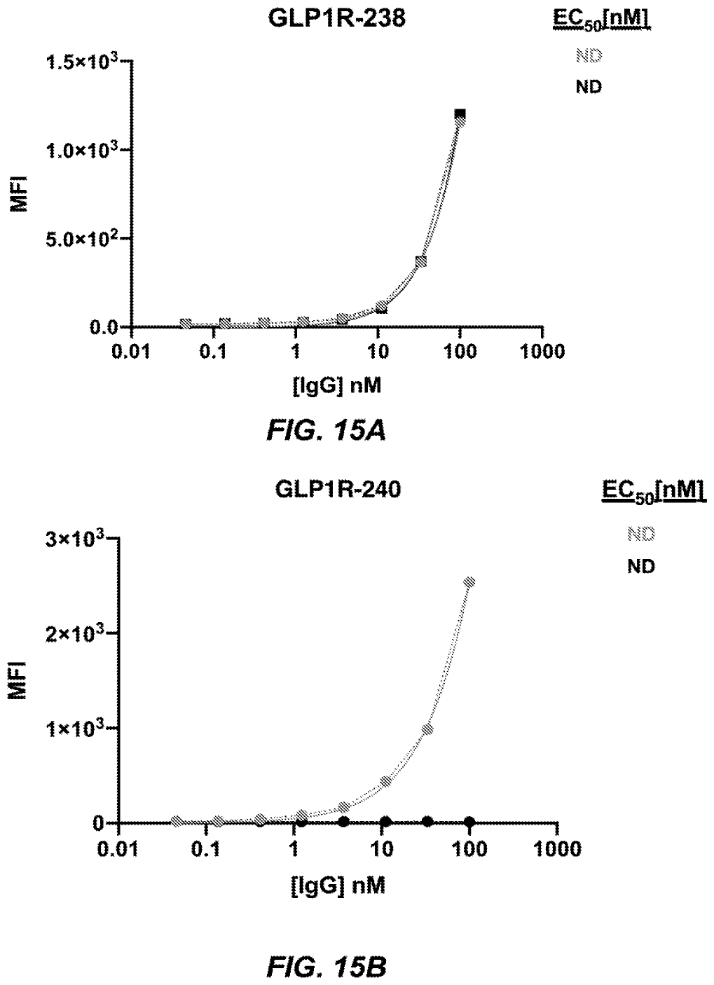

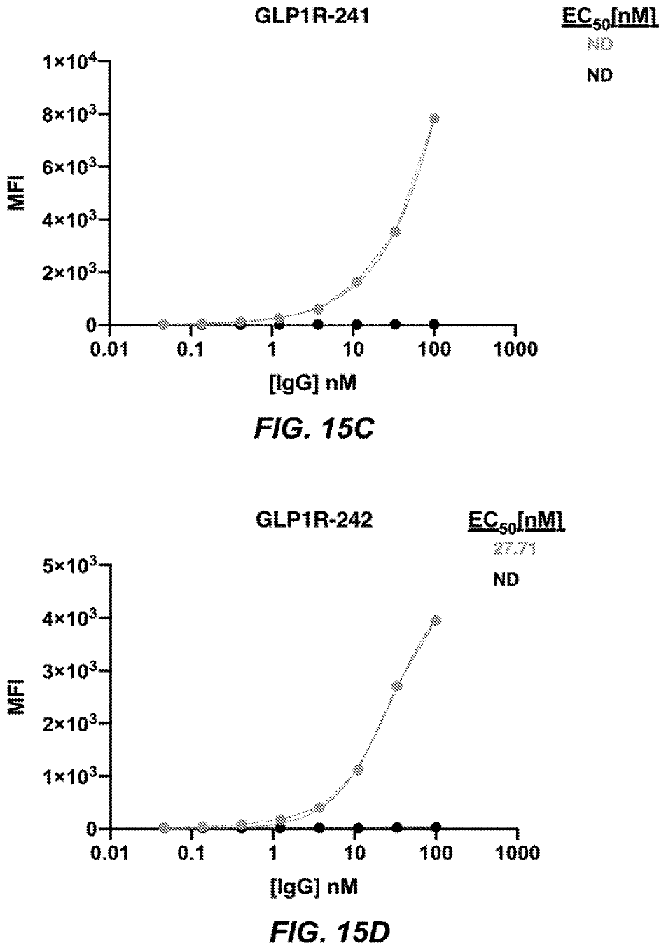

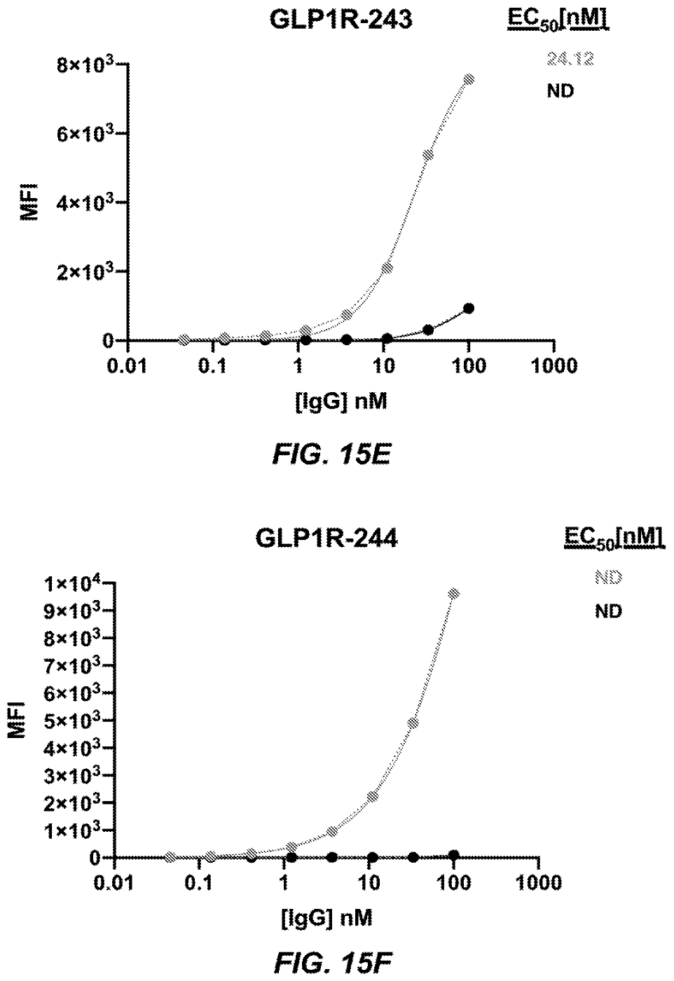

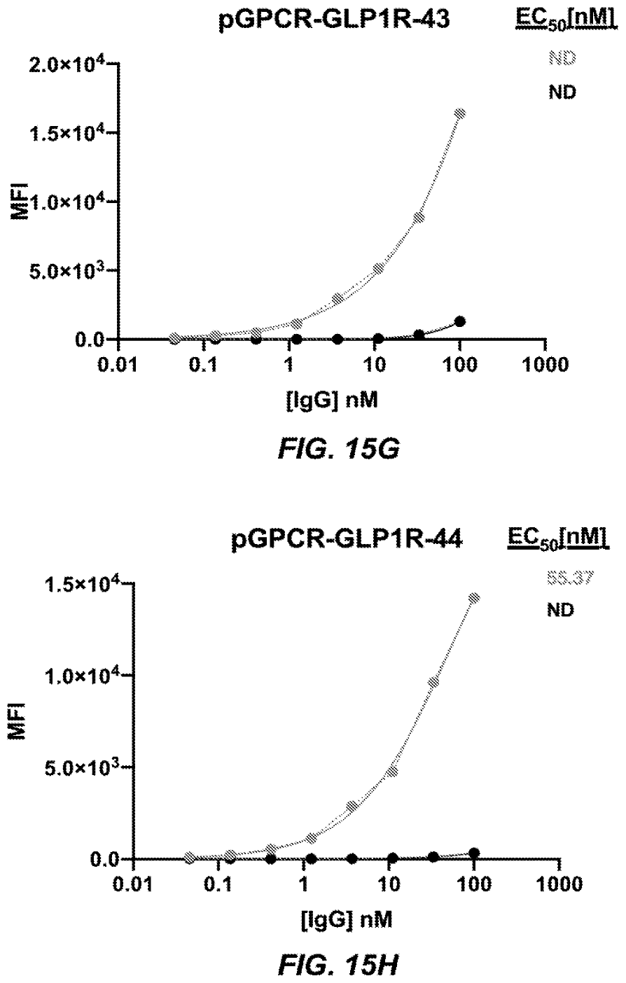

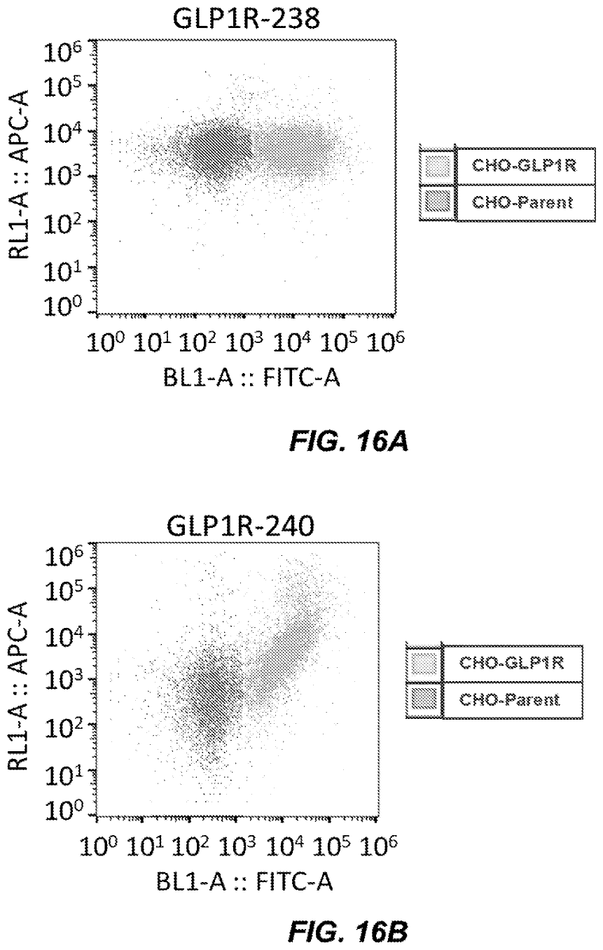

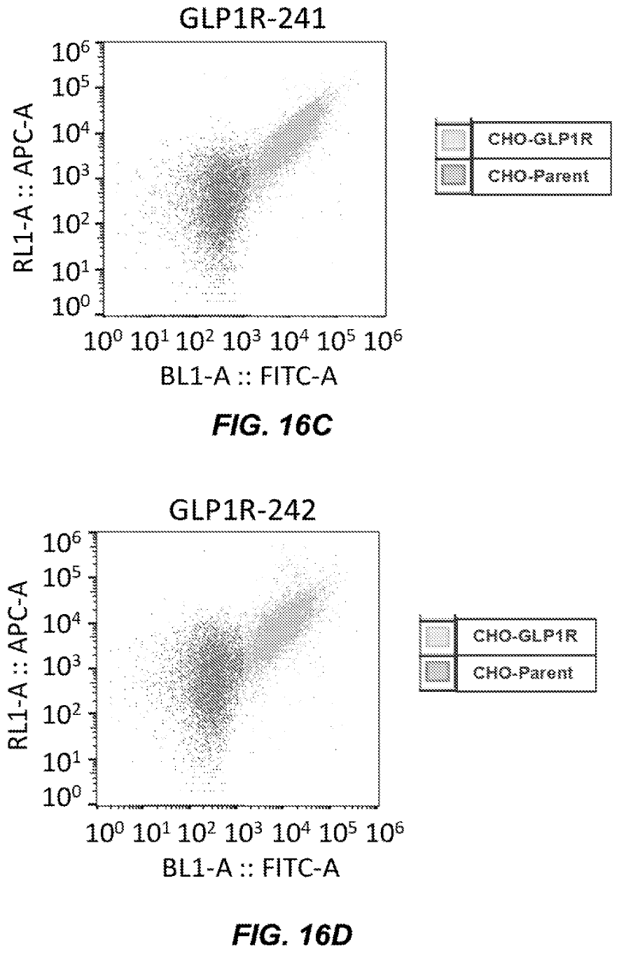

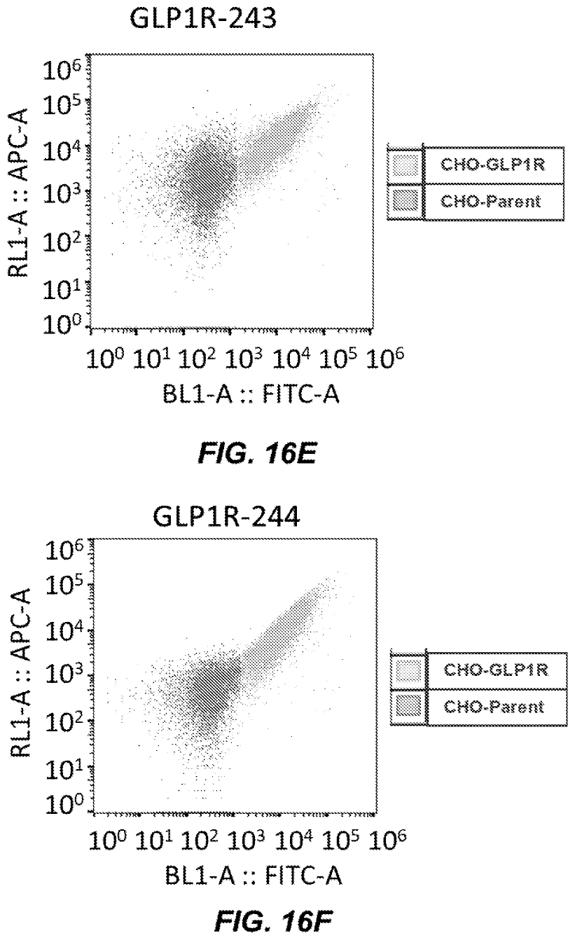

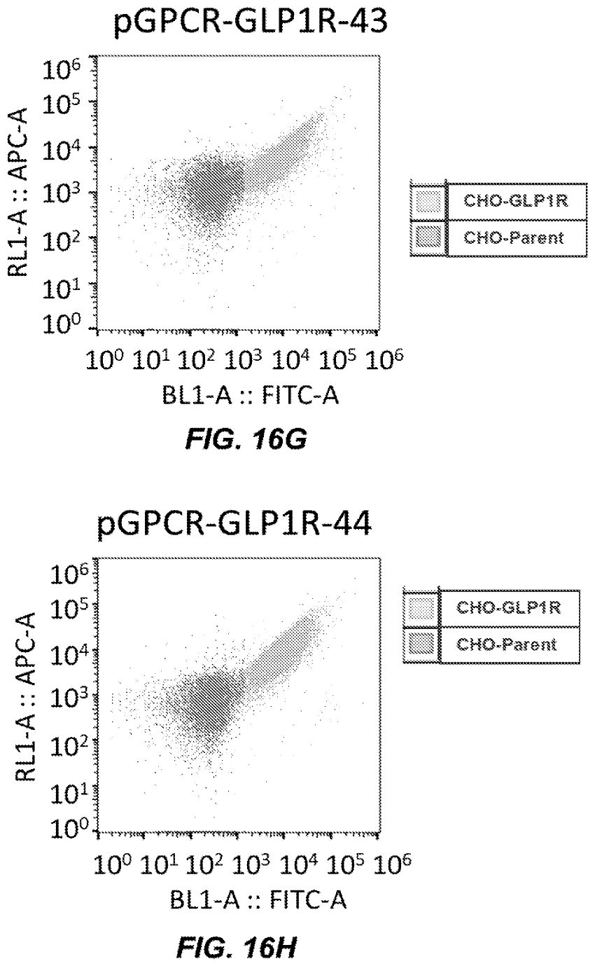

Briefly, flag-GLP1R-GFP expressing CHO cells (CHO-GLP1R) and CHO-parent cells were incubated with 100 nM IgG for 1 hour on ice, washed three times and incubated with Alexa 647 conjugated goat-anti-human antibody (1:200) for 30 minutes on ice, followed by three washes. All incubations and washes were performed in buffer containing PBS and 0.5% BSA. For titrations, IgG was serially diluted 1:3 starting from 100 nM. Cells were analyzed by flow cytometry and hits in which IgG was found to bind to CHO-GLP1R were identified by measuring the GFP signal against the Alexa 647 signal. GLP1R-238, GLP1R-240, GLP1R-241, GLP1R-242, GLP1R-243, and GLP1R-244 were found to bind to CHO-GLP1R. GLP1R-238 bound equally to CHO-GLP1R and CHO-parent cells and thus appears to be a non-specific binder. Analyses of binding assays with IgG titrations presented as binding curves plotting IgG concentrations against MFI (mean fluorescence intensity) are seen in FIGS. 15A-15H. Flow cytometry data of binding assays presented as dot plots with 100 nM IgG are seen in FIGS. 16A-16I.

### Functional Assays

All GLP1R scFv clones, as well as pGPCR-GLP1R-43 and pGPCR-GLP1R-44, were assayed for their potential effects on GLP1R signaling by performing cAMP assays (Eurofins DiscoverX Corporation). These assays involve CHO cells that were engineered to overexpress naturally Gαₛ-coupled wildtype GLP1R and were designed to detect changes in intracellular cAMP levels in response to agonist stimulation of the receptor. The technology involved in detecting cAMP levels was a no wash gain-of-signal competitive immunoassay based on Enzyme Fragment Complementation technology and produced a luminescent signal that was directly proportional to the amount of cAMP in the cells. Experiments were designed to determine agonist or allosteric activity of the IgGs. To test for agonist activity of the IgGs, cells were stimulated with IgGs (titrations 1:3 starting from 100 nM) or with the known agonist GLP1 (7-36) peptide (titrations 1:6 starting from 12.5 nM) for 30 minutes at 37° C. To test for allosteric activity of the IgGs, cells were incubated with IgGs at a fixed concentration of 100 nM for 1 hour at room temperature to allow binding, followed by stimulation with GLP1 (7-36) peptide (titrations 1:6 starting from 12.5 nM) for 30 minutes at 37° C. Intracellular cAMP levels were detected by following the assay kit instructions.

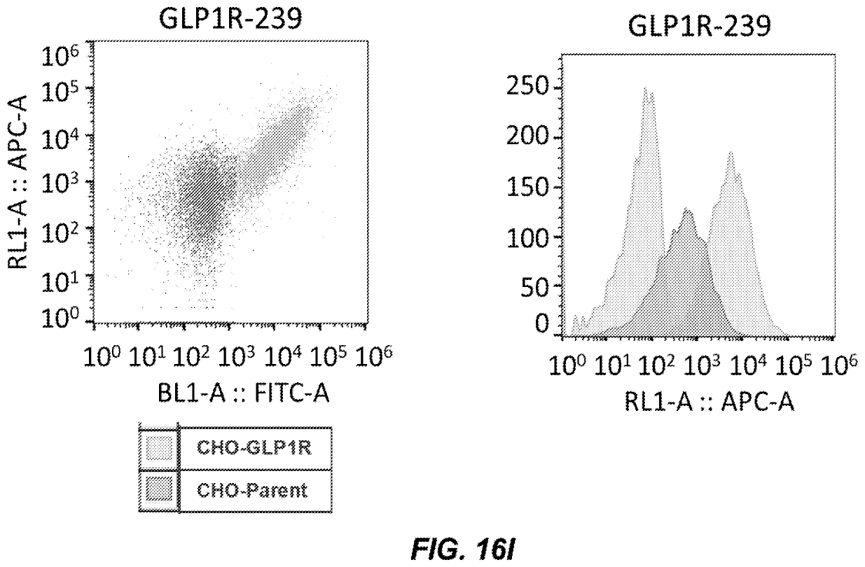

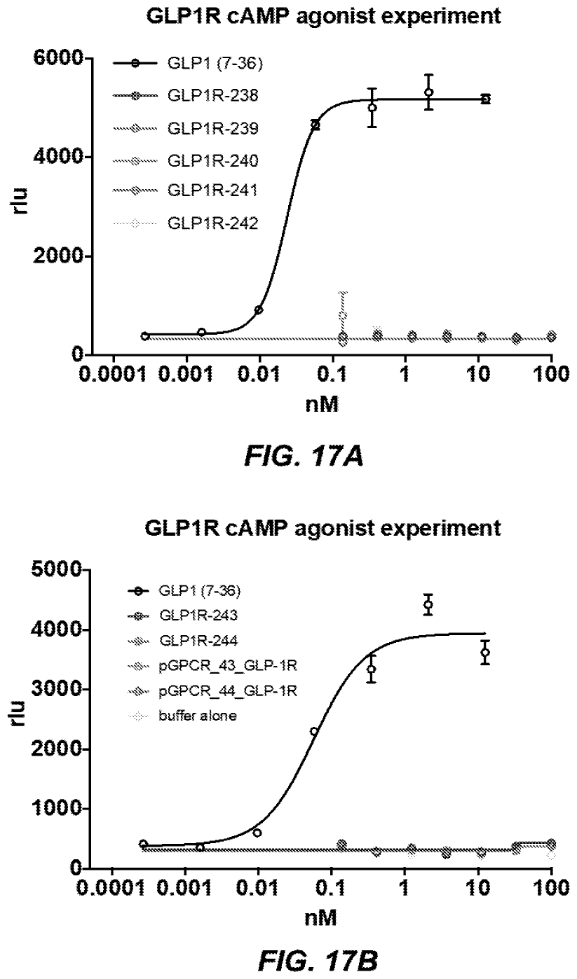

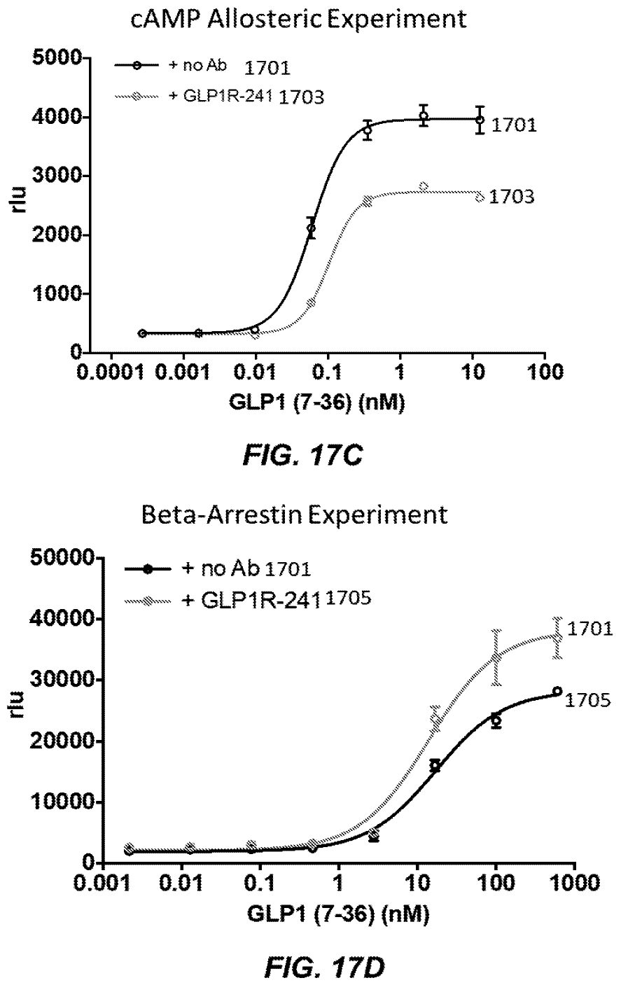

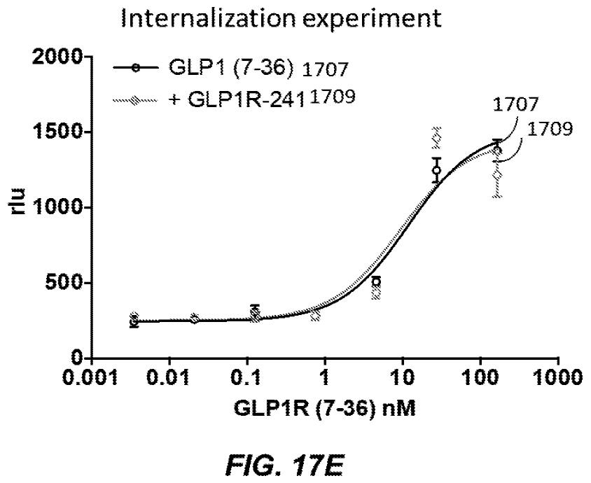

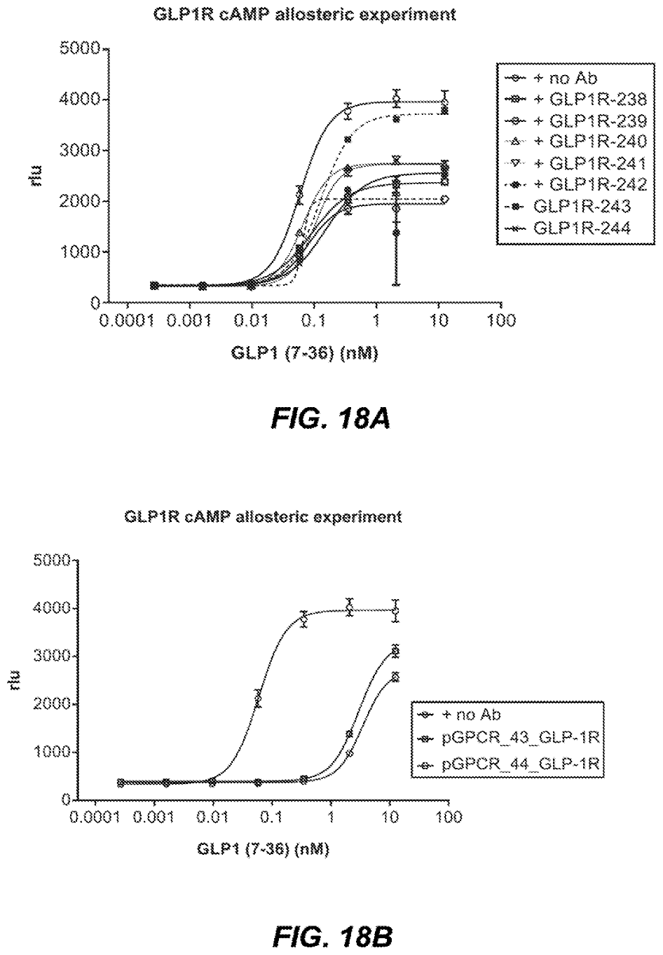

As seen in FIGS. 17A-17B, none of the IgGs initiated an agonist signal. GLP1R-241 was also tested for cAMP allosteric effect (FIG. 17C), beta-arrestin recruitment (FIG. 17D), and internalization (FIG. 17E). Several of the IgGs acted as negative allosteric modulators by changing the signaling response of these cells to GLP1 (7-36) in an inhibitory manner as seen in FIGS. 18A-18B. Table 18 shows the EC50 (nM) values corresponding to FIG. 18A and Table 19 shows the EC50 corresponding to FIG. 18B.

TABLE 18
EC50 (nM) Values

| | +no | +GLP1R- | +GLP1R- | +GLP1R- | +GLP1R- | +GLP1R- | GLP1R- | GLP1R- |
|---|---|---|---|---|---|---|---|---|
| | Ab | 238 | 239 | 240 | 241 | 242 | 243 | 244 |
| EC50 | 0.05946 | 0.08793 | 0.07995 | 0.06539 | 0.1027 | ~0.06532 | 0.1282 | 0.1536 |

TABLE 19
EC50 (nM) Values

| | | pGPCR- | pGPCR- |
|---|---|---|---|
| | +no Ab | 43-GLP1R | 44-GLP1R |
| EC50 | 0.05946 | 2.948 | 3.485 |

The data shows pharmacological and functional effects of GLP1R modulators.

### Example 13: GLP1R Agonists and Antagonists

This example illustrates identification of GLPIR agonists and antagonists.

Experiments were performed similarly to Example 12. Six GLP1R immunoglobulins (IgGs) were assayed for binding and functional assays to determine which clones were agonists or antagonists. The GLP1R IgGs tested included GLP1R-59-2, GLP1R-59-241, GLP1R-59-243, GLP1R-3, GLP1R-241, and GLP1R-2. GLP1R-241, GLP1R-3, and GLP1R-2 were previously described in Examples 10 and 12. Heavy chain sequences for GLP1R-59-2, GLP1R-59-241, GLP1R-59-243, GLP1R-43-8, and GLP1R-3 is seen in Table 20.

TABLE 20
Variable Heavy Chain Sequences

| SEQ | | |
|---|---|---|
| ID NO. | Variant | Variable Heavy Chain Sequence |
| 2317 | GLP1R- | QVQLVESGGGVVQPGRSLRLSCAASGFTFSNY |
| | 59-2 | GMSWVRQAPGKGLEWVAVISYDAGNKYYADSV |
| | | KGRFTISRDNSKNTLYLQMNSLRAEDTAVYYC |
| | | ARDMYYDFETVVEGIQWYEALKAGKLGEVVPA |
| | | DDAFDIWGQGTLVTVSSASTKGPSVFPLAPCS |
| | | RSTSESTAALGCLVKDYFPEPVTVSWNSGALT |
| | | SGVHTFPAVLQSSGLYSLSSVVTVPSSNFGTQ |
| | | TYTCNVDHKPSNTKVDKTVERKCCVECPPCPA |
| | | PPVAGPSVFLFPPKPKDTLMISRTPEVTCVVV |
| | | DVSHEDPEVQFNWYVDGVEVHNAKTKPREEQF |
| | | NSTFRVVSVLTVVHQDWLNGKEYKCKVSNKGL |
| | | PAPIEKTISKTKGQPREPQVYTLPPSREEMTK |
| | | NQVSLTCLVKGFYPSDIAVEWESNGQPENNYK |
| | | TTPPMLDSDGSFFLYSKLTVDKSRWQQGNVFS |
| | | CSVMHEALHNHYTQKSLSLSPGK |
| 2318 | GLP1R- | QVQLVQSGAEVKKPGSSVKVSCKASGGTFSDY |
| | 59-241 | AISWVRQAPGQGLEWMGGIIPIFGTTNYAQKF |
| | | QGRVTITADESTSTAYMELSSLRSEDTAVYYC |
| | | AKHMSMQGSVAGGTFTSDVSSYLEGQAAKEFI |
| | | AWLVKGGPSFIRADLVGDAFDVWGQGTLVTVS |
| | | SASASTKGPSVFPLAPCSRSTSESTAALGCLV |
| | | KDYFPEPVTVSWNSGALTSGVHTFPAVLQSSG |
| | | LYSLSSVVTVPSSNFGTQTYTCNVDHKPSNTK |
| | | VDKTVERKCCVECPPCPAPPVAGPSVFLFPPK |
| | | PKDTLMISRTPEVTCVVVDVSHEDPEVQFNWY |
| | | VDGVEVHNAKTKPREEQFNSTFRVVSVLTVVH |
| | | QDWLNGKEYKCKVSNKGLPAPIEKTISKTKGQ |
| | | PREPQVYTLPPSREEMTKNQVSLTCLVKGFYP |
| | | SDIAVEWESNGQPENNYKTTPPMLDSDGSFFL |
| | | YSKLTVDKSRWQQGNVFSCSVMHEALHNHYTQ |
| | | KSLSLSPG |
| 2319 | GLP1R- | QVQLVQSGAEVKKPGSSVKVSCKASGGTFSTY |
| | 59-243 | GINWVRQAPGQGLEWMGGIIPIFGTANYAQKF |
| | | QGRVTITADESTSTAYMELSSLRSEDTAVYYC |
| | | ARANQHFFGKGDNLAARDFINWLIQTKITDGS |
| | | NPGYHYYGMDVWGQGTLVTVSSASASTKGPSV |
| | | FPLAPCSRSTSESTAALGCLVKDYFPEPVTVS |
| | | WNSGALTSGVHTFPAVLQSSGLYSLSSVVTVP |
| | | SSNFGTQTYTCNVDHKPSNTKVDKTVERKCCV |
| | | ECPPCPAPPVAGPSVFLFPPKPKDTLMISRTP |
| | | EVTCVVVDVSHEDPEVQFNWYVDGVEVHNAKT |
| | | KPREEQFNSTFRVVSVLTVVHQDWLNGKEYKC |
| | | KVSNKGLPAPIEKTISKTKGQPREPQVYTLPP |
| | | SREEMTKNQVSLTCLVKGFYPSDIAVEWESNG |
| | | QPENNYKTTPPMLDSDGSFFLYSKLTVDKSRW |
| | | QQGNVFSCSVMHEALHNHYTQKSLSLSPG |
| 2320 | GLP1R-3 | QVQLVESGGGVVQPGRSLRLSCAASGFTFSSY |
| | | GMHWVRQAPGKGLEWVSFISYDESNKYYADSV |
| | | KGRFTISRDNSKNTLYLQMNSLRAEDTAVYYC |
| | | AKHMSMQEGAVTGEGQAAKEFIAWLVKGRVRA |
| | | DLVGDAFDVWGQGTLVTVSSASTKGPSVFPLA |
| | | PCSRSTSESTAALGCLVKDYFPEPVTVSWNSG |
| | | ALTSGVHTFPAVLQSSGLYSLSSVVTVPSSNF |
| | | GTQTYTCNVDHKPSNTKVDKTVERKCCVECPP |
| | | CPAPPVAGPSVFLFPPKPKDTLMISRTPEVTC |
| | | VVVDVSHEDPEVQFNWYVDGVEVHNAKTKPRE |
| | | EQFNSTFRVVSVLTVVHQDWLNGKEYKCKVSN |
| | | KGLPAPIEKTISKTKGQPREPQVYTLPPSREE |
| | | MTKNQVSLTCLVKGFYPSDIAVEWESNGQPEN |
| | | NYKTTPPMLDSDGSFFLYSKLTVDKSRWQQGN |
| | | VFSCSVMHEALHNHYTQKSLSLSPGK |
| 2321 | GLP1R- | MEWSWVFLFFLSVTTGVHSEVQLVESGGGLVQ |
| | 43-8 | AGGSLRLSCAASGSIFRINAMGWFRQAPGKER |
| | | EGVAAINNFGTTKYADSAKGRFTISADNAKNT |
| | | VYLQMNSLKPEDTAVYYCAAVRWGPHNDDRYD |
| | | WGQGTQVTVSSGGGGSEPKSSDKTHTCPPCPA |
| | | PELLGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LPAPIEKTISKAKGQPREPQVYTLPPSREEMT |
| | | KNQVSLTCLVKGFYPSDIAVEWESNGQPENNY |
| | | KTTPPVLDSDGSFFLYSKLTVDKSRWQQGNVF |
| | | SCSVMHEALHNHYTQKSLSLSPG |

The GLP1R IgGs were characterized for thermal ramp stability (Tₘ and T_{agg}). The UNcle platform was used to characterize the IgGs and the data is seen in Table 21.

TABLE 21
Thermal Ramp Stability Measurements

| | Average | | | Average | | | Average | % CV | SD |
|---|---|---|---|---|---|---|---|---|---|
| | Tml | % CV | | Tm2 | % CV | | Tagg | Tagg | Tagg |
| Sample | (°C) | Tm1 | SD Tm1 | (° C.) | Tm2 | SD Tm2 | 266 (° C.) | 266 | 266 |
| GLP1R-59-2 | 60.6 | 0.08 | 0.05 | 84.6 | 0.71 | 0.6 | 58.3 | 0.29 | 0.17 |
| GLP1R-59-241 | 66 | 6.52 | 4.3 | 73.6 | 0.41 | 0.3 | 57.8 | 0.69 | 0.4 |
| GLP1R-59-243 | 60.9 | 0.33 | 0.2 | 75.2 | 0.8 | 0.6 | 55.9 | 0.72 | 0.4 |
| GLP1R-3 | 66.7 | 0.6 | 0.4 | 73.5 | 0.54 | 0.4 | 68.4 | 0.58 | 0.4 |
| GLP1R-241 | 68.2 | 0.82 | 0.56 | 75.7 | 0.94 | 0.71 | 65.9 | 0.76 | 0.5 |
| GLP1R-2 | 61.8 | 1.17 | 0.72 | 74.8 | 1.27 | 0.95 | 60.5 | 0.12 | 0.07 |

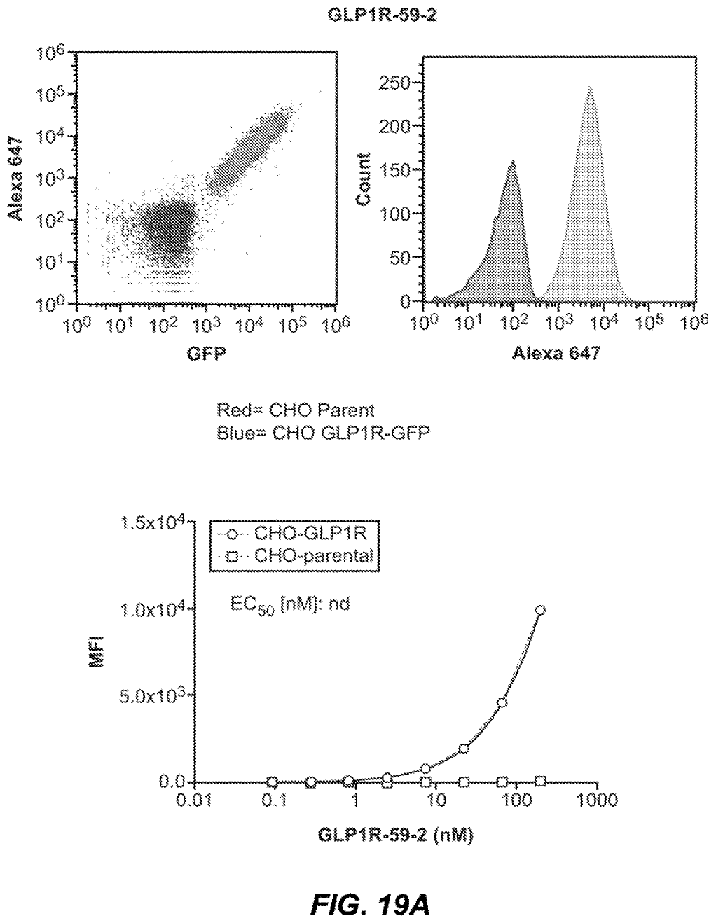

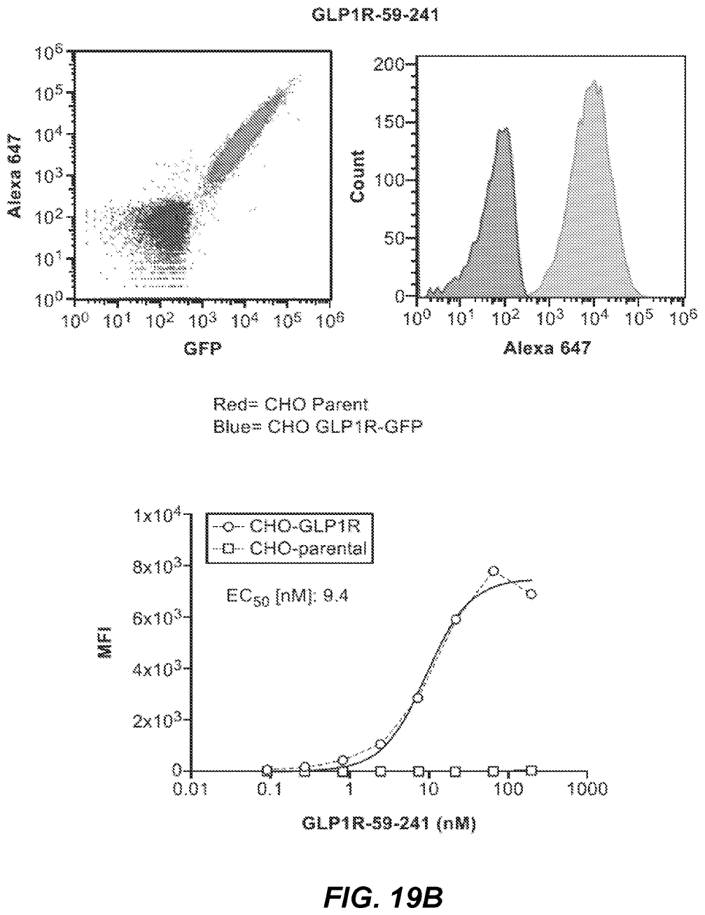

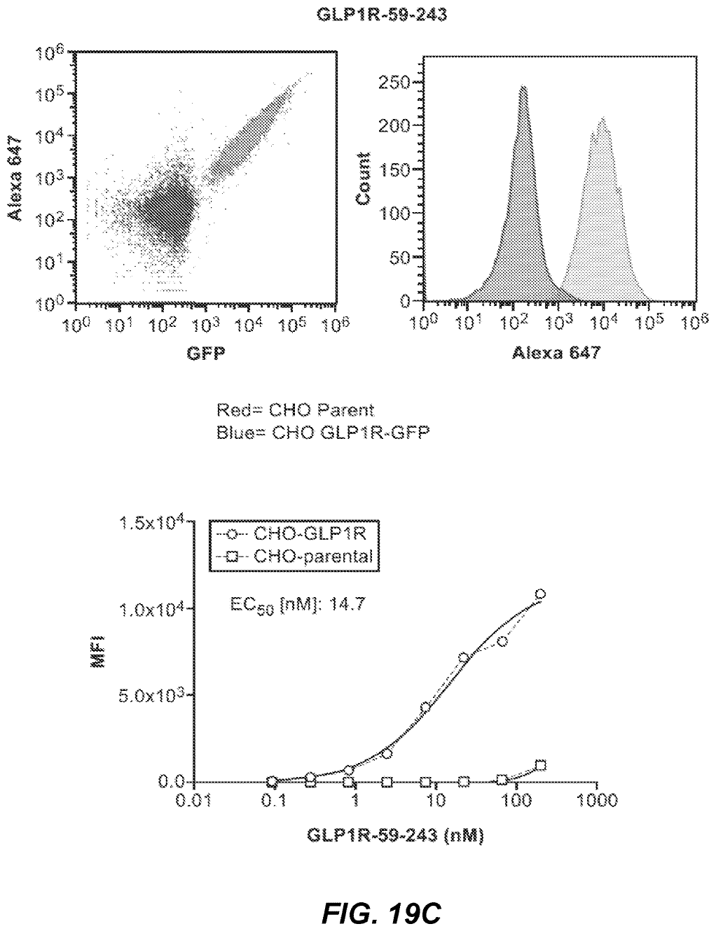

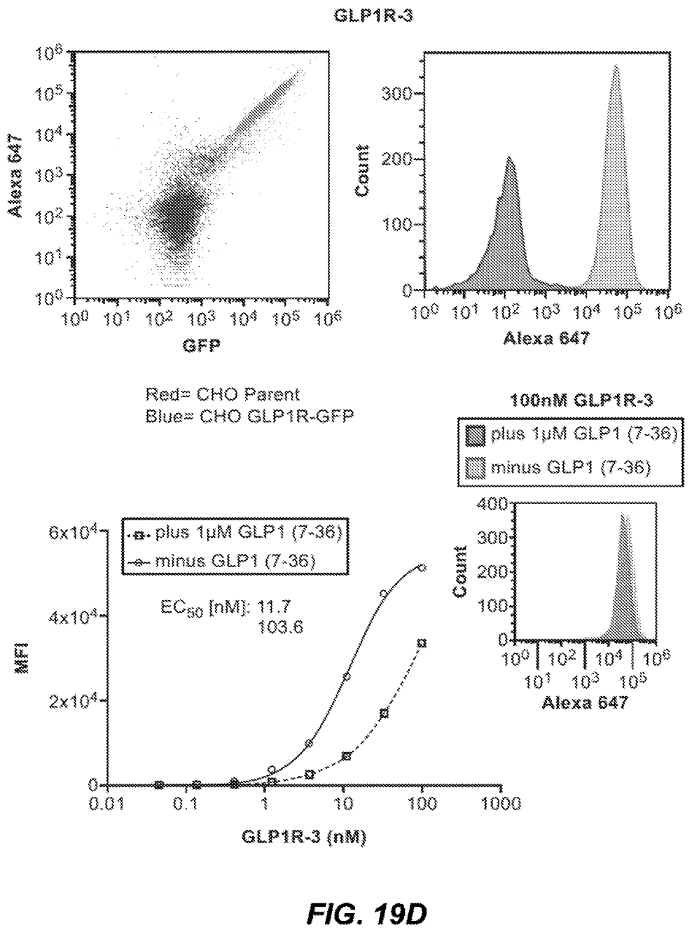

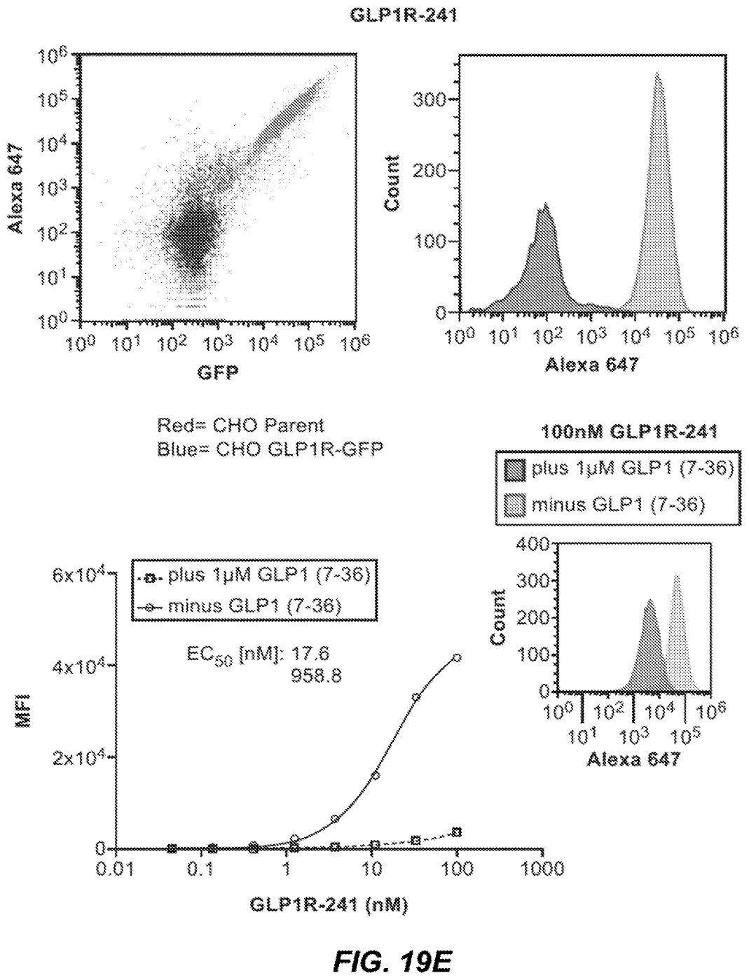

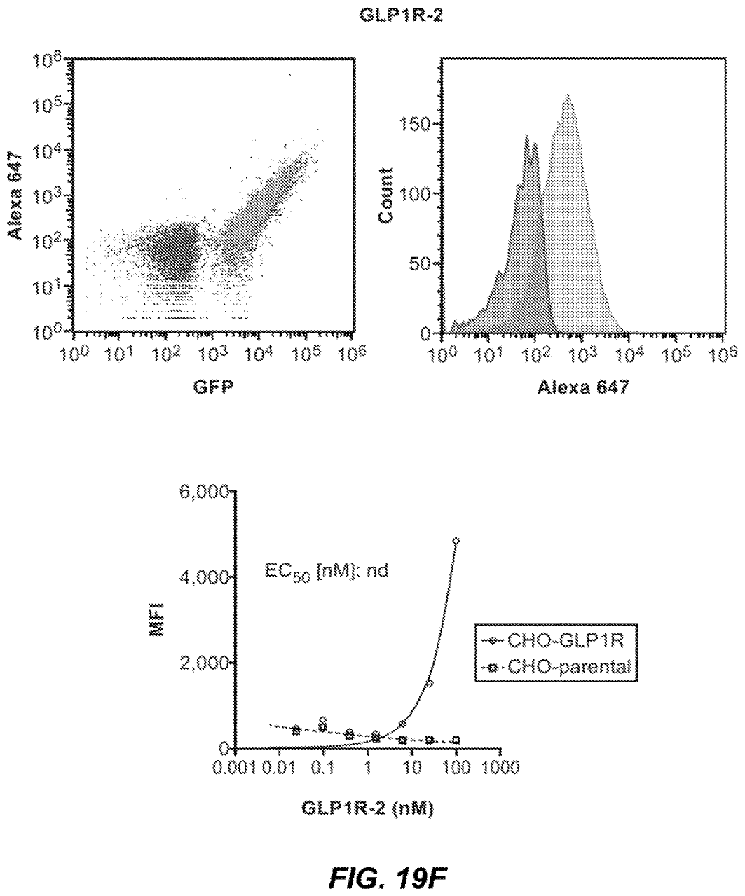

The GLP1R IgGs were then assayed in binding assays coupled to flow cytometry analysis using similar methods as described in Example 12. Briefly, stably expressing Flag-GLP1R-GFP CHO cells or CHO-parent cells were incubated with primary IgG (100 nM or 1:3 titrations). Secondary antibody incubation involved Alexa 647 conjugated goat-anti-human IgG. Flow cytometry measured the GFP signal against the Alexa 647 signal to identify IgGs that specifically bound to the target (GLP1R). Ligand competition assays involved co-incubating the primary IgG with 1 μM GLP1 (7-36). Data for GLP1R-59-2, GLP1R-59-241, GLP1R-59-243, GLP1R-3, GLP1R-241, and GLP1R-2 are seen in FIGS. 19A-19F.

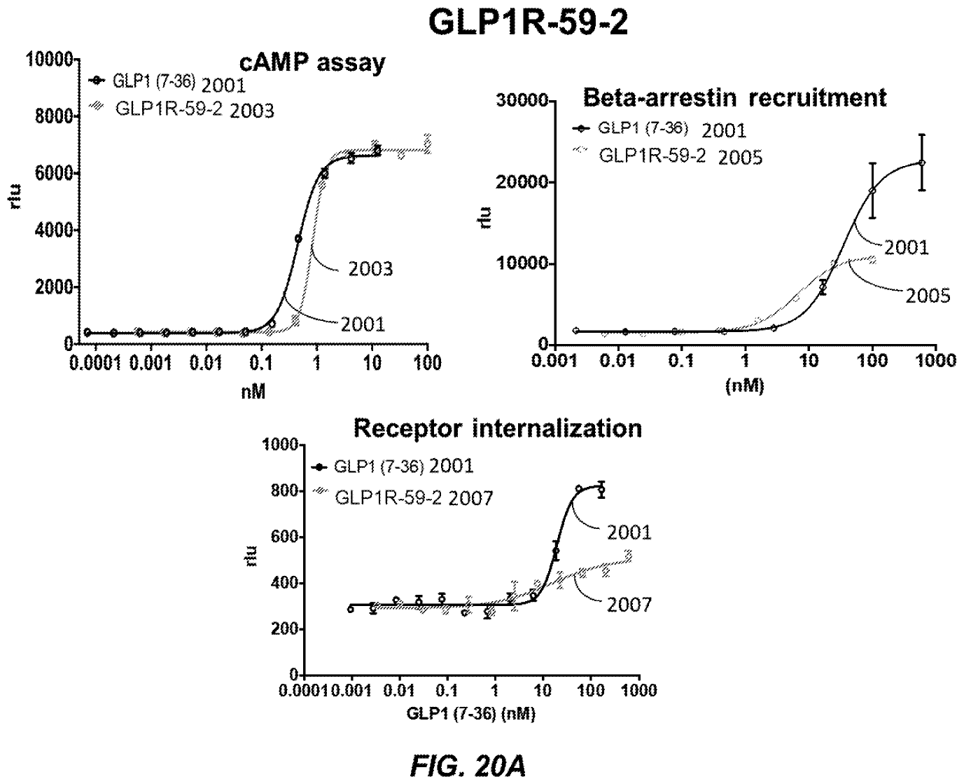

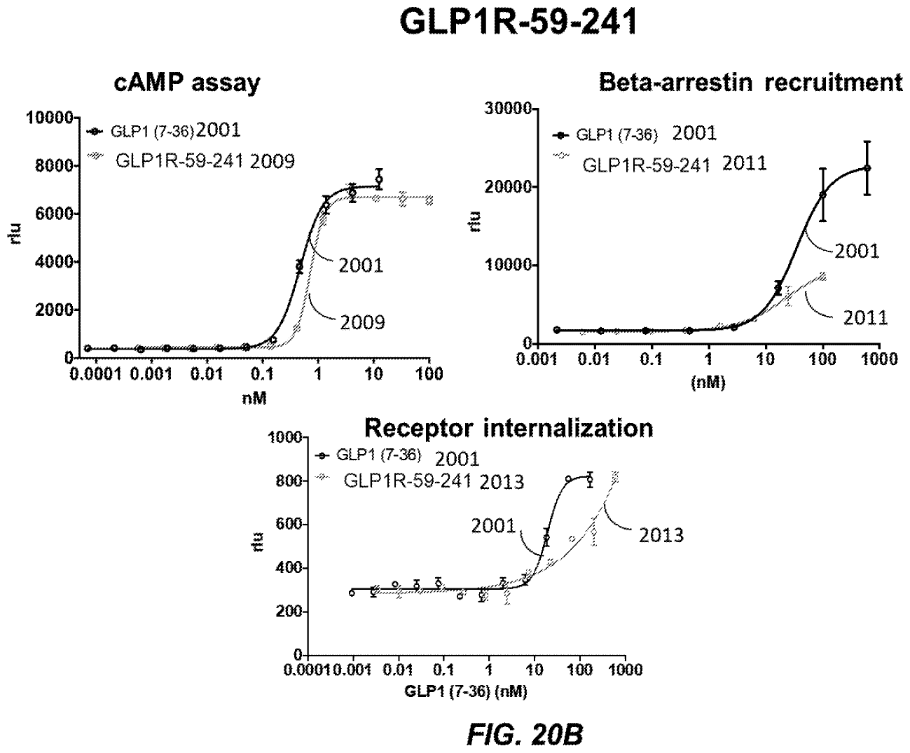

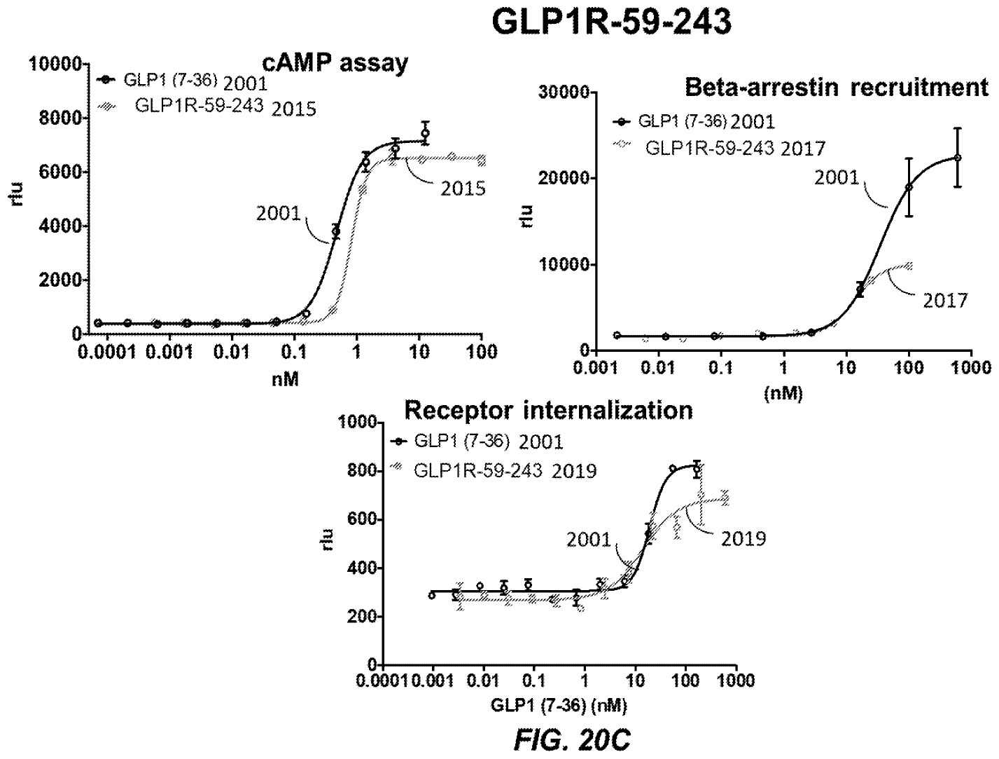

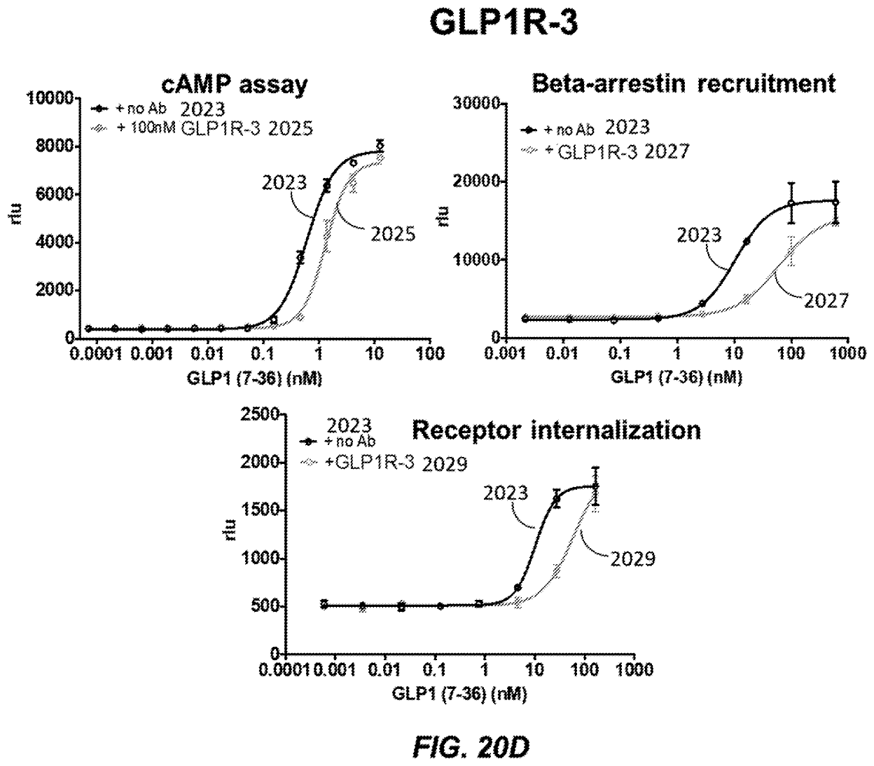

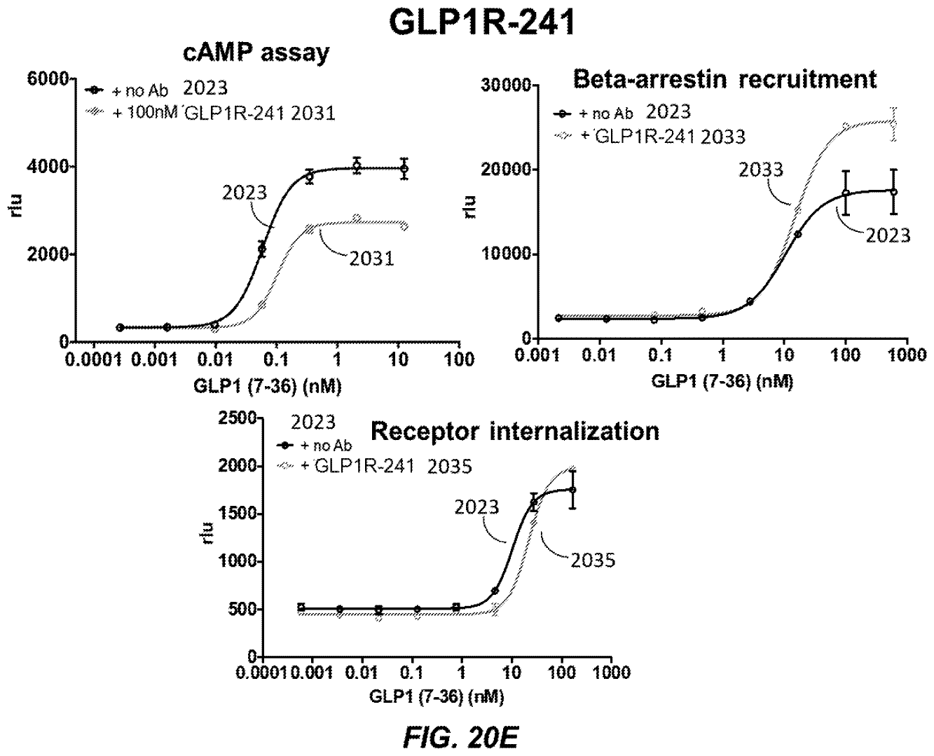

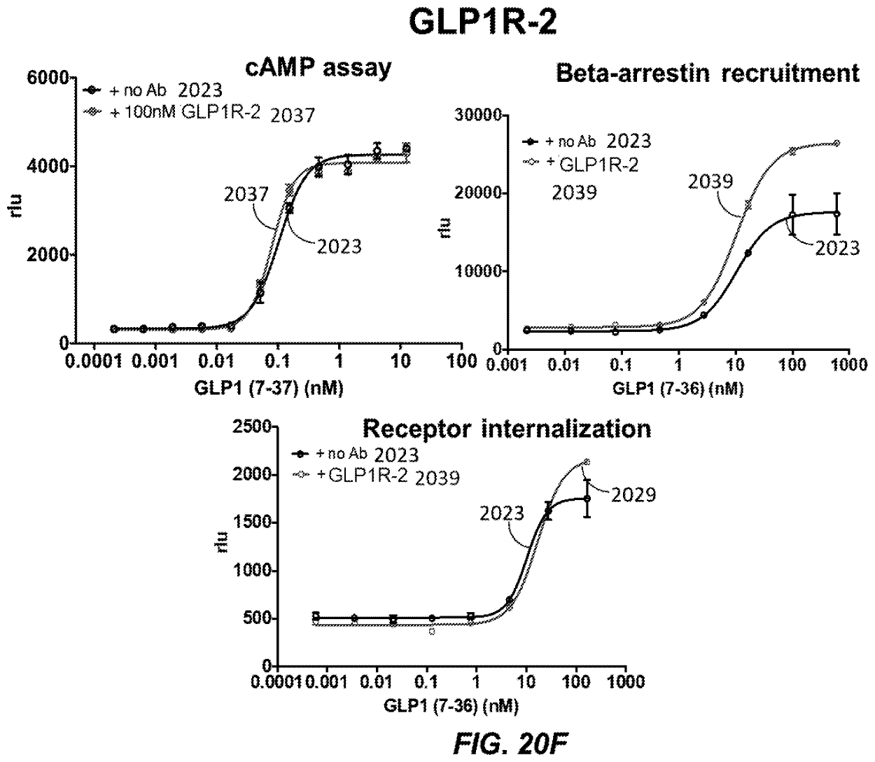

Functional assays were also performed using the GLP1R IgGs using similar methods as described in Example 12. Briefly, cAMP, beta-arrestin recruitment and activated receptor internalization assays were obtained from Eurofins DiscoverX and utilized untagged GLP-1R overexpressing CHO-Ki or U2OS cells. These were used to test for either agonist activity of the IgGs as compared with GLP1 (7-36) or for antagonistic activity of the IgGs by pre-incubating cells with IgGs and examining their effects on GLP1 (7-36)-induced signaling. For the cAMP assays, following GLP1 (7-36) or IgG stimulation, the cellular cAMP levels are measured using a homogenous, no wash, gain-of-signal competitive immunoassay based on Enzyme Fragment Complementation (EFC) technology. Data from the functional assays for GLP1R-59-2, GLP1R-59-241, GLP1R-59-243, GLP1R-3, GLP1R-241, and GLP1R-2 is seen in FIGS. 20A-20F. The EC50 (nM) data for GLP1R-59-2, GLP1R-59-241, GLP1R-59-243, GLP1R-3, GLP1R-241, and GLP1R-2 is seen in Tables 22-23. As seen in Table 23, the EC50 data for GLP1R-3 showed a 2.2-fold difference. The EC50 data for GLP1-241 showed a 1.7-fold difference. The EC50 data for GLP1R-2 showed a 0.8-fold difference.

TABLE 22
EC50 (nM) for GLP1R-59-2, GLP1R-59-241, and GLP1R-59-243

| GLP1R IgG | EC50 | GLP1 (7-36) EC50 |
|---|---|---|
| GLP1R-59-2 | 0.842 | 0.4503 |
| GLP1R-59-241 | 0.7223 | 0.4731 |
| GLP1R-59-243 | 0.8209 | 0.4731 |

TABLE 23
EC50 (nM) for GLP1R-3, GLP1R-241, and GLP1R-2

| GLP1R IgG (+100 nM) | EC50 | No Antibody EC50 |
|---|---|---|
| GLP1R-3 | 1.311 | 0.6053 |
| GLP1R-241 | 0.1027 | 0.05946 |
| GLP1R-2 | 0.07947 | 0.1031 |

GLP1R-3 was also assayed to determine specificity of GLP1R versus GL1P2R binding and determined to be specific for GLP1R over GLP2R (data not shown). Binding of GLP1R-3, GLP1R-59-242, and GLPTR-43-8 on mouse, macaca, and human GLP1R was determined. GLP1R-3 at 100 nM, GLP1R-59-242 at 100 nM, and GLP1R-43-8 at 100 nM were found to bind mouse, macaca, and human GLP1R (data not shown). GLP1R-3 was also found to bound human pancreatic precursor cells expressing endogenous GLP1R.

Binding of GLP1R-59-2, GLP1R-59-241, and GLPTR-59-243 on mouse, macaca, and human GLP1R was determined. GLP1R-59-2 at 100 nM, GLP1R-59-241 at 100 nM, and GLP1R-59-243 at 50 nM were found to bind mouse, macaca, and human GLP1R (data not shown). GLPTR-59-2 was also found to bound human pancreatic precursor cells expressing endogenous GLP1R.

This example shows GLP1R IgGs with agonistic and antagonist properties. Several of the IgGs induced cAMP signaling, beta-arresting recruitment, and receptor internalization similar to GLP1 (7-36).

### Example 14: VHH Libraries

Synthetic VHH libraries were developed. For the ‘VHH Ratio’ library with tailored CDR diversity, 2391 VHH sequences (iCAN database) were aligned using Clustal Omega to determine the consensus at each position and the framework was derived from the consensus at each position. The CDRs of all the 2391 sequences were analyzed for position-specific variation, and this diversity was introduced in the library design. For the ‘VHH Shuffle’ library with shuffled CDR diversity, the iCAN database was scanned for unique CDRs in the nanobody sequences. 1239 unique CDR1's, 1600 unique CDR2's, and 1608 unique CDR3's were identified and the framework was derived from the consensus at each framework position amongst the 2391 sequences in the iCAN database. Each of the unique CDR's was individually synthesized and shuffled in the consensus framework to generate a library with theoretical diversity of 3.2×10]. The library was then cloned in the phagemid vector using restriction enzyme digest. For the ‘VHH hShuffle’ library (a synthetic “human” VHH library with shuffled CDR diversity), the iCAN database was scanned for unique CDRs in the nanobody sequences. 1239 unique CDR1's, 1600 unique CDR2's, and 1608 unique CDR3's were identified and framework 1, 3, and 4 was derived from the human germline DP-47 framework. Framework 2 was derived from the consensus at each framework position amongst the 2391 sequences in the iCAN database. Each of the unique CDR's was individually synthesized and shuffled in the partially humanized framework using the NUGE tool to generate a library with theoretical diversity of 3.2×10]. The library was then cloned in the phagemid vector using the NUGE tool.

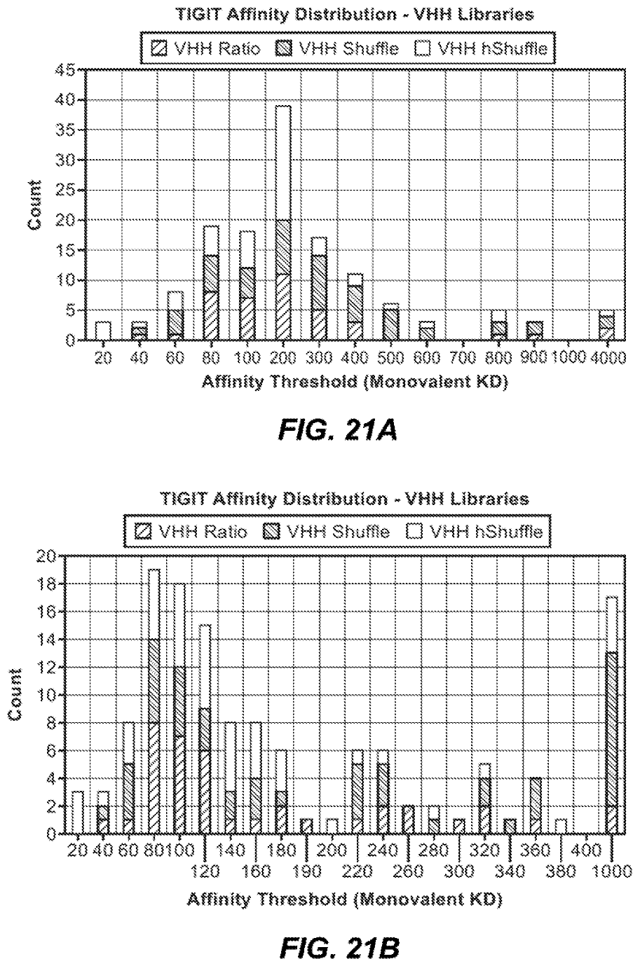

The Carterra SPR system was used to assess binding affinity and affinity distribution for VHH-Fc variants. VHH-Fc demonstrate a range of affinities for TIGIT, with a low end of 12 nM K_{D} and a high end of 1685 nM K_{D} (data not shown). Table 23A provides specific values for the VHH-Fc clones for ELISA, Protein A (mg/ml), and K_{D} (nM). FIG. 21A and FIG. 21B depict TIGIT affinity distribution for the VHH libraries, over the 20-4000 affinity threshold (FIG. 21A; monovalent K_{D}) and the 20-1000 affinity threshold (FIG. 21B; monovalent K_{D}). Out of the 140 VHH binders tested, 51 variants had affinity <100 nM, and 90 variants had affinity <200 nM.

TABLE 23A
ELISA, Protein A, and K_{D} of VHH-Fc Clones

| | | | ProA | |
|---|---|---|---|---|
| Clone | ELISA | Library | (mg/ml) | K_{D} (nM) |
| Variant 31-1 | 5.7 | VHH hShuffle | 0.29 | 12 |
| Variant 31-6 | 9.6 | VHH hShuffle | 0.29 | 14 |
| Variant 31-26 | 5.1 | VHH hShuffle | 0.31 | 19 |
| Variant 30-30 | 8 | VHH Shuffle | 0.11 | 23 |
| Variant 31-32 | 8 | VHH hShuffle | 0.25 | 27 |
| Variant 29-10 | 5 | VHH Ratio | 0.19 | 32 |
| Variant 29-7 | 7.3 | VHH Ratio | 0.28 | 41 |
| Variant 30-43 | 13.5 | VHH Shuffle | 0.18 | 44 |
| Variant 31-8 | 12.7 | VHH hShuffle | 0.29 | 45 |
| Variant 31-56 | 11.7 | VHH hShuffle | 0.26 | 46 |
| Variant 30-52 | 4.2 | VHH Shuffle | 0.22 | 49 |
| Variant 31-47 | 8.8 | VHH hShuffle | 0.23 | 53 |
| Variant 30-15 | 9.3 | VHH Shuffle | 0.26 | 55 |
| Variant 30-54 | 5.5 | VHH Shuffle | 0.3 | 58 |
| Variant 30-49 | 10.3 | VHH Shuffle | 0.26 | 62 |
| Variant 29-22 | 3.4 | VHH Ratio | 0.27 | 65 |
| Variant 29-30 | 9.2 | VHH Ratio | 0.28 | 65 |
| Variant 31-35 | 5.7 | VHH hShuffle | 0.24 | 66 |
| Variant 29-1 | 10.4 | VHH Ratio | 0.09 | 68 |
| Variant 29-6 | 6.8 | VHH Ratio | 0.29 | 69 |
| Variant 31-34 | 6 | VHH hShuffle | 0.32 | 70 |
| Variant 29-12 | 6.2 | VHH Ratio | 0.23 | 70 |
| Variant 30-1 | 5.4 | VHH Shuffle | 0.39 | 71 |
| Variant 29-33 | 3.9 | VHH Ratio | 0.15 | 74 |
| Variant 30-20 | 4.6 | VHH Shuffle | 0.19 | 74 |
| Variant 31-20 | 6.6 | VHH hShuffle | 0.37 | 74 |
| Variant 31-24 | 3.1 | VHH hShuffle | 0.15 | 75 |
| Variant 30-14 | 9.9 | VHH Shuffle | 0.19 | 75 |
| Variant 30-53 | 7.6 | VHH Shuffle | 0.24 | 78 |
| Variant 31-39 | 9.9 | VHH hShuffle | 0.32 | 78 |
| Variant 29-18 | 10.9 | VHH Ratio | 0.19 | 78 |
| Variant 30-9 | 8 | VHH Shuffle | 0.4 | 79 |
| Variant 29-34 | 8.6 | VHH Ratio | 0.21 | 80 |
| Variant 29-27 | 8.6 | VHH Ratio | 0.18 | 82 |
| Variant 29-20 | 5.9 | VHH Ratio | 0.26 | 83 |
| Variant 30-55 | 6 | VHH Shuffle | 0.41 | 85 |
| Variant 30-39 | 6.1 | VHH Shuffle | 0.07 | 88 |
| Variant 31-15 | 6.2 | VHH hShuffle | 0.32 | 88 |
| Variant 29-21 | 4.3 | VHH Ratio | 0.23 | 88 |
| Variant 29-37 | 5.3 | VHH Ratio | 0.26 | 89 |
| Variant 29-40 | 6.6 | VHH Ratio | 0.31 | 90 |
| Variant 31-30 | 3.2 | VHH hShuffle | 0.33 | 93 |
| Variant 31-10 | 12.3 | VHH hShuffle | 0.31 | 94 |
| Variant 29-3 | 13.6 | VHH Ratio | 0.11 | 94 |
| Variant 30-57 | 5.2 | VHH Shuffle | 0.24 | 95 |
| Variant 29-31 | 4.4 | VHH Ratio | 0.18 | 96 |
| Variant 31-27 | 8.1 | VHH hShuffle | 0.31 | 96 |
| Variant 31-33 | 6 | VHH hShuffle | 0.32 | 96 |
| Variant 30-40 | 7.1 | VHH Shuffle | 0.21 | 99 |
| Variant 31-18 | 4.1 | VHH hShuffle | 0.36 | 99 |
| Variant 30-5 | 9.3 | VHH Shuffle | 0.05 | 100 |

### Example 15: VHH Libraries for GLP1R

A VHH library for GLP1R was developed similar to methods described in Example 14. Briefly, stable cell lines expressing GLP1R were generated, and target expression was confirmed by FACS. Cells expressing >80% of the target were then used for cell-based selections. Five rounds of cell-based selections were carried out against cells stably overexpressing the target of interest. 10⁸ cells were used for each round of selection. Before selection on target expressing cells, phage from each round was first depleted on 10⁸ CHO background cells. Stringency of selections was increased by increasing the number of washes in subsequent rounds of selections. The cells were then eluted from phage using trypsin, and the phage was amplified for the next round of panning. A total of 1000 clones from round 4 and round 5 are sequenced by NGS to identify unique clones for reformatting as VHH-Fc.

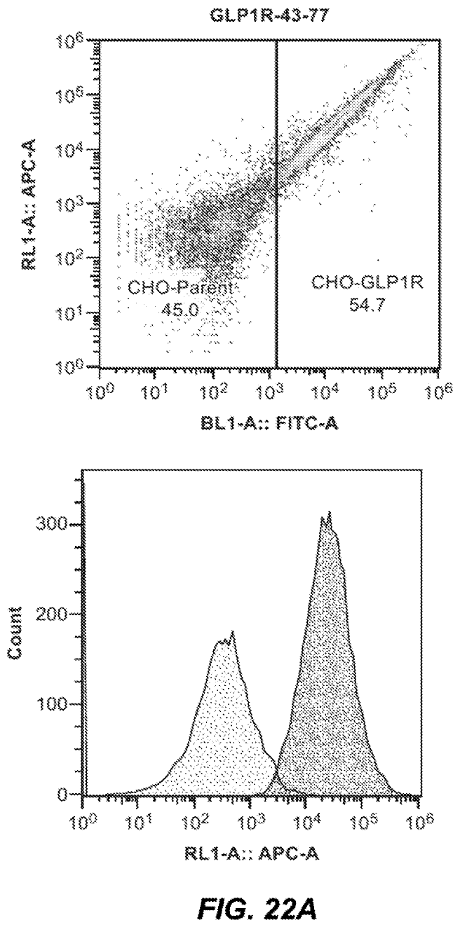

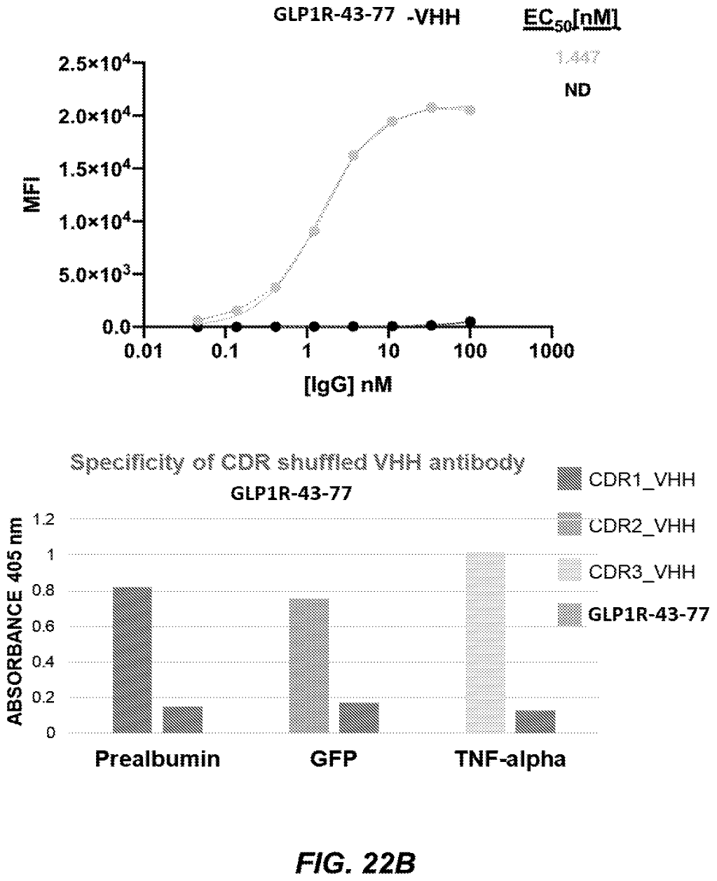

53 out of the 156 unique GLP1R VHH Fc binders had a target cell mean fluorescence intensity (MFI) value that was 2-fold over parental cells. The data for variant GLP1R-43-77 is seen in FIGS. 22A-22B and Tables 23B-24.

TABLE 23B
Panning Summary

| | | VHH-Fc FACS |
|---|---|---|
| | | binders |
| | Unique | (MFI values 2-fold |
| Library | Phage | over parental cells) |
| VHH hShuffle | 58 | 6 |
| VHH Ratio/Shuffle | 98 | 47 |

TABLE 24
GLP1R-43-77 Data

| Subset Name with Gating | | |
|---|---|---|
| Path | Count | Median: RL1-A |
| Sample E10.fcs/CHO-parent | 11261 | 237 |
| Sample E10.fcs/CHO-GLP1R | 13684 | 23439 |

### Example 16. GLP1R Libraries with Varied CDR's

A GLP1R library was created using a CDR randomization scheme.

Briefly, GLP1R libraries were designed based on GPCR antibody sequences. Over sixty different GPCR antibodies were analyzed and sequences from these GPCRs were modified using a CDR randomization scheme.

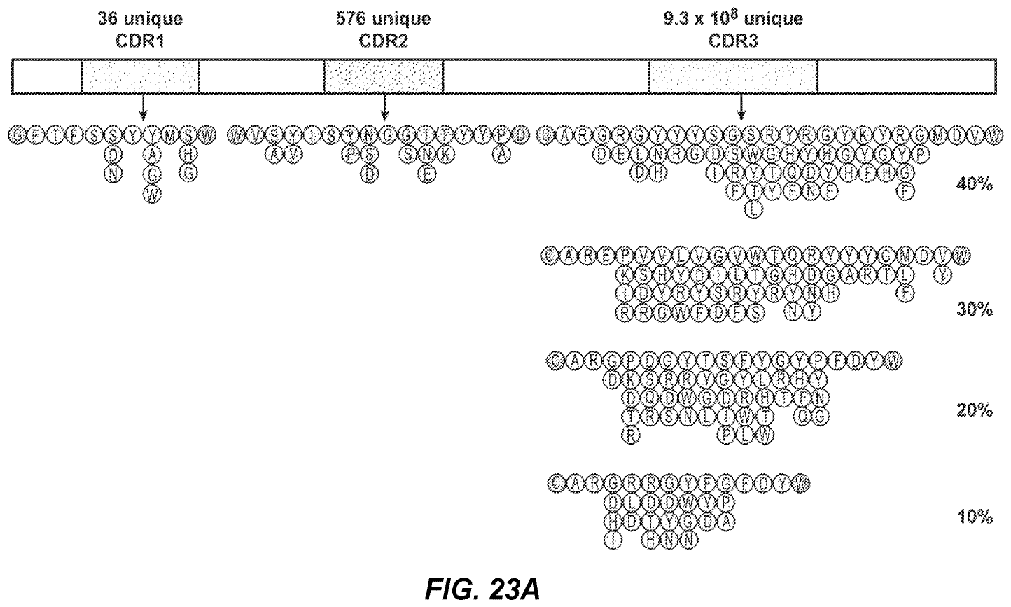

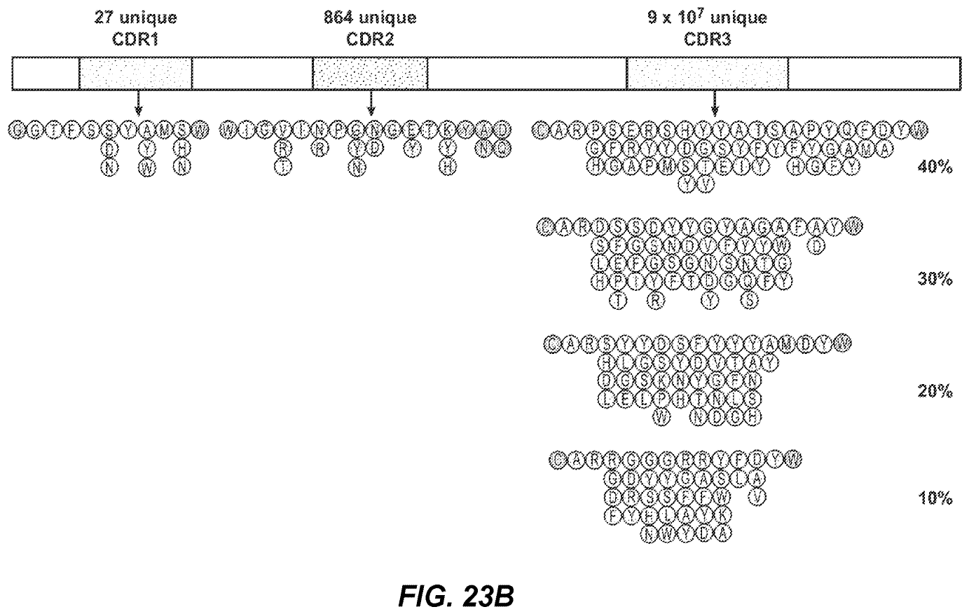

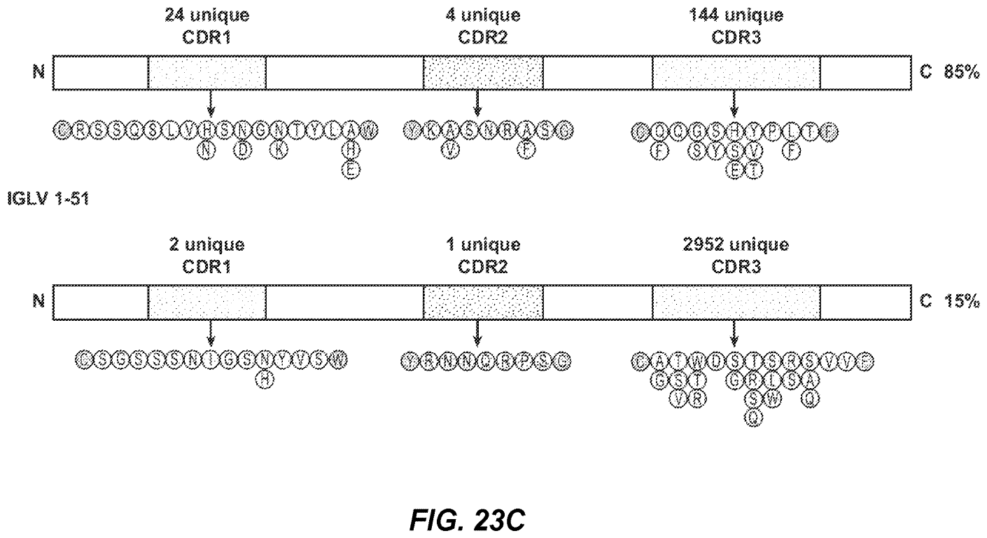

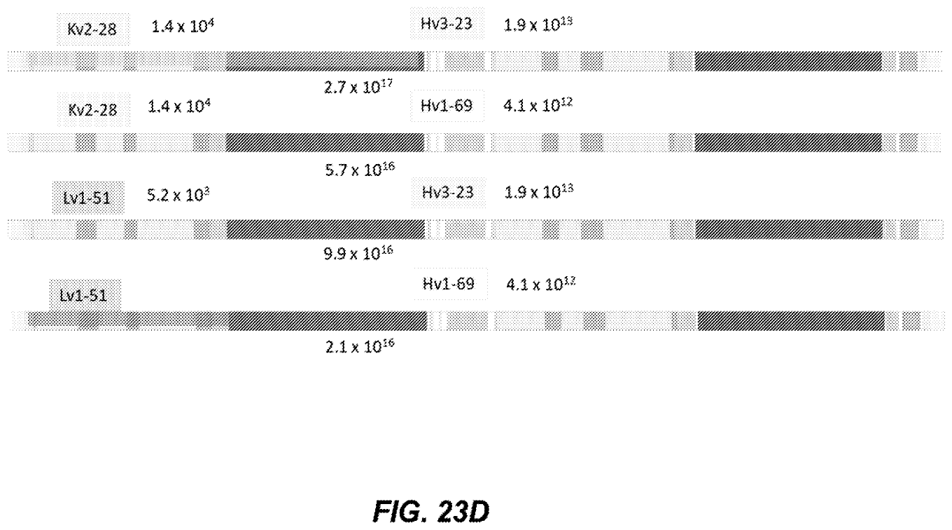

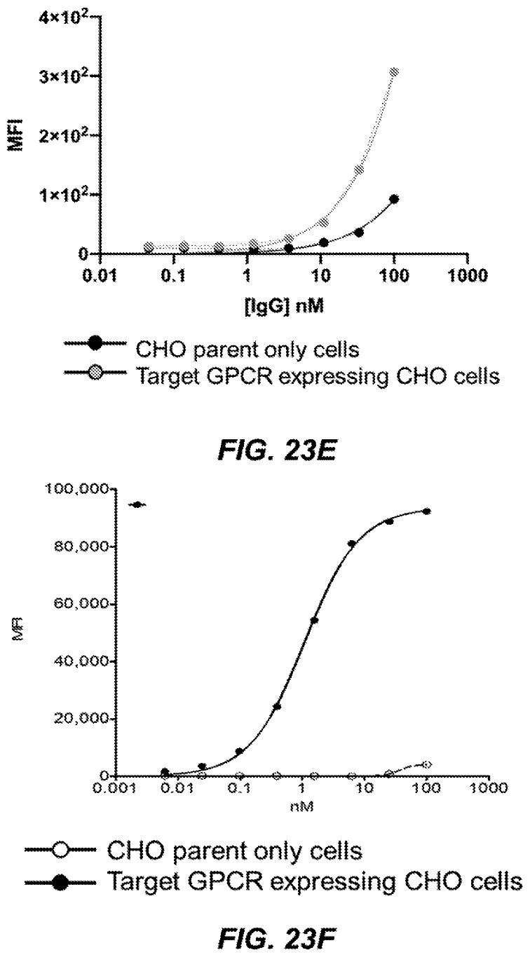

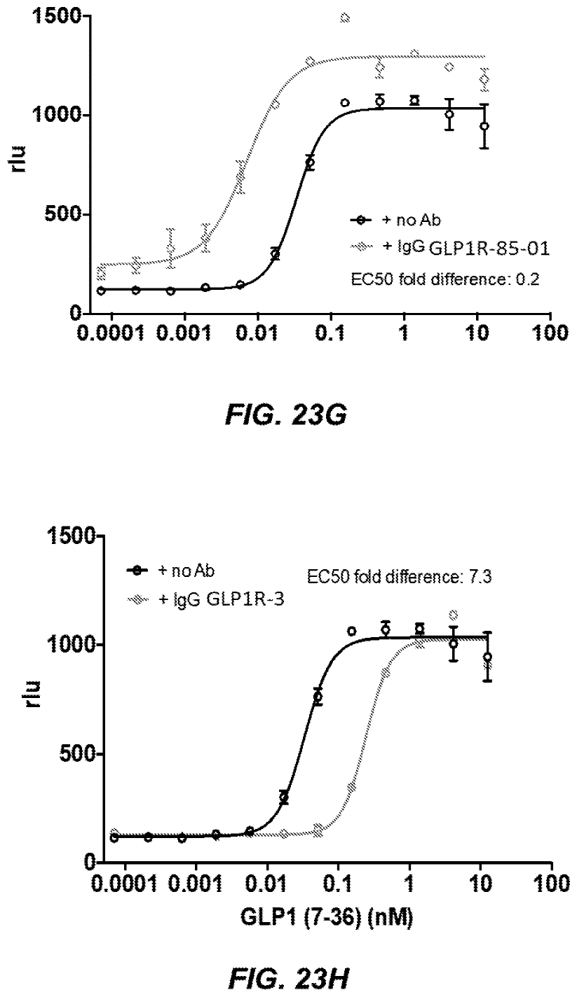

The heavy chain IGHV3-23 design is seen in FIG. 23A. As seen in FIG. 23A, IGHV3-23 CDRH3's had four distinctive lengths: 23 amino acids, 21 amino acids, 17 amino acids, and 12 amino acids, with each length having its residue diversity. The ratio for the four lengths were the following: 40% for the CDRH3 23 amino acids in length, 30% for the CDRH3 21 amino acids in length, 20% for the CDRH3 17 amino acids in length, and 10% for the CDRH3 12 amino acids in length. The CDRH3 diversity was determined to be 9.3×10⁸, and the full heavy chain IGHV3-23 diversity was 1.9×10¹³.

The heavy chain IGHV1-69 design is seen in FIG. 23B. As seen in FIG. 23B, IGHV1-69 CDRH3's had four distinctive lengths: 20 amino acids, 16 amino acids, 15 amino acids, and 12 amino acids, with each length having its residue diversity. The ratio for the four lengths were the following: 40% for the CDRH3 20 amino acids in length, 30% for the CDRH3 16 amino acids in length, 20% for the CDRH3 15 amino acids in length, and 10% for the CDRH3 12 amino acids in length. The CDRH3 diversity was determined to be 9×10⁷, and the full heavy chain IGHV-69 diversity is 4.1×10¹².

The light chains IGKV2-28 and IGLV1-51 design is seen in FIG. 23C. Antibody light chain CDR sequences were analyzed for position-specific variation. Two light chain frameworks were selected with fixed CDR lengths. The theoretical diversities were determined to be 13800 and 5180 for kappa and light chains, respectively.

The final theoretical diversity was determined to be 4.7×10¹⁷ and the final, generated Fab library had a diversity of 6×10⁹. See FIG. 23D.

The purified GLP1R IgGs were assayed to determine cell-based affinity measurements and for functional analysis. FACS binding was measured using purified GLP1R IgG. As seen in FIG. 23E, the GLP1R IgG bound selectively to GLP1R-expressing cells with affinities in the low nanomolar range, demonstrating an IgG that selectively binds target expressing cell with an affinity of 1.1 nM. FACS binding was also measured in GLP1R IgGs generated using methods described in Examples 4-10. As seen in FIG. 23F, GLP1R IgGs bind selectively to GLP1R-expressing cells with affinities in the low nanomolar range.

cAMP assays using purified GLP1R IgG demonstrated that presence of GLP1R IgGs resulted in a left shift of the dose response curve of the GLP1 agonist induced cAMP response in GLP1R expressing CHO cells as seen in FIG. 23G. GLP1R IgGs generated using methods described in Examples 4-10 also resulted in a left shift of the dose response curve of the receptor agonist induced cAMP response in GLP1R expressing CHO cells (FIG. 23H).

The data shows the design and generation of GLP1R IgGs with improved potency and function.

### Example 17. Oral Glucose Tolerance Mouse Model

The objective of this study was to evaluate the acute effects of a chimeric antibody GLP1R agonist and antagonist on glycemic control in a mouse model of diet induced obesity in C57BL/6J DIO mice. The test articles are seen below in Table 25.

TABLE 25
Test Article Identification

| | GLP1 | GLP1 | | Positive |
|---|---|---|---|---|
| | Agonist Ab | Antagonist Ab | Ab Control | Control |
| Identification | GLP1R-59-2 | GLP1R-3 | GLP1R-2 | Liraglutide |
| Physical | Clear Liquid | Clear Liquid | Clear Liquid | |
| Description | | | | |
| Purity | 95% | 95% | TBD | |
| Concentration | 2.7 mg/ml | 3.7 mg/ml | TBD | |
| Storage | Temperature set | Temperature set | Temperature set | Temperature set |
| Conditions | to maintain | to maintain | to maintain | to maintain |
| | 4° C. | 4° C. | 4° C. | 4° C. |
| Provided by | Sponsor | Sponsor | Sponsor | Testing Facility |

— = Not applicable.

For each test article, 7 different test article groups were generated as summarized in the following Table 26 with 8 animals per group.

TABLE 26
Experimental Design

| | | | Dose | Dose | | | | Number |
|---|---|---|---|---|---|---|---|---|
| Group | Test | Dose Level | Volume | Concentration | | Dose | | of |
| No. | Material | (mg/kg/day) | (mL/kg) | (mg/mL) | Diet | Regimen | Route | animals |
| 1 | GLP1R-2 | 0 | 5 | 0 | HFD | QD | SC | 8 |
| 2 | Liraglutide | 0.2 | 5 | 0.04 | HFD | QD | SC | 8 |
| 3 | GLP1R-2 | 10 | 5 | 2 | HFD | QD | SC | 8 |
| | Liraglutide | 0.2 | 5 | 0.04 | | | | |
| 4 | GLP1R-59-2 | 10 | 5 | 2 | HFD | QD | SC | 8 |
| 5 | GLP1R-59-2 | 10 | 5 | 2 | HFD | QD | SC | 8 |
| | Liraglutide | 0.2 | 5 | 0.04 | | | | |
| 6 | GLP1R-3 | 10 | 5 | 2 | HFD | QD | SC | 8 |
| 7 | GLP1R-3 | 10 | 5 | 2 | HFD | QD | SC | 8 |
| | Liraglutide | 0.2 | 5 | 0.04 | | | | |

No. = Number; ;
HFD = high fat diet;
QD = once daily;
SC = Subcutaneous injection

On Day 3 (all animals) and Day 1 (Group 1-7), a non-fasting blood glucose was determined by tail snip. Approximately 5-10 μL of blood was collected. The second drop of blood from the animal was placed on a blood glucose test strip and analyzed using a hand-held glucometer (Abbott Alpha Trak).

After a non-fasting blood glucose measurement was made on the day of the procedure, the animals were weighed, tails marked, and the animals placed in clean cages without food. The animals were fasted for 4 hours and a fasting blood glucose measurement was determined. The animals were then treated with the indicated test article(s) as shown in Table 26.

The oral glucose tolerance test (OGTT) was administered to each animal 60 minutes later. The animals were dosed via oral gavage with 2 g/kg glucose (10 mL/kg). Blood glucose was determined via tail snip with the second drop of blood from the animal placed on a hand-held glucometer (Abbott Alpha Trak) at the following times relative to the glucose dose: 0 (just prior to glucose dose), 15, 30, 60, 90, and 120 minutes. Additional blood samples were obtained at the 15 minute and 60 minute time points of the OGTT for estimation of serum insulin.

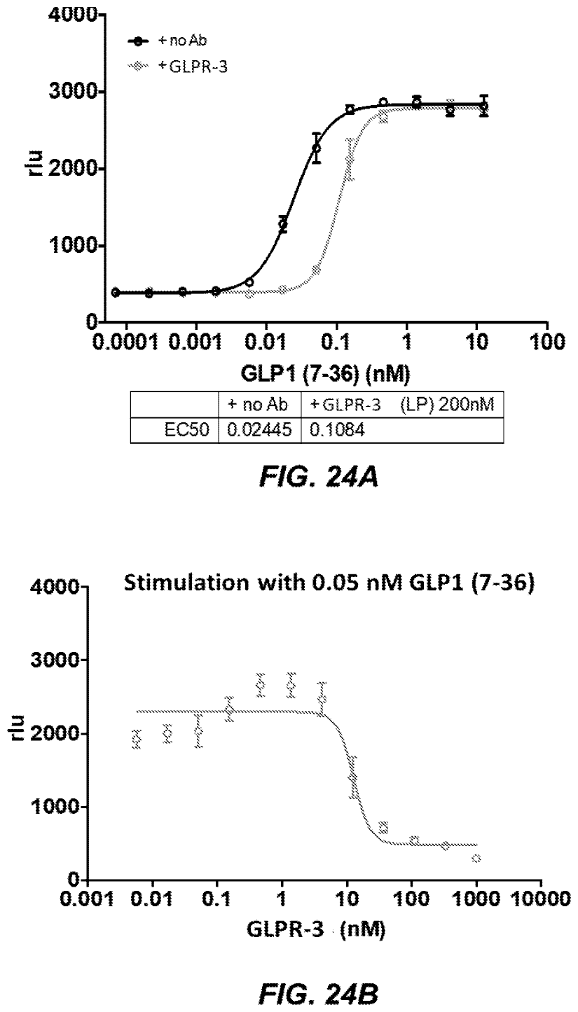

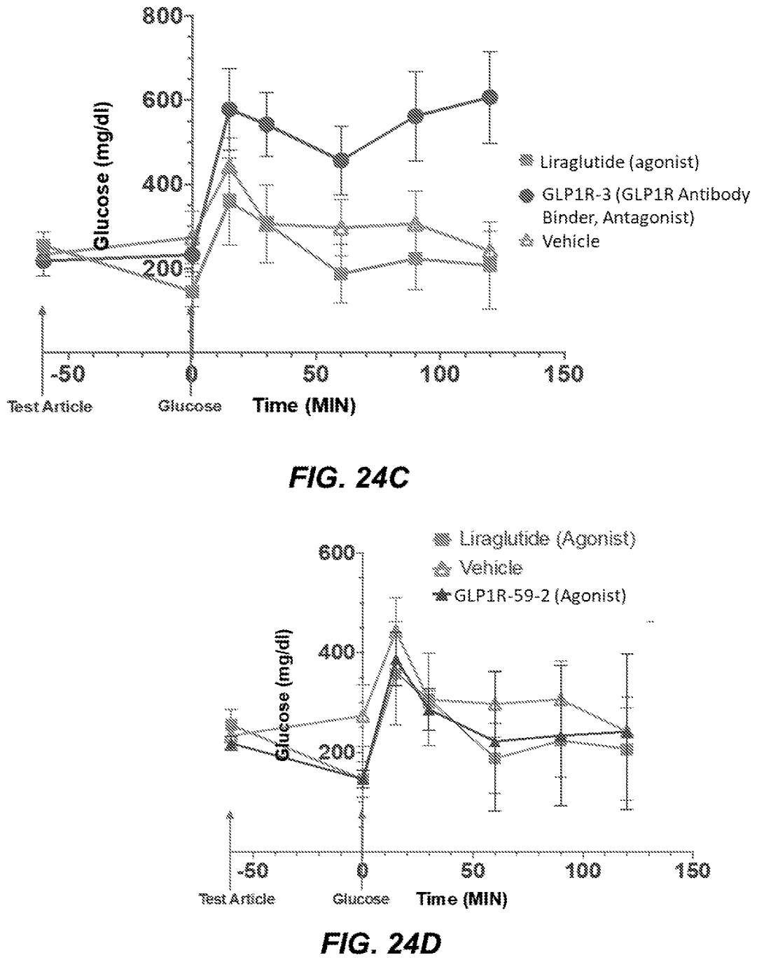

FIGS. 24A-24B show GLP1R-3 inhibits GLP1:GLP1R signaling (FIG. 24A) with complete inhibition at higher concentrations (FIG. 24B). As seen in FIG. 24C, GLP1R-3 dosed animals maintained sustained high glucose levels after glucose administration, indicating GLP1:GLP1R signal blockade. As seen in FIG. 24D, GLP1R-59-2 dose at 10 mg/kg exhibited a sustained, low glucose levels similar to liraglutide control.

The data shows that the GLP1R antibodies generated have functional effects in a mouse model for glucose tolerance.

### Example 18. GLP1R Agonists and Antagonists Effects in Wild-type Mice

The effects of GLP1R-59-2 (agonist) and GLP1R-3 (antagonist) in wild-type mice were determined in this Example.

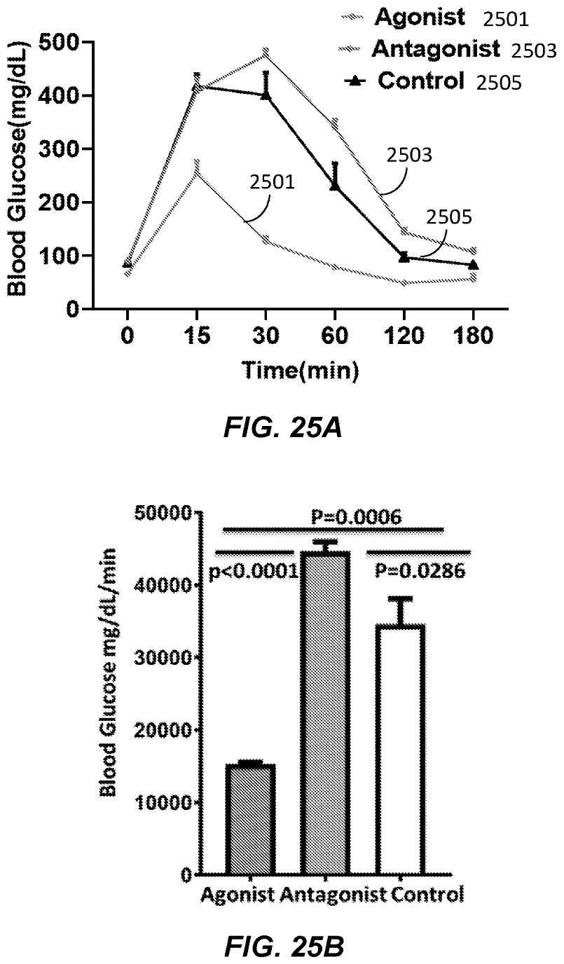

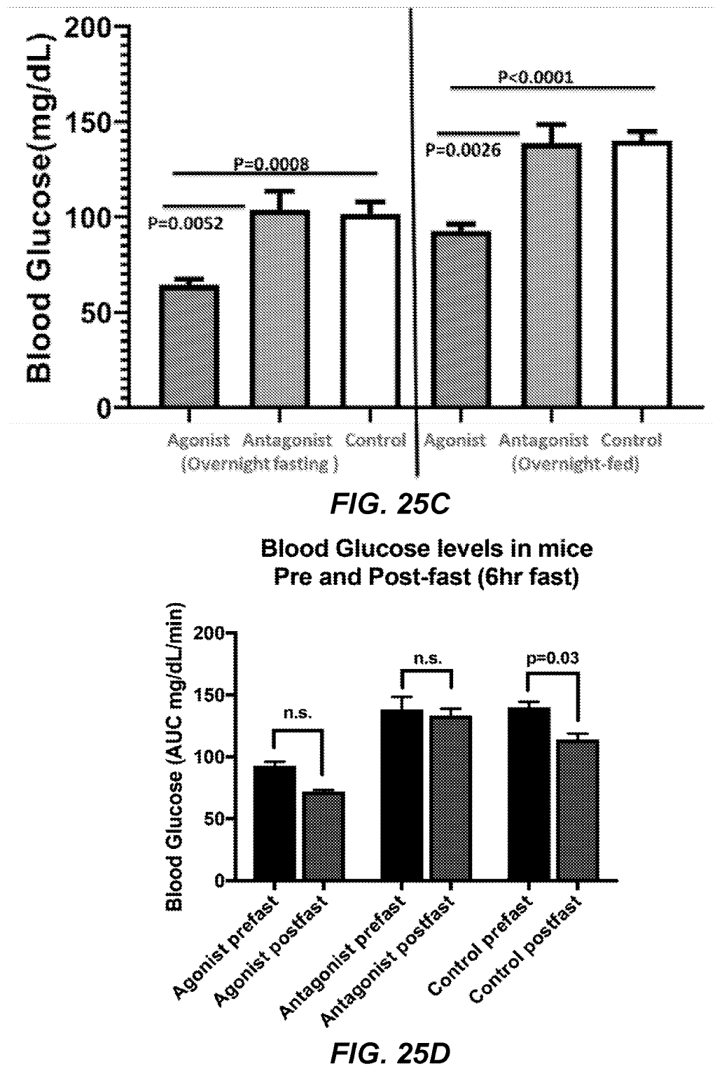

15 C57BL/6NHsd Mice were used and subjected to a Glucose Tolerance Test (GTT). The in vivo GTT test was performed on three groups of mice with 5 mice per group. All three groups were fasted for 13.5 hours before being weighed, time Zero Blood Glucose measured, and then injected i.p. with a 30% dextrose solution at a dose of 10 uL/gram body weight. Blood glucose measurements were recorded for each mouse at 15, 30, 60, 120, and 180 minutes after dextrose injection. A first group of mice were treated with GLP1R-59-2 at two doses: 10 mg/kg of GLP1R-59-2 at time of fasting (˜13.5 hrs. prior to GTT) and again two hours before start of GTT with 10 mg/kg of GLP1R-59-2. A second group of mice were treated with GLP1R-3 at two doses: 10 mg/kg of GLP1R-3 at time of fasting (˜13.5 hrs. prior to GTT) and again two hours before start of GTT with 10 mg/kg of GLP1R-3. A third group of mice were the control mice and were not treated. Data is seen for GLP1R-59-2 (agonist), GLP1R-3 (antagonist), and control in FIGS. 25A-25D. FIG. 25A shows the blood glucose levels in mice (y-axis) treated with GLP1R-59-2 (agonist), GLP1R-3 (antagonist), and control over time (in minutes, x-axis). FIG. 25B shows the blood glucose levels in mice (y-axis) treated with GLP1R-59-2 (agonist), GLP1R-3 (antagonist), and control. As seen in FIG. 25C, a significant reduction in blood glucose was observed in GLP1R-59-2 (agonist) treated mice in both the fasted (p=0.0008) and non-fasted (p<0.0001) mice compared to control. As seen in FIG. 25D, pre-dosed GLP1R-3 (antagonist) animals did not show decreased glucose in a 6 hour fast whereas control mice exhibited a decrease.

### Example 19. Exemplary Sequences

Exemplary sequences of GLP1R are seen in Table 27.

TABLE 27
GLP1R Sequences

| SEQ | GLP1R | |
|---|---|---|
| ID NO: | Variant | Sequence |
| 2411 | GLP1R-40- | EVQLVESGGGLVQPGGSLRLSCAASGFTCGDYTMGWFRQAPGKEREFLAAITSGGATTYDD |
| | 01 | NRKSRFTISADNSKNTAYLQMNSLKPEDTAVYYCWAALDGYGGRWGQGTLVTVSS |
| 2412 | GLP1R-40- | EVQLVESGGGLVQPGGSLRLSCAASGRTFRINRMGWFRQAPGKEREWVSTICSRGDTYYADS |
| | 02 | VKGRFTISADNSKNTAYLQMNSLKPEDTAVYYCAATLDGYSGSWGQGTLVTVSS |
| 2413 | GLP1R-40- | EVQLVESGGGLVQPGGSLRLSCAASGRDFRVKNMGWFRQAPGKEREFVARITWNGGSAYY |
| | 03 | ADSVKGRFTISADNSKNTAYLQMNSLKPEDTAVYYCAARILSRNWGQGTLVTVSS |
| 2414 | GLP1R-40- | EVQLVESGGGLVQPGGSLRLSCAASGFTFSFYTMGWFRQAPGKEREFVAAISSGGRTSYADS |
| | 04 | VKGRFTISADNSKNTAYLQMNSLKPEDTAVYYCAAALDGYEGSWGQGTLVTVSS |
| 2415 | GLP1R-40- | EVQLVESGGGLVQPGGSLRLSCAASGFTFSFYAMGWFRQAPGKEREFVAAISSGGRTRYADN |
| | 05 | VKGRFTISADNSKNTAYLQMNSLKPEDTAVYYCSAALDGYNGIWGQGTLVTVSS |
| 2416 | GLP1R-40- | EVQLVESGGGLVQPGGSLRLSCAASGHTSDTYIMGWFRQAPGKEREFVSLINWSSGKTIYAD |
| | 06 | SVKGRFTISADNSKNTAYLQMNSLKPEDTAVYYCAKGDYRGGYYYPQTSQWGQGTLVTVSS |
| 2417 | GLP1R-40- | EVQLVESGGGLVQPGGSLRLSCAASGFTFSSYPMGWFRQAPGKEREFVATIPSGGSTYYADS |
| | 07 | VKGRFTISADNSKNTAYLQMNSLKPEDTAVYYCAAALDGYNGSWGQGTLVTVSS |
| 2418 | GLP1R-40- | EVQLVESGGGLVQPGGSLRLSCAASGFTFGEFTMGWFRQAPGKERERVATITSGGSTNYADS |
| | 08 | VKGRFTISADNSKNTAYLQMNSLKPEDTAVYYCAAVVDDYSGSWGQGTLVTVSS |
| 2419 | GLP1R-40- | EVQLVESGGGLVQPGGSLRLSCAASGFTDGIDAMGWFRQAPGKEREVVAGIAWGDGITYYA |
| | 09 | DSVKGRFTISADNSKNTAYLQMNSLKPEDTAVYYCAASYNVYYNNWGQGTLVTVSS |
| 2420 | GLP1R-40- | EVQLVESGGGLVQPGGSLRLSCAASGRTFSSGVMGWFRQAPGKEREFVAAINRSGSTFYADS |
| | 10 | VKGRFTISADNSKNTAYLQMNSLKPEDTAVYYCAKTKRTGIFTTARMVDWGQGTLVTVSS |
| 2421 | GLP1R-40- | EVQLVESGGGLVQPGGSLRLSCAASGVTLDDYAMGWFRQAPGKEREFVAAINRSGSITYYA |
| | 11 | DSVKGRFTISADNSKNTAYLQMNSLKPEDTAVYYCAAYYTDYDEALEETRGSYDWGQGTLV |
| | | TVSS |
| 2422 | GLP1R-40- | EVQLVESGGGLVQPGGSLRLSCAASGLTFGIYAMGWFRQAPGKEREFVATISRSGASTYYAD |
| | 12 | SVKGRFTISADNSKNTAYLQMNSLKPEDTAVYYCAAIVTYNDYDRGHDWGQGTLVTVSS |
| 2423 | GLP1R-40- | EVQLVESGGGLVQPGGSLRLSCAASGFTFSSDGMGWFRQAPGKERELVAAINRSGSTFYADS |
| | 13 | VKGRFTISADNSKNTAYLQMNSLKPEDTAVYYCAKTARPGIFTTAPVEDWGQGTLVTVSS |
| 2424 | GLP1R-40- | EVQLVESGGGLVQPGGSLRLSCAASGFTCGNYTMGWFRQAPGKERESVASITSGGRTNYADS |
| | 14 | VKGRFTISADNSKNTAYLQMNSLKPEDTAVYYCAATLDGYTGSWGQGTLVTVSS |
| 2425 | GLP1R-40- | EVQLVESGGGLVQPGGSLRLSCAASGFTFNYYPMGWFRQAPGKEREWVATISRGGGTYYAD |
| | 15 | NVKGRFTISADNSKNTAYLQMNSLKPEDTAVYYCSAALDGYSGIWGQGTLVTVSS |
| 2426 | GLP1R-40- | EVQLVESGGGLVQPGGSLRLSCAASGIIGSFRTMGWFRQAPGKEREFVGFITGSGGTTYYADS |
| | 16 | VKGRFTISADNSKNTAYLQMNSLKPEDTAVYYCAAARRYGNLYNTNNYDWGQGTLVTVSS |
| 2427 | GLP1R-40- | EVQLVESGGGLVQPGGSLRLSCAASGITFRFKAMGWFRQAPGKEREFVAAISWRGGSTNYAD |
| | 17 | SVKGRFTISADNSKNTAYLQMNSLKPEDTAVYYCAAATLGEPLVKYTWGQGTLVTVSS |
| 2428 | GLP1R-40- | EVQLVESGGGLVQPGGSLRLSCAASGSFFSINAMGWFRQAPGKEREFVAGISSKGGSSTYYA |
| | 18 | DSVKGRFTISADNSKNTAYLQMNSLKPEDTAVYYCAAHRIVVGGTSVGDWRWGQGTLVTV |
| | | SS |
| 2429 | GLP1R-40- | EVQLVESGGGLVQPGGSLRLSCAASGSRFSGRFNILNMGWFRQAPGKEREFVAAISRSGDTTY |
| | 19 | YADSVKGRFTISADNSKNTAYLQMNSLKPEDTAVYYCAASLRNSGSNVEGRWGQGTLVTVS |
| | | S |
| 2430 | GLP1R-40- | EVQLVESGGGLVQPGGSLRLSCAASGGTSNSYRMGWFRQAPGKEREFVAVISWTGGSTYYA |
| | 20 | DSVKGRFTISADNSKNTAYLQMNSLKPEDTAVYYCAVALDGYSGSWGQGTLVTVSS |
| 2431 | GLP1R-40- | EVQLVESGGGLVQPGGSLRLSCAASGFNIGTYTMGWFRQAPGKEREFVAAIGSNGLANYAD |
| | 21 | NVKGRFTISADNSKNTAYLQMNSLKPEDTAVYYCSAALDGYSGTWGQGTLVTVSS |
| 2432 | GLP1R-40- | EVQLVESGGGLVQPGGSLRLSCAASGRTFSVYAMGWFRQAPGKEREFVAGIHSDGSTLYADS |
| | 22 | VKGRFTISADNSKNTAYLQMNSLKPEDTAVYYCAAVLDGYMGTWGQGTLVTVSS |
| 2433 | GLP1R-40- | EVQLVESGGGLVQPGGSLRLSCAASGNIKSIDVMGWFRQAPGKERELVAAVRWSGGITWYA |
| | 23 | DSVKGRFTISADNSKNTAYLQMNSLKPEDTAVYYCAAVVYYGDWEGSEPVQHEYDWGQGT |
| | | LVTVSS |
| 2434 | GLP1R-40- | EVQLVESGGGLVQPGGSLRLSCAASGFTFSNYAMGWFRQAPGKEREFVAAIYCSDGSTQYA |
| | 24 | DSVKGRFTISADNSKNTAYLQMNSLKPEDTAVYYCAEALDGYWGQGTLVTVSS |
| 2435 | GLP1R-40- | EVQLVESGGGLVQPGGSLRLSCAASGYTFRAYAMGWFRQAPGKEREMVAAMRWSGGITWY |
| | 25 | ADSVKGRFTISADNSKNTAYLQMNSLKPEDTAVYYCAAQGSLYDDYDGLPIKYDWGQGTLV |
| | | TVSS |
| 2436 | GLP1R-40- | EVQLVESGGGLVQPGGSLRLSCAASGLTFSSYAMGWFRQAPGKERECVTAIFSDGGTYYADN |
| | 26 | VKGRFTISADNSKNTAYLQMNSLKPEDTAVYYCAAALDGYNGYWGQGTLVTVSS |
| 2437 | GLP1R-40- | EVQLVESGGGLVQPGGSLRLSCAASGIHFAISTMGWFRQAPGKEREIVTAINWSGARTYYAD |
| | 27 | SVKGRFTISADNSKNTAYLQMNSLKPEDTAVYYCAAKFVNTDSTWSRSEMYTWGQGTLVTV |
| | | SS |
| 2438 | GLP1R-40- | EVQLVESGGGLVQPGGSLRLSCAASGLTFTSYAMGWFRQAPGKEREGVAVIDSDGTTYYAD |
| | 28 | SVKGRFTISADNSKNTAYLQMNSLKPEDTAVYYCAAYLDGYSGSWGQGTLVTVSS |
| 2439 | GLP1R-40- | EVQLVESGGGLVQPGGSLRLSCAASGRTFSSLPMGWFRQAPGKERELVAIRWSGGSTVYADS |
| | 29 | VKGRFTISADNSKNTAYLQMNSLKPEDTAVYYCAAIAYEEGVYRWGQGTLVTVSS |
| 2440 | GLP1R-40- | EVQLVESGGGLVQPGGSLRLSCAASGRTFSSGVMGWFRQAPGKEREFVAAINRSGSTFYADS |
| | 30 | VKGRFTISADNSKNTAYLQMNSLKPEDTAVYYCAKTKRTGIFTTWGQGTLVTVSS |
| 2441 | GLP1R-40- | EVQLVESGGGLVQPGGSLRLSCAASGFTFSSYAMGWFRQAPGKERELVAAISSGGSTSYADS |
| | 31 | VKGRFTISADNSKNTAYLQMNSLKPEDTAVYYCAAAMDGYSGSWGQGTLVTVSS |
| 2442 | GLP1R-40- | EVQLVESGGGLVQPGGSLRLSCAASGFTDGIDAMGWFRQAPGKEREYVAAISGSGSITNYAD |
| | 32 | SVKGRFTISADNSKNTAYLQMNSLKPEDTAVYYCAANGIESYGWGNRHFNWGQGTLVTVSS |
| 2443 | GLP1R-40- | EVQLVESGGGLVQPGGSLRLSCAASGFTDGIDAMGWFRQAPGKEREFVAAIRWSGGITWYA |
| | 33 | DSVKGRFTISADNSKNTAYLQMNSLKPEDTAVYYCAAAIFDVTDYERADWGQGTLVTVSS |
| 2444 | GLP1R-40- | EVQLVESGGGLVQPGGSLRLSCAASGFAFSGYAMGWFRQAPGKEREFVAAISWSGGITWYA |
| | 34 | DSVKGRFTISADNSKNTAYLQMNSLKPEDTAVYYCAFVTTNSDYDLGRDWGQGTLVTVSS |
| 2445 | GLP1R-40- | EVQLVESGGGLVQPGGSLRLSCAASGIPASIRTMGWFRQAPGKEREGVSWISSSDGSIYYADS |
| | 35 | VKGRFTISADNSKNTAYLQMNSLKPEDTAVYYCVAALDGYSGSWGQGTLVTVSS |
| 2446 | GLP1R-40- | EVQLVESGGGLVQPGGSLRLSCAASGRTFSSLPMGWFRQAPGKERELVAIRWSGGSTVYADS |
| | 36 | VKGRFTISADNSKNTAYLQMNSLKPEDTAVYYCAAIAYEEGVYRWDWGQGTLVTVSS |
| 2447 | GLP1R-40- | EVQLVESGGGLVQPGGSLRLSCAASGFNSGSYTMGWFRQAPGKEREGVSWISTTDGSTYYA |
| | 37 | DSVKGRFTISADNSKNTAYLQMNSLKPEDTAVYYCAAALDGYSGIWGQGTLVTVSS |
| 2448 | GLP1R-40- | EVQLVESGGGLVQPGGSLRLSCAASGFTFSVYAMGWFRQAPGKEREFVTAIDSESRTLYADS |
| | 38 | VKGRFTISADNSKNTAYLQMNSLKPEDTAVYYCAALLDGYLGTWGQGTLVTVSS |
| 2449 | GLP1R-40- | EVQLVESGGGLVQPGGSLRLSCAASGSVFKINVMGWFRQAPGKEREFLGSILWSDDSTNYAD |
| | 39 | SVKGRFTISADNSKNTAYLQMNSLKPEDTAVYYCAANLKQGSYGYRFNDWGQGTLVTVSS |
| 2450 | GLP1R-40- | EVQLVESGGGLVQPGGSLRLSCAASGTIVNIHVMGWFRQAPGKERELVAAITSGGSTSYADN |
| | 40 | VKGRFTISADNSKNTAYLQMNSLKPEDTAVYYCAASAIGSGALRHFEYDWGQGTLVTVSS |
| 2451 | GLP1R-40- | EVQLVESGGGLVQPGGSLRLSCAASGRSLGTYHMGWFRQAPGKEREGVSWISSSDGSTYYA |
| | 41 | DSVKGRFTISADNSKNTAYLQMNSLKPEDTAVYYCAVVLDGYSGSWGQGTLVTVSS |
| 2452 | GLP1R-40- | EVQLVESGGGLVQPGGSLRLSCAASGFTFDDTGMGWFRQAPGKEREFVAAIRWSGKETWYA |
| | 42 | DSVKGRFTISADNSKNTAYLQMNSLKPEDTAVYYCAAEDPSMYYTLEEYEYDWGQGTLVTV |
| | | SS |
| 2453 | GLP1R-40- | EVQLVESGGGLVQPGGSLRLSCAASGFTFSSYVMGWFRQAPGKERECVAAISSSDGRTYYAD |
| | 43 | SVKGRFTISADNSKNTAYLQMNSLKPEDTAVYYCAAALDGYSGNWGQGTLVTVSS |
| 2454 | GLP1R-40- | EVQLVESGGGLVQPGGSLRLSCAASGSIFRVNVMGWFRQAPGKEREFIATIFSGGDTDYADSV |
| | 44 | KGRFTISADNSKNTAYLQMNSLKPEDTAVYYCAAIAHEEGVYRWDWGQGTLVTVSS |
| 2455 | GLP1R-40- | EVQLVESGGGLVQPGGSLRLSCAASGFTCGDYTMGWFRQAPGKEREIVASITSGGRKNYADS |
| | 45 | VKGRFTISADNSKNTAYLQMNSLKPEDTAVYYCAAALDDYSGSWGQGTLVTVSS |
| 2456 | GLP1R-40- | EVQLVESGGGLVQPGGSLRLSCAASGHSFGNFPMGWFRQAPGKEREVIAAIDWSGGSTFYAD |
| | 46 | SVKGRFTISADNSKNTAYLQMNSLKPEDTAVYYCAAAKGIGVYGWGQGTLVTVSS |
| 2457 | GLP1R-40- | EVQLVESGGGLVQPGGSLRLSCAASGSSFRFRAMGWFRQAPGKEREFVAAINRGGKISHYAD |
| | 47 | SVKGRFTISADNSKNTAYLQMNSLKPEDTAVYYCAAYIRPDTYLSRDYRKYDWGQGTLVTV |
| | | SS |
| 2458 | GLP1R-40- | EVQLVESGGGLVQPGGSLRLSCAASGFTWGDYTMGWFRQAPGKEREGVAAIDSDGRTRYA |
| | 48 | DSVKGRFTISADNSKNTAYLQMNSLKPEDTAVYYCAAALDGYSGSWGQGTLVTVSS |
| 2459 | GLP1R-40- | EVQLVESGGGLVQPGGSLRLSCAASGNILSLNTMGWFRQAPGKEREFVAGISWSGGSTYYAD |
| | 49 | SVKGRFTISADNSKNTAYLQMNSLKPEDTAVYYCAAIVTYSDYDLGNDWGQGTLVTVSS |
| 2460 | GLP1R-40- | EVQLVESGGGLVQPGGSLRLSCAASGITFRRYDMGWFRQAPGKEREGVAYISSSDGSTYYAD |
| | 50 | SVKGRFTISADNSKNTAYLQMNSLKPEDTAVYYCAAVLDDYSGGWGQGTLVTVSS |
| 2461 | GLP1R-40- | EVQLVESGGGLVQPGGSLRLSCAASGLTLSNYAMGWFRQAPGKEREFVAAISRSGSSTYYAD |
| | 51 | SVKGRFTISADNSKNTAYLQMNSLKPEDTAVYYCAAEMSGISGWDWGQGTLVTVSS |
| 2462 | GLP1R-40- | EVQLVESGGGLVQPGGSLRLSCAASGYTTSINTMGWFRQAPGKEREVVAAISRTGGSTYYAD |
| | 52 | SVKGRFTISADNSKNTAYLQMNSLKPEDTAVYYCAASAIGSGALRRFEYDWGQGTLVTVSS |
| 2463 | GLP1R-40- | EVQLVESGGGLVQPGGSLRLSCAASGRTFSIDAMGWFRQAPGKEREFVAAIKPDGSITYYADS |
| | 53 | VKGRFTISADNSKNTAYLQMNSLKPEDTAVYYCAASASDYGLGLELFHDEYNWGQGTLVTV |
| | | SS |
| 2464 | GLP1R-40- | EVQLVESGGGLVQPGGSLRLSCAASGSIFSLNAMGWFRQAPGKERELVAGISSKGGSTYYAD |
| | 54 | SVKGRFTISADNSKNTAYLQMNSLKPEDTAVYYCAAFRGIMRPDWGQGTLVTVSS |
| 2465 | GLP1R-40- | EVQLVESGGGLVQPGGSLRLSCAASGFTFSSYRMGWFRQAPGKEREAVAAIASMGGLTYYA |
| | 55 | DSVKGRFTISADNSKNTAYLQMNSLKPEDTAVYYCAAALDGYIGSWGQGTLVTVSS |
| 2466 | GLP1R-40- | EVQLVESGGGLVQPGGSLRLSCAASGFTFGAFTMGWFRQAPGKERERVAAITCSGSTTYADS |
| | 56 | VKGRFTISADNSKNTAYLQMNSLKPEDTAVYYCSAALDGYNGSWGQGTLVTVSS |
| 2467 | GLP1R-40- | EVQLVESGGGLVQPGGSLRLSCAASGIPSTIRAMGWFRQAPGKERESVGRIYWRDDNTYYAD |
| | 57 | SVKGRFTISADNSKNTAYLQMNSLKPEDTAVYYCAAVLDGYSGSWGQGTLVTVSS |
| 2468 | GLP1R-40- | EVQLVESGGGLVQPGGSLRLSCAASGFTDGIDAMGWFRQAPGKEREVVAGIAWGDGITYYA |
| | 58 | DSVKGRFTISADNSKNTAYLQMNSLKPEDTAVYYCAASYNVYYNNYYYPISRDEYDWGQGT |
| | | LVTVSS |
| 2469 | GLP1R-43- | EVQLVESGGGLVQAGGSLRLSCAASGRTIVPYTMGWFRQAPGKEREVVASISWSGKSTYYA |
| | 1 | DSVRGRFTISADNAKNTVYLQMNSLKPEDTAVYYCAAQRRWSQDWGQGTQVTVSS |
| 2470 | GLP1R-43- | EVQLVESGGGLVQAGGSLRLSCAASGRTFSSYAMGWFRQAPGKEREFVAAISWSGGSTYYA |
| | 2 | DSVKGRFTISADNAKNTVYLQMNSLKPEDTAVYYCAAVPTGRGERDYWGQGTQVTVSS |
| 2471 | GLP1R-43- | EVQLVESGGGLVQAGGSLRLSCAASGFTFSNYAMGWFRQAPGKEREFVATITWSGSSTYYA |
| | 3 | DSVKGRFTISADNAKNTVYLQMNSLKPEDTAVYYCAAVPRLYREYGYWGQGTQVTVSS |
| 2472 | GLP1R-43- | EVQLVESGGGLVQAGGSLRLSCAASGSIFHINPMGWFRQAPGKEREfVAAINIFGTTNYADSV |
| | 4 | KGRFTISADNAKNTVYLQMNSLKPEDTAVYYCAAVDGGPLWDDGYDWGQGTQVTVSS |
| 2473 | GLP1R-43- | EVQLVESGGGLVQAGGSLRLSCAASGSIFRINAMGWFRQAPGKEREGVASINIFGTTKYADSV |
| | 5 | KGRFTISADNAKNTVYLQMNSLKPEDTAVYYCSAVGWGPHNDDRYDWGQGTQVTVSS |
| 2474 | GLP1R-43- | EVQLVESGGGLVQAGGSLRLSCAASGTTFSIYAMEWFRQAPGKERELVATISRSGGTTYYAD |
| | 6 | SVGGRFTISADNAKNTVYLQMNSLKPEDTAVYYCAAASWYYRDDYWGQGTQVTVSS |
| 2475 | GLP1R-43- | EVQLVESGGGLVQAGGSLRLSCAASGSIFRINAMGWFRQAPGKEREGVAAINNFGTTKYADS |
| | 7 | VKGRFTISADNAKNTVYLQMNSLKPEDTAVYYCSAVRWGPHNDDRYDWGQGTQVTVSS |
| 2476 | GLP1R-43- | EVQLVESGGGLVQAGGSLRLSCAASGSIFRINAMGWFRQAPGKEREGVAAINNFGTTKYADS |
| | 8 | AKGRFTISADNAKNTVYLQMNSLKPEDTAVYYCAAVRWGPHNDDRYDWGQGTQVTVSS |
| 2477 | GLP1R-43- | EVQLVESGGGLVQAGGSLRLSCAASGFILYGYAMGWFRQAPGKEREGVSSISPSDASTYYAD |
| | 9 | SVKGRFTISADNAKNTVYLQMNSLKPEDTAVYYCAAVLNTYSDSWGQGTQVTVSS |
| 2478 | GLP1R-43- | EVQLVESGGGLVQAGGSLRLSCAASGRTFSSYAMGWFRQAPGKEREGVTAISTSDGSTYYAD |
| | 10 | SVKGRFTISADNAKNTVYLQMNSLKPEDTAVYYCAAARDGYSGSWGQGTQVTVSS |
| 2479 | GLP1R-43- | EVQLVESGGGLVQAGGSLRLSCAASGYTITNSYRMGWFRQAPGKEREFVAGITMSGFNTRY |
| | 11 | ADSVKGRFTISADNAKNTVYLQMNSLKPEDTAVYYCAANRGLAGPAWGQGTQVTVSS |
| 2480 | GLP1R-43- | EVQLVESGGGLVQAGGSLRLSCAASGFTFDDNAMGWFRQAPGKEREFVSGISTSGSTTYYAD |
| | 12 | SVKGRFTISADNAKNTVYLQMNSLKPEDTAVYYCAAAAGGYDYWGQGTQVTVSS |
| 2481 | GLP1R-43- | EVQLVESGGGLVQAGGSLRLSCAASGRTFSYYHMGWFRQAPGKEREGVSWISSYYSSTYYA |
| | 13 | DSESGRFTISADNAKNTVYLQMNSLKPEDTAVYYCAAVLDGYSCSWGQGTQVTVSS |
| 2482 | GLP1R-43- | EVQLVESGGGLVQAGGSLRLSCAASGSPFRLYTMGWFRQAPGKEREVVAHIYSYGSINYADS |
| | 14 | VKGRFTISADNAKNTVYLQMNSLKPEDTAVYYCAAALWGHSGDWGQGTQVTVSS |
| 2483 | GLP1R-43- | EVQLVESGGGLVQAGGSLRLSCAASGSTFDTYGMGWFRQAPGKEREFVASITWSGSSTYYA |
| | 15 | DSVKGRFTISADNAKNTVYLQMNSLKPEDTAVYYCAANRIHWSGFYYWGQGTQVTVSS |
| 2484 | GLP1R-43- | EVQLVESGGGLVQAGGSLRLSCAASGRTSSPYTMGWFRQAPGKEREFVSAISWSGGSTVYAD |
| | 16 | SVKGRFTISADNAKNTVYLQMNSLKPEDTAVYYCALIRRAPYSRLETWGQGTQVTVSS |
| 2485 | GLP1R-43- | EVQLVESGGGLVQAGGSLRLSCAASGSIFPINAMGWFRQAPGKEREGVAAITNFGTTKYADS |
| | 17 | VKGRFTISADNAKNTVYLQMNSLKPEDTAVYYCAAVRWGPRNDDHYDWGQGTQVTVSS |
| 2486 | GLP1R-43- | EVQLVESGGGLVQAGGSLRLSCAASGRTFDTYAMGWFRQAPGKEREFVAAITWGGGRTYY |
| | 18 | ADSVKGRFTISADNAKNTVYLQMNSLKPEDTAVYYCAAVPRLYRDYDYWGQGTQVTVSS |
| 2487 | GLP1R-43- | EVQLVESGGGLVQAGGSLRLSCAASGRRFSAYGMGWFRQAPGKEREFVAAVSWDGRNTYY |
| | 19 | ADSVKGRFTISADNAKNTVYLQMNSLKPEDTAVYYCASTDDYGVDWGQGTQVTVSS |
| 2488 | GLP1R-43- | EVQLVESGGGLVQAGGSLRLSCAASGSTFDNYAMGWFRQAPGKEREFVSAISGDGGTTYYA |
| | 20 | DSVKGRFTISADNAKNTVYLQMNSLKPEDTAVYYCAAVPRLYRNRDYWGQGTQVTVSS |
| 2489 | GLP1R-43- | EVQLVESGGGLVQAGGSLRLSCAASGSIFRINAMGWFRQAPGKEREGVSWITSFDASTYYAD |
| | 21 | SVRGRFTISADNAKNTVYLQMNSLKPEDTAVYYCAAALDGYSGSWGQGTQVTVSS |
| 2490 | GLP1R-43- | EVQLVESGGGLVQAGGSLRLSCAASGRTFSNYAMGWFRQAPGKEREFVSTISTGGSSTYYAD |
| | 22 | SVKGRFTISADNAKNTVYLQMNSLKPEDTAVYYCAAVPTGRGRRDYWGQGTQVTVSS |
| 2491 | GLP1R-43- | EVQLVESGGGLVQAGGSLRLSCAASGRTFSSYAMGWFRQAPGKEREFVAAISWSGGSTYYA |
| | 23 | DSVKGRFTISADNAKNTVYLQMNSLKPEDTAVYYCAAVPVVPNTKDYWGQGTQVTVSS |
| 2492 | GLP1R-43- | EVQLVESGGGLVQAGGSLRLSCAASGNVFMIKDMGWFRQAPGKEREWVTAISWNGGSTDY |
| | 24 | ADSVKGRFTISADNAKNTVYLQMNSLKPEDTAVYYCAAIVTYSDYDLGNDWGQGTQVTVSS |
| 2493 | GLP1R-43- | EVQLVESGGGLVQAGGSLRLSCAASGFPFSIWPMGWFRQAPGKEREFIATIFSGGDTDYADSV |
| | 25 | KGRFTISADNAKNTVYLQMNSLKPEDTAVYYCAAIAYEEGVYRWDWGQGTQVTVSS |
| 2494 | GLP1R-43- | EVQLVESGGGLVQAGGSLRLSCAASGRGFSRYAMGWFRQAPGKEREFVAAIRWSGKETWY |
| | 26 | ADSVKGRFTISADNAKNTVYLQMNSLKPEDTAVYYCALGPVRRSRLEWGQGTQVTVSS |
| 2495 | GLP1R-43- | EVQLVESGGGLVQAGGSLRLSCAASGRTSDIYGMGWFRQAPGKEREFVARIYWSSGNTYYA |
| | 27 | DSVKGRFTISADNAKNTVYLQMNSLKPEDTAVYYCAAAYRFSDYSRPAGYDWGQGTQVTV |
| | | SS |
| 2496 | GLP1R-43- | EVQLVESGGGLVQAGGSLRLSCAASGNDFSFNSMGWFRQAPGKEREFLASVSWGFGSTYYA |
| | 28 | DSVKGRFTISADNAKNTVYLQMNSLKPEDTAVYYCARAYGNPTWGQGTQVTVSS |
| 2497 | GLP1R-43- | EVQLVESGGGLVQAGGSLRLSCAASGRTFTDYPMGWFRQAPGKERELESFVPINGTSTYYAD |
| | 29 | SDSGRFTISADNAKNTVYLQMNSLKPEDTAVYYCAAALDGYSCSWGQGTQVTVSS |
| 2498 | GLP1R-43- | EVQLVESGGGLVQAGGSLRLSCAASGRTFSIYAMGWFRQAPGKEREFVATISRGGSTTYYAD |
| | 30 | SVKGRFTISADNAKNTVYLQMNSLKPEDTAVYYCAAGPRSGKDYWGQGTQVTVSS |
| 2499 | GLP1R-43- | EVQLVESGGGLVQAGGSLRLSCAASGFIFQLYVMGWFRQAPGKEREGVTYINNIDGSTYYAY |
| | 31 | SVRGRFTISADNAKNTVYLQMNSLKPEDTAVYYCAAVRDGYSGSWGQGTQVTVSS |
| 2500 | GLP1R-43- | EVQLVESGGGLVQAGGSLRLSCAASGSTFSSYAMEWFRQAPGKERELVATISRSGGRTYYAD |
| | 32 | SVKGRFTISADNAKNTVYLQMNSLKPEDTAVYYCAAANWYYRYDYWGQGTQVTVSS |
| 2501 | GLP1R-43- | EVQLVESGGGLVQAGGSLRLSCAASGFPFRINAMGWFRQAPGKERELVTAISSSGSSTYYADS |
| | 33 | VKGRFTISADNAKNTVYLQMNSLKPEDTAVYYCAASGYYATYYGERDYWGQGTQVTVSS |
| 2502 | GLP1R-43- | EVQLVESGGGLVQAGGSLRLSCAASGFTLSSYTMGWFRQAPGKEREFVSAISRGGGNTYYAD |
| | 34 | SVKGRFTISADNAKNTVYLQMNSLKPEDTAVYYCAAVPSYAEYDYWGQGTQVTVSS |
| 2503 | GLP1R-43- | EVQLVESGGGLVQAGGSLRLSCAASGRTFSIYGMGWFRQAPGKEREGVAAINGGGDSTNYA |
| | 35 | DSVKGRFTISADNAKNTVYLQMNSLKPEDTAVYYCAAASASPYSGRNYWGQGTQVTVSS |
| 2504 | GLP1R-43- | EVQLVESGGGLVQAGGSLRLSCAASGLtfSTTVMGWFRQAPGKEREGDGYISITDGSTYYADS |
| | 36 | VKGRFTISADNAKNTVYLQMNSLKPEDTAVYYCSAALDGYSGSWGQGTQVTVSS |
| 2505 | GLP1R-43- | EVQLVESGGGLVQAGGSLRLSCAASGRTLENYRMGWFRQAPGKEREFVAAVSWSSGNAYY |
| | 37 | ADSVKGRFTISADNAKNTVYLQMNSLKPEDTAVYYCAANWKMLLGVENDWGQGTQVTVS |
| | | S |
| 2506 | GLP1R-43- | EVQLVESGGGLVQAGGSLRLSCAASGRTFSSYAMGWFRQAPGKEREFVAAISWSGGSTYYA |
| | 38 | DSVKGRFTISADNAKNTVYLQMNSLKPEDTAVYYCAAVPTVYGERDYWGQGTQVTVSS |
| 2507 | GLP1R-43- | EVQLVESGGGLVQAGGSLRLSCAASGSILSISPMGWFRQAPGKERELVAINFSWGTTDYADSv |
| | 39 | KGRFTISADNAKNTVYLQMNSLKPEDTAVYYCAAIAYEQGVYRWDWGQGTQVTVSS |
| 2508 | GLP1R-43- | EVQLVESGGGLVQAGGSLRLSCAASGRTFSSYAMGWFRQAPGKEREFVAAISWSGGSTYYA |
| | 40 | DSVKGRFTISADNAKNTVYLQMNSLKPEDTAVYYCAAERYRYSGYYARDSWGQGTQVTVS |
| | | S |
| 2509 | GLP1R-43- | EVQLVESGGGLVQAGGSLRLSCAASGFTLSDYAMGWFRQAPGKEREFVSAISRDGTTTYYA |
| | 41 | DSVKGRFTISADNAKNTVYLQMNSLKPEDTAVYYCAAVPTSQYATDYWGQGTQVTVSS |
| 2510 | GLP1R-43- | EVQLVESGGGLVQAGGSLRLSCAASGRDLDYYVMGWFRQAPGKERELVAIKFSGGTTDYAD |
| | 42 | SVKGRFTISADNAKNTVYLQMNSLKPEDTAVYYCADIAYEEGVYRWDWGQGTQVTVSS |
| 2511 | GLP1R-43- | EVQLVESGGGLVQAGGSLRLSCAASGSIFTFNAMGWFRQAPGKEREFVAGITRSAVSTSYAD |
| | 43 | SVKGRFTISADNAKNTVYLQMNSLKPEDTAVYYCAAFRGIMRPDWGQGTQVTVSS |
| 2512 | GLP1R-43- | EVQLVESGGGLVQAGGSLRLSCAASGRTFDSYAMGWFRQAPGKEREFVAAITSSGGNTYYA |
| | 44 | DSVKGRFTISADNAKNTVYLQMNSLKPEDTAVYYCAAVPARYGARDYWGQGTQVTVSS |
| 2513 | GLP1R-43- | EVQLVESGGGLVQAGGSLRLSCAASGRTFNNDHMGWFRQAPGKEREFVAVIEIGGATNYAD |
| | 45 | SVKGRFTISADNAKNTVYLQMNSLKPEDTAVYYCATWDGRQVWGQGTQVTVSS |
| 2514 | GLP1R-43- | EVQLVESGGGLVQAGGSLRLSCAASGGTFRKLAMGWFRQAPGKERELVAAIRWSGGITWYA |
| | 46 | DSVKGRFTISADNAKNTVYLQMNSLKPEDTAVYYCAATLAKGGGRWGQGTQVTVSS |
| 2515 | GLP1R-43- | EVQLVESGGGLVQAGGSLRLSCAASGRTFSSYAMGWFRQAPGKEREFVAAISWSGGSTYYA |
| | 47 | DSVKGRFTISADNAKNTVYLQMNSLKPEDTAVYYCAAVPRAPSDRDYWGQGTQVTVSS |
| 2516 | GLP1R-43- | EVQLVESGGGLVQAGGSLRLSCAASGRTFRIYAMGWFRQAPGKERELVSSISWNSGSTYYAD |
| | 48 | SVKGRFTISADNAKNTVYLQMNSLKPEDTAVYYCAAAAYSYTQGTTYESWGQGTQVTVSS |
| 2517 | GLP1R-43- | EVQLVESGGGLVQAGGSLRLSCAASGRTFTSYRMGWFRQAPGKEREWMGTIDYSGRTYYA |
| | 49 | DSVKGRFTISADNAKNTVYLQMNSLKPEDTAVYYCAAAMDGYSGSWGQGTQVTVSS |
| 2518 | GLP1R-43- | EVQLVESGGGLVQAGGSLRLSCAASGRTFSIYAMGWFRQAPGKEREFVAAINWNGDTTYYA |
| | 50 | DSVKGRFTISADNAKNTVYLQMNSLKPEDTAVYYCAAVPRYSDYDYWGQGTQVTVSS |
| 2519 | GLP1R-43- | EVQLVESGGGLVQAGGSLRLSCAASGRFFSTRVMGWFRQAPGKERELVAIKFSGGTTDYADS |
| | 51 | VKGRFTISADNAKNTVYLQMNSLKPEDTAVYYCAAIAHEEGVYRWDWGQGTQVTVSS |
| 2520 | GLP1R-43- | EVQLVESGGGLVQAGGSLRLSCAASGRTFSSYAMGWFRQAPGKEREFVAAISWSGGSTYYA |
| | 52 | DSVKGRFTISADNAKNTVYLQMNSLKPEDTAVYYCAAVPSVYGTRDYWGQGTQVTVSS |
| 2521 | GLP1R-43- | EVQLVESGGGLVQAGGSLRLSCAASGSTFSIDVMGWFRQAPGKEREGVSYISMSDGRTYYAD |
| | 53 | SVKGRFTISADNAKNTVYLQMNSLKPEDTAVYYCAAELDGYSGSWGQGTQVTVSS |
| 2522 | GLP1R-43- | EVQLVESGGGLVQAGGSLRLSCAASGLSFSGYTMGWFRQAPGKEREVVAAISRTGGSTYYA |
| | 54 | DSVKGRFTISADNAKNTVYLQMNSLKPEDTAVYYCALIQRRAPYSRLETWGQGTQVTVSS |
| 2523 | GLP1R-43- | EVQLVESGGGLVQAGGSLRLSCAASGSTLSIYGMGWFRQAPGKEREGVAAISWSDGSTSYAD |
| | 55 | SVKGRFTISADNAKNTVYLQMNSLKPEDTAVYYCAAVADIGLASDFDYWGQGTQVTVSS |
| 2524 | GLP1R-43- | EVQLVESGGGLVQAGGSLRLSCAASGSTFSNYAMGWFRQAPGKEREFVATITRSSGNTYYAD |
| | 56 | SVKGRFTISADNAKNTVYLQMNSLKPEDTAVYYCAAVPFKPYSYDYWGQGTQVTVSS |
| 2525 | GLP1R-43- | EVQLVESGGGLVQAGGSLRLSCAASGSTFSIYTMGWFRQAPGKEREFVAAISGSSDSTYYADS |
| | 57 | VKGRFTISADNAKNTVYLQMNSLKPEDTAVYYCATVPKTRYTRDYWGQGTQVTVSS |
| 2526 | GLP1R-43- | EVQLVESGGGLVQAGGSLRLSCAASGNTFSSYAMGWFRQAPGKEREFVAIISRSGGRTYYAD |
| | 58 | SVKGRFTISADNAKNTVYLQMNSLKPEDTAVYYCAAAPYNETNSWGQGTQVTVSS |
| 2527 | GLP1R-43- | EVQLVESGGGLVQAGGSLRLSCAASGSTFSTYAMGWFRQAPGKEREFVASISRSGGRTYYAD |
| | 59 | SVKGRFTISADNAKNTVYLQMNSLKPEDTAVYYCAAARYNERNSWGQGTQVTVSS |
| 2528 | GLP1R-43- | EVQLVESGGGLVQAGGSLRLSCAASGGTLNNNPMAMGWFRQAPGKEREFVVAIYWSNGKT |
| | 60 | PYADSVKRRFTISADNAKNTVYLQMNSLKPEDTAVYYCAAALDGYSGAWGQGTQVTVSS |
| 2529 | GLP1R-43- | EVQLVESGGGLVQAGGSLRLSCAASGRTFSSYAMGWFRQAPGKEREFVAAISWSGGSTYYA |
| | 61 | DSVKGRFTISADNAKNTVYLQMNSLKPEDTAVYYCAAVPRAPSERDYWGQGTQVTVSS |
| 2530 | GLP1R-43- | EVQLVESGGGLVQAGGSLRLSCAASGRTFNNNDMGWFRQAPGKEREFVAVIKLGGATTYDD |
| | 62 | YSEGRFTISADNAKNTVYLQMNSLKPEDTAVYYCATWDARHVWGQGTQVTVSS |
| 2531 | GLP1R-43- | EVQLVESGGGLVQAGGSLRLSCAASGRAFSYYNMGWFRQAPGKEREGVSWISSSDGSTYYA |
| | 63 | DSVKGRFTISADNAKNTVYLQMNSLKPEDTAVYYCAAVLDGCSGSWGQGTQVTVSS |
| 2532 | GLP1R-43- | EVQLVESGGGLVQAGGSLRLSCAASGSTFSTYAMGWFRQAPGKEREFVAAINRSGASTYYA |
| | 64 | DSVKGRFTISADNAKNTVYLQMNSLKPEDTAVYYCAAALLGGRGGCGKGYWGQGTQVTVS |
| | | S |
| 2533 | GLP1R-43- | EVQLVESGGGLVQAGGSLRLSCAASGSILDTYAMGWFRQAPGKERELVSGINTSGDTTYYAD |
| | 65 | SVKGRFTISADNAKNTVYLQMNSLKPEDTAVYYCAAVLAGYEYWGQGTQVTVSS |
| 2534 | GLP1R-43- | EVQLVESGGGLVQAGGSLRLSCAASGSTLSINAMGWFRQAPGKEREFVAHMSHDGTTNYAD |
| | 66 | SVKGRFTISADNAKNTVYLQMNSLKPEDTAVYYCARLPNYRWGQGTQVTVSS |
| 2535 | GLP1R-43- | EVQLVESGGGLVQAGGSLRLSCAASGSIFRLNAMGWFRQAPGKEREGVAAINNFDTTKYAD |
| | 67 | SSKGRFTISADNAKNTVYLQMNSLKPEDTAVYYCAAVRWGPRSDDRWGQGTQVTVSS |
| 2536 | GLP1R-43- | EVQLVESGGGLVQAGGSLRLSCAASGLTNPPFDNFPMGWFRQAPGKEREFVAVISWTGGSTY |
| | 68 | YADSVKGRFTISADNAKNTVYLQMNSLKPEDTAVYYCPAVYPRYYGDDDRPPVDWGQGTQ |
| | | VTVSS |
| 2537 | GLP1R-43- | EVQLVESGGGLVQAGGSLRLSCAASGPTFSKAVMGWFRQAPGKEREFVAAMNWSGRSTYY |
| | 69 | ADSVKGRFTISADNAKNTVYLQMNSLKPEDTAVYYCAATPAGRGGYWGQGTQVTVSS |
| 2538 | GLP1R-43- | EVQLVESGGGLVQAGGSLRLSCAASGSIFSDYAMGWFRQAPGKEREFVATINWGGGRTYYA |
| | 70 | DSVKGRFTISADNAKNTVYLQMNSLKPEDTAVYYCAAVPKTRYARDYWGQGTQVTVSS |
| 2539 | GLP1R-43- | EVQLVESGGGLVQAGGSLRLSCAASGFILSDYAMGWFRQAPGKEREFVAAISSSEASTYYAD |
| | 71 | SVKGRFTISADNAKNTVYLQMNSLKPEDTAVYYCAAVRFWAGYDSWGQGTQVTVSS |
| 2540 | GLP1R-43- | EVQLVESGGGLVQAGGSLRLSCAASGYTDYKYDMGWFRQAPGKEREFVAAISWGGGLTVY |
| | 72 | ADSVKGRFTISADNAKNTVYLQMNSLKPEDTAVYYCAAVATVTDYTGTYSDGWGQGTQVT |
| | | VSS |
| 2541 | GLP1R-43- | EVQLVESGGGLVQAGGSLRLSCAASGRTFSNYAMGWFRQAPGKEREFVATINWGGGNTYY |
| | 73 | ADSVKGRFTISADNAKNTVYLQMNSLKPEDTAVYYCAAVPKTRYAYDYWGQGTQVTVSS |
| 2542 | GLP1R-43- | EVQLVESGGGLVQAGGSLRLSCAASGRTFSRYYMGWFRQAPGKERELVAVILRGGSTNYAD |
| | 74 | SVKGRFTISADNAKNTVYLQMNSLKPEDTAVYYCAAARRYGNLYNTNNYDWGQGTQVTVS |
| | | S |
| 2543 | GLP1R-43- | EVQLVESGGGLVQAGGSLRLSCAASGSILSSYVMGWFRQAPGKEREFVSAISRSGTSTYYADS |
| | 75 | VKGRFTISADNAKNTVYLQMNSLKPEDTAVYYCAAVPKTRYDRDYWGQGTQVTVSS |
| 2544 | GLP1R-43- | EVQLVESGGGLVQAGGSLRLSCAASGFTLDNYAMGWFRQAPGKEREFVAAISWSGGSTYYA |
| | 76 | DSVKGRFTISADNAKNTVYLQMNSLKPEDTAVYYCAAVPKTRYSYDYWGQGTQVTVSS |
| 2545 | GLP1R-43- | EVQLVESGGGLVQAGGSLRLSCAASGNTYSYKVMGWFRQAPGKEREFVGIIIRNGDTTYYAD |
| | 77 | SVKGRFTISADNAKNTVYLQMNSLKPEDTAVYYCAASPKYMTAYERSYDWGQGTQVTVSS |
| 2546 | GLP1R-43- | EVQLVESGGGLVQAGGSLRLSCAASGSIFRNYAMGWFRQAPGKEREFVATITTSGGNTYYAD |
| | 78 | SVKGRFTISADNAKNTVYLQMNSLKPEDTAVYYCAAVPKTRYRRDYWGQGTQVTVSS |
| 2547 | GLP1R-43- | EVQLVESGGGLVQAGGSLRLSCAASGFTFGTTTMGWFRQAPGKEREVVAAITGSGRSTYYA |
| | 79 | DSVKGRFTISADNAKNTVYLQMNSLKPEDTAVYYCAASAIGSGALRRFEYDWGQGTQVTVS |
| | | S |
| 2548 | GLP1R-43- | EVQLVESGGGLVQAGGSLRLSCAASGGTFSAYAMGWFRQAPGKEREGVAAIRWDGGYTRY |
| | 80 | ADSVKGRFTISADNAKNTVYLQMNSLKPEDTAVYYCAATTPTTSYLPRSERQYEWGQGTQV |
| | | TVSS |
| 2549 | GLP1R-43- | EVQLVESGGGLVQAGGSLRLSCAASGRTFSSYAMGWFRQAPGKEREFVAAISWSGGSTYYA |
| | 81 | DSVKGRFTISADNAKNTVYLQMNSLKPEDTAVYYCAAVPSVYGERDYWGQGTQVTVSS |
| 2550 | GLP1R-43- | EVQLVESGGGLVQAGGSLRLSCAASGSFFSINAMGWFRQAPGKEREFVAGISQSGGSTAYAD |
| | 82 | SVKGRFTISADNAKNTVYLQMNSLKPEDTAVYYCAAHRIVVGGTSVGDWRWGQGTQVTVS |
| | | S |
| 2551 | GLP1R-43- | EVQLVESGGGLVQAGGSLRLSCAASGRTFSSYRMGWFRQAPGKEREMVASITSRKIPKYADS |
| | 83 | VKGRFTISADNAKNTVYLQMNSLKPEDTAVYYCAVWSGRDWGQGTQVTVSS |
| 2552 | GLP1R-43- | EVQLVESGGGLVQAGGSLRLSCAASGFTFRRYVMGWFRQAPGKEREFVAAISRDGDRTYYA |
| | 84 | DSVKGRFTISADNAKNTVYLQMNSLKPEDTAVYYCASTRLAGRWYRDSEYKWGQGTQVTV |
| | | SS |
| 2553 | GLP1R-43- | EVQLVESGGGLVQAGGSLRLSCAASGRTFSDNAMGWFRQAPGKEREFVATISRGGSRTSYAD |
| | 85 | SVKGRFTISADNAKNTVYLQMNSLKPEDTAVYYCAAGPRSGRDYWGQGTQVTVSS |
| 2554 | GLP1R-43- | EVQLVESGGGLVQAGGSLRLSCAASGFTFRSYAMGWFRQAPGKEREFVATITRNGDNTYYA |
| | 86 | DSVKGRFTISADNAKNTVYLQMNSLKPEDTAVYYCATVGTRYNYWGQGTQVTVSS |
| 2555 | GLP1R-43- | EVQLVESGGGLVQAGGSLRLSCAASGSTFSDYVMGWFRQAPGKERELISGITWNGDTTYYA |
| | 87 | DSVKGRFTISADNAKNTVYLQMNSLKPEDTAVYYCAVVRLGGYDYWGQGTQVTVSS |
| 2556 | GLP1R-43- | EVQLVESGGGLVQAGGSLRLSCAASGGIISNYHMGWFRQAPGKEREFVATITRSGGSTYYAD |
| | 88 | SVKGRFTISADNAKNTVYLQMNSLKPEDTAVYYCAMAGRGRWGQGTQVTVSS |
| 2557 | GLP1R-43- | EVQLVESGGGLVQAGGSLRLSCAASGFSFDDDYVMGWFRQAPGKERELVSAIGWSGASTYY |
| | 89 | ADSVKGRFTISADNAKNTVYLQMNSLKPEDTAVYYCAAYYTDYDEALEETRGSYDWGQGT |
| | | QVTVSS |
| 2558 | GLP1R-43- | EVQLVESGGGLVQAGGSLRLSCAASGSTFPIYAMGWFRQAPGKEREWVSGISSRDDTTYYAD |
| | 90 | SVKGRFTISADNAKNTVYLQMNSLKPEDTAVYYCSAHRIVFRGTSVGDWRWGQGTQVTVSS |
| 2559 | GLP1R-43- | EVQLVESGGGLVQAGGSLRLSCAASGRAFSYYNMGWFRQAPGKEREGVSWISSSDGSTYYA |
| | 91 | DSVKGRFTISADNAKNTVYLQMNSLKPEDTAVYYCAAVLDGYSGSWGQGTQVTVSS |
| 2560 | GLP1R-43- | EVQLVESGGGLVQAGGSLRLSCAASGSTFSIDVMGWFRQAPGKERELVAATGRRGGPTYYA |
| | 92 | DSVKGRFTISADNAKNTVYLQMNSLKPEDTAVYYCAARTSYSGTYDYGVDWGQGTQVTVS |
| | | S |
| 2561 | GLP1R-43- | EVQLVESGGGLVQAGGSLRLSCAASGGTFSSYAMGWFRQAPGKEREFVAAINWSGSITYYA |
| | 93 | DSVKGRFTISADNAKNTVYLQMNSLKPEDTAVYYCAVGRSGRDYWGQGTQVTVSS |
| 2562 | GLP1R-43- | EVQLVESGGGLVQAGGSLRLSCAASGSIFRINAMGWFRQAPGKEREGVAAINNFGTTKYADS |
| | 94 | VKGRFTISADNAKNTVYLQMNSLKPEDTAVYYCAAVRWGPRNDDRYDWGQGTQVTVSS |
| 2563 | GLP1R-43- | EVQLVESGGGLVQAGGSLRLSCAASGGTLNNNPMAMGWFRQAPGKEREFVVAIYWSNGKT |
| | 95 | QYADSVKGRFTISADNAKNTVYLQMNSLKPEDTAVYYCAAALDGYSGSWGQGTQVTVSS |
| 2564 | GLP1R-43- | EVQLVESGGGLVQAGGSLRLSCAASGRTFNNDHMGWFRQAPGKEREFVAVIEIGGATNYAD |
| | 96 | SVKGRFTISADNAKNTVYLQMNSLKPEDTAVYYCASWDGRQVWGQGTQVTVSS |
| 2565 | GLP1R-41- | EVQLVESGGGLVQPGGSLRLSCAASGRTFAMGWMGWFRQAPGKEREFVARVSWDGRNAY |
| | 01 | YANSRFGRFTISADNSKNTAYLQMNSLKPEDTAVYYCPRYVSPARDHGCWGQGTLVTVSS |
| 2566 | GLP1R-41- | EVQLVESGGGLVQPGGSLRLSCAASGLTISTYIMGWFRQAPGKEREFVAVVNWNGDSTYYA |
| | 02 | DSVKGRFTISADNSKNTAYLQMNSLKPEDTAVYYCAAYYTDYDEALEETRGSYDWGQGTLV |
| | | TVSS |
| 2567 | GLP1R-41- | EVQLVESGGGLVQPGGSLRLSCAASGTLFKINAMGWFRQAPGKERELVAAINRGGKITHYAD |
| | 03 | SVKGRFTISADNSKNTAYLQMNSLKPEDTAVYYCAASLRNSGSNVEGRWGQGTLVTVSS |
| 2568 | GLP1R-41- | EVQLVESGGGLVQPGGSLRLSCAASGVTLDLYAMGWFRQAPGKEREFVAAISPSAVTTYYA |
| | 04 | DSVKGRFTISADNSKNTAYLQMNSLKPEDTAVYYCAAYDYYSDYPLPDANEYEWGQGTLVT |
| | | VSS |
| 2569 | GLP1R-41- | EVQLVESGGGLVQPGGSLRLSCAASGRTFSDYIMGWFRQAPGKEREFVAVINRSGSTTYYAD |
| | 05 | SVKGRFTISADNSKNTAYLQMNSLKPEDTAVYYCAAVQAYSNSSDYYSQEGAYDWGQGTL |
| | | VTVSS |
| 2570 | GLP1R-41- | EVQLVESGGGLVQPGGSLRLSCAASGFTFSNYVMGWFRQAPGKEREGVSYISSSDGRTHYAD |
| | 06 | SVKGRFTISADNSKNTAYLQMNSLKPEDTAVYYCAAVLDGYNGSWGQGTLVTVSS |
| 2571 | GLP1R-41- | EVQLVESGGGLVQPGGSLRLSCAASGFTFSRFGMGWFRQAPGKEREGVAAIGSDGSTSYADS |
| | 07 | VKGRFTISADNSKNTAYLQMNSLKPEDTAVYYCASGRDRYARDLSEYEYVWGQGTLVTVSS |
| 2572 | GLP1R-41- | EVQLVESGGGLVQPGGSLRLSCAASGFTFRFNAMGWFRQAPGKEREFVAAINWRGSHPYYA |
| | 08 | DSVKGRFTISADNSKNTAYLQMNSLKPEDTAVYYCAAATLGEPLVKYTWGQGTLVTVSS |
| 2573 | GLP1R-41- | EVQLVESGGGLVQPGGSLRLSCAASGGTFGVYHMGWFRQAPGKEREFLASVTWGFGSTYYA |
| | 09 | DSVKGRFTISADNSKNTAYLQMNSLKPEDTAVYYCAATTTRSYDDTYRNSWVYNWGQGTL |
| | | VTVSS |
| 2574 | GLP1R-41- | EVQLVESGGGLVQPGGSLRLSCAASGFSFDDYAMGWFRQAPGKERELVAAIRWSGGITWYA |
| | 10 | DSVKGRFTISADNSKNTAYLQMNSLKPEDTAVYYCAAYGSGSDYLPMDWGQGTLVTVSS |
| 2575 | GLP1R-41- | EVQLVESGGGLVQPGGSLRLSCAASGPTFTIYAMGWFRQAPGKEREFVGAISMSGEDTIYADS |
| | 11 | EKGRFTISADNSKNTAYLQMNSLKPEDTAVYYCAAVQAYTSNTNYYNQEGAYDWGQGTLV |
| | | TVSS |
| 2576 | GLP1R-41- | EVQLVESGGGLVQPGGSLRLSCAASGPTFSNYYVGWFRQAPGKEREFVAAILCSGGITCYAD |
| | 12 | SVKGRFTISADNSKNTAYLQMNSLKPEDTAVYYCAAALDGYIGTWGQGTLVTVSS |
| 2577 | GLP1R-41- | EVQLVESGGGLVQPGGSLRLSCAASGGTFSSIGMGWFRQAPGKEREGVAAIGSDGSTSYADS |
| | 13 | VKGRFTISADNSKNTAYLQMNSLKPEDTAVYYCAAASDRYARVLTEYEYVWGQGTLVTVSS |
| 2578 | GLP1R-41- | EVQLVESGGGLVQPGGSLRLSCAASGVTFNNYGMGWFRQAPGKERELVAAIRWSGSATFYA |
| | 14 | DSVKGRFTISADNSKNTAYLQMNSLKPEDTAVYYCAADDGARGSWGQGTLVTVSS |
| 2579 | GLP1R-41- | EVQLVESGGGLVQPGGSLRLSCAASGRTFTMDGMGWFRQAPGKEREGVAAIGSDGSTSYAD |
| | 15 | SVKGRFTISADNSKNTAYLQMNSLKPEDTAVYYCAAGSNIGGSRWRYDWGQGTLVTVSS |
| 2580 | GLP1R-41- | EVQLVESGGGLVQPGGSLRLSCAASGGIFRFNAMGWFRQAPGKERELVAAISPAALTTYYAD |
| | 16 | SVKGRFTISADNSKNTAYLQMNSLKPEDTAVYYCAAYLPSPYYSSYYDSTKYEWGQGTLVT |
| | | VSS |
| 2581 | GLP1R-41- | EVQLVESGGGLVQPGGSLRLSCAASGSGFSPNVMGWFRQAPGKEREVVAAISWNGGSTYYA |
| | 17 | DSVKGRFTISADNSKNTAYLQMNSLKPEDTAVYYCAASAIGSGALRRFEYDWGQGTLVTVSS |
| 2582 | GLP1R-41- | EVQLVESGGGLVQPGGSLRLSCAASGFTFGFYAMGWFRQAPGKERELVAAISWSDASTYYA |
| | 18 | DSVKGRFTISADNSKNTAYLQMNSLKPEDTAVYYCALDNRRSYVDYYNVSEYDWGQGTLV |
| | | TVSS |
| 2583 | GLP1R-41- | EVQLVESGGGLVQPGGSLRLSCAASGFTFSIYPMGWFRQAPGKERECVSTIWSRGDTYYADN |
| | 19 | VKGRFTISADNSKNTAYLQMNSLKPEDTAVYYCAAALDGYSATWGQGTLVTVSS |
| 2584 | GLP1R-41- | EVQLVESGGGLVQPGGSLRLSCAASGFTFDYYAMGWFRQAPGKERELVAAISWSNDITYYA |
| | 20 | DSVKGRFTISADNSKNTAYLQMNSLKPEDTAVYYCALDNRRSYVDYYSVSEYDWGQGTLVT |
| | | VSS |
| 2585 | GLP1R-41- | EVQLVESGGGLVQPGGSLRLSCAASGGTFSTYTMGWFRQAPGKEREFVAGIYNDGTASYYA |
| | 21 | DSVKGRFTISADNSKNTAYLQMNSLKPEDTAVYYCAAFDGYTGNDWGQGTLVTVSS |
| 2586 | GLP1R-41- | EVQLVESGGGLVQPGGSLRLSCAASGVTLDLYAMGWFRQAPGKEREWVARMYLDGDYPYY |
| | 22 | ADSVKGRFTISADNSKNTAYLQMNSLKPEDTAVYYCAAVLDGYSGSWGQGTLVTVSS |
| 2587 | GLP1R-41- | EVQLVESGGGLVQPGGSLRLSCAASGRTISRYIMGWFRQAPGKERELVAAINRSGKSTYYAD |
| | 23 | SVKGRFTISADNSKNTAYLQMNSLKPEDTAVYYCASTRFAGRWYRDSEYKWGQGTLVTVSS |
| 2588 | GLP1R-41- | EVQLVESGGGLVQPGGSLRLSCAASGRTLSVYAMGWFRQAPGKEREFVAAVRWSGGITWY |
| | 24 | VDSVKGRFTISADNSKNTAYLQMNSLKPEDTAVYYCAAFDGYSGSDWGQGTLVTVSS |
| 2589 | GLP1R-41- | EVQLVESGGGLVQPGGSLRLSCAASGSIFSITEMGWFRQAPGKERELVAAIAVGGGITWYADS |
| | 25 | VKGRFTISADNSKNTAYLQMNSLKPEDTAVYYCAAHDVDDDESPYYSGGYYRALYDWGQG |
| | | TLVTVSS |
| 2590 | GLP1R-41- | EVQLVESGGGLVQPGGSLRLSCAASGSIYSLDAMGWFRQAPGKERELVAAISPAALTTYYAD |
| | 26 | SVKGRFTISADNSKNTAYLQMNSLKPEDTAVYYCAASMSLRPLDPASYSPDIQPYDWGQGTL |
| | | VTVSS |
| 2591 | GLP1R-41- | EVQLVESGGGLVQPGGSLRLSCAASGFTCGDYTMGWFRQAPGKERESVAAIDSDGRTHYAD |
| | 27 | SVISRFTISADNSKNTAYLQMNSLKPEDTAVYYCAAALDGYSGDWGQGTLVTVSS |
| 2592 | GLP1R-41- | EVQLVESGGGLVQPGGSLRLSCAASGRTLSfYAMGWFRQAPGKEREFVAAINRGGRISHYAD |
| | 28 | SVKGRFTISADNSKNTAYLQMNSLKPEDTAVYYCAAGRRYGSPPHDGSSYEWGQGTLVTVS |
| | | S |
| 2593 | GLP1R-41- | EVQLVESGGGLVQPGGSLRLSCAASGFTFDDYAMGWFRQAPGKEREFVAGISWTGGITYYA |
| | 29 | DSVKGRFTISADNSKNTAYLQMNSLKPEDTAVYYCAVNVGFEWGQGTLVTVSS |
| 2594 | GLP1R-41- | EVQLVESGGGLVQPGGSLRLSCAASGFTFDDYGMGWFRQAPGKEREGVAAIGSDGSTSYAD |
| | 30 | SVKGRFTISADNSKNTAYLQMNSLKPEDTAVYYCAATLRATITNFDEYVWGQGTLVTVSS |
| 2595 | GLP1R-41- | EVQLVESGGGLVQPGGSLRLSCAASGRTFNRYPMGWFRQAPGKEREFVAHMSHDGTTNYA |
| | 31 | DSVKGRFTISADNSKNTAYLQMNSLKPEDTAVYYCAAPGTRYYGSNQVNYNWGQGTLVTV |
| | | SS |
| 2596 | GLP1R-41- | EVQLVESGGGLVQPGGSLRLSCAASGSIFSFNAMGWFRQAPGKEREFVAGITRRGLSTSYADS |
| | 32 | VKGRFTISADNSKNTAYLQMNSLKPEDTAVYYCAAAKGIGVYGWGQGTLVTVSS |
| 2597 | GLP1R-41- | EVQLVESGGGLVQPGGSLRLSCAASGGSISSINAMGWFRQAPGKERELVAGIITSGDSTYYAD |
| | 33 | SVKGRFTISADNSKNTAYLQMNSLKPEDTAVYYCAAGSAYVAGVRRRNAYHWGQGTLVTV |
| | | SS |
| 2598 | GLP1R-41- | EVQLVESGGGLVQPGGSLRLSCAASGGTFSADVMGWFRQAPGKEREFVAAISTGSITIYADSV |
| | 34 | KGRFTISADNSKNTAYLQMNSLKPEDTAVYYCATYGYDSGLYFITDSNDYEWGQGTLVTVSS |
| 2599 | GLP1R-41- | EVQLVESGGGLVQPGGSLRLSCAASGFTFDDAAMGWFRQAPGKEREFVAAMRWRGGITWY |
| | 35 | ADSVKGRFTISADNSKNTAYLQMNSLKPEDTAVYYCAAQGTLYDDYDGLPIKYDWGQGTLV |
| | | TVSS |
| 2600 | GLP1R-41- | EVQLVESGGGLVQPGGSLRLSCAASGDIFNINAMGWFRQAPGKEREPVAAISPAALTTYYAD |
| | 36 | SVKGRFTISADNSKNTAYLQMNSLKPEDTAVYYCAATPIERLGLDAYEYDWGQGTLVTVSS |
| 2601 | GLP1R-41- | EVQLVESGGGLVQPGGSLRLSCAASGRTFSTYNMGWFRQAPGKEREFVAAINWSGGITWYA |
| | 37 | DSVKGRFTISADNSKNTAYLQMNSLKPEDTAVYYCAAEPPDSSWYLDGSPEFFKWGQGTLV |
| | | TVSS |
| 2602 | GLP1R-41- | EVQLVESGGGLVQPGGSLRLSCAASGSISVFDAMGWFRQAPGKERELVAGISGSGGDTYYAD |
| | 38 | SVKGRFTISADNSKNTAYLQMNSLKPEDTAVYYCAASPKYSTHSIFDASPYNWGQGTLVTVS |
| | | S |
| 2603 | GLP1R-41- | EVQLVESGGGLVQPGGSLRLSCAASGFTSDDYAMGWFRQAPGKEREFVAALRWSSSNIDYT |
| | 39 | YYADSVKGRFTISADNSKNTAYLQMNSLKPEDTAVYYCATDLSGHGDVSEYEYDWGQGTL |
| | | VTVSS |
| 2604 | GLP1R-41- | EVQLVESGGGLVQPGGSLRLSCAASGFTFSPNVMGWFRQAPGKEREFVAAITSSGETTWYAD |
| | 40 | SVKGRFTISADNSKNTAYLQMNSLKPEDTAVYYCAAEPYGSGSSLMSEYDWGQGTLVTVSS |
| 2605 | GLP1R-41- | EVQLVESGGGLVQPGGSLRLSCAASGRNLRMYRMGWFRQAPGKEREFVAAINWSGDNTHY |
| | 41 | ADSVKGRFTISADNSKNTAYLQMNSLKPEDTAVYYCAANWKMLLGVENDWGQGTLVTVSS |
| 2606 | GLP1R-41- | EVQLVESGGGLVQPGGSLRLSCAASGDTFNCYAMGWFRQAPGKEREFVAVINWSGDNTHY |
| | 42 | ADSVKGRFTISADNSKNTAYLQMNSLKPEDTAVYYCAAYYTDYDEALEETRGRYDWGQGT |
| | | LVTVSS |
| 2607 | GLP1R-41- | EVQLVESGGGLVQPGGSLRLSCAASGSISTINVMGWFRQAPGKEREFVAAISPSAVTTYYADS |
| | 43 | VKGRFTISADNSKNTAYLQMNSLKPEDTAVYYCATDLSGRGDVSEYEYDWGQGTLVTVSS |
| 2608 | GLP1R-41- | EVQLVESGGGLVQPGGSLRLSCAASGRTLSKYRMGWFRQAPGKEREFVAAIRWSGGITWYA |
| | 44 | DSVKGRFTISADNSKNTAYLQMNSLKPEDTAVYYCAIPHGIAGRITWGQGTLVTVSS |
| 2609 | GLP1R-41- | EVQLVESGGGLVQPGGSLRLSCAASGFTFGSYAMGWFRQAPGKERELVAGIDQSGGITWYA |
| | 45 | DSVKGRFTISADNSKNTAYLQMNSLKPEDTAVYYCAADDYLGGDNWYLGPYDWGQGTLVT |
| | | VSS |
| 2610 | GLP1R-41- | EVQLVESGGGLVQPGGSLRLSCAASGFTIDDYAMGWFRQAPGKEREFVAAVSGTGTIAYYA |
| | 46 | DSVKGRFTISADNSKNTAYLQMNSLKPEDTAVYYCAAYYIDYDEALEETRGSYDWGQGTLV |
| | | TVSS |
| 2611 | GLP1R-41- | EVQLVESGGGLVQPGGSLRLSCAASGRTFNNYVMGWFRQAPGKERELVAGITSGRDITYYA |
| | 47 | DSVKGRFTISADNSKNTAYLQMNSLKPEDTAVYYCAADGVLATTLNWDWGQGTLVTVSS |
| 2612 | GLP1R-41- | EVQLVESGGGLVQPGGSLRLSCAASGSGISFNAMGWFRQAPGKERELVAAISRSGDTTYYAD |
| | 48 | SVKGRFTISADNSKNTAYLQMNSLKPEDTAVYYCAADLTTWADGPYRWGQGTLVTVSS |
| 2613 | GLP1R-41- | EVQLVESGGGLVQPGGSLRLSCAASGRTFSfYAMGWFRQAPGKEREFVAAINRGGKISHYAD |
| | 49 | SVKGRFTISADNSKNTAYLQMNSLKPEDTAVYYCAAVRRYGNPPHDGSSYEWGQGTLVTVS |
| | | S |
| 2614 | GLP1R-41- | EVQLVESGGGLVQPGGSLRLSCAASGRTFSfYGMGWFRQAPGKERELVAIKFSGGTTDYADS |
| | 50 | vkGRFTISADNSKNTAYLQMNSLKPEDTAVYYCAAIAHEEGVYRWGQGTLVTVSS |
| 2615 | GLP1R-41- | EVQLVESGGGLVQPGGSLRLSCAASGGIFRFNAMGWFRQAPGKERELVAGISGSGGDTYYAD |
| | 51 | SVKGRFTISADNSKNTAYLQMNSLKPEDTAVYYCAAFRGIMRPDWGQGTLVTVSS |
| 2616 | GLP1R-41- | EVQLVESGGGLVQPGGSLRLSCAASGRTFSfYAMGWFRQAPGKEREFVAAINRGGKISHYAD |
| | 52 | SVKGRFTISADNSKNTAYLQMNSLKPEDTAVYYCAAVRRYGSPPHDGSSYEWGQGTLVTVS |
| | | S |
| 2617 | GLP1R-41- | EVQLVESGGGLVQPGGSLRLSCAASGSDFSLNAMGWFRQAPGKEREFVAAISWSGGSTLYA |
| | 53 | DSVKGRFTISADNSKNTAYLQMNSLKPEDTAVYYCASNESDAYNWGQGTLVTVSS |
| 2618 | GLP1R-41- | EVQLVESGGGLVQPGGSLRLSCAASGRTLVNYDMGWFRQAPGKEREFVAAIRWSGGITWYA |
| | 54 | DSVKGRFTISADNSKNTAYLQMNSLKPEDTAVYYCAAFRGIMLPPWGQGTLVTVSS |
| 2619 | GLP1R-41- | EVQLVESGGGLVQPGGSLRLSCAASGRTFEKDAMGWFRQAPGKEREMVAAIRWSGGITCYA |
| | 55 | DSVKGRFTISADNSKNTAYLQMNSLKPEDTAVYYCAAYGSLPDDYDGLECEYDWGQGTLVT |
| | | VSS |
| 2620 | GLP1R-41- | EVQLVESGGGLVQPGGSLRLSCAASGSFFKINAMGWFRQAPGKEREFVAGITRSGGSTYYAD |
| | 56 | SVKGRFTISADNSKNTAYLQMNSLKPEDTAVYYCAAESLGRWWGQGTLVTVSS |
| 2621 | GLP1R-41- | EVQLVESGGGLVQPGGSLRLSCAASGRTFSIDAMGWFRQAPGKEREFVAAIRWSGGITWYAD |
| | 57 | SVKGRFTISADNSKNTAYLQMNSLKPEDTAVYYCAASHDSDWGQGTLVTVSS |
| 2622 | GLP1R-41- | EVQLVESGGGLVQPGGSLRLSCAASGRTFSIDAMGWFRQAPGKEREFVAAIRWSGGITWYAD |
| | 58 | SVKGRFTISADNSKNTAYLQMNSLKPEDTAVYYCAASHDSDYGGTNANLYDWGQGTLVTV |
| | | SS |
| 2623 | GLP1R-41- | EVQLVESGGGLVQPGGSLRLSCAASGRTDRSNVMGWFRQAPGKEREFVAAINRSGSTFYADS |
| | 59 | VKGRFTISADNSKNTAYLQMNSLKPEDTAVYYCAKTKRTGIFTTARMVDWGQGTLVTVSS |
| 2624 | GLP1R-41- | EVQLVESGGGLVQPGGSLRLSCAASGSFFSINVMGWFRQAPGKERELVAATGRRGGPTYYA |
| | 60 | DSVKGRFTISADNSKNTAYLQMNSLKPEDTAVYYCAAHRIVVGGTSVGDWRWGQGTLVTV |
| | | SS |
| 2625 | GLP1R-41- | EVQLVESGGGLVQPGGSLRLSCAASGFTWGDYTMGWFRQAPGKEREGVAAIDSDGRTRYA |
| | 61 | DSVKGRFTISADNSKNTAYLQMNSLKPEDTAVYYCAAALDGYSGNWGQGTLVTVSS |
| 2626 | GLP1R-41- | EVQLVESGGGLVQPGGSLRLSCAASGNIFSLNTMGWFRQAPGKEREFVAAINCSGNHPYYAD |
| | 62 | SVKGRFTISADNSKNTAYLQMNSLKPEDTAVYYCAAIVTYSDDDGRDNWGQGTLVTVSS |
| 2627 | GLP1R-41- | EVQLVESGGGLVQPGGSLRLSCAASGSIFSINAMGWFRQAPGKEREFVAAVSGSGDDTYYAD |
| | 63 | SVKGRFTISADNSKNTAYLQMNSLKPEDTAVYYCAAVQAYSSSSDYYSQEGAYDWGQGTLV |
| | | TVSS |
| 2628 | GLP1R-41- | EVQLVESGGGLVQPGGSLRLSCAASGFTFPAYVMGWFRQAPGKERELLAVITRDGSTHYADS |
| | 64 | VKGRFTISADNSKNTAYLQMNSLKPEDTAVYYCAVNGRWRIWSSRNPWGQGTLVTVSS |
| 2629 | GLP1R-41- | EVQLVESGGGLVQPGGSLRLSCAASGFSFDDDYVMGWFRQAPGKERELVAVIGWGGKETW |
| | 65 | YADSVKGRFTISADNSKNTAYLQMNSLKPEDTAVYYCAAEDPSMGYYTLEEYEYDWGQGT |
| | | LVTVSS |
| 2630 | GLP1R-41- | EVQLVESGGGLVQPGGSLRLSCAASGPTFDTYVMGWFRQAPGKEREFVAAISMSGDDTAYA |
| | 66 | DSVKGRFTISADNSKNTAYLQMNSLKPEDTAVYYCATDLRGRGDVSEYEYDWGQGTLVTVS |
| | | S |
| 2631 | GLP1R-41- | EVQLVESGGGLVQPGGSLRLSCAASGRTFSIDAMGWFRQAPGKEREFVGAITWGGGNTYYA |
| | 67 | DSVKGRFTISADNSKNTAYLQMNSLKPEDTAVYYCAAIVTDGDYDGWGQGTLVTVSS |
| 2632 | GLP1R-41- | EVQLVESGGGLVQPGGSLRLSCAASGNTFSINVMGWFRQAPGKEREFVAAINWNGGSTDYA |
| | 68 | DSVKGRFTISADNSKNTAYLQMNSLKPEDTAVYYCAAIVTYSDYDLDNDWGQGTLVTVSS |
| 2633 | GLP1R-41- | EVQLVESGGGLVQPGGSLRLSCAASGFTFSTHWMGWFRQAPGKEREVVAVIYTSDGSTYYA |
| | 69 | DSVKGRFTISADNSKNTAYLQMNSLKPEDTAVYYCAANEYGLGSSIYAYKWGQGTLVTVSS |
| 2634 | GLP1R-41- | EVQLVESGGGLVQPGGSLRLSCAASGRTFSISAMGWFRQAPGKEREFVAAISRSGGTTYYAD |
| | 70 | SVKGRFTISADNSKNTAYLQMNSLKPEDTAVYYCATDEDYALGPNEYDWGQGTLVTVSS |
| 2635 | GLP1R-41- | EVQLVESGGGLVQPGGSLRLSCAASGSTFRINAMGWFRQAPGKERELVAAISPAALTTYYAD |
| | 71 | SVKGRFTISADNSKNTAYLQMNSLKPEDTAVYYCAAEPYGSGSLYDDYDGLPIKYDWGQGT |
| | | LVTVSS |
| 2636 | GLP1R-41- | EVQLVESGGGLVQPGGSLRLSCAASGFTDGIDAMGWFRQAPGKEREFVAAISWSNDITYYAD |
| | 72 | SVKGRFTISADNSKNTAYLQMNSLKPEDTAVYYCAAALSEVWRGSENLREGYDWGQGTLVT |
| | | VSS |
| 2637 | GLP1R-41- | EVQLVESGGGLVQPGGSLRLSCAASGLPVDYYAMGWFRQAPGKERELVAAISGSGDSTYYA |
| | 73 | DSVKGRFTISADNSKNTAYLQMNSLKPEDTAVYYCAAQTEDSASIFGYGMDWGQGTLVTVS |
| | | S |
| 2638 | GLP1R-41- | EVQLVESGGGLVQPGGSLRLSCAASGRTLSTVNMGWFRQAPGKEREFVGAISRSGETTWYA |
| | 74 | DSVKGRFTISADNSKNTAYLQMNSLKPEDTAVYYCAVDCPDYYSDYECPLEWGQGTLVTVS |
| | | S |
| 2639 | GLP1R-41- | EVQLVESGGGLVQPGGSLRLSCAASGFSFDDYAMGWFRQAPGKERELVAAVRWSGGITWY |
| | 75 | ADSVKGRFTISADNSKNTAYLQMNSLKPEDTAVYYCAAGDTGGAAYGWGQGTLVTVSS |
| 2640 | GLP1R-41- | EVQLVESGGGLVQPGGSLRLSCAASGSTLSINAMGWFRQAPGKEREGVSWISSSDGSTYYAD |
| | 76 | SVKGRFTISADNSKNTAYLQMNSLKPEDTAVYYCAAALDGYSGRWGQGTLVTVSS |
| 2641 | GLP1R-41- | EVQLVESGGGLVQPGGSLRLSCAASGSSVSIDAMGWFRQAPGKEREFVAGISRSGDTTYYAD |
| | 77 | SVKGRFTISADNSKNTAYLQMNSLKPEDTAVYYCAASYNVYYNNYYYPISRDEYDWGQGTL |
| | | VTVSS |
| 2642 | GLP1R-41- | EVQLVESGGGLVQPGGSLRLSCAASGSIFRVNVMGWFRQAPGKERELVAVTWSGGSTNYAD |
| | 78 | SVKGRFTISADNSKNTAYLQMNSLKPEDTAVYYCAAIAYEEGVYRWDWGQGTLVTVSS |
| 2643 | GLP1R-41- | EVQLVESGGGLVQPGGSLRLSCAASGRTFSfYAMGWFRQAPGKEREFVAVVNWSGRRTYYA |
| | 79 | DSVKGRFTISADNSKNTAYLQMNSLKPEDTAVYYCAASSRMGVDDPETYGWGQGTLVTVSS |
| 2644 | GLP1R-41- | EVQLVESGGGLVQPGGSLRLSCAASGFTFDDAAMGWFRQAPGKEREFVAAVRWRGGITWY |
| | 80 | ADSVKGRFTISADNSKNTAYLQMNSLKPEDTAVYYCAAQGSLYDDYDGLPIKYDWGQGTLV |
| | | TVSS |
| 2645 | GLP1R-41- | EVQLVESGGGLVQPGGSLRLSCAASGSIFRINAMGWFRQAPGKERELVASISRFGRTNYADSV |
| | 81 | KGRFTISADNSKNTAYLQMNSLKPEDTAVYYCAANGIESWGQGTLVTVSS |
| 2646 | GLP1R-41- | EVQLVESGGGLVQPGGSLRLSCAASGFTWGDYTMGWFRQAPGKEREFVASITSGGRMWYA |
| | 82 | DSVKGRFTISADNSKNTAYLQMNSLKPEDTAVYYCAAALDGYSGSWGQGTLVTVSS |
| 2647 | GLP1R-41- | EVQLVESGGGLVQPGGSLRLSCAASGFRFSSYGMGWFRQAPGKEREGVAAIGSDGSTSYADS |
| | 83 | VKGRFTISADNSKNTAYLQMNSLKPEDTAVYYCASWDGRQVWGQGTLVTVSS |
| 2648 | GLP1R-41- | EVQLVESGGGLVQPGGSLRLSCAASGRTFDNYNMGWFRQAPGKEREFVAAISWNGVTIYYA |
| | 84 | DSVKGRFTISADNSKNTAYLQMNSLKPEDTAVYYCAAQGSLYDDWGQGTLVTVSS |
| 2649 | GLP1R-41- | EVQLVESGGGLVQPGGSLRLSCAASGFTFSTYSMGWFRQAPGKEREFVAAISSGGLKAYADS |
| | 85 | VKGRFTISADNSKNTAYLQMNSLKPEDTAVYYCAAALDDYSGSWGQGTLVTVSS |
| 2650 | GLP1R-41- | EVQLVESGGGLVQPGGSLRLSCAASGYTFRAYVMGWFRQAPGKERELLAVITRDGSTHYAD |
| | 86 | SVKGRFTISADNSKNTAYLQMNSLKPEDTAVYYCAVNGRWRSWSSRNPWGQGTLVTVSS |
| 2651 | GLP1R-41- | EVQLVESGGGLVQPGGSLRLSCAASGRTFSIYAMGWFRQAPGKEREFVAAISRGSNSTDYAD |
| | 87 | SVKGRFTISADNSKNTAYLQMNSLKPEDTAVYYCAAIVTYTDYDLWGQGTLVTVSS |
| 2652 | GLP1R-41- | EVQLVESGGGLVQPGGSLRLSCAASGRTISSYAMGWFRQAPGKERELVAAISKSSISTYYADS |
| | 88 | VKGRFTISADNSKNTAYLQMNSLKPEDTAVYYCALGPVRRSRLEWGQGTLVTVSS |
| 2653 | GLP1R-41- | EVQLVESGGGLVQPGGSLRLSCAASGPTFDTYVMGWFRQAPGKEREFVAAISWTGDSSSDG |
| | 89 | DTYYADSVKGRFTISADNSKNTAYLQMNSLKPEDTAVYYCAAAIFDVTDYERADWGQGTLV |
| | | TVSS |
| 2654 | GLP1R-41- | EVQLVESGGGLVQPGGSLRLSCAASGFTLGNYAMGWFRQAPGKERELVSAITWSDGSSYYA |
| | 90 | DSVKGRFTISADNSKNTAYLQMNSLKPEDTAVYYCASTRFAGRWGQGTLVTVSS |
| 2655 | GLP1R-41- | EVQLVESGGGLVQPGGSLRLSCAASGNIDRLYAMGWFRQAPGKEREPVAAISPAAVTAGMT |
| | 91 | YYADSVKGRFTISADNSKNTAYLQMNSLKPEDTAVYYCAAYGSGSYYYTDDELDWGQGTL |
| | | VTVSS |
| 2656 | GLP1R-41- | EVQLVESGGGLVQPGGSLRLSCAASGRTFGRRAMGWFRQAPGKERELVAAIRWSGKETWY |
| | 92 | ADSVKGRFTISADNSKNTAYLQMNSLKPEDTAVYYCAAGNGGRTYGHSRARYEWGQGTLV |
| | | TVSS |
| 2657 | GLP1R-41- | EVQLVESGGGLVQPGGSLRLSCAASGRTFSIGAMGWFRQAPGKEREYVGSITWRGGNTYYA |
| | 93 | DSVKGRFTISADNSKNTAYLQMNSLKPEDTAVYYCAAGVTGGAAYGWGQGTLVTVSS |
| 2658 | GLP1R-41- | EVQLVESGGGLVQPGGSLRLSCAASGLTFSTYWMGWFRQAPGKEREVVAVIYTSDGSTYYA |
| | 94 | DSVKGRFTISADNSKNTAYLQMNSLKPEDTAVYYCATIDGSWREWGQGTLVTVSS |
| 2659 | GLP1R-41- | EVQLVESGGGLVQPGGSLRLSCAASGFGIDlyAMGWFRQAPGKEREFVAAISGSGDDTYYAD |
| | 95 | SVKGRFTISADNSKNTAYLQMNSLKPEDTAVYYCAASASDYGLGLELFHDEYNWGQGTLVT |
| | | VSS |
| 2660 | GLP1R-41- | EVQLVESGGGLVQPGGSLRLSCAASGNILSLNTMGWFRQAPGKEREFVASVTWGFGSTSYAD |
| | 96 | SVKGRFTISADNSKNTAYLQMNSLKPEDTAVYYCAAIVTYSDYDLGNDWGQGTLVTVSS |
| 2661 | GLP1R-41- | EVQLVESGGGLVQPGGSLRLSCAASGSIYSLDAMGWFRQAPGKEREFVAAISPAALTTYYAD |
| | 97 | SVKGRFTISADNSKNTAYLQMNSLKPEDTAVYYCAGSSRIYIYSDSLSERSYDWGQGTLVTVS |
| | | S |
| 2662 | GLP1R-41- | EVQLVESGGGLVQPGGSLRLSCAASGRTFSfYGMGWFRQAPGKERELVAIKFSGGTTDYADS |
| | 98 | VKGRFTISADNSKNTAYLQMNSLKPEDTAVYYCAAIAHEEGVYRWDWGQGTLVTVSS |
| 2663 | GLP1R-41- | EVQLVESGGGLVQPGGSLRLSCAASGRTFSKYAMGWFRQAPGKEREFVAAIRWSGGTTFYA |
| | 99 | DSVKGRFTISADNSKNTAYLQMNSLKPEDTAVYYCAAGGWGTGRYNWGQGTLVTVSS |
| 2664 | GLP1R-44- | EVQLVESGGGLVQPGGSLRLSCAASGSIFSIYAMDWFRQAPGKEREFVAAISSDDSTTYYADS |
| | 01 | VKGRFTISADNSKNTAYLQMNSLKPEDTAVYYCTAVLPAYDDWGQGTLVTVSS |
| 2665 | GLP1R-44- | EVQLVESGGGLVQPGGSLRLSCAASGFNSGSYTMGWFRQAPGKEREGVSYISSSDGRTYYAD |
| | 02 | SVKGRFTISADNSKNTAYLQMNSLKPEDTAVYYCAAGLNGAAAAWGQGTLVTVSS |
| 2666 | GLP1R-44- | EVQLVESGGGLVQPGGSLRLSCAASGRTFSNGPMGWFRQAPGKEREFVAHISTGGATNYADS |
| | 03 | VKGRFTISADNSKNTAYLQMNSLKPEDTAVYYCASWDGRQGWGQGTLVTVSS |
| 2667 | GLP1R-44- | EVQLVESGGGLVQPGGSLRLSCAASGRALSSYSMGWFRQAPGKEREFVALITRSGGTTFYAD |
| | 04 | SVKGRFTISADNSKNTAYLQMNSLKPEDTAVYYCALDNRHSYVDWGQGTLVTVSS |
| 2668 | GLP1R-44- | EVQLVESGGGLVQPGGSLRLSCAASGSIGSINAMGWFRQAPGKEREFVAAISWSGGATNYAD |
| | 05 | SVKGRFTISADNSKNTAYLQMNSLKPEDTAVYYCAASVAYSDYDLGNDWGQGTLVTVSS |
| 2669 | GLP1R-44- | EVQLVESGGGLVQPGGSLRLSCAASGLSFDDYAMGWFRQAPGKEREFVAAISGRSGNTYYA |
| | 06 | DSVKGRFTISADNSKNTAYLQMNSLKPEDTAVYYCALIQRRAPYSRLETWGQGTLVTVSS |
| 2670 | GLP1R-44- | EVQLVESGGGLVQPGGSLRLSCAASGFTFSIYAMGWFRQAPGKEREGVAAISWSGGTTYYAD |
| | 07 | SVKGRFTISADNSKNTAYLQMNSLKPEDTAVYYCAAAAGWVAEYGYWGQGTLVTVSS |
| 2671 | GLP1R-44- | EVQLVESGGGLVQPGGSLRLSCAASGGTFSSYAMGWFRQAPGKEREFVATISSNGNTTYYAD |
| | 08 | SVKGRFTISADNSKNTAYLQMNSLKPEDTAVYYCAADLRVLRLRRYEYNYWGQGTLVTVSS |
| 2672 | GLP1R-44- | EVQLVESGGGLVQPGGSLRLSCAASGFTFRSNAMGWFRQAPGKEREGVAAISTSGGITYYAD |
| | 09 | SVKGRFTISADNSKNTAYLQMNSLKPEDTAVYYCAAERDGYGYWGQGTLVTVSS |
| 2673 | GLP1R-44- | EVQLVESGGGLVQPGGSLRLSCAASGFTFDDYAMGWFRQAPGKERELVAGISWNGGITYYA |
| | 10 | DSVKGRFTISADNSKNTAYLQMNSLKPEDTAVYYCAVVRAGYDYWGQGTLVTVSS |
| 2674 | GLP1R-44- | EVQLVESGGGLVQPGGSLRLSCAASGSTFSIYAMGWFRQAPGKEREWVATISWSGGSTNYA |
| | 11 | DSVKGRFTISADNSKNTAYLQMNSLKPEDTAVYYCAVGRSGRDYWGQGTLVTVSS |
| 2675 | GLP1R-44- | EVQLVESGGGLVQPGGSLRLSCAASGRAFESYAMGWFRQAPGKEREFVAAIRWSGGSTYYA |
| | 12 | DSVKGRFTISADNSKNTAYLQMNSLKPEDTAVYYCATGGWGTGRYNWGQGTLVTVSS |
| 2676 | GLP1R-44- | EVQLVESGGGLVQPGGSLRLSCAASGRIFSDYAMGWFRQAPGKEREFVATINGDGDSTNYAD |
| | 13 | SVKGRFTISADNSKNTAYLQMNSLKPEDTAVYYCAANTYWYYTYDSWGQGTLVTVSS |
| 2677 | GLP1R-44- | EVQLVESGGGLVQPGGSLRLSCAASGRIFSDYAMGWFRQAPGKEREFVATINGDGDSTNYAD |
| | 14 | SVKGRFTISADNSKNTAYLQMNSLKPEDTAVYYCAANTYCNYTYDSWGQGTLVTVSS |
| 2678 | GLP1R-44- | EVQLVESGGGLVQPGGSLRLSCAASGRTLSRSNMGWFRQAPGKEREFVAAVRWSGGITWYA |
| | 15 | DSVKGRFTISADNSKNTAYLQMNSLKPEDTAVYYCALGPVRRSRLEWGQGTLVTVSS |
| 2679 | GLP1R-44- | EVQLVESGGGLVQPGGSLRLSCAASGFTFSTYAMGWFRQAPGKEREFVAAITWSGGSTNYA |
| | 16 | DSVKGRFTISADNSKNTAYLQMNSLKPEDTAVYYCAAGRAGRDSWGQGTLVTVSS |
| 2680 | GLP1R-44- | EVQLVESGGGLVQPGGSLRLSCAASGRTFNSYAMGWFRQAPGKEREFVAGITRSAVSTSYAD |
| | 17 | SVKGRFTISADNSKNTAYLQMNSLKPEDTAVYYCAAFRGIMRPDWGQGTLVTVSS |
| 2681 | GLP1R-44- | EVQLVESGGGLVQPGGSLRLSCAASGFTFRNYVMGWFRQAPGKEREFVASITWSGGTTYYA |
| | 18 | DSVKGRFTISADNSKNTAYLQMNSLKPEDTAVYYCAAGRGSGRDYWGQGTLVTVSS |
| 2682 | GLP1R-44- | EVQLVESGGGLVQPGGSLRLSCAASGRALSSNSMGWFRQAPGKEREFVALITRSGGTTFYAD |
| | 19 | SVKGRFTISADNSKNTAYLQMNSLKPEDTAVYYCALNNRRRYVDWGQGTLVTVSS |
| 2683 | GLP1R-44- | EVQLVESGGGLVQPGGSLRLSCAASGRTFSSYAMGWFRQAPGKEREFVAAISWSGGSTYYA |
| | 20 | DSVKGRFTISADNSKNTAYLQMNSLKPEDTAVYYCAVGRNGRDYWGQGTLVTVSS |
| 2684 | GLP1R-44- | EVQLVESGGGLVQPGGSLRLSCAASGSTFSIYAMGWFRQAPGKEREFVAAISWSGGNTYYAD |
| | 21 | SVKGRFTISADNSKNTAYLQMNSLKPEDTAVYYCAAVPTIAYNTGYDYWGQGTLVTVSS |
| 2685 | GLP1R-44- | EVQLVESGGGLVQPGGSLRLSCAASGRTFDDYAMGWFRQAPGKERELVSGITWSGGSTYYA |
| | 22 | DSVKGRFTISADNSKNTAYLQMNSLKPEDTAVYYCAAVLGYDGYDYWGQGTLVTVSS |
| 2686 | GLP1R-44- | EVQLVESGGGLVQPGGSLRLSCAASGRTFSIYAMGWFRQAPGKERELVSAISTDDGSTYYAD |
| | 23 | SVKGRFTISADNSKNTAYLQMNSLKPEDTAVYYCAAALPDDTYLATTYDYWGQGTLVTVSS |
| 2687 | GLP1R-44- | EVQLVESGGGLVQPGGSLRLSCAASGSIFSDNVMGWFRQAPGKEREMVAAIRWSGGITWYA |
| | 24 | DSVKGRFTISADNSKNTAYLQMNSLKPEDTAVYYCATDLSGRGDVSEYEYDWGQGTLVTVS |
| | | S |
| 2688 | GLP1R-44- | EVQLVESGGGLVQPGGSLRLSCAASGEIASIIAMGWFRQAPGKEREWVSAINSGGDTYYADS |
| | 25 | VKGRFTISADNSKNTAYLQMNSLKPEDTAVYYCAADRSRTIWPDWGQGTLVTVSS |
| 2689 | GLP1R-44- | EVQLVESGGGLVQPGGSLRLSCAASGRTFSVSTMGWFRQAPGKEREIVAAITWSGSATYYAD |
| | 26 | SVKGRFTISADNSKNTAYLQMNSLKPEDTAVYYCAAQRRWSQDWGQGTLVTVSS |
| 2690 | GLP1R-44- | EVQLVESGGGLVQPGGSLRLSCAASGRTFSSYAMGWFRQAPGKERELVAGITGGGSSTYYAD |
| | 27 | SVKGRFTISADNSKNTAYLQMNSLKPEDTAVYYCAAVTRYGYDYWGQGTLVTVSS |
| 2691 | GLP1R-44- | EVQLVESGGGLVQPGGSLRLSCAASGIPFRSRTMGWFRQAPGKEREFVAGITRNSIRTRYADS |
| | 28 | VKGRFTISADNSKNTAYLQMNSLKPEDTAVYYCAAAPRRPYLPIRIRDYIWGQGTLVTVSS |
| 2692 | GLP1R-44- | EVQLVESGGGLVQPGGSLRLSCAASGRTIVPYTMGWFRQAPGKEREFVAAISWSGASTIYAD |
| | 29 | SVKGRFTISADNSKNTAYLQMNSLKPEDTAVYYCAIGGTLYDRRRFEWGQGTLVTVSS |
| 2693 | GLP1R-44- | EVQLVESGGGLVQPGGSLRLSCAASGFTFSNNAMGWFRQAPGKEREGVAAINGSGSITYYAD |
| | 30 | SVKGRFTISADNSKNTAYLQMNSLKPEDTAVYYCAAARDDYGYWGQGTLVTVSS |
| 2694 | GLP1R-44- | EVQLVESGGGLVQPGGSLRLSCAASGRTFSIYGMGWFRQAPGKEREGVAGISWSDGSTSYAD |
| | 31 | SVKGRFTISADNSKNTAYLQMNSLKPEDTAVYYCAAASDASFDYWGQGTLVTVSS |
| 2695 | GLP1R-44- | EVQLVESGGGLVQPGGSLRLSCAASGGTFSDYGMGWFRQAPGKEREGVASISWNDGSTSYA |
| | 32 | DSVKGRFTISADNSKNTAYLQMNSLKPEDTAVYYCAAATADYDYWGQGTLVTVSS |
| 2696 | GLP1R-44- | EVQLVESGGGLVQPGGSLRLSCAASGSTFSTYAMGWFRQAPGKERELVAAISWSSGTTYYAD |
| | 33 | SVKGRFTISADNSKNTAYLQMNSLKPEDTAVYYCAAVLVTSDGVSEYNYWGQGTLVTVSS |
| 2697 | GLP1R-44- | EVQLVESGGGLVQPGGSLRLSCAASGFLFDSYAMGWFRQAPGKEREPVAAISPAALTTYYAD |
| | 34 | SVKGRFTISADNSKNTAYLQMNSLKPEDTAVYYCAAYYTDYDEALEETRGSYDWGQGTLVT |
| | | VSS |
| 2698 | GLP1R-44- | EVQLVESGGGLVQPGGSLRLSCAASGFTLSNYAMGWFRQAPGKEREGVAAISWNSGSTYYA |
| | 35 | DSVKGRFTISADNSKNTAYLQMNSLKPEDTAVYYCATDARRYGYWGQGTLVTVSS |
| 2699 | GLP1R-44- | EVQLVESGGGLVQPGGSLRLSCAASGSTFGNYAMGWFRQAPGKEREFVAAISRSGSITYYAD |
| | 36 | SVKGRFTISADNSKNTAYLQMNSLKPEDTAVYYCATDEDYALGPNEYDWGQGTLVTVSS |
| 2700 | GLP1R-44- | EVQLVESGGGLVQPGGSLRLSCAASGRTFSIYAMGWFRQAPGKERELVAGISWGGDSTYYA |
| | 37 | DSVKGRFTISADNSKNTAYLQMNSLKPEDTAVYYCAAVAGNGYDYWGQGTLVTVSS |
| 2701 | GLP1R-44- | EVQLVESGGGLVQPGGSLRLSCAASGFNSGSYTMGWFRQAPGKEREGVSYISSSDGRTYYAD |
| | 38 | SVKGRFTISADNSKNTAYLQMNSLKPEDTAVYYCAAALDGYSGSWGQGTLVTVSS |
| 2702 | GLP1R-44- | EVQLVESGGGLVQPGGSLRLSCAASGLTFWTSGMGWFRQAPGKEREYVAAISRSGSLKGYA |
| | 39 | DSVKGRFTISADNSKNTAYLQMNSLKPEDTAVYYCATVATALIWGQGTLVTVSS |
| 2703 | GLP1R-44- | EVQLVESGGGLVQPGGSLRLSCAASGFTFSINAMGWFRQAPGKERELVSGISWGGGSTYYAD |
| | 40 | SVKGRFTISADNSKNTAYLQMNSLKPEDTAVYYCAAVNEDGFDYWGQGTLVTVSS |
| 2704 | GLP1R-44- | EVQLVESGGGLVQPGGSLRLSCAASGFTFDDNAMGWFRQAPGKERELVAAISTSGSNTYYA |
| | 41 | DSVKGRFTISADNSKNTAYLQMNSLKPEDTAVYYCAAELREYGYWGQGTLVTVSS |
| 2705 | GLP1R-44- | EVQLVESGGGLVQPGGSLRLSCAASGRTFTSYNMGWFRQAPGKEREFLGSILWSDDSTNYAD |
| | 42 | SVKGRFTISADNSKNTAYLQMNSLKPEDTAVYYCASWDGRQVWGQGTLVTVSS |
| 2706 | GLP1R-44- | EVQLVESGGGLVQPGGSLRLSCAASGFTFRNYVMGWFRQAPGKEREFVAAINWNGSITYYA |
| | 43 | DSVKGRFTISADNSKNTAYLQMNSLKPEDTAVYYCAAGRSARNYWGQGTLVTVSS |
| 2707 | GLP1R-44- | EVQLVESGGGLVQPGGSLRLSCAASGRTFSSYAMGWFRQAPGKEREFVAAISTSGGITYYAD |
| | 44 | SVKGRFTISADNSKNTAYLQMNSLKPEDTAVYYCATDRIEYSRGGYDYWGQGTLVTVSS |
| 2708 | GLP1R-44- | EVQLVESGGGLVQPGGSLRLSCAASGSTFRKYAMGWFRQAPGKEREFVAAISSGGGSTNYA |
| | 45 | DSVKGRFTISADNSKNTAYLQMNSLKPEDTAVYYCAAGRYRERDSWGQGTLVTVSS |
| 2709 | GLP1R-44- | EVQLVESGGGLVQPGGSLRLSCAASGSTFSIYAMGWFRQAPGKEREFVAAISWSGDTTYYAD |
| | 46 | SVKGRFTISADNSKNTAYLQMNSLKPEDTAVYYCAIDLPDDTYLATEYDYWGQGTLVTVSS |
| 2710 | GLP1R-44- | EVQLVESGGGLVQPGGSLRLSCAASGSGFSPNVMGWFRQAPGKERELVAIKFSGGTTDYADS |
| | 47 | VKGRFTISADNSKNTAYLQMNSLKPEDTAVYYCAAIAYEEGVYRWDWGQGTLVTVSS |
| 2711 | GLP1R-44- | EVQLVESGGGLVQPGGSLRLSCAASGRTLTNHDMGWFRQAPGKEREGVSYISMSDGRTYYA |
| | 48 | DSVKGRFTISADNSKNTAYLQMNSLKPEDTAVYYCAAVLDGYSGSWGQGTLVTVSS |
| 2712 | GLP1R-44- | EVQLVESGGGLVQPGGSLRLSCAASGSTFSIYAMGWFRQAPGKEREFVAAISRSGDSTYYAD |
| | 49 | SVKGRFTISADNSKNTAYLQMNSLKPEDTAVYYCAAVTLDNYGYWGQGTLVTVSS |
| 2713 | GLP1R-44- | EVQLVESGGGLVQPGGSLRLSCAASGGTASSYHMGWFRQAPGKEREFVAFIHRSGTSTYYAD |
| | 50 | SVKGRFTISADNSKNTAYLQMNSLKPEDTAVYYCAADSITDRRSVAVAHTSYYWGQGTLVT |
| | | VSS |
| 2714 | GLP1R-44- | EVQLVESGGGLVQPGGSLRLSCAASGLTFSTYAMGWFRQAPGKEREIVAAITWSGGITYYAD |
| | 51 | SVKGRFTISADNSKNTAYLQMNSLKPEDTAVYYCAAHGSILLDRIEWGQGTLVTVSS |
| 2715 | GLP1R-44- | EVQLVESGGGLVQPGGSLRLSCAASGGTFSIYAMGWFRQAPGKERELVAAISSSGSITYYADS |
| | 52 | VKGRFTISADNSKNTAYLQMNSLKPEDTAVYYCAAAAALDGPGDMYDYWGQGTLVTVSS |
| 2716 | GLP1R-44- | EVQLVESGGGLVQPGGSLRLSCAASGGTFDNYAMGWFRQAPGKERELVSGINSDGGSTYYA |
| | 53 | DSVKGRFTISADNSKNTAYLQMNSLKPEDTAVYYCAAVPISSPSDRNYWGQGTLVTVSS |
| 2717 | GLP1R-44- | EVQLVESGGGLVQPGGSLRLSCAASGRTFSLTAMGWFRQAPGKEREFVAAISPAALTTYYAD |
| | 54 | SVKGRFTISADNSKNTAYLQMNSLKPEDTAVYYCASRRAFRLSSDYEWGQGTLVTVSS |
| 2718 | GLP1R-44- | EVQLVESGGGLVQPGGSLRLSCAASGRNLRMYRMGWFRQAPGKEREFVAAVNWNGDSTYY |
| | 55 | ADSVKGRFTISADNSKNTAYLQMNSLKPEDTAVYYCAANWKMLLGVENDWGQGTLVTVSS |
| 2719 | GLP1R-44- | EVQLVESGGGLVQPGGSLRLSCAASGFTFDIYAMGWFRQAPGKERELVAGISSSGGSTYYAD |
| | 56 | SVKGRFTISADNSKNTAYLQMNSLKPEDTAVYYCAAVLGTYDYWGQGTLVTVSS |
| 2720 | GLP1R-44- | EVQLVESGGGLVQPGGSLRLSCAASGRTFDIYAMGWFRQAPGKERELVAAINRDDSSTYYAD |
| | 57 | SVKGRFTISADNSKNTAYLQMNSLKPEDTAVYYCAAVAGLGNYNYWGQGTLVTVSS |
| 2721 | GLP1R-44- | EVQLVESGGGLVQPGGSLRLSCAASGRSFSFNAMGWFRQAPGKERELVAAITKLGFRNYADS |
| | 58 | VKGRFTISADNSKNTAYLQMNSLKPEDTAVYYCAASIEGVSGRWGQGTLVTVSS |
| 2722 | GLP1R-44- | EVQLVESGGGLVQPGGSLRLSCAASGSFFSINAMGWFRQAPGKERELVSASTWNGGYTYYA |
| | 59 | DSVKGRFTISADNSKNTAYLQMNSLKPEDTAVYYCAAHRIVVGGTSVGDWRWGQGTLVTV |
| | | SS |
| 2723 | GLP1R-44- | EVQLVESGGGLVQPGGSLRLSCAASGRTFSDYAMGWFRQAPGKEREFVAGITSSGGYTYYA |
| | 60 | DSVKGRFTISADNSKNTAYLQMNSLKPEDTAVYYCAAVVYYGDWEGSEPVQHEYDWGQGT |
| | | LVTVSS |
| 2724 | GLP1R-44- | EVQLVESGGGLVQPGGSLRLSCAASGSIFSRNAMGWFRQAPGKEREFVAAIRWSGKETWYA |
| | 61 | DSVKGRFTISADNSKNTAYLQMNSLKPEDTAVYYCAKTKRTGIFTTARMVDWGQGTLVTVS |
| | | S |
| 2725 | GLP1R-44- | EVQLVESGGGLVQPGGSLRLSCAASGGTFDTYAMGWFRQAPGKEREFVAGISGDGTITYYAD |
| | 62 | SVKGRFTISADNSKNTAYLQMNSLKPEDTAVYYCATDNPYWSGYNYWGQGTLVTVSS |
| 2726 | GLP1R-44- | EVQLVESGGGLVQPGGSLRLSCAASGGTFSNYAMGWFRQAPGKERELVSGINSDGGSTYYA |
| | 63 | DSVKGRFTISADNSKNTAYLQMNSLKPEDTAVYYCAAVSTNDGYDYWGQGTLVTVSS |
| 2727 | GLP1R-44- | EVQLVESGGGLVQPGGSLRLSCAASGGIYRVNTMGWFRQAPGKERELVAIKFSGGTTDYADS |
| | 64 | VKGRFTISADNSKNTAYLQMNSLKPEDTAVYYCAAIAHEEGVYRWDWGQGTLVTVSS |
| 2728 | GLP1R-44- | EVQLVESGGGLVQPGGSLRLSCAASGFTFSTYAMGWFRQAPGKERELVAGISSSGSSTYYAD |
| | 65 | SVKGRFTISADNSKNTAYLQMNSLKPEDTAVYYCAVVSDGGYDYWGQGTLVTVSS |
| 2729 | GLP1R-44- | EVQLVESGGGLVQPGGSLRLSCAASGRTSSIYNMGWFRQAPGKEREFVAAISRSGRSTSYADS |
| | 66 | VKGRFTISADNSKNTAYLQMNSLKPEDTAVYYCAAIVTYSDYDLGNDWGQGTLVTVSS |
| 2730 | GLP1R-44- | EVQLVESGGGLVQPGGSLRLSCAASGRALSSYSMGWFRQAPGKEREFVALITRSGGTTFYAD |
| | 67 | SVKGRFTISADNSKNTAYLQMNSLKPEDTAVYYCALDNRRSYVDWGQGTLVTVSS |
| 2731 | GLP1R-44- | EVQLVESGGGLVQPGGSLRLSCAASGRALSRYGMVWFRQAPGKEREFVAAINRGGKISHYA |
| | 68 | DSVKGRFTISADNSKNTAYLQMNSLKPEDTAVYYCAAGNGGRNYGHSRARYEWGQGTLVT |
| | | VSS |
| 2732 | GLP1R-44- | EVQLVESGGGLVQPGGSLRLSCAASGFKFNDSYMRWFRQAPGKEREFVVAINWSSGSTYYA |
| | 69 | DSVKGRFTISADNSKNTAYLQMNSLKPEDTAVYYCAVVNGPIFWGQGTLVTVSS |
| 2733 | GLP1R-44- | EVQLVESGGGLVQPGGSLRLSCAASGRTLSDYALGWFRQAPGKERELVSGINTSGDTTYYAD |
| | 70 | SVKGRFTISADNSKNTAYLQMNSLKPEDTAVYYCAVVTSSYDYWGQGTLVTVSS |
| 2734 | GLP1R-44- | EVQLVESGGGLVQPGGSLRLSCAASGSTFDIYGMGWFRQAPGKEREGVAAITGDGSSTSYAD |
| | 71 | SVKGRFTISADNSKNTAYLQMNSLKPEDTAVYYCAADNDTEYGYWGQGTLVTVSS |
| 2735 | GLP1R-44- | EVQLVESGGGLVQPGGSLRLSCAASGGTLDIYAMGWFRQAPGKEREFVAAISWSGSTTYYA |
| | 72 | DSVKGRFTISADNSKNTAYLQMNSLKPEDTAVYYCAAVLGYDRDYWGQGTLVTVSS |
| 2736 | GLP1R-44- | EVQLVESGGGLVQPGGSLRLSCAASGRPYSYDAMGWFRQAPGKEREIVAAISRTGSSIYYAD |
| | 73 | SVKGRFTISADNSKNTAYLQMNSLKPEDTAVYYCAAQGSLYDDYDGLPIKYDWGQGTLVTV |
| | | SS |
| 2737 | GLP1R-44- | EVQLVESGGGLVQPGGSLRLSCAASGRTFRTYGMGWFRQAPGKEREGVAAISWSGNSTSYA |
| | 74 | DSVKGRFTISADNSKNTAYLQMNSLKPEDTAVYYCAARLSKRGNRSSRDYWGQGTLVTVSS |
| 2738 | GLP1R-44- | EVQLVESGGGLVQPGGSLRLSCAASGSTFDNYAMGWFRQAPGKERELVAGINWSDSSTYYA |
| | 75 | DSVKGRFTISADNSKNTAYLQMNSLKPEDTAVYYCAAVAGWGEYDYWGQGTLVTVSS |
| 2739 | GLP1R-44- | EVQLVESGGGLVQPGGSLRLSCAASGSTFSIYAMGWFRQAPGKERELVAGINWSDSSTYYAD |
| | 76 | SVKGRFTISADNSKNTAYLQMNSLKPEDTAVYYCAAVTDYDEYNYWGQGTLVTVSS |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-55. (canceled)

56. An antibody or antibody fragment that binds GLP1R, wherein the antibody or antibody fragment comprises a heavy chain comprising a heavy chain variable region (VH) and a light chain comprising a light chain variable region (VL), wherein:
(a) the heavy chain comprises the amino acid sequence of residues 20 to 488 of SEQ ID NO: 2303 and the light chain comprises the amino acid sequence of residues 21 to 203 of SEQ ID NO: 2310;
(b) the heavy chain comprises the amino acid sequence of residues 20 to 499 of SEQ ID NO: 2304 and the light chain comprises the amino acid sequence of residues 21 to 236 of SEQ ID NO: 2311; or
(c) the heavy chain comprises the amino acid sequence of residues 20 to 496 of SEQ ID NO: 2308 and the light chain comprises the amino acid sequence of residues 21 to 235 of SEQ ID NO: 2315.

57. The antibody or antibody fragment of claim 56, wherein the heavy chain comprises the amino acid sequence of residues 20 to 488 of SEQ ID NO: 2303 and the light chain comprises the amino acid sequence of residues 21 to 203 of SEQ ID NO: 2310.

58. The antibody or antibody fragment of claim 56, wherein the heavy chain comprises the amino acid sequence of residues 20 to 499 of SEQ ID NO: 2304 and the light chain comprises the amino acid sequence of residues 21 to 236 of SEQ ID NO: 2311.

59. The antibody or antibody fragment of claim 56, wherein the heavy chain comprises the amino acid sequence of residues 20 to 496 of SEQ ID NO: 2308 and the light chain comprises the amino acid sequence of residues 21 to 235 of SEQ ID NO: 2315.

60. The antibody or antibody fragment of claim 56, wherein the antibody is a monoclonal antibody, a polyclonal antibody, a bi-specific antibody, a multispecific antibody, a grafted antibody, a humanized antibody, a synthetic antibody, a chimeric antibody, or an antigen-binding fragments thereof.

61. The antibody or antibody fragment of claim 56, wherein the antibody or antibody fragment is chimeric or humanized.

62. The antibody or antibody fragment of claim 56, wherein the antibody has an EC50 less than about 25 nM, 20 nM, or 10 nM in a cAMP assay.

63. The antibody or antibody fragment of claim 56, wherein the antibody is an antagonist of GLP1R.

64. A pharmaceutical composition comprising the antibody or antibody fragment of claim 56.

65. A method of treating a metabolic disorder comprising administering the antibody or antibody fragment of claim 56.

66. An isolated nucleic acid comprising a sequence that encodes the antibody or antibody fragment of claim 56.

67. An expression vector comprising the nucleic acid of claim 66.

68. An isolated cell comprising the nucleic acid of claim 66 or the expression vector of claim 67.
